(19) United States
(12) Patent Application Publication
(10) Pub. No.: US 2021/0024631 A1
(43) Pub. Date: Jan. 28, 2021

(54) BI-FUNCTIONAL PROTEINS AND CONSTRUCTION THEREOF

(71) Applicants: Orionis Biosciences, Inc., Waltham, MA (US); Orionis Biosciences BV, Ghent (BE)

(72) Inventors: Nikolai KLEY, Waltham, MA (US); Erik DEPLA, Ghent (BE); Lennart ZABEAU, Ghent (BE); Jan TAVERNIER, Balegem (BE)

(21) Appl. No.: 17/042,512

(22) PCT Filed: Mar. 28, 2019
(86) PCT No.: PCT/US19/24714

§ 371 Date: Sep. 28, 2020

Related U.S. Application Data
(60) Provisional application No. 62/649,238, filed on Mar. 28, 2018.
(60) Provisional application No. 62/649,264, filed on Mar. 28, 2018.
(60) Provisional application No. 62/649,248, filed on Mar. 28, 2018.

(51) Int. Cl. C07K 16/28; C07K 14/56; C07K 14/545; C07K 14/565; C07K 14/55

(52) U.S. Cl. CPC C07K 16/2815; C07K 14/56; C07K 14/545; C07K 16/2851; C07K 14/565; C07K 16/2818; A61K 38/00; C07K 16/2863; C07K 16/2896; C07K 14/55; C07K 2317/52; C07K 2317/622; C07K 16/2827

(57) ABSTRACT

The present invention relates, in part, to Fc-based chimeric protein complexes and their use as therapeutic agents. The present invention further relates to pharmaceutical compositions comprising the Fc-based chimeric protein complexes and their use in the treatment of various diseases.

### CROSS-REFERENCE TO RELATED APPLICATIONS

This application claims the benefit of U.S. Provisional Patent Application Nos. 62/649,238, filed Mar. 28, 2018; 62/649,248, filed Mar. 28, 2018; and 62/649,264, filed Mar. 28, 2018; the entire contents of all of which are hereby incorporated by reference in their entirety.

### FIELD

The present invention relates, in part, to fragment crystallizable region (Fc)-based chimeric protein complexes and their use as therapeutic agents.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ORN-042PC_Sequence_listing, date recorded: Mar. 27, 2019; file size: 1,521,030 bytes).

### BACKGROUND

Effector function-encoding biologics represent a class of biologics with many potential therapeutic applications. In order for such agents to be useful for the treatment of disease, maximizing their tolerability and therapeutic index is of critical importance, in particular when encoding potent effector functions (e.g., cytokines, many of which are systemically toxic if administered to humans as such). Thus, there is a need for engineering such agents with high inherent safety profile, which requires targeted delivery of an effector function to select target site(s) (e.g. antigen on a cell type of interest) with high precision, and in a regulated fashion.

An example of such agents, is a chimeric protein having a signaling agent (e.g., cytokine), connected to a targeting element, in which the signaling agent is wild type or modified (e.g. by mutation) to cause an attenuation of the signaling agent's activity (e.g., substantially reducing its ability to interact with/engage its receptor) in a manner such that its effector function can be recovered upon binding of the targeting element to its target (e.g., antigen on target cell).

However, such chimeric proteins are amenable to therapeutic use only if certain conditions are met, e.g., the ability to be produced in a large scale, an in vivo half-life that ensures adequate time of exposure to the drug to elicit a therapeutically beneficial effect, a proper size to avoid rapid clearance or limited tissue penetrance and bio-distribution, and other properties that ensure adequate solubility, stability and storage without significant loss of function. Importantly all, or substantially most, of the above properties should be achieved without a loss of the conditional targeting of the effector function and retention of conditional engagement of a modified signaling agent with its receptor. Often, it is difficult to achieve all these objectives with chimeric proteins encoded or represented by a single, contiguous polypeptide chain. There is a need in the art where such desirable properties of the biologic can be achieved while maintaining the tolerability and therapeutic index of the biologic.

There is a need in the art where such desirable properties of the biologic can be achieved while maintaining the tolerability and therapeutic index of the biologic. Further, there is a need for effector function-encoding biologics that are amenable to production use as a therapy to the treatment or prevention of disease.

### SUMMARY

The present technology provides fragment crystallizable region (Fc)-based chimeric protein complexes in which most, if not all, of the above outlined requirements are met. These constructs encode biological therapeutic agents whose effector function can be delivered in a highly precise fashion to a target of choice and without, or with a mitigated amount of systemic adverse events, thereby limiting systemic cross-reactivities and associated adverse events, while also providing features that impart pharmaceutical properties enabling the production of therapeutic agents with, for example, desired in vivo exposure time (e.g. half-life), size (e.g. for biodistribution and clearance characteristics), as well as large scale production and/or purification for commercial production (e.g. having adequate solubility, purity, stability and storage properties).

In some aspects, the present technology relates to Fc-based chimeric protein complexes comprising a targeting moiety that comprises a recognition domain which recognizes and/or binds to a target, a wild type or modified signaling agent, wherein the modified signaling agent has one or more mutations that confer improved safety relative to a wild type signaling agent, and an Fc domain, having one or more Fc chains. In some embodiments, the Fc domain has one or more mutations that reduce or eliminate an effector function of the Fc domain, promote Fc chain pairing of the Fc domain, and/or stabilize a hinge region in the Fc domain. In some embodiments, the one or more Fc chains of the Fc domain have one or more mutations that reduce or eliminate an effector function of the Fc domain, promote Fc chain pairing of the Fc domain, and/or stabilize a hinge region in the Fc domain.

In some embodiments, such Fc-based chimeric protein complexes are heterodimeric. In some embodiments, the Fc-based chimeric protein complexes are heterodimeric and the targeting moiety and the signaling agent are oriented in trans. In some embodiments, the Fc-based chimeric protein complexes are heterodimeric and pairing is via Ridgway knob-in-hole construction (as described herein). In some embodiments, the Fc-based chimeric protein complexes are heterodimeric and pairing is via Merchant knob-in-hole construction (as described herein).

In some embodiments, such Fc-based chimeric protein complexes are homodimeric.

In some embodiments, the one or more mutations in the modified signaling agent reduces the affinity or activity at the signaling agent's receptor relative to a wild type signaling agent. In some embodiments, the targeting moiety restores the affinity or activity of the modified signaling agent.

In some embodiments, the Fc-based chimeric protein complexes comprise one or more additional targeting moieties and/or wild type or modified signaling agents. In some embodiments, the Fc-based chimeric protein complexes are multispecific. In some embodiments, the targeting moieties are a single domain antibody (VHH).

In another aspect, the present technology relates to the use of Fc-based chimeric protein complexes to treat or prevent various diseases and disorders. In some embodiments, the Fc-based chimeric protein complexes are used to treat cancer, infections, metabolic diseases, (neuro)degenerative diseases, and cardiovascular diseases and immune disorders.

### BRIEF DESCRIPTION OF DRAWINGS

FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 20A-E, 38, 46A-D, 47, and 49 show various non-limiting illustrative schematics of the Fc-based chimeric protein complexes of the present invention. In embodiments, each schematic is a composition of the present invention. Where applicable in the figures, “TM” refers to a “targeting moiety” as described herein, “SA” refers to a “signaling agent” as described herein, “ [symbol] ” is an optional “linker” as described herein, the two long parallel rectangles are human Fc domains, having one or more Fc chains, e.g. from IgG1, from IgG2, or from IgG4, as described herein and optionally with effector knock-out and/or stabilization mutations as also described herein, and the two long parallel rectangles with one having a protrusion and the other having an indentation are human Fc domains, having one or more Fc chains, e.g. from IgG1, from IgG2, or from IgG4 as described herein, with knob-in-hole and/or ionic pair (a/k/a charged pairs, ionic bond, or charged residue pair) mutations as described herein and optionally with effector knock-out and/or stabilization mutations as also described herein.

FIGS. 1A-F show illustrative homodimeric 2-chain complexes. These figures show illustrative configurations for the homodimeric 2-chain complexes.

FIGS. 2A-H show illustrative homodimeric 2-chain complexes with two targeting moieties (TM) (as described herein, more targeting moieties may be present in some embodiments). In embodiments, the position of TM1 and TM2 are interchangeable. In embodiments, the constructs shown in the box (i.e., FIGS. 2B and 2C) have signaling agent (SA) between TM1 and TM2 or between TM1 and Fc.

FIGS. 3A-H show illustrative homodimeric 2-chain complexes with two signaling agents (as described herein, more signaling agents may be present in some embodiments). In embodiments, the position of SA1 and SA2 are interchangeable. In embodiments, the constructs shown in the box (i.e., FIGS. 3G and 3H) have TM between SA1 and SA2 or TM at N- or C-terminus.

FIGS. 4A-D show illustrative heterodimeric 2-chain complexes with split TM and SA chains, namely the TM on the knob chain of the Fc and the SA on hole chain of the Fc.

FIGS. 5A-F show illustrative heterodimeric 2-chain complexes with split TM and SA chains, namely with both TMs on the knob chain of the Fc and with SA on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 6A-J show illustrative heterodimeric 2-chain complexes with split TM and SA chains, namely with TM on the knob chain of the Fc and with a SA on the hole chain of the Fc, with two signaling agents (as described herein, more signaling agents may be present in some embodiments). In these orientations/configurations, one SA is on the knob chain and one SA is on the hole chain. In embodiments, the position of SA1 and SA2 are interchangeable.

FIGS. 7A-D show illustrative heterodimeric 2-chain complexes with split TM and SA chains, namely the SA on the knob chain of the Fc and the TM on hole chain of the Fc.

FIGS. 8A-F show illustrative heterodimeric 2-chain complexes with split TM and SA chains, namely with SA on the knob chain of the Fc and both TMs on hole chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 9A-J show illustrative heterodimeric 2-chain complexes with split TM and SA chains, namely with SA on the knob chain of the Fc and TM on hole chain of the Fc, with two signaling agents (as described herein, more signaling agents may be present in some embodiments). In these orientations/configurations, one SA is on the knob chain and one SA is on the hole chain. In embodiments, the position of SA1 and SA2 are interchangeable.

FIGS. 10A-F show illustrative heterodimeric 2-chain complexes with TM and SA on the same chain, namely the SA and TM both on the knob chain of the Fc.

FIGS. 11A-L show illustrative heterodimeric 2-chain complexes with a TM and a SA on the same chain, namely with SA and with TM both on the knob chain of the Fc, with two targeting moieties (as described herein, more targeting moieties may be present in some embodiments). In embodiments, the position of TM1 and TM2 are interchangeable. In some embodiments, TM1 and TM2 can be identical.

FIGS. 12A-L show illustrative heterodimeric 2-chain complexes with a TM and a SA on the same chain, namely with SA and with TM both on the knob chain of the Fc, with two signaling agents (as described herein, more signaling agents may be present in some embodiments). In embodiments, the position of SA1 and SA2 are interchangeable.

FIGS. 13A-F show illustrative heterodimeric 2-chain complexes with TM and SA on the same chain, namely the SA and TM both on the hole chain of the Fc.

FIGS. 14A-L show illustrative heterodimeric 2-chain complexes with a TM and a SA on the same chain, namely with SA and with TM both on the hole chain of the Fc, with two targeting moieties (as described herein, more targeting moieties are present in some embodiments). In embodiments, the position of TM1 and TM2 are interchangeable. In embodiments, TM1 and TM2 can be identical.

FIGS. 15A-L show illustrative heterodimeric 2-chain complexes with a TM and a SA on the same chain, namely with SA and with TM both on the hole chain of the Fc, with two signaling agents (as described herein, more signaling agents may be present in some embodiments). In embodiments, the position of SA1 and SA2 are interchangeable.

FIGS. 16A-J show illustrative heterodimeric 2-chain complexes with two targeting moieties (as described herein, more targeting moieties may be present in some embodiments) and with SA on knob Fc and TM on each chain. In embodiments, TM1 and TM2 can be identical.

FIGS. 17A-J show illustrative heterodimeric 2-chain complexes with two targeting moieties (as described herein, more targeting moieties may be present in some embodiments) and with SA on hole Fc and TM on each chain. In embodiments, TM1 and TM2 can be identical.

FIGS. 18A-F show illustrative heterodimeric 2-chain complexes with two signaling agents (as described herein, more signaling agents may be present in some embodiments) and with split SA and TM chains: SA on knob and TM on hole Fc.

FIGS. 19A-F show illustrative heterodimeric 2-chain complexes with two signaling agents (as described herein, more signaling agents may be present in some embodiments) and with split SA and TM chains: TM on knob and SA on hole Fc.

FIGS. 20A-E show five variations of homodimeric or heterodimeric Fc-based chimeric protein complexes that were constructed as described in Example 1. In these illustrative constructs, anti-human C-type lectin domain containing 9A (Clec9A) VHHs were the targeting moiety and human interferon alpha 2 with a R149A mutation was the signaling agent.

FIG. 21 shows an SDS-PAGE gel resolving purified proteins of FIGS. 20A-E.

FIG. 22 shows a freezing-thawing stability experiment.

FIG. 23 shows biological activity of the heterodimeric Fc-based chimeric protein complexes.

FIG. 24 shows plasma concentrations of Fc-AcTaferons (Fc-AFNs) after intravenous administration in mouse. Average values of 3 individual samples per time point time (+SEM) are plotted.

FIG. 25 shows tumor growth curves in humanized mice after treatment with buffer or two different Fc-AFN constructs. Average values (in mm³) of 6 animals per time point time (+SEM) are plotted.

FIG. 26 shows biological activity of linker-length variants on HL116-hClec9A cells. HL116-hClec9A cells were stimulated for 6 hours with a serial dilution of linker length variants. Average luciferase values (±STDEV) of triplicate measurements are plotted.

FIG. 27 shows biological activity of effector-variants on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 28 shows binding of (complement component 1q) C1q to Fc domain (CH2 and CH3 domains) of the human IgG1 heavy chain and the hinge region (hIgG1) or Fc Actaferons (AFNs) with different effector-mutations in bio-layer interferometry (BLI).

FIG. 29 shows effect of Interferon (IFN) mutations on biological activity in HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 30 shows biological activity of different Fc formats on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 31 shows biological activity of Fc AFNs with different knob in hole (KiH) on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 32 shows biological activity of mono- and bivalent Fc AFNs on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 33 shows relative binding of mono- and bivalent targeted Fc AFN to HL116-hClec9A cells.

FIG. 34 shows biological activity of Fc AFNs on HL116 and HL116-hClec9A cells. Parental HL116 or the derived HL116-hClec9A cells were stimulated for 6 hours with a serial dilution of Fc AFNs. Average luciferase values (±STDEV) of duplicate measurements are plotted.

FIG. 35 shows plasma concentrations of Fc-AFNs after intravenous administration in mouse. Average values of 3 individual samples per time point time (+SEM) are plotted.

FIG. 36 shows tumor growth curves in humanized mice after treatment with buffer or four different Fc-AFN constructs. Average values (in mm³) of 5 animals per time point time (+SEM) are plotted.

FIG. 37 shows tumor growth curves in humanized mice after treatment with buffer or increasing doses of a single Fc-AFN construct. Average values (in mm³) of 5 animals per time point time (+SEM) are plotted.

FIG. 38 shows schematic representation and biological activity of PD-L1 VHH AFN variants. Parental HL116 cells were stimulated for 6 hours with a serial dilution of Fc AFNs. Average luciferase values (±STDEV) of triplicate measurements are plotted.

FIG. 39 shows tumor growth curves in humanized mice after treatment with PBS, PD-L1 inhibitor (atezolizumab) or PD-L1 targeted Fc-AFN. Average values (in mm³) of 6 animals per time point time (+SEM) are plotted.

FIG. 40 shows biological activity of IFNa2 and Clec4C VHH Fc AFN on HL116 and HL116-hClec4C cells. Parental HL116 or the derived HL116-hClec4C cells were stimulated for 6 hours with a serial dilution of Fc AFNs. Average luciferase values (±STDEV) of triplicate measurements are plotted.

FIG. 41 shows biological activity of IFNa2 and CD20 VHH Fc AFN on HL116 and HL116-hCD20 cells. Parental HL116 or the derived HL116-hCD20 cells were stimulated for 6 hours with a serial dilution of Fc AFNs. Average luciferase values (±STDEV) of triplicate measurements are plotted.

FIG. 42 shows biological activity of CD13 VHH Fc AFN on parental HL116 cells. Parental HL116 were stimulated for 6 hours with a serial dilution of CD13 Fc AFNs in the presence or absence of an excess CD13 VHH. Average luciferase values (±STDEV) of triplicate measurements are plotted.

FIG. 43 shows biological activity of IFNa2 and FAP VHH Fc AFNs on HL116 and HL116-hFAP cells. Parental HL116 or the derived HL116-hFAP cells were stimulated for 6 hours with a serial dilution of Fc AFNs. Average luciferase values (±STDEV) of triplicate measurements are plotted.

FIG. 44 shows biological activity of IFNa2 and CD8 VHH Fc AFN on HL116 and HL116-hCD8 cells. Parental HL116 or the derived HL116-hCD8 cells were stimulated for 6 hours with a serial dilution of Fc AFNs. Average luciferase values (±STDEV) of triplicate measurements are plotted.

FIG. 45 shows relative binding of bispecific CLEC9A and PD-L1 targeting Fc AFN to parental PD-L1 positive HL116 cells (top) and HL116-hClec9A cells (expressing both targets; bottom), including competition with the free PD-L1 VHH 2LIG99

FIGS. 46A-D show illustrative heterodimeric 2-chain complexes with two targeting moieties (as described herein, more targeting moieties are present in some embodiments) and with SA on knob Fc and TM on each chain. Each targeting moiety is present in 2 copies and the positions of TM1 and TM2 are interchangeable.

FIG. 47 shows schematic representation and biological activity of bi-specific Clec9A-PD-L1 Fc AFN variants. Parental HL116 (left) and HL116-hClec9A (right) cells were stimulated for 6 hours with a serial dilution of Fc AFNs. Average luciferase values (±STDEV) of triplicate measurements are plotted.

FIG. 48 shows tumor growth curves in humanized mice after treatment with PBS, mono- or bispecific Fc-AFNs. Average values (in mm²) of 4-5 animals per time point time (+SEM) are plotted.

FIG. 49 shows schematic representation and biological activity of bi-specific Clec4C-CD8 Fc AFN variants. Parental HL116, HL116-hClec4C and HL116-hCD8 cells were stimulated for 6 hours with a serial dilution of Fc AFNs. Average luciferase values (±STDEV) of triplicate measurements are plotted.

FIG. 50 shows biological activity of IFNa2 and scFv Xcr1 Fc AFN on HL116 and HL116-hXcr1 cells. Parental HL116 or the derived HL116-hXcr1 cells were stimulated for 6 hours with a serial dilution of Fc AFNs. Average luciferase values (±STDEV) of triplicate measurements are plotted.

FIG. 51 shows biological activity of IFNa2 and scFv CD20 Fc AFN on HL116 and HL116-hCD20 cells. Parental HL116 or the derived HL116-hCD20 cells were stimulated for 6 hours with a serial dilution of Fc AFNs. Average luciferase values (±STDEV) of triplicate measurements are plotted.

FIG. 52 shows biological activity of tumor necrosis factor (TNFα) and CD20 Fc AcTafactors (AFRs) on HEK-Dual and HEK-Dual-hCD20 cells. Parental HEK-Dual or the derived HEK-Dual-hCD20 cells were stimulated overnight with a serial dilution of Fc AFRs. Average luciferase values (±STDEV) of triplicate measurements are plotted.

FIG. 53 shows pSTAT1 upon FMS-like tyrosine kinase 3 ligand (FLT3L)-Fc-AFN in transient transfected Hek293T cells. FLT3 or MOCK (empty vector) transfected Hek293T cells were stimulated as indicated and stained for pSTAT1. Average % of pSTAT1 positive cells (±STDEV) of duplicate measurements are plotted.

FIG. 54 shows biological activity of IFNa2 and the extracellular (ec) portion of programmed cell death protein 1 (PD-1) (PD-1ec) Fc AFN on HL116 cells. Parental HL116 cells were stimulated for 6 hours with a serial dilution of Fc AFNs in the presence or absence of an excess neutralizing VHH. Average luciferase values (±STDEV) of triplicate measurements are plotted.

FIG. 55 shows biological activity of IFNa2 and PD-L1ec Fc AFN on HL116 and HL116-hPD-1 cells. Parental HL116 or the derived HL116-hPD-1 cells were stimulated for 6 hours with a serial dilution of Fc AFNs. Average luciferase values (±STDEV) of triplicate measurements are plotted.

FIG. 56 shows biological activity of NGR peptide based Fc AFN targeting CD13. Parental HL116 cells were stimulated for 6 hours with a serial dilution of Fc AFNs. Average luciferase values (±STDEV) of duplicate measurements are plotted. Specificity is demonstrated by comparison with CLEC9A targeted Fc AFN (R1CHCL50) FIG. 57 shows biological activity of targeted wild type and mutant IFNa2 on C-Type Lectin Domain Family 4 Member C (Clec4C) positive and negative cells. Peripheral blood mononuclear cells (PBMC's) from healthy donors were stained with Clec4C Ab and stimulated with Clec4C-targeted wild type or mutant IFNa2 for 15 minutes. After fixation and permeabilization, cells were stained with a pSTAT1 Ab. Data are plotted as percentage of pSTAT1 positive cells.

FIG. 58 shows biological activity of targeted wild type and mutant IFNa2 on CD8 positive and negative cells. Peripheral blood mononuclear cells (PBMC's) from healthy donors were stained with CD8 Ab and stimulated with CD8-targeted wild type or mutant IFNa2 for 15 minutes. After fixation and permeabilization, cells were stained with a pSTAT1 Ab. Data are plotted as percentage of pSTAT1 positive cells.

FIG. 59 shows biological activity of targeted wild type and mutant IFNa2 on CD19 positive (i.e. B-cells) and negative cells. PBMC's from healthy donors were stained with CD19 Ab and stimulated with CD20-targeted wild type or mutant IFNa2 for 15 minutes. After fixation and permeabilization, cells were stained with a pSTAT1 Ab. Data are plotted as percentage of pSTAT1 positive cells.

FIG. 60 shows an overview of point mutation in IFNa2. This Figure shows the sequence of mature human IFNa2 (SEQ ID NO: 2) from Amino Acid No. 30-39 and 142-165.

FIG. 61 shows biological activity of different Clec9A VHH Fc AFNs on HL116 and HL116-hClec9A cells. Parental HL116 or the derived HL116-hClec9A cells were stimulated for 6 hours with indicated concentrations of Fc AFNs. Average luciferase values (±STDEV) of triplicate measurements are plotted. For each mutant the bars are, left to right, 1000 ng/ml, 10 ng/ml, 0.1 ng/ml, and not stimulated

FIG. 62 shows biological activity of IFNa1 and Clec9A VHH Fc AFN on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 63 shows biological activity of IFNb and Clec9A VHH Fc AFN on HL116 and HL116-hClec9A cells. Parental HL116 or the derived HL116-hClec9A cells were stimulated for 6 hours with a serial dilution of Fc AFNs. Average luciferase values (±STDEV) of triplicate measurements are plotted.

FIG. 64 shows biological activity of AcTaleukin (ALN) on transient transfected HEK-Blue IL-1β cells. MOCK (an empty vector) or human CD8 transfected cells were stimulated overnight with a serial dilution wild type IL-1β or ALN. Average luciferase values (±STDEV) of triplicate measurements are plotted.

FIG. 65 shows biological activity of TNFα and CD20 VHH Fc AFR on HEK-Dual and HEK-Dual-hCD20 cells. Parental HEK-Dual or the derived HEK-Dual-hCD20 cells were stimulated overnight with a serial dilution of Fc AFR. Average luciferase values (±STDEV) of triplicate measurements are plotted.

FIG. 66 shows biological activity of IFNa2 and bi-AcTakine on HL116 and HL116-hCD8 cells. Parental HL116 or the derived HL116-hCD8 cells were stimulated for 6 hours with a serial dilution of bi-AcTakine. Average luciferase values (±STDEV) of triplicate measurements are plotted.

FIG. 67 shows biological activity of bi-AcTakine on transient transfected HEK-Blue IL-1β cells. MOCK or human CD8 transfected cells were stimulated overnight with a serial dilution wild type IL-1β or bi-AcTakine. Average luciferase values (±STDEV) of triplicate measurements are plotted.

FIG. 68 shows biological activity of Clec9A Fc AFN variant based on human IgG1 and IgG4. Parental HL116 and HL116-hClec9A cells were stimulated for 6 hours with a serial dilution of Fc AFNs. Average luciferase values (±STDEV) of triplicate measurements are plotted.

FIG. 69 shows the plasma concentrations of a CLEC9A AFN (which is not an Fc-based chimeric protein complex) after intravenous administration in mouse. Average values of 3 individual samples per time point time (+SEM) are plotted.

### DETAILED DESCRIPTION

The present technology is based, in part, on the discovery of signaling agents, that are optionally modified to have reduced affinity or activity for one or more of its receptors, and targeting moieties that recognize and bind to a specific target. In some embodiments, one or more signaling agents and one or more targeting moieties are linked and/or conjugated and/or fused to Fc-based chimeric proteins that can pair to form Fc-based chimeric protein complexes. Such Fc-based chimeric protein complexes, surprisingly, have dramatically improved half-lives in vivo, as compared to chimeras lacking an Fc and, especially in the heterodimer configuration as described herein, are particularly amendable to production, purification, and pharmaceutical formulation due to enhanced solubility, stability and other drug-like properties. Accordingly, the present Fc-based chimeric protein complex engineering approach yields agents that are particularly suited for use as therapies.

In some embodiments, these Fc-based chimeric protein complexes may bind and directly or indirectly recruit immune cells to sites in need of therapeutic action (e.g. a tumor or tumor microenvironment). In some embodiments, the Fc-based chimeric protein complexes enhance tumor antigen presentation for elicitation of effective antitumor immune response. In some embodiments these Fc-based chimeric protein complexes may bind tumor cells, tumor microenvironment-associated cells or stromal targets. In some embodiments. these Fc-based chimeric protein complexes may bind to tissue-specific and/or cell-specific specific markers (e.g. antigens, targets) associated with disease-affected or disease-associated organs, tissues and cells. In some embodiments these Fc-based chimeric protein complexes may bind to more than one target/protein marker/antigen present on the same or different cells. In some embodiments these Fc-based chimeric protein complexes may bind to two or more cell types. In some embodiments these Fc-based chimeric protein complexes may bind to more than one cell type and promote formation of a cell complex (e.g. an immune cell and a tumor cell).

In some embodiments, the Fc-based chimeric protein complexes modulate antigen presentation. In some embodiments, the Fc-based chimeric protein complexes temper the immune response to avoid or reduce autoimmunity. In some embodiments, the Fc-based chimeric protein complexes provide immunosuppression. In some embodiments, the Fc-based chimeric protein complexes cause an increase a ratio of Tregs to CD8+ T cells and/or CD4+ T cells in a patient. In some embodiments, the present methods relate to reduction of auto-reactive T cells in a patient.

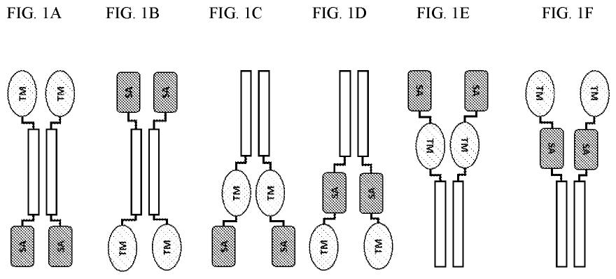

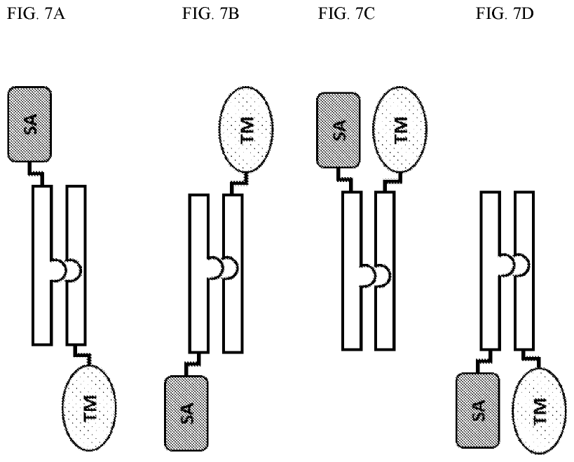

In some embodiments, the Fc-based chimeric protein complexes are a complex of proteins formed, for example, by disulfide bonding and/or ionic pairing. In embodiments, the complex of proteins includes one or more fusion proteins. In some embodiments, the Fc-based chimeric protein complex has a configuration and/or orientation/configuration as shown in any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 20A-E, 38, 46A-D, 47 and 49. In some embodiments, the Fc-based chimeric protein complex has a configuration and/or orientation/configuration as shown in FIG. 7B.

The present technology provides pharmaceutical compositions comprising the Fc-based chimeric protein complexes and their use in the treatment of various diseases, including, e.g., cancer, autoimmune, neurodegenerative diseases, metabolic diseases, cardiovascular diseases and degenerative diseases.

### Fc Domains

The fragment crystallizable domain (Fc domain) is the tail region of an antibody that interacts with Fc receptors located on the cell surface of cells that are involved in the immune system, e.g., B lymphocytes, dendritic cells, natural killer cells, macrophages, neutrophils, eosinophils, basophils, and mast cells. In IgG, IgA and IgD antibody isotypes, the Fc domain is composed of two identical protein chains, derived from the second and third constant domains of the antibody's two heavy chains. In IgM and IgE antibody isotypes, the Fc domain contains three heavy chain constant domains (C_{H} domains 2-4) in each polypeptide chain.

In some embodiments, the Fc-based chimeric protein complex of the present technology include(s) chimeric proteins with Fc domains that promotes formation of such protein complexes. In some embodiments, the Fc domains are from selected from IgG, IgA, IgD, IgM, or IgE. In some embodiments, the Fc domains are from selected from IgG1, IgG2, IgG3, or IgG4.

In some embodiments, the Fc domains are from selected from human IgG, IgA, IgD, IgM, or IgE. In some embodiments, the Fc domains are from selected from human IgG1, IgG2, IgG3, or IgG4.

In some embodiments, the Fc domains of the Fc-based chimeric protein complex comprise the CH2 and CH3 regions of IgG. In some embodiments, the IgG is human IgG. In some embodiments, the human IgG is selected from IgG1, IgG2, IgG3, or IgG4.

In some embodiments, the Fc domains comprise one or more mutations. In some embodiments, the mutation(s) to the Fc domains reduces or eliminates the effector function the Fc domains. In some embodiments, the mutated Fc domain has reduced affinity or binding to a target receptor. By way of example, in some embodiments, the mutation to the Fc domains reduces or eliminates the binding of the Fc domains to FcγR. In some embodiments, the FcγR is selected from FcγRI; FcγRIIa, 131 R/R; FcγRIIa, 131 H/H, FcγRIIb; and FcγRIII. In some embodiments, the mutation to the Fc domains reduces or eliminated binding to complement proteins, such as, e.g., C1q. In some embodiments, the mutation to the Fc domains reduces or eliminated binding to both FcγR and complement proteins, such as, e.g., C1q.

In some embodiments, the Fc domains comprise the LALA mutation to reduce or eliminate the effector function of the Fc domains. By way of example, in some embodiments, the LALA mutation comprises L234A and L235A substitutions in human IgG (e.g., IgG1) (wherein the numbering is based on the commonly used numbering of the CH2 residues for human IgG1 according to EU convention (PNAS, Edelman et al., 1969; 63 (1) 78-85)).

In some embodiments, the Fc domains of human IgG comprise a mutation at one or more of L234, L235, K322, D265, P329, and P331 to reduce or eliminate the effector function of the Fc domains. By way of example, in some embodiments, the mutations are selected from L234A, L234F, L235A, L235E, L235Q, K322A, K322Q, D265A, P329G, P329A, P331G, and P331S.

In some embodiments, the Fc domains comprise the FALA mutation to reduce or eliminate the effector function of the Fc domains. By way of example, in some embodiments, the FALA mutation comprises F234A and L235A substitutions in human IgG4.

In some embodiments, the Fc domains of human IgG4 comprise a mutation at one or more of F234, L235, K322, D265, and P329 to reduce or eliminate the effector function of the Fc domains. By way of example, in some embodiments, the mutations are selected from F234A, L235A, L235E, L235Q, K322A, K322Q, D265A, P329G, and P329A.

In some embodiments, the mutation(s) to the Fc domain stabilize a hinge region in the Fc domain. By way of example, in some embodiments, the Fc domain comprises a mutation at S228 of human IgG to stabilize a hinge region. In some embodiments, the mutation is S228P.

In some embodiments, the mutation(s) to the Fc domain promote chain pairing in the Fc domain. In some embodiments, chain pairing is promoted by ionic pairing (a/k/a charged pairs, ionic bond, or charged residue pair).

In some embodiments, the Fc domain comprises a mutation at one more of the following amino acid residues of IgG to promote of ionic pairing: D356, E357, L368, K370, K392, D399, and K409.

By way of example, in some embodiments, the human IgG Fc domain comprise one of the mutation combinations in Table 1 to promote of ionic pairing.

TABLE 1

| Substitution(s) | Substitution(s) |
|---|---|
| on one Fc Chain | on other Fc Chain |
| D356K D399K | K392D K409D |
| E357R L368R | K370D K409D |
| E357R L368K | K370D K409D |
| E357R D399K | K370D K409D |
| E357R | K370D |
| L368R D399K | K392D K409D |
| L368K D399K | K392D K409D |
| L368R D399K | K409D |
| L368K D399K | K409D |
| L368R | K409D |
| L368K | K409D |
| K370D K409D | E357R D399K |
| K370D K409D | E357R L368R |
| K370D K409D | E357R L368K |
| K370D K409D | E357R D399K |
| K370D K409D | E357R L368R |
| K370D K409D | E357R L368K |
| K370D | E357R |
| K370D | E357R |
| K392D K409D | D356K D399K |
| K392D K409D | L368R D399K |
| K392D K409D | L368K D399K |
| K392D K409D | D399K |
| D399K | K392D K409D |
| D399K | K409D |
| K409D | L368R |
| K409D | L368K |
| K409D | L368R D399K |
| K409D | L368K D399K |
| K409D | L368R |
| K409D | L368K |
| K409D | L368R D399K |
| K409D | L368K D399K |
| K409D | D399K |

In some embodiments, chain pairing of the individual Fc-domains in a chimeric protein complex is promoted by knob-in-hole mutations. In some embodiments, the Fc domain comprises one or more mutations to allow for a knob-in-hole interaction in the Fc domain. In some embodiments, a first Fc chain is engineered to express the “knob” and a second Fc chain is engineered to express the complementary “hole.” By way of example, in some embodiments, human IgG Fc domain comprises the mutations of Table 2 to allow for a knob-in-hole interaction.

TABLE 2

| Substitution(s) | Substitution(s) |
|---|---|
| on one Fc Chain | on other Fc Chain |
| T366Y | Y407T |
| T366Y/F405A | T394W/Y407T |
| T366W | Y407A |
| T366W | Y407V |
| T366Y | Y407A |
| T366Y | Y407V |
| T366Y | Y407T |

In some embodiments, the Fc domains in the Fc-based chimeric protein complexes of the present technology comprise any combination of the above-disclosed mutations. By way of example, in some embodiments, the Fc domain comprises mutations that promote ionic pairing and/or a knob-in-hole interaction. By way of example, in some embodiments, the Fc domain comprises mutations that have one or more of the following properties: promote ionic pairing, induce a knob-in-hole interaction, reduce or eliminate the effector function of the Fc domain, and cause Fc stabilization (e.g. at hinge).

By way of example, in some embodiments, a human IgG Fc domain comprise mutations disclosed in Table 3, which promote ionic pairing and/or promote a knob-in-hole interaction in the Fc domain.

TABLE 3

| Substitution(s) | Substitution(s) |
|---|---|
| on one Fc Chain | on other Fc Chain |
| T366W K370D | E357R Y407A |
| T366W K370D | E357R Y407V |
| T366W K409D | L368R Y407A |
| T366W K409D | L368R Y407V |
| T366W K409D | L368K Y407A |
| T366W K409D | L368K Y407V |
| T366W K409D | L368R D399K Y407A |
| T366W K409D | L368R D399K Y407V |
| T366W K409D | L368K D399K Y407A |
| T366W K409D | L368K D399K Y407V |
| T366W K409D | D399K Y407A |
| T366W K409D | D399K Y407V |
| T366W K392D K409D | D399K Y407A |
| T366W K392D K409D | D399K Y407V |
| T366W K392D K409D | D356K D399K Y407A |
| T366W K392D K409D | D356K D399K Y407V |
| T366W K370D K409D | E357R D399K Y407A |
| T366W K370D K409D | E357R D399K Y407V |
| T366W K370D K409D | E357R L368R Y407A |
| T366W K370D K409D | E357R L368R Y407V |
| T366W K370D K409D | E357R L368K Y407A |
| T366W K370D K409D | E357R L368K Y407V |
| T366W K392D K409D | L368R D399K Y407A |
| T366W K392D K409D | L368R D399K Y407V |
| T366W K392D K409D | L368K D399K Y407A |
| T366W K392D K409D | L368K D399K Y407V |
| E357R T366W | K370D Y407A |
| E357R T366W | K370D Y407V |
| T366W L368R | Y407A K409D |
| T366W L368R | Y407V K409D |
| T366W L368K | Y407A K409D |
| T366W L368K | Y407V K409D |
| T366W L368R D399K | Y407A K409D |
| T366W L368R D399K | Y407V K409D |
| T366W L368K D399K | Y407A K409D |
| T366W L368K D399K | Y407V K409D |
| T366W D399K | Y407A K409D |
| T366W D399K | Y407V K409D |
| 1366W D399K | K392D Y407A K409D |
| T366W D399K | K392D Y407V K409D |
| T366W D356K D399K | K392D Y407A K409D |
| T366W D356K D399K | K392D Y407V K409D |
| E357R T366W D399K | K370D Y407A K409D |
| E357R T366W D399K | K370D Y407V K409D |
| E357R T366W L368R | K370D Y407A K409D |
| E357R T366W L368R | K370D Y407V K409D |
| E357R T366W L368K | K370D Y407A K409D |
| E357R T366W L368K | K370D Y407V K409D |
| T366W L368R D399K | K392D Y407A K409D |
| T366W L368R D399K | K392D Y407V K409D |
| T366W L368K D399K | K392D Y407A K409D |

By way of example, in some embodiments, human IgG Fc domains comprise mutations disclosed in Table 4, which promote ionic pairing, promote a knob-in-hole interaction, or a combination thereof of the Fc domains. In embodiments, the “Chain 1” and “Chain 2” of Table 4 can be interchanged (e.g. Chain 1 can have Y407T and Chain 2 can have T366Y).

TABLE 4

| Chain 1 mutation | Chain 2 mutation | Reference | IgG |
|---|---|---|---|
| T366Y | Y407T | Ridgway et al., 1996 Protein | IgG1 |
| | | Engineering, Design and Selection, | |
| | | Volume 9, Issue 7, 1 Jul. 1996, | |
| | | Pages 617-62 | |
| T366Y/F405A | T394W/Y407T | Ridgway et al., 1996 Protein | IgG1 |
| | | Engineering, Design and Selection, | |
| | | Volume 9, Issue 7, 1 Jul. 1996, | |
| | | Pages 617-62 | |
| T366W | Y407A | Atwell et al., 1997 JMB | IgG1 |
| | | Volume 270, Issue 1, 4 Jul. 1997, | |
| | | Pages 26-35 | |
| T366W | T3665/L368V/Y407A | Atwell et al., 1997 JMB | IgG1 |
| | | Volume 270, Issue 1, 4 Jul. 1997, | |
| | | Pages 26-35 | |
| T366W | L368A/Y407A | Atwell et al., 1997 JMB | IgG1 |
| | | Volume 270, Issue 1, 4 Jul. 1997, | |
| | | Pages 26-35 | |
| T366W | T366S/L368A/Y407A | Atwell et al., 1997 JMB | IgG1 |
| | | Volume 270, Issue 1, 4 Jul. 1997, | |
| | | Pages 26-35 | |
| T366W | T366S/L368G/Y407V | Atwell et al., 1997 JMB | IgG1 |
| | | Volume 270, Issue 1, 4 Jul. 1997, | |
| | | Pages 26-35 | |
| T366W/D3990 | T366S/L368A/ | Merchant et al., 1998 Nature | IgG1 |
| | K392C/Y407V | Biotechnology volume 16, | |
| | | pages 677-681 (1998) | |
| T366W/K3920 | T366S/L368A/ | Merchant et al., 1998 Nature | IgG1 |
| | D399C/Y407V | Biotechnology volume 16, | |
| | | pages 677-681 (1998) | |
| S354C/T366W | Y349C/T366S/ | Merchant et al., 1998 Nature | IgG1 |
| | L368A/Y407V | Biotechnology volume 16, | |
| | | pages 677-681 (1998) | |
| Y349C/T366W | S354C/T366S/ | Merchant et al., 1998 Nature | IgG1 |
| | L368A/Y407V | Biotechnology volume 16, | |
| | | pages 677-681 (1998) | |
| E356C/T366W | Y349C/T366S/ | Merchant et al., 1998 Nature | IgG1 |
| | L368A/Y407V | Biotechnology volume 16, | |
| | | pages 677-681 (1998) | |
| Y349C/T366W | E356C/T366S/ | Merchant et al., 1998 Nature | IgG1 |
| | L368A/Y407V | Biotechnology volume 16, | |
| | | pages 677-681 (1998) | |
| E357C/T366W | Y349C/T366S/ | Merchant et al., 1998 Nature | IgG1 |
| | L368A/Y407V | Biotechnology volume 16, | |
| | | pages 677-681 (1998) | |
| Y349C/T366W | E357C/T366S/ | Merchant et al., 1998 Nature | IgG1 |
| | L368A/Y407V | Biotechnology volume 16, | |
| | | pages 677-681 (1998) | |
| D339R | K409E | Gunasekaran et al., 2010 The Journal of | IgG1 |
| | | Biological Chemistry 285, 19637-19646. | |
| D339K | K409E | Gunasekaran et al., 2010 The Journal of | IgG1 |
| | | Biological Chemistry 285, 19637-19646. | |
| D339R | K409D | Gunasekaran et al., 2010 The Journal of | IgG1 |
| | | Biological Chemistry 285, 19637-19646. | |
| D339K | K409D | Gunasekaran et al., 2010 The Journal of | IgG1 |
| | | Biological Chemistry 285, 19637-19646. | |
| D339K | K360D/K409E | Gunasekaran et al., 2010 The Journal of | IgG1 |
| | | Biological Chemistry 285, 19637-19646. | |
| D339K | K392D/K409E | Gunasekaran et al., 2010 The Journal of | IgG1 |
| | | Biological Chemistry 285, 19637-19646. | |
| D339K/E356K | K392D/K409E | Gunasekaran et al., 2010 The Journal of | IgG1 |
| | | Biological Chemistry 285, 19637-19646. | |
| D339K/E357K | K392D/K409E | Gunasekaran et al., 2010 The Journal of | IgG1 |
| | | Biological Chemistry 285, 19637-19646. | |
| D339K/E356K | K409E/K439D | Gunasekaran et al., 2010 The Journal of | IgG1 |
| | | Biological Chemistry 285, 19637-19646. | |
| D339K/E357K | K370D/K409E | Gunasekaran et al., 2010 The Journal of | IgG1 |
| | | Biological Chemistry 285, 19637-19646. | |
| D339K/E356K/E357K | K370D/K392D/K409E | Gunasekaran et al., 2010 The Journal of | IgG1 |
| | | Biological Chemistry 285, 19637-19646. | |
| S364H/F405A | Y349T/T394F | Moore et al., 2011 mAbs, 3:6, 546-557 | IgG1 |
| S364H/T394F | Y349T/F405A | Moore et al., 2011 mAbs, 3:6, 546-557 | IgG1 |
| D221R/P228R/K409R | D221E/P228E/L368E | Strop et al., 2012 JMB Volume 420, | IgG1 |
| | | Issue 3, 13 Jul. 2012, Pages 204-219 | |
| C223R/E225R/ | C223E/P228E/L368E | Strop et al., 2012 JMB Volume 420, | IgG2 |
| P228R/K409R | | Issue 3, 13 Jul. 2012, Pages 204-219 | |
| F405L | K409R | Labrijn et al. 2013 PNAS March 26, | IgG1 |
| | | 2013. 110 (13) 5145-5150 | |
| F405A/Y407V | T394W | Von Kreudenstein et al., 2013 mAbs | IgG1 |
| | | Volume 5, 2013 - Issue 5, pp.644-654 | |
| F405A/Y407V | T366I/T394W | Von Kreudenstein et al., 2013 mAbs | IgG1 |
| | | Volume 5, 2013 - Issue 5, pp.644-654 | |
| F405A/Y407V | T366L/T394W | Von Kreudenstein et al., 2013 mAbs | IgG1 |
| | | Volume 5, 2013 - Issue 5, pp.644-654 | |
| F405A/Y407V | T366L/K392M/T394W | Von Kreudenstein et al., 2013 mAbs | IgG1 |
| | | Volume 5, 2013 - Issue 5, pp.644-654 | |
| L351Y/F405A/Y407V | T366L/K392M/T394W | Von Kreudenstein et al., 2013 mAbs | IgG1 |
| | | Volume 5, 2013 - Issue 5, pp.644-654 | |
| T350V/L351Y/ | T350V/T366L/ | Von Kreudenstein et al., 2013 mAbs | IgG1 |
| F405A/Y407V | K392M/T394W | Volume 5, 2013 - Issue 5, pp.644-654 | |
| T350V/L351Y/ | T350V/T366L/ | Von Kreudenstein et al., 2013 mAbs | IgG1 |
| F405A/Y407V | K392L/T394W | Volume 5, 2013 - Issue 5, pp.644-654 | |
| K409W | D339V/F405T | Choi et al., 2013 PNAS Jan. 2, 2013. | IgG1 |
| | | 110 (1) 270-275 | |
| K360E | Q347R | Choi et al., 2013 PNAS Jan. 2, 2013. | IgG1 |
| | | 110 (1) 270-275 | |
| K360E/K409W | D339V/Q347R/F405T | Choi et al., 2013 PNAS Jan. 2, 2013. | IgG1 |
| | | 110 (1) 270-275 | |
| Y349C/K360E/K409W | D339V/Q347R/ | Choi et al., 2013 PNAS Jan. 2, 2013. | IgG1 |
| | S3540/F405T | 110 (1) 270-275 | |
| K392A/K409D | E356K/D399K | Leaver-Fey et al., 2016 Structure | IgG1 |
| | | Volume 24, Issue 4, 5 Apr. 2016, | |
| | | Pages 641-651 | |
| T366W | T3665/L358A/Y407A | Leaver-Fey et al., 2016 Structure | IgG1 |
| | | Volume 24, Issue 4, 5 Apr. 2016, | |
| | | Pages 641-651 | |
| D339M/Y407A | T336V/K409V | Leaver-Fey et al., 2016 Structure | IgG1 |
| | | Volume 24, Issue 4, 5 Apr. 2016, | |
| | | Pages 641-651 | |
| D339M/K360D/ | T336V/E345R/ | Leaver-Fey et al., 2016 Structure | IgG1 |
| Y407A | Q347R/K409V | Volume 24, Issue 4, 5 Apr. 2016, | |
| | | Pages 641-651 | |
| Y349S/T366V/ | E357D/S364Q/Y407A | Leaver-Fey et al., 2016 Structure | IgG1 |
| K370Y/K409V | | Volume 24, Issue 4, 5 Apr. 2016, | |
| | | Pages 641-651 | |
| Y349S/T366M/ | E356G/E357D/ | Leaver-Fey et al., 2016 Structure | IgG1 |
| K370Y/K409V | S364Q/Y407A | Volume 24, Issue 4, 5 Apr. 2016, | |
| | | Pages 641-651 | |
| Y349S/T366M/ | E357D/5364R/Y407A | Leaver-Fey et al., 2016 Structure | IgG1 |
| K370Y/K409V | | Volume 24, Issue 4, 5 Apr. 2016, | |
| | | Pages 641-651 | |

And any combination as described in Tables 1-3 of US20150284475A1

By way of example, in some embodiments, a human IgG Fc domains comprise mutations disclosed in Table 5, which reduce or eliminate FcγR and/or complement binding in the Fc domain. In embodiments, the table 5 mutations are in both chains.

TABLE 5

| Chain 1 mutation | Reference | IgG |
|---|---|---|
| L234A/L235A | Alegre etal., 1994 | IgG1 |
| | Transplantation 57: | |
| | 1537-1543 | |
| F234A/L235A | Alegre et al., 1994 | IgG4 |
| | Transplantation 57: | |
| | 1537-1543 | |
| L235E | Morgan et al., 1995 | IgG1 |
| | Immunology. 1995 Oct; | |
| | 86 (2): 319-324. | |
| L235E | Morgan et al., 1995 | IgG4 |
| | Immunology. 1995 Oct; | |
| | 86 (2): 319-324. | |
| L235A | Morgan et al., 1995 | IgG1 |
| | Immunology. 1995 Oct; | |
| | 86 (2): 319-324. | |
| G237A | Morgan et al., 1995 | IgG1 |
| | Immunology. 1995 Oct; | |
| | 86 (2): 319-324. | |
| N297H | Tao and Morrison, | IgG1 |
| | J. Immunol. 1989; 143: | |
| | 2595-2601 | |
| N297Q | Tao and Morrison, | IgG1 |
| | J. Immunol. 1989; 143: | |
| | 2595-2601 | |
| N297K | Tao and Morrison, | IgG3 |
| | J. Immunol. 1989; 143: | |
| | 2595-2601 | |
| N297Q | Tao and Morrison, | IgG3 |
| | J. Immunol. 1989; 143: | |
| | 2595-2601 | |
| D265A | ldusogie et al., 2000 J | IgG1 |
| | Immunol Apr. 15, 2000, 164 | |
| | (8) 4178-4184 | |
| D270A, V, K | ldusogie et al., 2000 J | IgG1 |
| | Immunol Apr. 15, 2000, 164 | |
| | (8) 4178-4184 | |
| K322A, L, M, D, E | ldusogie et al., 2000 J | IgG1 |
| | Immunol Apr. 15, 2000, 164 | |
| | (8) 4178-4184 | |
| P329A, X | ldusogie et al., 2000 J | IgG1 |
| | Immunol Apr. 15, 2000, 164 | |
| | (8) 4178-4184 | |
| P331A, S, G, X | ldusogie et al., 2000 J | IgG1 |
| | Immunol Apr. 15, 2000, 164 | |
| | (8) 4178-4184 | |
| D265A | ldusogie et al., 2000 J | IgG1 |
| | Immunol Apr. 15, 2000, 164 | |
| | (8) 4178-4184 | |
| L234A | Hezareh et al., 2001 J. Virol. | IgG1 |
| | December 2001 vol. 75 no. | |
| | 24 12161-12168 | |
| L234A/L235A | Hezareh et al., 2001 J. Virol. | IgG1 |
| | December 2001 vol. 75 no. | |
| | 24 12161-12168 | |
| L234F/L235E/P331S | Oganesyan et al., 2008 Acta | IaG1 |
| | Cryst. (2008). D64, 700-704 | |
| H268Q/V309L/ | An et al., 2009 mAbs | IgG1 |
| A330S/P331S | Volume 1, 2009 - Issue 6, | |
| | pp. 572-579 | |
| G236R/L328R | Moore et al., 2011 mAbs | IgG1 |
| | Volume 3, 2011 - Issue 6, | |
| | pp. 546-557 | |
| N297G | Couch et al., 2013 Sci. | IgG1 |
| | Transl. Med., 5 (2013) | |
| | 183ra57, 1-12 | |
| N297G/D265A | Couch et al., 2013 Sci. | IgG1 |
| | Transl. Med., 5 (2013) | |
| | 183ra57, 1-12 | |
| V234A/G237A/P328S/ | Vafa et al., 2014 Methods | IgG2 |
| H268A/V309L/A330S/ | Volume 65, Issue 1, | |
| P331S | 1 Jan. 2014, Pages 114-126 | |
| L234A/L235A/P329G | Lo et al., 2016 The Journal | IgG1 |
| | of Biological Chemistry | |
| | 292, 3900-3908 | |
| N297D | Schlothauer et al., 2016 | IgG1 |
| | Protein Engineering, Design | |
| | and Selection, Volume 29, | |
| | Issue 10, 1 Oct. 2016, | |
| | Pages 457-466 | |
| S228P/L235E | Schlothauer et al., 2016 | IgG4 |
| | Protein Engineering, Design | |
| | and Selection, Volume 29, | |
| | Issue 10, 1 Oct. 2016, | |
| | Pages 457-466 | |
| S228P/L235E/P329G | Schlothauer et al., 2016 | IgG4 |
| | Protein Engineering, Design | |
| | and Selection, Volume 29, | |
| | Issue 10, 1 Oct. 2016, | |
| | Pages 457-466 | |
| L234F/L235A/K322Q | Borrok et al., 2017 J Pharm | IgG1 |
| | Sci April 2017 Volume 106, | |
| | Issue 4, Pages 1008-1017 | |
| L234F/L235Q/P331G | Borrok et al., 2017 J Pharm | IgG1 |
| | Sci April 2017 Volume 106, | |
| | Issue 4, Pages 1008-1017 | |
| L234F/L235Q/K322Q | Borrok et al., 2017 J Pharm | IgG1 |
| | Sci April 2017 Volume 106, | |
| | Issue 4, Pages 1008-1017 | |
| L234A/L235A/ | Tam et al., 2017 Open | IgG1 |
| G237A/P328S/ | Access | |
| H268A/A330S/ | Antibodies 2017, 6 (3), 12; | |
| P331S | doi:10.3390/antib6030012 | |
| S228P/F234A/L235A | Tam et al., 2017 Open | IgG4 |
| | Access | |
| | Antibodies 2017, 6 (3), 12; | |
| | doi:10.3390/antib6030012 | |
| S228P/F234A/ | Tam et al., 2017 Open | IgG4 |
| L235A/G237A/ | Access | |
| P238S | Antibodies 2017, 6 (3), 12; | |
| | doi:10.3390/antib6030012 | |
| S228P/F234A/ | Tam et al., 2017 Open | IgG4 |
| L235A/G236 [symbol] / | Access | |
| G237A/P238S | Antibodies 2017, 6 (3), 12; | |
| | doi:10.3390/antib6030012 | |

In some embodiments, the Fc domains in the Fc-based chimeric protein complexes of the present technology are homodimeric, i.e., the Fc domain in the chimeric protein complex comprises two identical protein chains.

In some embodiments, the Fc domains in the Fc-based chimeric protein complexes of the present technology are heterodimeric, i.e., the Fc domain in the chimeric protein complex comprises two non-identical protein chains.

In some embodiments, heterodimeric Fc domains are engineered using ionic pairing and/or knob-in-hole mutations described herein. In some embodiments, the heterodimeric Fc-based chimeric protein complexes have a trans orientation/configuration. In a trans orientation/configuration, the targeting moiety and signaling agent are, in embodiments, not found on the same polypeptide chain in the present Fc-based chimeric protein complexes. In some embodiments, the signaling agent and targeting moiety are on the same end (N-terminus or C-terminus) of the Fc domain. In some embodiments, the signaling agent and targeting moiety are on different ends (N-terminus or C-terminus) of the Fc domain.

In some embodiments, heterodimeric Fc domains are engineered using ionic pairing and/or knob-in-hole mutations described herein. In some embodiments, the heterodimeric Fc-based chimeric protein complexes have a trans orientation.

In a trans orientation, the targeting moiety and signaling agent are, in embodiments, not found on the same polypeptide chain in the present Fc-based chimeric protein complexes. In a trans orientation, the targeting moiety and signaling agent are, in embodiments, found on separate polypeptide chains in the Fc-based chimeric protein complexes. In a cis orientation, the targeting moiety and signaling agent are, in embodiments, found on the same polypeptide chain in the Fc-based chimeric protein complexes.

In some embodiments, where more than one targeting moiety is present in the heterodimeric protein complexes described herein, one targeting moiety may be in trans orientation (relative to the signaling agent), whereas another targeting moiety may be in cis orientation (relative to the signaling agent). In some embodiments, the signaling agent and target moiety are on the same ends/sides (N-terminal or C-terminal ends) of an Fc domain. In some embodiments, the signaling agent and targeting moiety are on different sides/ends of an Fc domain (N-terminal and C-terminal ends).

In some embodiments, where more than one targeting moiety is present in the heterodimeric protein complexes described herein, the targeting moieties may be found on the same Fc chain or on two different Fc chains in the heterodimeric protein complex (in the latter case the targeting moieties would be in trans relative to each other, as they are on different Fc chains). In some embodiments, where more than one targeting moiety is present on the same Fc chain, the targeting moieties may be on the same or different sides/ends of an Fc chain (N-terminal or/and C-terminal ends).

In some embodiments, where more than one signaling agent is present in the heterodimeric protein complexes described herein, the signaling agents may be found on the same Fc chain or on two different Fc chains in the heterodimeric protein complex (in the latter case the signaling agents would be in trans relative to each other, as they are on different Fc chains). In some embodiments, where more than one signaling agent is present on the same Fc chain, the signaling agents may be on the same or different sides/ends of an Fc chain (N-terminal or/and C-terminal ends).

In some embodiments, where more than one signaling agent is present in the heterodimeric protein complexes described herein, one signaling agent may be in trans orientation (as relates to the targeting moiety), whereas another signaling agent may be in cis orientation (as relates to the targeting moiety).

In some embodiments, the Fc domains include or start with the core hinge region of wild-type human IgG1, which contains the sequence Cys-Pro-Pro-Cys (SEQ ID NO: 1341). In some embodiments, the Fc domains also include the upper hinge, or parts thereof (e.g., DKTHTCPPC (SEQ ID NO: 1342; see WO2009053368), EPKSCDKTHTCPPC (SEQ ID NO: 1343), or EPKSSDKTHTCPPC (SEQ ID NO: 1344; see Lo et al., Protein Engineering vol. 11 no. 6 pp. 495-500, 1998)).

### Signaling Agents (SA)

In some embodiments, the Fc-based chimeric protein complexes of the present technology include one or more signaling agents (SA). The signaling agents, as disclosed herein, can be a wild type signaling agent or a modified signaling agent.

In various embodiments, the Fc-based chimeric protein complex comprises a wild type signaling agent that has improved target selectivity and safety relative to a signaling agent which is not fused to an Fc, or a signaling agent which is not in the context of a complex, e.g., without limitation, a heterodimeric complex. In various embodiments, the Fc-based chimeric protein complex comprises a wild type signaling agent that has improved target selective activity relative to a signaling agent which is not fused to an Fc, or a signaling agent which is not in the context of a complex, e.g., without limitation, a heterodimeric complex. In various embodiments, the Fc-based chimeric protein complex allows for conditional activity.

In various embodiments, the Fc-based chimeric protein complex comprises a wild type signaling agent that has one or more of attenuated activity such as one or more of reduced binding affinity, reduced endogenous activity, and reduced specific bioactivity as compared to the signaling agent which is not fused to an Fc, or a signaling agent which is not in the context of a complex, e.g., without limitation, a heterodimeric complex.

In various embodiments, the Fc-based chimeric protein complex comprises a wild type signaling agent that has improved safety, e.g. reduced systemic toxicity, reduced side effects, and reduced off-target effects relative to a signaling agent which is not fused to an Fc, or a signaling agent which is not in the context of a complex, e.g., without limitation, a heterodimeric complex. In various embodiments, improved safety means that the present Fc-based chimeric protein provides lower toxicity (e.g. systemic toxicity and/or tissue/organ-associated toxicities); and/or lessened or substantially eliminated side effects; and/or increased tolerability, lessened or substantially eliminated adverse events; and/or reduced or substantially eliminated off-target effects; and/or an increased therapeutic window of the wild type signaling agent as compared to the signaling agent which is not fused to an Fc, or a signaling agent which is not in the context of a complex, e.g., without limitation, a heterodimeric complex.

In some embodiments, the reduced affinity or activity at the receptor is restorable by inclusion in the present complex having one or more of the targeting moieties as described herein.

In various embodiments, the Fc-based chimeric protein complex comprises a wild type signaling agent that has reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity. In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor as compared to the signaling agent which is not fused to an Fc, or a signaling agent which is not in the context of a complex, e.g., without limitation, a heterodimeric complex. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower as compared to the signaling agent which is not fused to an Fc, or a signaling agent which is not in the context of a complex, e.g., without limitation, a heterodimeric complex.

In various embodiments, the Fc-based chimeric protein complex comprises a wild type signaling agent that has reduced endogenous activity of the signaling agent to about 75%, or about 70%, or about 60%, or about 50%, or about 40%, or about 30%, or about 25%, or about 20%, or about 10%, or about 5%, or about 3%, or about 1%, e.g., as compared to the signaling agent which is not fused to an Fc, or a signaling agent which is not in the context of a complex, e.g., without limitation, a heterodimeric complex.

In various embodiments, the signaling agent has one or more mutations that confer improved target selectivity and safety relative to a wild type signaling agent. In various embodiments, the signaling agent has one or more mutations that confer improved target selective activity relative to a wild type signaling agent. In various embodiments, the signaling agent has one or more mutations that allow for conditional activity.

In various embodiments, the signaling agent is modified to have reduced affinity or activity for one or more of its receptors, which allows for attenuation of activity (inclusive of agonism or antagonism) and/or prevents non-specific signaling or undesirable sequestration of the Fc-based chimeric protein complex.

In various embodiments, the signaling agent is agonistic in its wild type form and bears one or more mutations that attenuate its agonistic activity.

In various embodiments, the signaling agent is antagonistic in its wild type form and bears one or more mutations that attenuate its antagonistic activity. In various embodiments, the signaling agent is antagonistic due to one or more mutations, e.g. an agonistic signaling agent is converted to an antagonistic signaling agent and, such a converted signaling agent, optionally, also bears one or more mutations that attenuate its antagonistic activity (e.g. as described in WO 2015/007520, the entire contents of which are hereby incorporated by reference).

Accordingly, in various embodiments, the signaling agent is a modified (e.g. mutant) form (e.g., having one or more mutations) of a wild type signaling agent. In various embodiments, the modifications (e.g. mutations) allow for the modified signaling agent to have one or more of attenuated activity such as one or more of reduced binding affinity, reduced endogenous activity, and reduced specific bioactivity as compared to the unmodified or unmutated signaling agent, i.e. the wild type form of the signaling agent (e.g. comparing the same signaling agent in a wild type form versus a modified or mutant form). In some embodiments, the mutations which attenuate or reduce binding or affinity include those mutations which substantially reduce or ablate binding or activity. In some embodiments, the mutations which attenuate or reduce binding or affinity are different from those mutations which substantially reduce or ablate binding or activity. Consequentially, in various embodiments, the mutations allow for the signaling agent to have improved safety, e.g. reduced systemic toxicity, reduced side effects, and reduced off-target effects relative to unmutated, i.e. wild type, signaling agent (e.g. comparing the same signaling agent in a wild type form versus a modified (e.g. mutant) form).

As described herein, the signaling agent may have improved safety due to one of more modifications, e.g. mutations. In various embodiments, improved safety means that the present Fc-based chimeric protein provides lower toxicity (e.g. systemic toxicity and/or tissue/organ-associated toxicities); and/or lessened or substantially eliminated side effects; and/or increased tolerability, lessened or substantially eliminated adverse events; and/or reduced or substantially eliminated off-target effects; and/or an increased therapeutic window.

In various embodiments, the signaling agent is modified to have one or more mutations that reduce its binding affinity or activity for one or more of its receptors. In some embodiments, the signaling agent is modified to have one or more mutations that substantially reduce or ablate binding affinity or activity for the receptors. In some embodiments, the activity provided by the wild type signaling agent is agonism at the receptor (e.g. activation of a cellular effect at a site of therapy). For example, the wild type signaling agent may activate its receptor. In such embodiments, the mutations result in the modified signaling agent to have reduced or ablated activating activity at the receptor. For example, the mutations may result in the modified signaling agent to deliver a reduced activating signal to a target cell or the activating signal could be ablated. In some embodiments, the activity provided by the wild type signaling agent is antagonism at the receptor (e.g. blocking or dampening of a cellular effect at a site of therapy). For example, the wild type signaling agent may antagonize or inhibit the receptor. In these embodiments, the mutations result in the modified signaling agent to have a reduced or ablated antagonizing activity at the receptor. For example, the mutations may result in the modified signaling agent to deliver a reduced inhibitory signal to a target cell or the inhibitory signal could be ablated. In various embodiments, the signaling agent is antagonistic due to one or more mutations, e.g. an agonistic signaling agent is converted to an antagonistic signaling agent (e.g. as described in WO 2015/007520, the entire contents of which are hereby incorporated by reference) and, such a converted signaling agent, optionally, also bears one or more mutations that reduce its binding affinity or activity for one or more of its receptors or that substantially reduce or ablate binding affinity or activity for one or more of its receptors.

In some embodiments, the reduced affinity or activity at the receptor is restorable by inclusion in the present complex having one or more of the targeting moieties as described herein. In other embodiments, the reduced affinity or activity at the receptor is not substantially restorable by the activity of one or more of the targeting moieties.

In various embodiments, the Fc-based chimeric protein complex of the present technology reduces off-target effects because their signaling agents have mutations that weaken or ablate binding affinity or activity at a receptor.

In various embodiments, this reduction in side effects is observed relative with, for example, the wild type signaling agents. In various embodiments, the signaling agent is active on target cells because the targeting moiety(ies) compensates for the missing/insufficient binding (e.g., without limitation and/or avidity) required for substantial activation. In various embodiments, the wild type or modified signaling agent is substantially inactive en route to the site of therapeutic activity and has its effect substantially on specifically targeted cell types which greatly reduces cross-reactivities and/or potentially associated side effects.

In some embodiments, the signaling agent may include one or more mutations that attenuate or reduce binding or affinity for one receptor (i.e., a therapeutic receptor) and one or more mutations that substantially reduce or ablate binding or activity at a second receptor. In such embodiments, these mutations may be at the same or at different positions (i.e., the same mutation or multiple mutations). In some embodiments, the mutation(s) that reduce binding and/or activity at one receptor is different from the mutation(s) that substantially reduce or ablate at another receptor. In some embodiments, the mutation(s) that reduce binding and/or activity at one receptor is the same as the mutation(s) that substantially reduce or ablate at another receptor. In some embodiments, the present Fc-based chimeric protein complexes have a modified signaling agent that has both mutations that attenuate binding and/or activity at a therapeutic receptor and therefore allow for a more controlled, on-target therapeutic effect (e.g. relative wild type signaling agent) and mutations that substantially reduce or ablate binding and/or activity at another receptor and therefore reduce side effects (e.g. relative to wild type signaling agent).

In some embodiments, the substantial reduction or ablation of binding or activity is not substantially restorable with a targeting moiety described herein. In some embodiments, the substantial reduction or ablation of binding or activity is restorable with a targeting moiety. In various embodiments, substantially reducing or ablating binding or activity at a second receptor also may prevent deleterious effects that are mediated by the other receptor. Alternatively, or in addition, substantially reducing or ablating binding or activity at the other receptor causes the therapeutic effect to improve as there is a reduced or eliminated sequestration of the therapeutic Fc-based chimeric protein complexes away from the site of therapeutic action. For instance, in some embodiments, this obviates the need of high doses of the present Fc-based chimeric protein complexes that compensate for loss at the other receptor. Such ability to reduce dose further provides a lower likelihood of side effects.

In various embodiments, the modified signaling agent comprises one or more mutations that cause the signaling agent to have reduced, substantially reduced, or ablated affinity, e.g. binding (e.g. K_{D}) and/or activation (for instance, when the modified signaling agent is an agonist of its receptor, measurable as, for example, K_{A} and/or EC₅₀) and/or inhibition (for instance, when the modified signaling agent is an antagonist of its receptor, measurable as, for example, K_{I} and/or IC₅₀), for one or more of its receptors. In various embodiments, the reduced affinity at the signaling agent's receptor allows for attenuation of activity (inclusive of agonism or antagonism). In such embodiments, the modified signaling agent has about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, about 95%, or about 10%-20%, about 20%-40%, about 50%, about 40%-60%, about 60%-80%, about 80%-100% of the affinity for the receptor relative to the wild type signaling agent. In some embodiments, the binding affinity is at least about 2-fold lower, about 3-fold lower, about 4-fold lower, about 5-fold lower, about 6-fold lower, about 7-fold lower, about 8-fold lower, about 9-fold lower, at least about 10-fold lower, at least about 15-fold lower, at least about 20-fold lower, at least about 25-fold lower, at least about 30-fold lower, at least about 35-fold lower, at least about 40-fold lower, at least about 45-fold lower, at least about 50-fold lower, at least about 100-fold lower, at least about 150-fold lower, or about 10-50-fold lower, about 50-100-fold lower, about 100-150-fold lower, about 150-200-fold lower, or more than 200-fold lower relative to the wild type signaling agent.

In embodiments wherein the Fc-based chimeric protein complex comprises a modified signaling agent having mutations that reduce binding at one receptor and substantially reduce or ablate binding at a second receptor, the attenuation or reduction in binding affinity of the modified signaling agent for one receptor is less than the substantial reduction or ablation in affinity for the other receptor. In some embodiments, the attenuation or reduction in binding affinity of the modified signaling agent for one receptor is less than the substantial reduction or ablation in affinity for the other receptor by about 1%, or about 3%, about 5%, about 10%, about 15%, about 20%, about 25%, about 30%, about 35%, about 40%, about 45%, about 50%, about 60%, about 65%, about 70%, about 75%, about 80%, about 85%, about 90%, or about 95%. In various embodiments, substantial reduction or ablation refers to a greater reduction in binding affinity and/or activity than attenuation or reduction.

In various embodiments, the modified signaling agent comprises one or more mutations that reduce the endogenous activity of the signaling agent to about 75%, or about 70%, or about 60%, or about 50%, or about 40%, or about 30%, or about 25%, or about 20%, or about 10%, or about 5%, or about 3%, or about 1%, e.g., relative to the wild type signaling agent.

In some embodiments, the modified signaling agent comprises one or more mutations that cause the signaling agent to have reduced affinity for its receptor that is lower than the binding affinity of the targeting moiety(ies) for its(their) receptor(s). In some embodiments, this binding affinity differential is between signaling agent/receptor and targeting moiety/receptor on the same cell. In some embodiments, this binding affinity differential allows for the signaling agent, e.g. mutated signaling agent, to have localized, on-target effects and to minimize off-target effects that underlie side effects that are observed with wild type signaling agent. In some embodiments, this binding affinity is at least about 2-fold, or at least about 5-fold, or at least about 10-fold, or at least about 15-fold lower, or at least about 25-fold, or at least about 50-fold lower, or at least about 100-fold, or at least about 150-fold.

Receptor binding activity may be measured using methods known in the art. For example, affinity and/or binding activity may be assessed by Scatchard plot analysis and computer-fitting of binding data (e.g. Scatchard, The attractions of proteins for small molecules and ions. Ann NY Acad Sci 51: 660-672, 1949) or by reflectometric interference spectroscopy under flow through conditions, as described by Brecht et al. Biosens Bioelectron 1993; 8:387-392, the entire contents of all of which are hereby incorporated by reference.

The amino acid sequences of the wild type signaling agents described herein are well known in the art. Accordingly, in various embodiments the modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known wild type amino acid sequences of the signaling agents described herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments the modified signaling agent comprises an amino acid sequence that has at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with any amino acid sequences of the signaling agents described herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, or about 99% sequence identity).

In various embodiments, the modified signaling agent comprises an amino acid sequence having one or more amino acid mutations. In some embodiments, the one or more amino acid mutations may be independently selected from substitutions, insertions, deletions, and truncations. In some embodiments, the amino acid mutations are amino acid substitutions, and may include conservative and/or non-conservative substitutions, as described elsewhere herein.

In various embodiments, the modified signaling comprises a truncation of one or more amino acids, e.g. an N-terminal truncation and/or a C-terminal truncation.

In various embodiments, the substitutions may also include non-classical amino acids as described elsewhere herein.

As described herein, the modified signaling agents bear mutations that affect affinity and/or activity at one or more receptors. In various embodiments, there is reduced affinity and/or activity at a therapeutic receptor, e.g. a receptor through which a desired therapeutic effect is mediated (e.g. agonism or antagonism). In various embodiments, the modified signaling agents bear mutations that substantially reduce or ablate affinity and/or activity at a receptor, e.g. a receptor through which a desired therapeutic effect is not mediated (e.g. as the result of promiscuity of binding). The receptors of any signaling agents, as described herein, are known in the art.

Illustrative mutations which provide reduced affinity and/or activity (e.g. agonistic) at a receptor are found in WO 2013/107791 and PCT/EP2017/061544 (e.g. with regard to interferons), WO 2015/007542 (e.g. with regard to interleukins), and WO 2015/007903 (e.g. with regard to TNF), the entire contents of each of which are hereby incorporated by reference. Illustrative mutations which provide reduced affinity and/or activity (e.g. antagonistic) at a therapeutic receptor are found in WO 2015/007520, the entire contents of which are hereby incorporated by reference.

In various embodiments, the signaling agent is an immune-modulating agent, e.g. one or more of an interleukin, interferon, and tumor necrosis factor.

In some embodiments, the signaling agent is a wild type interleukin or a modified interleukin, including for example IL-1; IL-2; IL-3; IL-4; IL-5; IL-6; IL-7; IL-8; IL-9; IL-10; IL-11; IL-12; IL-13; IL-14; IL-15; IL-16; IL-17; IL-18; IL- 19; IL-20; IL-21; IL-22; IL-23; IL-24; IL-25; IL-26; IL-27; IL-28; IL-29; IL-30; IL-31; IL-32; IL-33; IL-35; IL-36 or a fragment, variant, analogue, or family-member thereof. Interleukins are a group of multi-functional cytokines synthesized by lymphocytes, monocytes, and macrophages. Known functions include stimulating proliferation of immune cells (e.g., T helper cells, B cells, eosinophils, and lymphocytes), chemotaxis of neutrophils and T lymphocytes, and/or inhibition of interferons. Interleukin activity can be determined using assays known in the art: Matthews et al., in Lymphokines and Interferons: A Practical Approach, Clemens et al., eds, IRL Press, Washington, D.C. 1987, pp. 221-225; and Orencole & Dinarello (1989) Cytokine 1, 14-20.

In some embodiments, the signaling agent is a wild type interferon or a modified version of an interferon such as interferon types I, II, and III. Illustrative interferons, including for example, interferon-α-1, 2, 4, 5, 6, 7, 8, 10, 13, 14, 16, 17, and 21, interferon-β and interferon-γ, interferon κ, interferon ε, interferon [symbol] , and interferon ω.

In some embodiments, the signaling agent is a wild type tumor necrosis factor (TNF) or a modified version of a tumor necrosis factor (TNF) or a protein in the TNF family, including but not limited to, TNF-α, TNF-β, LT-β, CD40L, CD27L, CD30L, FASL, 4-1BBL, OX40L, and TRAIL.

In some embodiments, the modified signaling agent comprises one or more mutations that cause the signaling agent to have reduced affinity and/or activity for a type I cytokine receptor, a type II cytokine receptor, a chemokine receptor, a receptor in the Tumor Necrosis Factor Receptor (TNFR) superfamily, TGF-beta Receptors, a receptor in the immunoglobulin (Ig) superfamily, and/or a receptor in the tyrosine kinase superfamily.

In various embodiments, the receptor for the signaling agent is a Type I cytokine receptor. Type I cytokine receptors are known in the art and include, but are not limited to receptors for IL2 (beta-subunit), IL3, IL4, IL5, IL6, IL7, IL9, IL11, IL12, GM-CSF, G-CSF, LIF, CNTF, and also the receptors for Thrombopoietin (TPO), Prolactin, and Growth hormone. Illustrative type I cytokine receptors include, but are not limited to, GM-CSF receptor, G-CSF receptor, LIF receptor, CNTF receptor, TPO receptor, and type I IL receptors.

In various embodiments, the receptor for the signaling agent is a Type II cytokine receptor. Type II cytokine receptors are multimeric receptors composed of heterologous subunits, and are receptors mainly for interferons. This family of receptors includes, but is not limited to, receptors for interferon-α, interferon-β and interferon-γ, IL10, 1L22, and tissue factor. Illustrative type II cytokine receptors include, but are not limited to, IFN-α receptor (e.g. IFNAR1 and IFNAR2), IFN-β receptor, IFN-γ receptor (e.g. IFNGR1 and IFNGR2), and type II IL receptors.

In various embodiments, the receptor for the signaling agent is a G protein-coupled receptor. Chemokine receptors are G protein-coupled receptors with seven transmembrane structure and coupled to G-protein for signal transduction. Chemokine receptors include, but are not limited to, CC chemokine receptors, CXC chemokine receptors, CX3C chemokine receptors, and XC chemokine receptor (XCR1). Exemplary chemokine receptors include, but are not limited to, CCR1, CCR2, CCR3, CCR4, CCR5, CCR6, CCR7, CCR8, CCR9, CCR10, CXCR1, CXCR2, CXCR3, CXCR3B, CXCR4, CXCR5, CSCR6, CXCR7, XCR1, and CX3CR1.

In various embodiments, the receptor for the signaling agent is a TNFR family member. Tumor necrosis factor receptor (TNFR) family members share a cysteine-rich domain (CRD) formed of three disulfide bonds surrounding a core motif of CXXCXXC creating an elongated molecule. Exemplary tumor necrosis factor receptor family members include: CDI 20a (TNFRSFIA), CD 120b (TNFRSFIB), Lymphotoxin beta receptor (LTBR, TNFRSF3), CD 134 (TNFRSF4), CD40 (CD40, TNFRSF5), FAS (FAS, TNFRSF6), TNFRSF6B (TNFRSF6B), CD27 (CD27, TNFRSF7), CD30 (TNFRSF8), CD137 (TNFRSF9), TNFRSFIOA (TNFRSFIOA), TNFRSFIOB, (TNFRSFIOB), TNFRSFIOC (TNFRSFIOC), TNFRSFIOD (TNFRSFIOD), RANK (TNFRSFI IA), Osteoprotegerin (TNFRSFI IB), TNFRSF12A (TNFRSF12A), TNFRSF13B (TNFRSF13B), TNFRSF13C (TNFRSF13C), TNFRSF14 (TNFRSF14), Nerve growth factor receptor (NGFR, TNFRSF16), TNFRSF17 (TNFRSF17), TNFRSF18 (TNFRSF18), TNFRSF19 (TNFRSF19), TNFRSF21 (TNFRSF21), and TNFRSF25 (TNFRSF25). In an embodiment, the TNFR family member is CD120a (TNFRSF1A) or TNF-R1. In another embodiment, the TNFR family member is CD 120b (TNFRSFIB) or TNF-R2.

In various embodiments, the receptor for the signaling agent is a TGF-beta receptor. TGF-beta receptors are single pass serine/threonine kinase receptors. TGF-beta receptors include, but are not limited to, TGFBR1, TGFBR2, and TGFBR3.

In various embodiments, the receptor for the signaling agent is an Ig superfamily receptor. Receptors in the immunoglobulin (Ig) superfamily share structural homology with immunoglobulins. Receptors in the Ig superfamily include, but are not limited to, interleukin-1 receptors, CSF-1R, PDGFR (e.g. PDGFRA and PDGFRB), and SCFR.

In various embodiments, the receptor for the signaling agent is a tyrosine kinase superfamily receptor. Receptors in the tyrosine kinase superfamily are well known in the art. There are about 58 known receptor tyrosine kinases (RTKs), grouped into 20 subfamilies. Receptors in the tyrosine kinase superfamily include, but are not limited to, FGF receptors and their various isoforms such as FGFR1, FGFR2, FGFR3, FGFR4, and FGFR5.

In embodiments, the interferon is a type I interferon. In embodiments, the type I interferon is selected from IFN-α2, IFN-α1, IFN-β, IFN-γ, Consensus IFN, IFN-ε, IFN-κ, IFN-τ, IFN-δ, and IFN-v.

In some embodiments, the signaling agent is a wild type interferon α or a modified interferon α. In embodiments, the modified IFN-α agent has reduced affinity and/or activity for the IFN-α/β receptor (IFNAR), i.e., IFNAR1 and/or IFNAR2 chains. In some embodiments, the modified IFN-α agent has substantially reduced or ablated affinity and/or activity for the IFN-α/β receptor (IFNAR), i.e., IFNAR1 and/or IFNAR2 chains.

Mutant forms of interferon α2 are known to the person skilled in the art. In an illustrative embodiment, the modified signaling agent is the allelic form IFN-α2a having the amino acid sequence of:

IFN-α2a:
(SEQ ID NO: 1)
CDLPQTHSLGSRRTLMLLAQMRKISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE.

In an illustrative embodiment, the modified signaling agent is the allelic form IFN-α2b having the amino acid sequence of (which differs from IFN-α2a at amino acid position 23):

IFN-α2b:
(SEQ ID NO: 2)
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE.

In some embodiments, said IFN-α2 mutant (IFN-α2a or IFN-α2b) is mutated at one or more amino acids at positions 144-154, such as amino acid positions 148, 149 and/or 153. In some embodiments, the IFN-α2 mutant comprises one or more mutations selected from L153A, R149A, and M148A. Such mutants are described, for example, in WO2013/107791 and Piehler et al., (2000) J. Biol. Chem, 275:40425-33, the entire contents of all of which are hereby incorporated by reference.

In some embodiments, the IFN-α2 mutants have reduced affinity and/or activity for IFNAR1. In some embodiments, the IFN-α2 mutant comprises one or more mutations selected from F64A, N65A, T69A, L80A, Y85A, and Y89A, as described in WO2010/030671, the entire contents of which is hereby incorporated by reference.

In some embodiments, the IFN-α2 mutant comprises one or more mutations selected from K133A, R144A, R149A, and L153A as described in WO2008/124086, the entire contents of which is hereby incorporated by reference.

In some embodiments, the IFN-α2 mutant comprises one or more mutations selected from R120E and R120E/K121E, as described in WO2015/007520 and WO2010/030671, the entire contents of which are hereby incorporated by reference. In such embodiments, said IFN-α2 mutant antagonizes wildtype IFN-α2 activity. In such embodiments, said mutant IFN-α2 has reduced affinity and/or activity for IFNAR1 while affinity and/or activity of IFNR2 is retained.

In some embodiments, the human IFN-α2 mutant comprises (1) one or more mutations selected from R120E and R120E/K121E, which, without wishing to be bound by theory, create an antagonistic effect and (2) one or more mutations selected from K133A, R144A, R149A, and L153A, which, without wishing to be bound by theory, allow for an attenuated effect at, for example, IFNAR2. In an embodiment, the human IFN-α2 mutant comprises R120E and L153A.

In some embodiments, the human IFN-α2 mutant comprises one or more mutations selected from, L15A, A19W, R22A, R23A, S25A, L26A, F27A, L30A, L30V, K31A, D32A, R33K, R33A, R33Q, H34A, D35A, Q40A, D114R, L117A, R120A, R120E, R125A, R125E, K131A, E132A, K133A, K134A, R144A, A145G, A145M, M148A, R149A, S152A, L153A, and N156A as disclosed in WO 2013/059885 and WO 2016/065409, the entire disclosures of which are hereby incorporated by reference. In some embodiments, the human IFN-α2 mutant comprises the mutations H57Y, E58N, Q61S, and/or L30A as disclosed in WO 2013/059885. In some embodiments, the human IFN-α2 mutant comprises the mutations H57Y, E58N, Q61S, and/or R33A as disclosed in WO 2013/059885. In some embodiments, the human IFN-α2 mutant comprises the mutations H57Y, E58N, Q61S, and/or M148A as disclosed in WO 2013/059885. In some embodiments, the human IFN-α2 mutant comprises the mutations H57Y, E58N, Q61S, and/or L153A as disclosed in WO 2013/059885. In some embodiments, the human IFN-α2 mutant comprises the mutations N65A, L80A, Y85A, and/or Y89A as disclosed in WO 2013/059885. In some embodiments, the human IFN-α2 mutant comprises the mutations N65A, L80A, Y85A, Y89A, and/or D114A as disclosed in WO 2013/059885. In some embodiments, the human IFN-α2 mutant comprises one or more mutations selected from R144X₁, A145X₂, R33A and T106X₃, wherein X₁ is selected from A, S, T, Y, L, and I, and wherein X₂ is selected from G, H, Y, K, and D and wherein X₃ is selected from A and E.

In some embodiments, the human IFN-α2 mutant comprises one or more mutations at one of positions R33, R144, A145, M148, and L153. In some embodiments, the human IFN-α2 mutant comprises one or more mutations selected from R33A, R144A, R144I, R144L, R144S, R144T, R144Y, A145D, A145G, A145H, A145K, A145Y, M148A, and L153A.

In some embodiments, the human IFN-α2 mutant comprises one or more mutations selected from L15A, R22A, R23A, S25A, L26A, F27A, L30A, L30V, K31A, D32A, R33A, R33K, R33Q, H34A, Q40A, D113R, L116A, R119A, R119E, R124A, R124E, K130A, E131A, K132A, K133A, M147A, R148A, S 149A, L152A, N155A, (L30A, H57Y, E58N and Q61S), (M147A, H57Y, E58N and Q61S), (L152A, H57Y, E58N and Q61S), (R143A, H57Y, E58N and Q61S), (N65A, L80A, Y85A and Y89A,) (N65A, L80A, Y85A, Y89A and D113A), (N65A, L80A, Y85A, Y89A and L116A), (N65A, L80A, Y85A, Y89A and RI 190A), (Y85A, Y89A and D113A), (D113A and RI119A), (L116A and R119A), (L116A, R119A and K120A), (R119A and K120A), (R119E and K120E), replacement of R at position 143 with A, D, E, G, H, I, K, L, N, Q, S, T, V or Y, replacement of A at position 144 with D, E, G, H, I, K, L, M, N, Q, S, T, V or Y, and deletion of residues L160 to E164.

In some embodiments, the human IFN-α2 mutant comprises a mutation which does not permit O-linked glycosylation at a position when, e.g., produced in mammalian cell culture. In some embodiments, the human IFN-α2 mutant comprises a mutation at T106. In some embodiments, T106 is substituted with A, C, D, E, F, G, H, I, K, L, M, N, P, Q, R, S, V, W, or Y.

In some embodiments, the human IFN-α2 mutant is a mutant of the IFN-α2-1b variant. Mutations in the IFN-α2-1b variant are disclosed in WO 2015/168474, the entire disclosures of which are hereby incorporated by reference. By way of example, in some embodiments IFN-α2-1b comprises one or more of the following mutations: H58A, E59A, R145A, M149A, and R150A.

In some embodiments, the signaling agent is a wild type interferon α1 or a modified interferon α1. In some embodiments, the present invention provides a chimeric protein that includes a wild type IFNα1. In various embodiments, the wild-type IFNα1 comprises the following amino acid sequence:

(SEQ ID NO: 1562)
CDLPETHSLDNRRTLMLLAQMSRISPSSCLMDRHDFGFPQEEFDGNQFQK
APAISVLHELIQQIFNLFTTKDSSAAWDEDLLDKFCTELYQQLNDLEACV
MQEERVGETPLMNADSILAVKKYFRRITLYLTEKKYSPCAWEVVRAEIMR
SLSLSTNLQERLRRKE.

In various embodiments, the chimeric protein of the invention comprises a modified version of IFNα1, i.e., an IFNα1 variant including a IFNα1 mutant, as a signaling agent. In various embodiments, the IFNα1 variant encompasses mutants, functional derivatives, analogs, precursors, isoforms, splice variants, or fragments of the interferon.

In some embodiments, the IFNα1 interferon is modified to have a mutation at one or more amino acids at positions L15, A19, R23, S25, L30, D32, R33, H34, Q40, C86, D115, L118, K121, R126, E133, K134, K135, R145, A146, M149, R150, S153, L154, and N157 with reference to SEQ ID NO: 1562. The mutations can optionally be a hydrophobic mutation and can be, e.g., selected from alanine, valine, leucine, and isoleucine. In some embodiments, the IFNα1 interferon is modified to have a one or more mutations selected from L15A, A19W, R23A, S25A, L30A, L30V, D32A, R33K, R33A, R33Q, H34A, Q40A, C86S, C86A, D115R, L118A, K121A, K121E, R126A, R126E, E133A, K134A, K135A, R145A, R145D, R145E, R145G, R145H, R1451, R145K, R145L, R145N, R145Q, R145S, R145T, R145V, R145Y, A146D, A146E, A146G, A146H, A1461, A146K, A146L, A146M, A146N, A146Q, A146R, A146S, A146T, A146V, A146Y, M149A, R150A, S153A, L154A, and N157A with reference to SEQ ID NO: 1562. In some embodiments, the IFNα1 mutant comprises one or more multiple mutations selected from L30A/H58Y/E59N_Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121A/K122A, R121A/K122A, and R121E/K122E with reference to SEQ ID NO: 1562).

In an embodiment, the IFNα1 interferon is modified to have a mutation at amino acid position C86 with reference to SEQ ID NO: 1562. The mutation at position C86 can be, e.g., C86S or C86A. These C86 mutants of IFNα1 are called reduced cysteine based aggregation mutants.

In some embodiments, the signaling agent is a wild type interferon β or modified interferon β. In such embodiments, the modified interferon β agent has reduced affinity and/or activity for the IFN-α/β receptor (IFNAR), i.e., IFNAR1 and/or IFNAR2 chains. In some embodiments, the modified interferon β agent has substantially reduced or ablated affinity and/or activity for the IFN-α/β receptor (IFNAR), i.e., IFNAR1 and/or IFNAR2 chains.

In an illustrative embodiment, the modified signaling agent is IFN-β. In various embodiments, the IFN-β encompasses functional derivatives, analogs, precursors, isoforms, splice variants, or fragments of IFN-β. In various embodiments, the IFN-β encompasses IFN-β derived from any species. In an embodiment, the Fc-based chimeric protein complex comprises a modified version of mouse IFN-β. In another embodiment, the Fc-based chimeric protein complex comprises a modified version of human IFN-β. Human IFN-β is a polypeptide with a molecular weight of about 22 kDa comprising 166 amino acid residues. The amino acid sequence of human IFN-β is:

(SEQ ID NO: 3)
MSYNLLGFLQRSSNFQCQKLLWQLNGRLEYCLKDRMNFDIPEEIKQLQQF
QKEDAALTIYEMLQNIFAIFRQDSSSTGWNETIVENLLANVYHQINHLKT
VLEEKLEKEDFTRGKLMSSLHLKRYYGRILHYLKAKEYSHCAWTIVRVEI
LRNFYFINRLTGYLRN.

In some embodiments, the human IFN-β is IFN-β-1a that is a glycosylated form of human IFN-β. In some embodiments, the human IFN-β is IFN-β-1b that is a non-glycosylated form of human IFN-β that has a Met-1 deletion and a Cys-17 to Ser mutation.

In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, I95, N96, K123, and R124. In some embodiments, the one or more mutations are substitutions selected from F67G, F67S, R71A, L88G, L88S, Y92G, Y92S, I95A, N96G, K123G, and R124G. In an embodiment, the modified IFN-β comprises the F67G mutation. In an embodiment, the modified IFN-β comprises the K123G mutation. In an embodiment, the modified IFN-β comprises the F67G and R71A mutations. In an embodiment, the modified IFN-β comprises the L88G and Y92G mutations. In an embodiment, the modified IFN-β comprises the Y92G, I95A, and N96G mutations. In an embodiment, the modified IFN-β comprises the K123G and R124G mutations. In an embodiment, the modified IFN-β comprises the F67G, L88G, and Y92G mutations. In an embodiment, the modified IFN-β comprises the F67S, L88S, and Y92S mutations.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G.

In an embodiment, the modified IFN-β comprises the W22G mutation. In an embodiment, the modified IFN-β comprises the L32A mutation. In an embodiment, the modified IFN-β comprises the L32G mutation. In an embodiment, the modified IFN-β comprises the R35A mutation. In an embodiment, the modified IFN-β comprises the R35G mutation. In an embodiment, the modified IFN-β comprises the V148G mutation. In an embodiment, the modified IFN-β comprises the R152A mutation. In an embodiment, the modified IFN-β comprises the R152G mutation. In an embodiment, the modified IFN-β comprises the Y155G mutation. In an embodiment, the modified IFN-β comprises the W22G and R27G mutations. In an embodiment, the modified IFN-β comprises the L32A and R35A mutation. In an embodiment, the modified IFN-β comprises the L151G and R152A mutations. In an embodiment, the modified IFN-β comprises the V148G and R152A mutations.

In some embodiments, the modified IFN-β has one or more of the following mutations: R35A, R35T, E42K, M621, G78S, A141Y, A142T, E149K, and R152H. In some embodiments, the modified IFN-β has one or more of the following mutations: R35A, R35T, E42K, M621, G78S, A141Y, A142T, E149K, and R152H in combination with C17S or C17A.

In some embodiments, the modified IFN-β has one or more of the following mutations: R35A, R35T, E42K, M621, G78S, A141Y, A142T, E149K, and R152H in combination with any of the other IFN-β mutations described herein.

The crystal structure of human IFN-β is known and is described in Karpusas et al., (1998) PNAS, 94(22): 11813-11818. Specifically, the structure of human IFN-β has been shown to include five α-helices (i.e., A, B, C, D, and E) and four loop regions that connect these helices (i.e., AB, BC, CD, and DE loops). In various embodiments, the modified IFN-β has one or more mutations in the A, B, C, D, E helices and/or the AB, BC, CD, and DE loops which reduce its binding affinity or activity at a therapeutic receptor such as IFNAR. Exemplary mutations are described in WO2000/023114 and US20150011732, the entire contents of which are hereby incorporated by reference. In an exemplary embodiment, the modified IFN-β is human IFN-β comprising alanine substitutions at amino acid positions 15, 16, 18, 19, 22, and/or 23. In an exemplary embodiment, the modified IFN-β is human IFN-β comprising alanine substitutions at amino acid positions 28-30, 32, and 33. In an exemplary embodiment, the modified IFN-β is human IFN-β comprising alanine substitutions at amino acid positions 36, 37, 39, and 42. In an exemplary embodiment, the modified IFN-β is human IFN-β comprising alanine substitutions at amino acid positions 64 and 67 and a serine substitution at position 68. In an exemplary embodiment, the modified IFN-β is human IFN-β comprising alanine substitutions at amino acid positions 71-73. In an exemplary embodiment, the modified IFN-β is human IFN-β comprising alanine substitutions at amino acid positions 92, 96, 99, and 100. In an exemplary embodiment, the modified IFN-β is human IFN-β comprising alanine substitutions at amino acid positions 128, 130, 131, and 134. In an exemplary embodiment, the modified IFN-β is human IFN-β comprising alanine substitutions at amino acid positions 149, 153, 156, and 159. In some embodiments, the mutant IFNβ comprises SEQ ID NO:3 and a mutation at W22, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at W22, the mutation being an aliphatic hydrophobic residue selected from glycine (G), alanine (A), leucine (L), isoleucine (I), methionine (M), and valine (V) and a mutation at R27, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L32, the mutation being an aliphatic hydrophobic residue selected from glycine (G), alanine (A), isoleucine (I), methionine (M), and valine (V).

In some embodiments, the mutant IFNβ comprises SEQ ID NO: 3 and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L32, the mutation being an aliphatic hydrophobic residue selected from glycine (G), alanine (A), isoleucine (I), methionine (M), and valine (V) and a mutation at R35, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at F67, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at F67, the mutation being an aliphatic hydrophobic residue selected from glycine (G), alanine (A), leucine (L), isoleucine (I), methionine (M), and valine (V) and a mutation at R71, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L88, the mutation being an aliphatic hydrophobic residue selected from glycine (G), alanine (A), isoleucine (I), methionine (M), and valine (V).

In some embodiments, the mutant IFNβ comprises SEQ ID NO: 3 and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at F67, the mutation being an aliphatic hydrophobic residue selected from glycine (G), alanine (A), leucine (L), isoleucine (I), methionine (M), and valine (V) and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L88, the mutation being an aliphatic hydrophobic residue selected from glycine (G), alanine (A), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at I95, the mutation being an aliphatic hydrophobic residue selected from glycine (G), alanine (A), leucine (L), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at N96, the mutation being an aliphatic hydrophobic residue selected from glycine (G), alanine (A), leucine (L), isoleucine (I), methionine (M), and valine (V) and a mutation at Y92, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at Y92, the mutation being an aliphatic hydrophobic residue selected from glycine (G), alanine (A), leucine (L), isoleucine (I), methionine (M), and valine (V) and a mutation at I95, the mutation being an aliphatic hydrophobic residue selected from glycine (G), alanine (A), leucine (L), methionine (M), and valine (V) and a mutation at N96, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at K123, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at K123, the mutation being an aliphatic hydrophobic residue selected from glycine (G), alanine (A), leucine (L), isoleucine (I), methionine (M), and valine (V) and a mutation at R124, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L151, the mutation being an aliphatic hydrophobic residue selected from glycine (G), alanine (A), isoleucine (I), methionine (M), and valine (V).

In some embodiments, the mutant IFNβ comprises SEQ ID NO: 3 and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at L151, the mutation being an aliphatic hydrophobic residue selected from glycine (G), alanine (A), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at V148, the mutation being an aliphatic hydrophobic residue selected from glycine (G), alanine (A), leucine (L), isoleucine (I), and methionine (M).

In some embodiments, the mutant IFNβ comprises SEQ ID NO: 3 and a mutation at V148, the mutation being an aliphatic hydrophobic residue selected from glycine (G), alanine (A), leucine (L), isoleucine (I), methionine (M), and valine (V) and a mutation at R152, the mutation being an aliphatic hydrophobic residue selected from glycine (G), alanine (A), leucine (L), isoleucine (I), methionine (M), and valine (V).

In some embodiments, the mutant IFNβ comprises SEQ ID NO: 3 and a mutation at Y155, the mutation being an aliphatic hydrophobic residue selected from glycine (G), alanine (A), leucine (L), isoleucine (I), methionine (M), and valine (V).

In some embodiments, the present invention relates to an Fc-based chimeric protein complex comprising: (a) a modified IFN-β, having the amino acid sequence of SEQ ID NO: 3 and a mutation at position W22, wherein the mutation is an aliphatic hydrophobic residue; and (b) one or more targeting moieties, said targeting moieties comprising recognition domains which specifically bind to antigens or receptors of interest, the modified IFN-β and the one or more targeting moieties are optionally connected with one or more linkers. In various embodiments the mutation at position W22 is aliphatic hydrophobic residue is selected from G, A, L, I, M, and V. In various embodiments, the mutation at position W22 is G.

Additional exemplary IFNβ mutants are provided in PCT/EP2017/061544, the entire disclosure of which is incorporated by reference herein.

In some embodiments, the signaling agent is a wild type or modified interferon γ. In such embodiments, the modified interferon γ agent has reduced affinity and/or activity for the interferon-gamma receptor (IFNGR), i.e., IFNGR1 and IFNGR2 chains. In some embodiments, the modified interferon γ agent has substantially reduced or ablated affinity and/or activity for the interferon-gamma receptor (IFNGR), i.e., IFNGR1 and/or IFNGR2 chains.

For example, the mutant IFN-γ can include a mutation, by way of non-limiting example, a truncation. In embodiments, the mutant IFN-γ has a truncation at the C-terminus, e.g. of about 5 to about 20 amino acid residues, or of about 16 amino acid residues, or of about 15 amino acid residues, or of about 14 amino acid residues, or of about 7 amino acid residues, or of about 5 amino acid residues. In embodiments, the mutant IFN-γ has one or more mutations at positions Q1, V5, E9, K12, H19, S20, V22, A23, D24, N25, G26, T27, L30, K108, H111, E112, I114, Q115, A118, E119, and K125. In embodiments, the mutant IFN-γ has one or more mutations are substitutions selected from V5E, S20E, V22A, A23G, A23F, D24G, G26Q, H111A, H111D, 1114A, Q115A, and A118G. In embodiments, the mutant IFN-γ comprises the V22A mutation. In embodiments, the mutant IFN-γ comprises the A23G mutation. In embodiments, the mutant IFN-γ comprises the D24G mutation. In embodiments, the mutant IFN-γ comprises the H111A mutation or the H111D mutation. In embodiments, the mutant IFN-γ comprises the 1114A mutation. In embodiments, the mutant IFN-γ comprises the Q115A mutation. In embodiments, the mutant IFN-γ comprises the A118G mutation. In embodiments, the mutant IFN-γ comprises the A23G mutation and the D24G mutation. In embodiments, the mutant IFN-γ comprises the 1114A mutation and the A118G mutation. IFN-γ is shown in SEQ ID NO: 1563 below and all mutations are relative to SEQ ID NO: 1563.

In some embodiments, the wild type or modified signaling agent is a consensus interferon. The consensus interferon is generated by scanning the sequences of several human non-allelic IFN-α subtypes and assigning the most frequently observed amino acid in each corresponding position. The consensus interferon differs from IFN-α2b at 20 out of 166 amino acids (88% homology), and comparison with IFN-β shows identity at over 30% of the amino acid positions. In various embodiments, the consensus interferon comprises the following amino acid sequence:

(SEQ ID NO: 4)
MCDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEAC
VIQEVGVEETPLMNVDSILAVKKYFQRITLYLTEKKYSPCAWEWRAEIMR
SFSLSTNLQERLRRKE.

In some embodiments, the consensus interferon comprises the amino acid sequence of SEQ ID NO: 5, which differs from the amino acid sequence of SEQ ID NO: 4 by one amino acid, i.e., SEQ ID NO: 5 lacks the initial methionine residue of SEQ ID NO: 4:

(SEQ ID NO: 5)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRAEIMR
SFSLSTNLQERLRRKE.

In various embodiments, the consensus interferon comprises a wild type or modified version of the consensus interferon, i.e., a consensus interferon variant, as a signaling agent. In various embodiments, the consensus interferon variant encompasses functional derivatives, analogs, precursors, isoforms, splice variants, or fragments of the consensus interferon.

In an embodiment, the consensus interferon variants are selected form the consensus interferon variants disclosed in U.S. Pat. Nos. 4,695,623, 4,897,471, 5,541,293, and 8,496,921, the entire contents of all of which are hereby incorporated by reference. For example, the consensus interferon variant may comprise the amino acid sequence of IFN-CON₂ or IFN-CON₃ as disclosed in U.S. Pat. Nos. 4,695,623, 4,897,471, and 5,541,293. In an embodiment, the consensus interferon variant comprises the amino acid sequence of IFN-CON₂:

(SEQ ID NO: 6)
CDLPQTHSLGNRRTLMLLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEWRAEIMRS
FSLSTNLQERLRRKE.

In an embodiment, the consensus interferon variant comprises the amino acid sequence of IFN-CON₃:

(SEQ ID NO: 7)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNEDSILAVRKYFQRITLYLTEKKYSPCAWEVVRAEIMR
SFSLSTNLQERLRRKE.

In an embodiment, the consensus interferon variant comprises the amino acid sequence of any one of the variants disclosed in U.S. Pat. No. 8,496,921. For example, the consensus variant may comprise the amino acid sequence of:

(SEQ ID NO: 8)
MCDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEAC
VIQEVGVEETPLMNEDSILAVRKYFQRITLYLTEKKYSPCAWEWRAEIMR
SFSLSTNLQERLRRKE.

In another embodiment, the consensus interferon variant may comprise the amino acid sequence of:

(SEQ ID NO: 9)
MCDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEAC
VIQEVGVEETPLMNEDSILAVRKYFQRITLYLTEKKYSPCAWEVVRAEIM
RSFSLCTNLQERLRRKE.

In some embodiments, the consensus interferon variant may be PEGylated, i.e., comprises a PEG moiety. In an embodiment, the consensus interferon variant may comprise a PEG moiety attached at the S156C position of SEQ ID NO: 9.

In some embodiments, the engineered interferon is a variant of human IFN-α2a, with an insertion of Asp at approximately position 41 in the sequence Glu-Glu-Phe-Gly-Asn-Gln (SEQ ID NO: 10) to yield Glu-Glu-Phe-Asp-Gly-Asn-Gln (SEQ ID NO: 11) (which resulted in a renumbering of the sequence relative to IFN-α2a sequence) and the following mutations of Arg23Lys, Leu26Pro, Glu53Gln, Thr54Ala, Pro56Ser, Asp86Glu, Ile104Thr, Gly106Glu, Thr110Glu, Lys117Asn, Arg125Lys, and Lys136Thr. All embodiments herein that describe consensus interferons apply equally to this engineered interferon

In various embodiments, the consensus interferon variant comprises an amino acid sequence having one or more amino acid mutations. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In various embodiments, the consensus interferon is modified to have one or more mutations. In some embodiments, the mutations allow for the consensus interferon variant to have one or more of attenuated activity such as one or more of reduced binding affinity, reduced endogenous activity, and reduced specific bioactivity relative to unmutated, e.g., the wild type form of the consensus interferon (e.g., the consensus interferon having an amino acid sequence of SEQ ID NO: 4 or 5). For instance, the one or more of attenuated activity such as reduced binding affinity, reduced endogenous activity, and reduced specific bioactivity relative to unmutated, e.g. the wild type form of the consensus interferon, may be at a therapeutic receptor such as IFNAR. Consequentially, in various embodiments, the mutations allow for the consensus interferon variant to have reduced systemic toxicity, reduced side effects, and reduced off-target effects relative to unmutated, e.g. the wild type form of the consensus interferon.

In various embodiments, the consensus interferon is modified to have a mutation that reduces its binding affinity or activity at a therapeutic receptor such as IFNAR. In some embodiments, the activity provided by the consensus interferon is agonism at the therapeutic receptor (e.g. activation of a cellular effect at a site of therapy). For example, the consensus interferon may activate the therapeutic receptor. In such embodiments, the mutation results in the consensus interferon variant to have reduced activating activity at the therapeutic receptor.

In some embodiments, the reduced affinity or activity at the therapeutic receptor is restorable by inclusion in the present complex having one or more of the targeting moieties as described herein. In other embodiments, the reduced affinity or activity at the therapeutic receptor is not substantially restorable by inclusion in the present complex having one or more of the targeting moieties as described herein. In various embodiments, the therapeutic Fc-based chimeric protein complexes of the present invention reduce off-target effects because the consensus interferon variant has mutations that weaken binding affinity or activity at a therapeutic receptor. In various embodiments, this reduces side effects observed with, for example, the wild type consensus interferon. In various embodiments, the consensus interferon variant is substantially inactive en route to the site of therapeutic activity and has its effect substantially on specifically targeted cell types which greatly reduces undesired side effects.

In various embodiments, the consensus interferon variant has one or more mutations that cause the consensus interferon variant to have attenuated or reduced affinity, e.g. binding (e.g. K_{D}) and/or activation (measurable as, for example, K_{A} and/or EC₅₀) for one or more therapeutic receptors. In various embodiments, the reduced affinity at the therapeutic receptor allows for attenuation of activity and/or signaling from the therapeutic receptor.

In various embodiments, the consensus interferon variant has one or more mutations that reduce its binding to or its affinity for the IFNAR1 subunit of IFNAR. In one embodiment, the consensus interferon variant has reduced affinity and/or activity at IFNAR1. In some embodiments, the consensus interferon variant has one or more mutations that reduce its binding to or its affinity for the IFNAR2 subunit of IFNAR. In some embodiments, the consensus interferon variant has one or more mutations that reduce its binding to or its affinity for both IFNAR1 and IFNAR2 subunits.

In some embodiments, the consensus interferon variant has one or more mutations that reduce its binding to or its affinity for IFNAR1 and one or more mutations that substantially reduce or ablate binding to or its affinity for IFNAR2. In some embodiments, Fc-based chimeric protein complexes with such consensus interferon variant can provide target-selective IFNAR1 activity (e.g. IFNAR1 activity is restorable via targeting through the targeting moiety, e.g., SIRPα).

In some embodiments, the consensus interferon variant has one or more mutations that reduce its binding to or its affinity for IFNAR2 and one or more mutations that substantially reduce or ablate binding to or its affinity for IFNAR1. In some embodiments, Fc-based chimeric protein complexes with such consensus interferon variant can provide target-selective IFNAR2 activity (e.g. IFNAR2 activity is restorable via targeting through the targeting moiety, e.g., SIRPα).

In some embodiments, the consensus interferon variant has one or more mutations that reduce its binding to or its affinity for IFNAR1 and one or more mutations that reduce its binding to or its affinity for IFNAR2. In some embodiments, Fc-based chimeric protein complexes with such consensus interferon variant can provide target-selective IFNAR1 and/or IFNAR2 activity (e.g. IFNAR1 and/IFNAR2 activity is restorable via targeting through the targeting moiety, e.g., SIRPα).

In some embodiments, the consensus interferon is modified to have a mutation at one or more amino acids at positions 145-155, such as amino acid positions 149, 150 and/or 154, with reference to SEQ ID NO:5. In some embodiments, the consensus interferon is modified to have a mutation at one or more amino acids at positions 145-155, such as amino acid positions 149, 150 and/or 154, with reference to SEQ ID NO: 5, the substitutions optionally being hydrophobic and selected from alanine, valine, leucine, and isoleucine. In some embodiments, the consensus interferon mutant comprises one or more mutations selected from M149A, R150A, and L154A, and, with reference to SEQ ID NO: 5.

In an embodiment, the consensus interferon is modified to have a mutation at amino acid position 121 (i.e., K121), with reference to SEQ ID NO: 5. In an embodiment, the consensus interferon comprises a K121E mutation, with reference to SEQ ID NO: 5.

In various embodiments, the wild type or modified signaling agent is selected from a wild type or modified versions of cytokines, growth factors, and hormones. Illustrative examples of such cytokines, growth factors, and hormones include, but are not limited to, lymphokines, monokines, traditional polypeptide hormones, such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and tumor necrosis factor-β; mullerian-inhibiting substance; mouse gonadotropin-associated peptide; inhibin; activin; vascular endothelial growth factor; integrin; thrombopoietin (TPO); nerve growth factors such as NGF-α; platelet-growth factor; transforming growth factors (TGFs) such as TGF-α and TGF-β; insulin-like growth factor-I and -II; osteo inductive factors; interferons such as, for example, interferon-α, interferon-β and interferon-γ (and interferon type I, II, and III), colony stimulating factors (CSFs) such as macrophage-CSF (M-CSF); granulocyte-macrophage-CSF (GM-CSF); and granulocyte-CSF (G-CSF); interleukins (ILs) such as, for example, IL-1β, IL-1α, IL-2, IL-3, IL-4, IL-5, IL-6, IL-7, IL-8, IL-9, IL-10, IL-11, IL-12, IL-13, IL-15, and IL-18; a tumor necrosis factor such as, for example, TNF-α or TNF-β; and other polypeptide factors including, for example, LIF and kit ligand (KL). As used herein, cytokines, growth factors, and hormones include proteins obtained from natural sources or produced from recombinant bacterial, eukaryotic or mammalian cell culture systems and biologically active equivalents of the native sequence cytokines.

In some embodiments, the signaling agent is a wild type or modified version of a growth factor selected from, but not limited to, transforming growth factors (TGFs) such as TGF-α and TGF-β (and subtypes thereof including the various subtypes of TGF-β including TGFβ1, TGFβ2, and TGFβ3), epidermal growth factor (EGF), insulin-like growth factor such as insulin-like growth factor-I and -II, fibroblast growth factor (FGF), heregulin, platelet-derived growth factor (PDGF), vascular endothelial growth factor (VEGF).

In an embodiment, the growth factor is a modified version of a fibroblast growth factor (FGF). Illustrative FGFs include, but are not limited to, FGF1, FGF2, FGF3, FGF4, FGF5, FGF6, FGF7, FGF8, FGF9, FGF10, FGF11, FGF12, FGF13, FGF14, murine FGF15, FGF16, FGF17, FGF18, FGF19, FGF20, FGF21, FGF22, and FGF23.

In some embodiments, the wild type or modified signaling agent is vascular endothelial growth factor (VEGF). VEGF is a potent growth factor that plays major roles in physiological but also pathological angiogenesis, regulates vascular permeability and can act as a growth factor on cells expressing VEGF receptors. Additional functions include, among others, stimulation of cell migration in macrophage lineage and endothelial cells. Several members of the VEGF family of growth factors exist, as well as at least three receptors (VEGFR-1, VEGFR-2, and VEGFR-3). Members of the VEGF family can bind and activate more than one VEGFR type. For example, VEGF-A binds VEGFR-1 and -2, while VEGF-C can bind VEGFR-2 and -3. VEGFR-1 and -2 activation regulates angiogenesis while VEGFR-3 activation is associated with lymphangiogenesis. The major pro-angiogenic signal is generated from activation of VEGFR-2. VEGFR-1 activation has been reported to be possibly associated with negative role in angiogenesis. It has also been reported that VEGFR-1 signaling is important for progression of tumors in vivo via bone marrow-derived VEGFR-1 positive cells (contributing to formation of premetastatic niche in the bone). Several therapies based on VEGF-A directed/neutralizing therapeutic antibodies have been developed, primarily for use in treatment of various human tumors relying on angiogenesis. These are not without side effects though. This may not be surprising considering that these operate as general, non-cell/tissue specific VEGF/VEGFR interaction inhibitors. Hence, it would be desirable to restrict VEGF (e.g. VEGF-A)/VEGFR-2 inhibition to specific target cells (e.g. tumor vasculature endothelial cells).

In some embodiments, the VEGF is VEGF-A, VEGF-B, VEGF-C, VEGF-D, or VEGF-E and isoforms thereof including the various isoforms of VEGF-A such as VEGF₁₂₁, VEGF₁₂₁b, VEGF₁₄₅, VEGF₁₆₅, VEGF₁₆₅b, VEGF₁₈₉, and VEGF₂₀₆. In some embodiments, the modified signaling agent has reduced affinity and/or activity for VEGFR-1 (Flt-1) and/or VEGFR-2 (KDR/Flk-1). In some embodiments, the modified signaling agent has substantially reduced or ablated affinity and/or activity for VEGFR-1 (Flt-1) and/or VEGFR-2 (KDR/Flk-1). In an embodiment, the modified signaling agent has reduced affinity and/or activity for VEGFR-2 (KDR/Flk-1) and/or substantially reduced or ablated affinity and/or activity for VEGFR-1 (Flt-1). Such an embodiment finds use, for example, in wound healing methods or treatment of ischemia-related diseases (without wishing to be bound by theory, mediated by VEGFR-2's effects on endothelial cell function and angiogenesis). In various embodiments, binding to VEGFR-1 (Flt-1), which is linked to cancers and pro-inflammatory activities, is avoided. In various embodiments, VEGFR-1 (Flt-1) acts a decoy receptor and therefore substantially reduces or ablates affinity at this receptor avoids sequestration of the therapeutic agent. In an embodiment, the modified signaling agent has substantially reduced or ablated affinity and/or activity for VEGFR-1 (Flt-1) and/or substantially reduced or ablated affinity and/or activity for VEGFR-2 (KDR/Flk-1). In some embodiments, the VEGF is VEGF-C or VEGF-D. In such embodiments, the modified signaling agent has reduced affinity and/or activity for VEGFR-3. Alternatively, the modified signaling agent has substantially reduced or ablated affinity and/or activity for VEGFR-3.

Proangiogenic therapies are also important in various diseases (e.g. ischemic heart disease, bleeding etc.), and include VEGF-based therapeutics. Activation of VEGFR-2 is proangiogenic (acting on endothelial cells). Activation of VEFGR-1 can cause stimulation of migration of inflammatory cells (including, for example, macrophages) and lead to inflammation associated hypervascular permeability. Activation of VEFGR-1 can also promote bone marrow associated tumor niche formation. Thus, VEGF based therapeutic selective for VEGFR-2 activation would be desirable in this case. In addition, cell specific targeting, e.g. to endothelial cells, would be desirable.

In some embodiments, the modified signaling agent has reduced affinity and/or activity (e.g. antagonistic) for VEGFR-2 and/or has substantially reduced or ablated affinity and/or activity for VEGFR-1. When targeted to tumor vasculature endothelial cells via a targeting moiety that binds to a tumor endothelial cell marker (e.g. PSMA and others), such construct inhibits VEGFR-2 activation specifically on such marker-positive cells, while not activating VEGFR-1 en route and on target cells (if activity ablated), thus eliminating induction of inflammatory responses, for example. This would provide a more selective and safe anti-angiogenic therapy for many tumor types as compared to VEGF-A neutralizing therapies.

In some embodiments, the modified signaling agent has reduced affinity and/or activity (e.g. agonistic) for VEGFR-2 and/or has substantially reduced or ablated affinity and/or activity for VEGFR-1. Through targeting to vascular endothelial cells, such construct, in some embodiments, promotes angiogenesis without causing VEGFR-1 associated induction of inflammatory responses. Hence, such a construct would have targeted proangiogenic effects with substantially reduced risk of side effects caused by systemic activation of VEGFR-2 as well as VEGR-1.

In an illustrative embodiment, the modified signaling agent is a modified VEGF₁₆₅, the wild type amino acid sequence of VEGF₁₆₅ is:

(SEQ ID NO: 12)
APMAEGGGQNHHEVVKFMDVYQRSYCHPIETLVDIFQEYPDEIEYIFKPS
CVPLMRCGGCCNDEGLECVPTEESNITMQIMRIKPHQGQHIGEMSFLQHN
KCECRPKKDRARQENPCGPCSERRKHLFVQDPQTCKCSCKNTDSRCKARQ
LELNERTCRCDKPRR.

In another illustrative embodiment, the modified signaling agent is a modified VEGF₁₆₅b, the wild type amino acid sequence of VEGF₁₆₅b is:

(SEQ ID NO: 13)
APMAEGGGQNHHEVVKFMDVYQRSYCHPIETLVDIFQEYPDEIEYIFKPS
CVPLMRCGGCCNDEGLECVPTEESNITMQIMRIKPHQGQHIGEMSFLQHN
KCECRPKKDRARQENPCGPCSERRKHLFVQDPQTCKCSCKNTDSRCKARQ
LELNERTCRSLTRKD.

In these embodiments, the modified signaling agent has a mutation at amino acid 183 (e.g., a substitution mutation at I83, e.g., I83K, I83R, or I83H). Without wishing to be bound by theory, it is believed that such mutations may result in reduced receptor binding affinity. See, for example, U.S. Pat. No. 9,078,860, the entire contents of which are hereby incorporated by reference.

In some embodiments, the signaling agent is a wild type or modified version of a hormone selected from, but not limited to, human chorionic gonadotropin, gonadotropin releasing hormone, an androgen, an estrogen, thyroid-stimulating hormone, follicle-stimulating hormone, luteinizing hormone, prolactin, growth hormone, adrenocorticotropic hormone, antidiuretic hormone, oxytocin, thyrotropin-releasing hormone, growth hormone releasing hormone, corticotropin-releasing hormone, somatostatin, dopamine, melatonin, thyroxine, calcitonin, parathyroid hormone, glucocorticoids, mineralocorticoids, adrenaline, noradrenaline, progesterone, insulin, glucagon, amylin, calcitriol, calciferol, atrial-natriuretic peptide, gastrin, secretin, cholecystokinin, neuropeptide Y, ghrelin, PYY3-36, insulin-like growth factor (IGF), leptin, thrombopoietin, erythropoietin (EPO), and angiotensinogen.

In some embodiments, the wild type or modified signaling agent is TNF-α. TNF is a pleiotropic cytokine with many diverse functions, including regulation of cell growth, differentiation, apoptosis, tumorigenesis, viral replication, autoimmunity, immune cell functions and trafficking, inflammation, and septic shock. It binds to two distinct membrane receptors on target cells: TNFR1 (p55) and TNFR2 (p75). TNFR1 exhibits a very broad expression pattern whereas TNFR2 is expressed preferentially on certain populations of lymphocytes, Tregs, endothelial cells, certain neurons, microglia, cardiac myocytes and mesenchymal stem cells. Very distinct biological pathways are activated in response to receptor activation, although there is also some overlap. As a general rule, without wishing to be bound by theory, TNFR1 signaling is associated with induction of apoptosis (cell death) and TNFR2 signaling is associated with activation of cell survival signals (e.g. activation of NFkB pathway). Administration of TNF is systemically toxic, and this is largely due to TNFR1 engagement. However, it should be noted that activation of TNFR2 is also associated with a broad range of activities and, as with TNFR1, in the context of developing TNF based therapeutics, control over TNF targeting and activity is important.

In some embodiments, the modified signaling agent has reduced affinity and/or activity for TNFR1 and/or TNFR2. In some embodiments, the modified signaling agent has substantially reduced or ablated affinity and/or activity for TNFR1 and/or TNFR2. TNFR1 is expressed in most tissues, and is involved in cell death signaling while, by contrast, TNFR2 is involved in cell survival signaling. Accordingly, in embodiments directed to methods of treating cancer, the modified signaling agent has reduced affinity and/or activity for TNFR1 and/or substantially reduced or ablated affinity and/or activity for TNFR2. In these embodiments, the Fc-based chimeric protein complexes may be targeted to a cell for which apoptosis is desired, e.g. a tumor cell or a tumor vasculature endothelial cell. In embodiments directed to methods of promoting cell survival, for example, in neurogenesis for the treatment of neurodegenerative disorders, the modified signaling agent has reduced affinity and/or activity for TNFR2 and/or substantially reduced or ablated affinity and/or activity for TNFR1. Stated another way, the present Fc-based chimeric protein complexes, in some embodiments, comprise modified TNF-α agent that allows of favoring either death or survival signals.

In some embodiments, the Fc-based chimeric protein complex has a modified TNF having reduced affinity and/or activity for TNFR1 and/or substantially reduced or ablated affinity and/or activity for TNFR2. Such an Fc-based chimeric protein complex, in some embodiments, is a more potent inducer of apoptosis as compared to a wild type TNF and/or an Fc-based chimeric protein complex bearing only mutation(s) causing reduced affinity and/or activity for TNFR1. Such an Fc-based chimeric protein complex, in some embodiments, finds use in inducing tumor cell death or a tumor vasculature endothelial cell death (e.g. in the treatment of cancers). Also, in some embodiments, these Fc-based chimeric protein complexes avoid or reduce activation of T_{reg} cells via TNFR2, for example, thus further supporting TNFR1-mediated antitumor activity in vivo.

In some embodiments, the Fc-based chimeric protein complex has a modified TNF having reduced affinity and/or activity for TNFR2 and/or substantially reduced or ablated affinity and/or activity for TNFR1. Such an Fc-based chimeric protein complex, in some embodiments, is a more potent activator of cell survival in some cell types, which may be a specific therapeutic objective in various disease settings, including without limitation, stimulation of neurogenesis. In addition, such a TNFR2-favoring Fc-based chimeric protein complexes also are useful in the treatment of autoimmune diseases (e.g. Crohn's, diabetes, MS, colitis etc. and many others described herein). In some embodiments, the Fc-based chimeric protein complex is targeted to auto-reactive T cells. In some embodiments, the Fc-based chimeric protein complex promotes T_{reg} cell activation and indirect suppression of cytotoxic T cells.

In some embodiments, the Fc-based chimeric protein complex causes the death of auto-reactive T cells, e.g. by activation of TNFR2 and/or avoidance TNFR1 (e.g. a modified TNF having reduced affinity and/or activity for TNFR2 and/or substantially reduced or ablated affinity and/or activity for TNFR1). Without wishing to be bound by theory these auto-reactive T cells, have their apoptosis/survival signals altered e.g. by NFkB pathway activity/signaling alterations. In some embodiments, the Fc-based chimeric protein complex causes the death of autoreactive T cells having lesions or modifications in the NFκB pathway, which underlie an imbalance of their cell death (apoptosis)/survival signaling properties and, optionally, altered susceptibility to certain death-inducing signals (e.g., TNFR2 activation).

In some embodiments, a TNFR-2 based Fc-based chimeric protein complex has additional therapeutic applications in diseases, including autoimmune disease, various heart disease, de-myelinating and neurodegenerative disorders, and infectious disease, among others.

In an embodiment, the wild type TNF-α has the amino acid sequence of:

(SEQ ID NO: 14)
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAEAKPWYEPIYLGGVFQLEKGDRLSAEINRPDYLDFAESGQV
YFGIIAL.

In such embodiments, the modified TNF-α agent has mutations at one or more amino acid positions 29, 31, 32, 84, 85, 86, 87, 88, 89, 145, 146 and 147 which produces a modified TNF-α with reduced receptor binding affinity. See, for example, U.S. Pat. No. 7,993,636, the entire contents of which are hereby incorporated by reference.

In some embodiments, the modified human TNF-α moiety has mutations at one or more amino acid positions R32, N34, Q67, H73, L75, T77, S86, Y87, V91, 197, T105, P106, A109, P113, Y115, E127, N137, D143, A145, and E146 as described, for example, in WO/2015/007903, the entire contents of which is hereby incorporated by reference (numbering according to the human TNF sequence, Genbank accession number BAG70306, version BAG70306.1 GI: 197692685). In some embodiments, the modified human TNF-α moiety has substitution mutations selected from L29S, R32G, R32W, N34G, Q67G, H73G, L75G, L75A, L75S, T77A, S86G, S86T, Y87Q, Y87L, Y87A, Y87F, Y87H, V91G, V91A, 197A, 197Q, 197S, T105G, P106G, A109Y, P113G, Y115G, Y115A, E127G, N137G, D143N, A145G, A145R, A145T, E146D, E146K, and S147D. In some embodiments, the human TNF-α moiety has a mutation selected from Y87Q, Y87L, Y87A, Y87F, and Y87H. In another embodiment, the human TNF-α moiety has a mutation selected from 197A, 197Q, and 197S. In a further embodiment, the human TNF-α moiety has a mutation selected from Y115A and Y115G. In some embodiments, the human TNF-α moiety has an E146K mutation. In some embodiments, the human TNF-α moiety has an Y87H and an E146K mutation. In some embodiments, the human TNF-α moiety has an Y87H and an A145R mutation. In some embodiments, the human TNF-α moiety has a R32W and a S86T mutation. In some embodiments, the human TNF-α moiety has a R32W and an E146K mutation. In some embodiments, the human TNF-α moiety has a L29S and a R32W mutation. In some embodiments, the human TNF-α moiety has a D143N and an A145R mutation. In some embodiments, the human TNF-α moiety has a D143N and an A145R mutation. In some embodiments, the human TNF-α moiety has an A145T, an E146D, and a S147D mutation. In some embodiments, the human TNF-α moiety has an A145T and a S147D mutation.

In some embodiments, the modified TNF-α agent has one or more mutations selected from N39Y, S147Y, and Y87H, as described in WO2008/124086, the entire contents of which is hereby incorporated by reference.

In some embodiments, the modified human TNF-α moiety has mutations that provide receptor selectivity as described in PCT/IB2016/001668, the entire contents of which are hereby incorporated by reference. In some embodiments, the mutations to TNF are TNF-R1 selective. In some embodiments, the mutations to TNF which are TNF-R1 selective are at one or more of positions R32, S86, and E146. In some embodiments, the mutations to TNF which are TNF-R1 selective are one or more of R32W, S86T, and E146K. In some embodiments, the mutations to TNF which are TNF-R1 selective are one or more of R32W, R32W/S86T, R32W/E146K and E146K.

In some embodiments, the mutations to TNF are TNF-R2 selective. In some embodiments, the mutations to TNF which are TNF-R2 selective are at one or more of positions A145, E146, and S147. In some embodiments, the mutations to TNF which are TNF-R2 selective are one or more of A145T, A145R, E146D, and S147D. In some embodiments, the mutations to TNF which are TNF-R2 selective are one or more of A145R, A145T/S147D, and A145T/E146D/S147D.

In an embodiment, the wild type or modified signaling agent is TNF-6. TNF-6 can form a homotrimer or a heterotrimer with LT-β (LT-α1β2). In some embodiments, the modified signaling agent has substantially reduced or ablated affinity and/or activity for TNFR1 and/or TNFR2 and/or herpes virus entry mediator (HEVM) and/or LT-6R.

In an embodiment, the wild type TNF-β has the amino acid sequence of:

(SEQ ID NO: 15)
LPGVGLTPSAAQTARQHPKMHLAHSNLKPAAHLIGDPSKQNSLLWRANTD
RAFLQDGFSLSNNSLLVPTSGIYFVYSQWFSGKAYSPKATSSPLYLAHEV
QLFSSQYPFHVPLLSSQKMVYPGLQEPWLHSMYHGAAFQLTQGDQLSTHT
DGIPHLVLSPSTVFFGAFAL.

In such embodiments, the modified TNF-β agent may comprise mutations at one or more amino acids at positions 106-113, which produce a modified TNF-β with reduced receptor binding affinity to TNFR2. In an embodiment, the modified signaling agent has one or more substitution mutations at amino acid positions 106-113. In illustrative embodiments, the substitution mutations are selected from Q107E, Q107D, S106E, S106D, Q107R, Q107N, Q107E/S106E, Q107E/S106D, Q107D/S106E, and Q107D/S106D. In another embodiment, the modified signaling agent has an insertion of about 1 to about 3 amino acids at positions 106-113.

In some embodiments, the wild type or modified agent is a TNF family member (e.g. TNF-alpha, TNF-beta) which can be a single chain trimeric version as described in WO 2015/007903 and PCT/IB2016/001668, the entire contents of which are incorporated by reference.

In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR1. In these embodiments, the modified agent is a TNF family member (e.g. TNF-alpha, TNF-beta) which also, optionally, has substantially reduced or ablated affinity and/or activity for TNFR2. In some embodiments, the modified agent is a TNF family member (e.g. TNF-alpha, TNF-beta) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TNFR2. In these embodiments, the modified agent is a TNF family member (e.g. TNF-alpha, TNF-beta) which also, optionally, has substantially reduced or ablated affinity and/or activity for TNFR1. The constructs of such embodiments find use in, for example, methods of dampening TNF response in a cell specific manner. In some embodiments, the antagonistic TNF family member (e.g. TNF-alpha, TNF-beta) is a single chain trimeric version as described in WO 2015/007903.

In an embodiment, the wild type or modified signaling agent is TRAIL. In some embodiments, the modified TRAIL agent has reduced affinity and/or activity for DR4 (TRAIL-RI) and/or DR5 (TRAIL-RII) and/or DcR1 and/or DcR2.

In some embodiments, the modified TRAIL agent has substantially reduced or ablated affinity and/or activity for DR4 (TRAIL-RI) and/or DR5 (TRAIL-RII) and/or DcR1 and/or DcR2.

In an embodiment, the wild type TRAIL has the amino acid sequence of:

(SEQ ID NO: 16)
MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG.

In such embodiments, the modified TRAIL agent may comprise a mutation at amino acid positions T127-R132, E144-R149, E155-H161, Y189-Y209, T214-1220, K224-A226, W231, E236-L239, E249-K251, T261-H264 and H270-E271 (Numbering based on the human sequence, Genbank accession number NP_003801, version 10 NP_003801.1, GI: 4507593; see above).

such embodiments, the modified TRAIL agent may comprise a truncation, e.g., without limitation, as described in, for example, Trebing et al., (2014) Cell Death and Disease, 5:e1035, the entire disclosure of which is hereby incorporated by reference

In some embodiments, the modified TRAIL agent may comprise one or more mutations that substantially reduce its affinity and/or activity for TRAIL-R1. In such embodiments, the modified TRAIL agent may specifically bind to TRIL-R2. Exemplary mutations include mutations at one or more amino acid positions Y189, R191, Q193, H264, 1266, and D267. For example, the mutations may be one or more of Y189Q, R191K, Q193R, H264R, 1266L and D267Q.

In an embodiment, the modified TRAIL agent comprises the mutations Y189Q, R191K, Q193R, H264R, I266L and D267Q.

In some embodiments, the modified TRAIL agent may comprise one or more mutations that substantially reduce its affinity and/or activity for TRAIL-R2. In such embodiments, the modified TRAIL agent may specifically bind to TRIL-R1. Exemplary mutations include mutations at one or more amino acid positions G131, R149, S159, N199, K201, and S215. For example, the mutations may be one or more of G131R, R1491, S159R, N199R, K201H, and S215D. In an embodiment, the modified TRAIL agent comprises the mutations G131R, R1491, S159R, N199R, K201H, and S215D. Additional TRAIL mutations are described in, for example, Trebing et al., (2014) Cell Death and Disease, 5:e1035, the entire disclosure of which is hereby incorporated by reference.

In an embodiment, the wild type or modified signaling agent is TGFα. In such embodiments, the modified TGFα agent has reduced affinity and/or activity for the epidermal growth factor receptor (EGFR). In some embodiments, the modified TGFα agent has substantially reduced or ablated affinity and/or activity for the epidermal growth factor receptor (EGFR).

In an embodiment, the wild type or modified signaling agent is TGFβ. In such embodiments, the modified signaling agent has reduced affinity and/or activity for TGFBR1 and/or TGFBR2. In some embodiments, the modified signaling agent has substantially reduced or ablated affinity and/or activity for TGFBR1 and/or TGFBR2. In some embodiments, the modified signaling agent optionally has reduced or substantially reduced or ablated affinity and/or activity for TGFBR3 which, without wishing to be bound by theory, may act as a reservoir of ligand for TGF-beta receptors. In some embodiments, the TGFβ may favor TGFBR1 over TGFBR2 or TGFBR2 over TGFBR1. Similarly, LAP, without wishing to be bound by theory, may act as a reservoir of ligand for TGF-beta receptors. In some embodiments, the modified signaling agent has reduced affinity and/or activity for TGFBR1 and/or TGFBR2 and/or substantially reduced or ablated affinity and/or activity for Latency Associated Peptide (LAP). In some embodiments, such Fc-based chimeric protein complexes find use in Camurati-Engelmann disease, or other diseases associated with inappropriate TGβ signaling.

In some embodiments, the wild type or modified agent is a TGF family member (e.g. TGFα, TGβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at one or more of TGFBR1, TGFBR2, TGFBR3. In these embodiments, the modified agent is a TGF family member (e.g. TGFα, TGβ) which also, optionally, has substantially reduced or ablated affinity and/or activity at one or more of TGFBR1, TGFBR2, and TGFBR3.

In some embodiments, the modified agent is a TGF family member (e.g. TGFα, TGβ) which has reduced affinity and/or activity, i.e. antagonistic activity (e.g. natural antagonistic activity or antagonistic activity that is the result of one or more mutations, see, e.g., WO 2015/007520, the entire contents of which are hereby incorporated by reference) at TGFBR1 and/or TGFBR2. In these embodiments, the modified agent is a TGF family member (e.g. TGFα, TGβ) which also, optionally, has substantially reduced or ablated affinity and/or activity at TGFBR3.

In an embodiment, the wild type or modified signaling agent is an interleukin. In an embodiment, the modified signaling agent is IL-1. In an embodiment, the modified signaling agent is IL-1α or IL-1β. In some embodiments, the modified signaling agent has reduced affinity and/or activity for IL-1R1 and/or IL-1RAcP. In some embodiments, the modified signaling agent has substantially reduced or ablated affinity and/or activity for IL-1R1 and/or IL-1RAcP. In some embodiments, the modified signaling agent has reduced affinity and/or activity for IL-1R2. In some embodiments, the modified signaling agent has substantially reduced or ablated affinity and/or activity for IL-1R2.

For instance, in some embodiments, the present modified IL-1 agents avoid interaction at IL-1R2 and therefore substantially reduce its function as a decoy and/or sink for therapeutic agents.

In an embodiment, the wild type IL-1β has the amino acid sequence of:

(SEQ ID NO: 17)
APVRSLNCTLRDSQQKSLVMSGPYELKALHLQGQDMEQQVVFSMSFVQGE
ESNDKIPVALGLKEKNLYLSCVLKDDKPTLQLESVDPKNYPKKKMEKRFV
FNKIElNNKLEFESAQFPNWYISTSQAENMPVFLGGTKGGQDITDFTMQF
VSS.

IL-1β is a proinflammatory cytokine and an important immune system regulator. It is a potent activator of CD4 T cell responses, increases proportion of Th17 cells and expansion of IFNγ and IL-4 producing cells. IL-1β is also a potent regulator of CD8⁺ T cells, enhancing antigen-specific CD8⁺ T cell expansion, differentiation, migration to periphery and memory. IL-1β receptors comprise IL-1R1 and IL-1R2. Binding to and signaling through the IL-1R1 constitutes the mechanism whereby IL-1β mediates many of its biological (and pathological) activities. IL1-R2 can function as a decoy receptor, thereby reducing IL-1β availability for interaction and signaling through the IL-1R1.

In some embodiments, the modified IL-1β has reduced affinity and/or activity (e.g. agonistic activity) for IL-1R1. In some embodiments, the modified IL-1β has substantially reduced or ablated affinity and/or activity for IL-1R2. In such embodiments, there is restorable IL-1β/IL-1R1 signaling and prevention of loss of therapeutic Fc-based chimeric protein complexes at IL-R2 and therefore a reduction in dose of IL-1β that is required (e.g. relative to wild type or an Fc-based chimeric protein complex bearing only an attenuation mutation for IL-R1). Such constructs find use in, for example, methods of treating cancer, including, for example, stimulating the immune system to mount an anti-cancer response.

In some embodiments, the modified IL-1β has reduced affinity and/or activity (e.g. antagonistic activity, e.g. natural antagonistic activity or antagonistic activity that is the result of one or more mutations, see, e.g., WO 2015/007520, the entire contents of which are hereby incorporated by reference) for IL-1R1. In some embodiments, the modified IL-1β has substantially reduced or ablated affinity and/or activity for IL-1R2. In such embodiments, there is the IL-1β/IL-1R1 signaling is not restorable and prevention of loss of therapeutic Fc-based chimeric protein complexes at IL-R2 and therefore a reduction in dose of IL-1β that is required (e.g. relative to wild type or an Fc-based chimeric protein complex bearing only an attenuation mutation for IL-R1). Such constructs find use in, for example, methods of treating autoimmune diseases, including, for example, suppressing the immune system.

In such embodiments, the modified signaling agent has a deletion of amino acids 52-54 which produces a modified human IL-1β with reduced binding affinity for type I IL-1R and reduced biological activity. See, for example, WO 1994/000491, the entire contents of which are hereby incorporated by reference. In some embodiments, the modified human IL-1β has one or more substitution mutations selected from A117G/P118G, R120X, L122A, T125G/L126G, R127G, Q130X, Q131G, K132A, S137G/Q138Y, L145G, H146X, L145A/L147A, Q148X, Q148G/Q150G, Q150G/D151A, M152G, F162A, F162A/Q164E, F166A, Q164E/E167K, N169G/D170G, I172A, V174A, K208E, K209X, K209A/K210A, K219X, E221X, E221 S/N224A, N224S/K225S, E244K, N245Q (where X can be any change in amino acid, e.g., a non-conservative change), which exhibit reduced binding to IL-1R, as described, for example, in WO2015/007542 and WO/2015/007536, the entire contents of which is hereby incorporated by reference (numbering base on the human IL-1 β sequence, Genbank accession number NP_000567, version NP-000567.1, GI: 10835145). In some embodiments, the modified human IL-1β may have one or more mutations selected from R120A, R120G, Q130A, Q130W, H146A, H146G, H146E, H146N, H146R, Q148E, Q148G, Q148L, K209A, K209D, K219S, K219Q, E221S and E221K. In an embodiment, the modified human IL-1β comprises the mutations Q131G and Q148G. In an embodiment, the modified human IL-1β comprises the mutations Q148G and K208E. In an embodiment, the modified human IL-1β comprises the mutations R120G and Q131G. In an embodiment, the modified human IL-1β comprises the mutations R120G and H146A. In an embodiment, the modified human IL-1β comprises the mutations R120G and H146N. In an embodiment, the modified human IL-1β comprises the mutations R120G and H146R. In an embodiment, the modified human IL-1β comprises the mutations R120G and H146E. In an embodiment, the modified human IL-1β comprises the mutations R120G and H146G. In an embodiment, the modified human IL-1β comprises the mutations R120G and K208E. In an embodiment, the modified human IL-1β comprises the mutations R120G, F162A, and Q164E.

In an embodiment, the wild type or modified signaling agent is IL-2. In such an embodiment, the modified signaling agent has reduced affinity and/or activity for IL-2Rα and/or IL-2Rβ and/or IL-2Rγ. In some embodiments, the modified signaling agent has reduced affinity and/or activity for IL-2Rβ and/or IL-2Rγ. In some embodiments, the modified signaling agent has substantially reduced or ablated affinity and/or activity for IL-2Rα. Such embodiments may be relevant for treatment of cancer, for instance when the modified IL-2 is agonistic at IL-2Rβ and/or IL-2Rγ. For instance, the present constructs may favor attenuated activation of CD8⁺ T cells (which can provide an anti-tumor effect), which have IL2 receptors β and γ and disfavor T_{regs} (which can provide an immune suppressive, pro-tumor effect), which have IL2 receptors α, β, and γ. Further, in some embodiments, the preferences for IL-2Rβ and/or IL-2Rγ over IL-2Rα avoid IL-2 side effects such as pulmonary edema. Also, IL-2-based Fc-based chimeric protein complexes are useful for the treatment of diseases (e.g., autoimmune disease), for instance when the modified IL-2 is antagonistic (e.g. natural antagonistic activity or antagonistic activity that is the result of one or more mutations, see, e.g., WO 2015/007520, the entire contents of which are hereby incorporated by reference) at IL-2Rβ and/or IL-2Rγ. For instance, the present constructs may favor attenuated suppression of CH′ T cells (and therefore dampen the immune response), which have IL2 receptors β and γ and disfavor T_{regs} which have IL2 receptors α, β, and γ. Alternatively, in some embodiments, the Fc-based chimeric protein complexes bearing IL-2 favor the activation of T_{regs}, and therefore immune suppression, and activation of disfavor of CD8⁺ T cells. For instance, these constructs find use in the treatment of diseases or diseases that would benefit from immune suppression, e.g., autoimmune disorders.

In some embodiments, the Fc-based chimeric protein complex has targeting moieties as described herein directed to CD8⁺ T cells as well as a modified IL-2 agent having reduced affinity and/or activity for IL-2Rβ and/or IL-2Rγ and/or substantially reduced or ablated affinity and/or activity for IL-2Rα. In some embodiments, these constructs provide targeted CD8⁺ T cell activity and are generally inactive (or have substantially reduced activity) towards T_{reg} cells. In some embodiments, such constructs have enhanced immune stimulatory effect compared to wild type IL-2 (e.g., without wishing to be bound by theory, by not stimulating Tregs), whilst eliminating or reducing the systemic toxicity associated with IL-2.

In an embodiment, the wild type IL-2 has the amino acid sequence of:

(SEQ ID NO: 18)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT.

In such embodiments, the modified IL-2 agent has one or more mutations at amino acids L72 (L72G, L72A, L72S, L72T, L72Q, L72E, L72N, L72D, L72R, or L72K), F42 (F42A, F42G, F42S, F42T, F42Q, F42E, F42N, F42D, F42R, or F42K) and Y45 (Y45A, Y45G, Y45S, Y45T, Y45Q, Y45E, Y45N, Y45D, Y45R or Y45K). Without wishing to be bound by theory, it is believed that these modified IL-2 agents have reduced affinity for the high-affinity IL-2 receptor and preserves affinity to the intermediate-affinity IL-2 receptor, as compared to the wild-type IL-2. See, for example, US Patent Publication No. 2012/0244112, the entire contents of which are hereby incorporated by reference.

In some embodiments, the modified IL-2 agent has one or more mutations at amino acids R38, F42, Y45, and E62. For example, the modified IL-2 agent may comprise one or more of R38A, F42A, Y45A, and E62A. In some embodiments, the modified IL-2 agent may comprise a mutation at C125. For example, the mutation may be C125S. In such embodiments, the modified IL-2 agent may have substantially reduced affinity and/or activity for IL-2Rα, as described in, for example, Carmenate et al. (2013) The Journal of Immunology, 190:6230-6238, the entire disclosure of which is hereby incorporated by reference. In some embodiments, the modified IL-2 agent with mutations at R38, F42, Y45, and/or E62 is able to induce an expansion of effector cells including CD8+ T cells and NK cells but not Treg cells. In some embodiments, the modified IL-2 agent with mutations at R38, F42, Y45, and/or E62 is less toxic than wildtype IL-2 agents. An Fc-based chimeric protein complex comprising the modified IL-2 agent with substantially reduced affinity and/or activity for IL-2Rα may find application in oncology for example.

In some embodiments, the modified IL-2 signaling agent comprises a deletion of the Ala at the N-terminus of SEQ ID NO: 18. In some embodiments, the modified IL-2 agent comprises the substitution of a Ser for the Cys at position 125 of SEQ ID NO: 18. In some embodiments, the modified IL-2 agent comprises a deletion of the Ala at the N-terminus and the substitution of a Ser for the Cys at position 125 of SEQ ID NO: 18.

In other embodiments, the modified IL-2 agent may have substantially reduced affinity and/or activity for IL-2Rβ, as described in, for example, WO2016/025385, the entire disclosure of which is hereby incorporated by reference. In such embodiments, the modified IL-2 agent may induce an expansion of Treg cells but not effector cells such as CD8+ T cells and NK cells. An Fc-based chimeric protein complex comprising the modified IL-2 agent with substantially reduced affnity and/or activity for IL-2Rβ may find application in the treatment of autoimmune disease for example. In some embodiments, the modified IL-2 agent may comprise one or more mutations at amino acids N88, D20, and/or A126. For example, the modified IL-2 agent may comprise one or more of N88R, N88I, N88G, D2OH, Q126L, and Q126F.

In various embodiments, the modified IL-2 agent may comprise a mutation at D109 or C125. For example, the mutation may be D109C or C125S. In some embodiments, the modified IL-2 with a mutation at D109 or C125 may be utilized for attachment to a PEG moiety.

In an embodiment, the wild type or modified signaling agent is IL-3. In some embodiments, the modified signaling agent has reduced affinity and/or activity for the IL-3 receptor, which is a heterodimer with a unique alpha chain paired with the common beta (beta c or CD131) subunit. In some embodiments, the modified signaling agent has substantially reduced or ablated affinity and/or activity for the IL-3 receptor, which is a heterodimer with a unique alpha chain paired with the common beta (beta c or CD131) subunit.

In an embodiment, the wild type or modified signaling agent is IL-4. In such an embodiment, the modified signaling agent has reduced affinity and/or activity for type 1 and/or type 2 IL-4 receptors. In such an embodiment, the modified signaling agent has substantially reduced or ablated affinity and/or activity for type 1 and/or type 2 IL-4 receptors. Type 1 IL-4 receptors are composed of the IL-4Ra subunit with a common γ chain and specifically bind IL-4. Type 2 IL-4 receptors include an IL-4Ra subunit bound to a different subunit known as IL-13Ra1. In some embodiments, the modified signaling agent has substantially reduced or ablated affinity and/or activity the type 2 IL-4 receptors.

In an embodiment, the wild type IL-4 has the amino acid sequence of:

(SEQ ID NO: 19)
HKCIDITLQEIIKTLNSLTEQKTLCTELTVTDIFAASKNTTEKETFCRAA
TVLRQFYSHHEKDTIRCLGATAQQFHRHKQUIRFLKRLDRNLWGLAGLNS
CPVKEANQSTLENFLERLKTIMREKYSKOSS.

In such embodiments, the modified IL-4 agent has one or more mutations at amino acids R121 (R121A, R121D, R121E, R121F, R121H, R1211, R121K, R121N, R121P, R121T, R121W), E122 (E122F), Y124 (Y124A, Y124Q, Y124R, Y124S, Y124T) and S125 (S125A). Without wishing to be bound by theory, it is believed that these modified IL-4 agents maintain the activity mediated by the type I receptor, but significantly reduces the biological activity mediated by the other receptors. See, for example, U.S. Pat. No. 6,433,157, the entire contents of which are hereby incorporated by reference.

In an embodiment, the wild type or modified signaling agent is IL-6. IL-6 signals through a cell-surface type I cytokine receptor complex including the ligand-binding IL-6R chain (CD126), and the signal-transducing component gp130. IL-6 may also bind to a soluble form of IL-6R (sIL-6R), which is the extracellular portion of IL-6R. The sIL-6R/IL-6 complex may be involved in neurites outgrowth and survival of neurons and, hence, may be important in nerve regeneration through remyelination. Accordingly, in some embodiments, the modified signaling agent has reduced affinity and/or activity for IL-6R/gp130 and/or sIL-6R. In some embodiments, the modified signaling agent has substantially reduced or ablated affinity and/or activity for IL-6R/gp130 and/or sIL-6R.

In an embodiment, the wild type IL-6 has the amino acid sequence of:

(SEQ ID NO: 20)
APVPPGEDSKDVAAPHRQPLTSSERIDKQIRYILDGISALRKETCNKSNM
CESSKEALAENNLNLPKMAEKDGCFQSGFNEETCLVKIITGLLEFEVYLE
YLQNRFESSEEQARAVQMSTKVLIQFLQKKAKNLDAITTPDPTTNASLTT
KLQAQNQWLQDMTTHLILRSFKEFLQSSLRALRQM.

In such embodiments, the modified signaling agent has one or more mutations at amino acids 58, 160, 163, 171 or 177. Without wishing to be bound by theory, it is believed that these modified IL-6 agents exhibit reduced binding affinity to IL-6Ralpha and reduced biological activity. See, for example, WO 97/10338, the entire contents of which are hereby incorporated by reference.

In an embodiment, the wild type or modified signaling agent is IL-10. In such an embodiment, the modified signaling agent has reduced affinity and/or activity for IL-10 receptor-1 and IL-10 receptor-2. In some embodiments, the modified signaling agent has substantially reduced or ablated affinity and/or activity for IL-10 receptor-1 and IL-10 receptor-2

In an embodiment, the wild type or modified signaling agent is IL-11. In such an embodiment, the modified signaling agent has reduced affinity and/or activity for IL-11Rα and/or IL-11Rβ and/or gp130. In such an embodiment, the modified signaling agent has substantially reduced or ablated affinity and/or activity for IL-11Rα and/or IL-11Rβ and/or gp130.

In an embodiment, the wild type or modified signaling agent is IL-12. In such an embodiment, the modified signaling agent has reduced affinity and/or activity for IL-12Rβ1 and/or IL-12Rβ2. In such an embodiment, the modified signaling agent has substantially reduced or ablated affinity and/or activity for IL-12Rβ1 and/or IL-12Rβ2. In an embodiment, the wild type or modified signaling agent is IL-13. In such an embodiment, the modified signaling agent has reduced affinity and/or activity for the IL-4 receptor (IL-4Rα) and IL-13Rα1. In some embodiments, the modified signaling agent has substantially reduced or ablated affinity and/or activity for IL-4 receptor (IL-4Rα) or IL-13Rα1.

In an embodiment, the wild type IL-13 has the amino acid sequence of:

(SEQ ID NO: 21)
SPGPVPPSTALRELIEELVNITQNQKAPLCNGSMVWSINLTAGMYCAALE
SLINVSGCSAIEKTQRMLSGFCPHKVSAGQFSSLHVRDTKIEVAQFVKDL
LLHLKKLFREGRFN.

In such embodiments, the modified IL-13 agent has one or more mutations at amino acids 13, 16, 17, 66, 69, 99, 102, 104, 105, 106, 107, 108, 109, 112, 113 and 114. Without wishing to be bound by theory, it is believed that these modified IL-13 agents exhibit reduced biological activity. See, for example, WO 2002/018422, the entire contents of which are hereby incorporated by reference.

In an embodiment, the signaling agent is a wild type or modified IL-15. In embodiments, the modified IL-15 has reduced affinity and/or activity for interleukin 15 receptor.

In an embodiment, the wild type IL-15 has the amino acid sequence of: SEQ ID NO: 1564.

In such embodiments, the modified IL-15 agent has one or more mutations at amino acids S7, D8, K10, K11, E46, L47, V49, 150, D61, N65, L66, I67, I68, L69, N72, Q108.

In an embodiment, the wild type or modified signaling agent is IL-18. In some embodiments, the modified signaling agent has reduced affinity and/or activity for IL-18Rα and/or IL-18Rβ. In some embodiments, the modified signaling agent has substantially reduced or ablated affinity and/or activity for IL-18Rα and/or IL-18Rβ. In some embodiments, the modified signaling agent has substantially reduced or ablated affinity and/or activity for IL-18Rα type II, which is an isoform of IL-18Rα that lacks the TIR domain required for signaling.

In an embodiment, the wild type IL-18 has the amino acid sequence of:

(SEQ ID NO: 22)
MAAEPVEDNCINFVAMKFIDNTLYFIAEDDENLESDYFGKLESKLSVIRN
LNDQVLFIDQGNRPLFEDMTDSDCRDNAPRTIFIISMYKDSQPRGMAVTI
SVKCEKISTLSCENKIISFKEMNPPDNIKDTKSDIIFFQRSVPGHDNKMQ
FESSSYEGYFLACEKERDLFKLILKKEDELGDRSIMFTVQNEDL.

In such embodiments, the modified IL-18 agent may comprise one or more mutations in amino acids or amino acid regions selected from Y37-K44, R49-Q54, D59-R63, E67-C74, R80, M87-A97, N 127-K129, Q139-M149, K165-K171, R183 and Q190-N191, as described in WO/2015/007542, the entire contents of which are hereby incorporated by reference (numbering based on the human IL-18 sequence, Genbank accession number AAV38697, version AAV38697.1, GI: 54696650).

In an embodiment, the wild type or modified signaling agent is IL-33. In such an embodiment, the modified signaling agent has reduced affinity and/or activity for the ST-2 receptor and IL-1RAcP. In some embodiments, the modified signaling agent has substantially reduced or ablated affinity and/or activity for the ST-2 receptor and IL-1RAcP.

In an embodiment, the wild type IL-33 has the amino acid sequence of:

(SEQ ID NO: 23)
MKPKMKYSTNKISTAKWKNTASKALCFKLGKSQQKAKEVCPMYFMKLRSG
LMIKKEACYFRRETTKRPSLKTGRKHKRHLVLAACQQQSTVECFAFGISG
VQKYTRALHDSSITGISPITEYLASLSTYNDQSITFALEDESYEIYVEDL
KKDEKKDKVLLSYYESQHPSNESGDGVDGKMLMVTLSPTKDFWLHANNKE
HSVELHKCEKPLPDQAFFVLHNMHSNCVSFECKTDPGVFIGVKDNHLALI
KVDSSENLCTENILFKLSET.

In such embodiments, the modified IL-33 agent may comprise one or more mutations in amino acids or amino acid regions selected from I113-Y122, S127-E139, E144-D157, Y163-M183, E200, Q215, L220-C227 and T260-E269, as described in WO/2015/007542, the entire contents of which are hereby incorporated by reference (numbering based on the human sequence, Genbank accession number NP_254274, version NP_254274.1, GI:15559209).

In an embodiment, the modified signaling agent is epidermal growth factor (EGF). EGF is a member of a family of potent growth factors. Members include EGF, HB-EGF, and others such as TGFalpha, amphiregulin, neuregulins, epiregulin, betacellulin. EGF family receptors include EGFR (ErbB1), ErbB2, ErbB3 and ErbB4. These may function as homodimeric and/or heterodimeric receptor subtypes. The different EGF family members exhibit differential selectivity for the various receptor subtypes. For example, EGF associates with ErbB1/ErbB1, ErbB1/ErbB2, ErbB4/ErbB2 and some other heterodimeric subtypes. HB-EGF has a similar pattern, although it also associates with ErbB4/4. Modulation of EGF (EGF-like) growth factor signaling, positively or negatively, is of considerable therapeutic interest. For example, inhibition of EGFRs signaling is of interest in the treatment of various cancers where EGFR signaling constitutes a major growth-promoting signal. Alternatively, stimulation of EGFRs signaling is of therapeutic interest in, for example, promoting wound healing (acute and chronic), oral mucositis (a major side-effect of various cancer therapies, including, without limitation radiation therapy).

In some embodiments, the modified signaling agent has reduced affinity and/or activity for ErbB1, ErbB2, ErbB3, and/or ErbB4. Such embodiments find use, for example, in methods of treating wounds. In some embodiments, the modified signaling agent binds to one or more ErbB1, ErbB2, ErbB3, and ErbB4 and antagonizes the activity of the receptor. In such embodiments, the modified signaling agent has reduced affinity and/or activity for ErbB1, ErbB2, ErbB3, and/or ErbB4 which allows for the activity of the receptor to be antagonized in an attenuated fashion. Such embodiments find use in, for example, treatments of cancer. In an embodiment, the modified signaling agent has reduced affinity and/or activity for ErbB1. ErbB1 is the therapeutic target of kinase inhibitors—most have side effects because they are not very selective (e.g., gefitinib, erlotinib, afatinib, brigatinib and icotinib). In some embodiments, attenuated antagonistic ErbB1 signaling is more on-target and has less side effects than other agents targeting receptors for EGF.

In some embodiments, the modified signaling agent has reduced affinity and/or activity (e.g. antagonistic e.g. natural antagonistic activity or antagonistic activity that is the result of one or more mutations, see, e.g., WO 2015/007520, the entire contents of which are hereby incorporated by reference) for ErbB1 and/or substantially reduced or ablated affinity and/or activity for ErbB4 or other subtypes it may interact with. Through specific targeting via the targeting moiety, cell-selective suppression (antagonism e.g. natural antagonistic activity or antagonistic activity that is the result of one or more mutations, see, e.g., WO 2015/007520, the entire contents of which are hereby incorporated by reference) of ErbB1/ErbB1 receptor activation would be achieved—while not engaging other receptor subtypes potentially associated with inhibition-associated side effects. Hence, in contrast to EGFR kinase inhibitors, which inhibit EGFR activity in all cell types in the body, such a construct would provide a cell-selective (e.g., tumor cell with activated EGFR signaling due to amplification of receptor, overexpression etc.) anti-EGFR (ErbB1) drug effect with reduced side effects.

In some embodiments, the modified signaling agent has reduced affinity and/or activity (e.g. agonistic) for ErbB4 and/or other subtypes it may interact with. Through targeting to specific target cells through the targeting moiety, a selective activation of ErbB1 signaling is achieved (e.g. epithelial cells). Such a construct finds use, in some embodiments, in the treatment of wounds (promoting would healing) with reduced side effects, especially for treatment of chronic conditions and application other than topical application of a therapeutic (e.g. systemic wound healing).

In an embodiment, the wild type or modified signaling agent is insulin or insulin analogs. In some embodiments, the modified insulin or insulin analog has reduced affinity and/or activity for the insulin receptor and/or IGF1 or IGF2 receptor. In some embodiments, the modified insulin or insulin analog has substantially reduced or ablated affinity and/or activity for the insulin receptor and/or IGF1 or IGF2 receptor. Attenuated response at the insulin receptor allows for the control of diabetes, obesity, metabolic disorders and the like while directing away from IGF1 or IGF2 receptor avoids pro-cancer effects.

In an embodiment, the wild type or modified signaling agent is insulin-like growth factor-I or insulin-like growth factor-II (IGF-1 or IGF-2). In an embodiment, the modified signaling agent is IGF-1. In such an embodiment, the modified signaling agent has reduced affinity and/or activity for the insulin receptor and/or IGF1 receptor. In an embodiment, the modified signaling agent may bind to the IGF1 receptor and antagonize the activity of the receptor. In such an embodiment, the modified signaling agent has reduced affinity and/or activity for IGF1 receptor that allows for the activity of the receptor to be antagonized in an attenuated fashion. In some embodiments, the modified signaling agent has substantially reduced or ablated affinity and/or activity for the insulin receptor and/or IGF1 receptor. In some embodiments, the modified signaling agent has reduced affinity and/or activity for IGF2 receptor that allows for the activity of the receptor to be antagonized in an attenuated fashion. In an embodiment, the modified signaling agent has substantially reduced or ablated affinity and/or activity for the insulin receptor and accordingly does not interfere with insulin signaling. In various embodiments, this applies to cancer treatment. In various embodiments, the present agents may prevent IR isoform A from causing resistance to cancer treatments.

In some embodiments, the wild type or modified signaling agent is EPO. In various embodiments, the modified EPO agent has reduced affinity and/or activity for the EPO receptor (EPOR) receptor and/or the ephrin receptor (EphR) relative to wild type EPO or other EPO based agents described herein. In some embodiments, the modified EPO agent has substantially reduced or ablated affinity and/or activity for the EPO receptor (EPOR) receptor and/or the Eph receptor (EphR). Illustrative EPO receptors include, but are not limited to, an EPOR homodimer or an EPOR/CD131 heterodimer. Also included as an EPO receptor is beta-common receptor (βcR). Illustrative Eph receptors include, but are not limited to, EPHA1, EPHA2, EPHA3, EPHA4, EPHA5, EPHA6, EPHA7, EPHA8, EPHA9, EPHA10, EPHB1, EPHB2, EPHB3, EPHB4, EPHB5, and EPHB6. In some embodiments, the modified EPO protein comprises one or more mutations that cause the EPO protein to have reduced affinity for receptors that comprise one or more different EPO receptors or Eph receptors (e.g. heterodimer, heterotrimers, etc., including by way of non-limitation: EPOR-EPHB4, EPOR-βcR-EPOR). Also provided are the receptors of EP Patent Publication No. 2492355 the entire contents of which are hereby incorporated by reference, including by way of non-limitation, NEPORs.

In some embodiments, the human EPO has the amino acid sequence of (the first 27 amino acids are the signal peptide):

(SEQ ID NO: 24)
MGVHECPAWLWLLLSLLSLPLGLPVLGAPPRLICDSRVLERYLLEAKEAE
NITTGCAEHCSLNENITVPDTKVNFYAWKRMEVGQQAVEVWQGLALLSEA
VLRGQALLVNSSQPWEPLQLHVDKAVSGLRSLTTLLRALGAQKEAISPPD
AASAAPLRTITADTFRKLFRVYSNFLRGKLKLYTGEACRTGDR.

In some embodiments, the human EPO protein is the mature form of EPO (with the signal peptide being cleaved off) which is a glycoprotein of 166 amino acid residues having the sequence of:

(SEQ ID NO: 25)
APPRLICDSRVLERYLLEAKEAENITTGCAEHCSLNENITVPDTKVNFYA
WKRMEVGQQAVEVWQGLALLSEAVLRGQALLVNSSQPWEPLQLHVDKAVS
GLRSLTTLLRALGAQKEAISPPDAASAAPLRTITADTFRKLFRVYSNFLR
GKLKLYTGEACRTGDR.

The structure of the human EPO protein is predicted to comprise four-helix bundles including helices A, B, C, and D. In various embodiments, the modified EPO protein comprises one or more mutations located in four regions of the EPO protein which are important for bioactivity, i.e., amino acid residues 10-20, 44-51, 96-108, and 142-156. In some embodiments, the one or more mutations are located at residues 11-15, 44-51, 100-108, and 147-151. These residues are localized to helix A (Val11, Arg14, and Tyr15), helix C (Ser100, Arg103, Ser104, and Leu108), helix D (Asn147, Arg150, Gly151, and Leu155), and the A/B connecting loop (residues 42-51). In some embodiments, the modified EPO protein comprises mutations in residues between amino acids 41-52 and amino acids 147, 150, 151, and 155. Without wishing to be bound by theory, it is believed that mutations of these residues have substantial effects on both receptor binding and in vitro biological activity. In some embodiments, the modified EPO protein comprises mutations at residues 11, 14, 15, 100, 103, 104, and 108. Without wishing to be bound by theory, it is believed that mutations of these residues have modest effects on receptor binding activity and much greater effects on in vitro biological activity. Illustrative substitutions include, but are not limited to, one or more of Val11Ser, Arg14Ala, Arg14Gln, Tyr15Ile, Pro42Asn, Thr44Ile, Lys45Asp, Val46Ala, Tyr51Phe, Ser100Glu, Ser100Thr, Arg103Ala, Ser104Ile, Ser104Ala, Leu108Lys, Asn147Lys, Arg150Ala, Gly151Ala, and Leu155Ala.

In some embodiments, the modified EPO protein comprises mutations that effect bioactivity and not binding, e.g. those listed in Eliot, et al. Mapping of the Active Site of Recombinant Human Erythropoietin Jan. 15, 1997; Blood: 89 (2), the entire contents of which are hereby incorporated by reference.

In some embodiments, the modified EPO protein comprises one or more mutations involving surface residues of the EPO protein that are involved in receptor contact. Without wishing to be bound by theory, it is believed that mutations of these surface residues are less likely to affect protein folding thereby retaining some biological activity. Illustrative surface residues that may be mutated include, but are not limited to, residues 147 and 150. In illustrative embodiments, the mutations are substitutions including, one or more of N147A, N147K, R150A and R150E.

In some embodiments, the modified EPO protein comprises one or more mutations at residues N59, E62, L67, and L70, and one or more mutations that affect disulfide bond formation. Without wishing to be bound by theory, it is believed that these mutations affect folding and/or are predicted be in buried positions and thus affects biological activity indirectly.

In an embodiment, the modified EPO protein comprises a K20E substitution which significantly reduces receptor binding. See Elliott, et al., (1997) Blood, 89:493-502, the entire contents of which are hereby incorporated by reference.

Additional EPO mutations that may be incorporated into the chimeric EPO protein of the invention are disclosed in, for example, Elliott, et al., (1997) Blood, 89:493-502, the entire contents of which are hereby incorporated by reference and Taylor et al., (2010) PEDS, 23(4): 251-260, the entire contents of which are hereby incorporated by reference.

In some embodiments, the signaling agent is a toxin or toxic enzyme. In some embodiments, the toxin or toxic enzyme is derived from plants and bacteria. Illustrative toxins or toxic enzymes include, but are not limited to, the diphtheria toxin, Pseudomonas toxin, anthrax toxin, ribosome-inactivating proteins (RIPs) such as ricin and saporin, modeccin, abrin, gelonin, and poke weed antiviral protein. Additional toxins include those disclosed in Mathew et al., (2009) Cancer Sci 100(8): 1359-65, the entire disclosures are hereby incorporated by reference. In such embodiments, the Fc-based chimeric protein complex of the invention may be utilized to induce cell death in cell-type specific manner. In such embodiments, the toxin may be modified, e.g. mutated, to reduce affinity and/or activity of the toxin for an attenuated effect, as described with other signaling agents herein.

### Targeting Moieties (TM)

In some embodiments, the targeting moiety is a protein-based agent capable of specific binding, such as an antibody or derivatives thereof.

In some embodiments, the targeting moiety comprises antibody derivatives or formats. In some embodiments, the targeting moiety of the present Fc-based chimeric protein complex is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; a Microbody; a peptide aptamer; an alterases; a plastic antibodies; a phylomer; a stradobodies; a maxibodies; an evibody; a fynomer, an armadillo repeat protein, a Kunitz domain, an avimer, an atrimer, a probody, an immunobody, a triomab, a troybody; a pepbody; a vaccibody, a UniBody; affimers, a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In one embodiment, the targeting moiety comprises a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO 2016/113555 and WO2016/113557, the entire disclosure of which is incorporated by reference.

In various embodiments, the target (e.g. antigen, receptor) of interest can be found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In some embodiments, the recognition domains specifically bind to a target (e.g. antigen, receptor) of interest and effectively, directly or indirectly, recruit one of more immune cells. In some embodiments, the target (e.g. antigen, receptor) of interest can be found on one or more tumor cells. In some embodiments, the present Fc-based chimeric protein complexes may directly or indirectly recruit an immune cell, e.g., in some embodiments, to a therapeutic site (e.g. a locus with one or more disease cell or cell to be modulated for a therapeutic effect). In some embodiments, the present Fc-based chimeric protein complexes may directly or indirectly recruit an immune cell, e.g. an immune cell that can kill and/or suppress a tumor cell, to a site of action (such as, by way of non-limiting example, the tumor microenvironment).

In various embodiments, the targeting moieties can directly or indirectly recruit cells, such as disease cells and/or effector cells. In some embodiments, the present Fc-based chimeric protein complexes are capable of, or find use in methods involving, shifting the balance of immune cells in favor of immune attack of a tumor. For instance, the present Fc-based chimeric protein complexes can shift the ratio of immune cells at a site of clinical importance in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, the present Fc-based chimeric protein complex is capable of increasing a ratio of effector T cells to regulatory T cells.

For example, in some embodiments, the recognition domains specifically bind to a target (e.g. antigen, receptor) associated with T cells. In some embodiments, the recognition domains directly or indirectly recruit T cells. In an embodiment, the recognition domains specifically bind to effector T cells. In some embodiments, the recognition domain directly or indirectly recruits effector T cells, e.g., in some embodiments, to a therapeutic site (e.g. a locus with one or more disease cell or cell to be modulated for a therapeutic effect). Illustrative effector T cells include cytotoxic T cells (e.g. αβ TCR, CD3⁺, CD8⁺, CD45RO⁺); CD4⁺ effector T cells (e.g. αβ TCR, CD3⁺, CD4⁺, CCR7⁺, CD62Lhi, IL⁻7R/CD127⁺); CD8⁺ effector T cells (e.g. αβ TCR, CD3⁺, CD8⁺, CCR7⁺, CD62Lhi, IL⁻7R/CD127⁺); effector memory T cells (e.g. CD62Llow, CD44⁺, TCR, CD3⁺, IL⁻7R/CD127⁺, IL-15R⁺, CCR7low); central memory T cells (e.g. CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺CD62L⁻) and late effector memory T cells (CD27⁻CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g. CD44(low)CD62L(high)CD122(high)sca(⁺); TH1 effector T-cells (e.g. CXCR3⁺, CXCR6⁺ and CCR5⁺; or αβ TCR, CD3⁺, CD4⁺, IL-12R⁺, IFNγR⁺, CXCR3⁺), TH2 effector T cells (e.g. CCR3⁺, CCR4⁺ and CCR8⁺, or αβTCR, CD3⁺, CD4⁺, IL-4R⁺, IL-33R⁺, CCR4⁺, IL-17RB⁺, CRTH2⁺); TH9 effector T cells (e.g. αβ TCR, CD3⁺, CD4⁺); TH17 effector T cells (e.g. αβ TCR, CD3⁺, CD4⁺, IL-23R⁺, CCR6⁺, IL-1R⁺); CD4⁺CD45RO⁺CCR7⁺ effector T cells, ICOS⁺ effector T cells; CD4⁺CD45RO⁺CCR7(⁻) effector T cells; and effector T cells secreting IL-2, IL-4 and/or IFN-γ.

Illustrative T cell antigens of interest include, for example (and inclusive of the extracellular domains, where applicable): CD8, CD3, SLAMF4, IL-2Rα, 4-1BB/TNFRSF9, IL-2 R β, ALCAM, B7-1, IL-4 R, B7-H3, BLAME/SLAMFS, CEACAM1, IL-6 R, CCR3, IL-7 Rα, CCR4, CXCRI/IL-S RA, CCR5, CCR6, IL-10Rα, CCR7, IL-I 0 R β, CCRS, IL-12 R β1, CCR9, IL-12 R β2, CD2, IL-13 R α 1, IL-13, CD3, CD4, ILT2/CDS5j, ILT3/CDS5k, ILT4/CDS5d, ILT5/CDS5a, lutegrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1 and TSLP R. In various embodiments, a targeting moiety of the Fc-based chimeric protein complex binds one or more of these illustrative T cell antigens.

In various embodiments, the targeting moiety of the present Fc-based chimeric protein complex is a protein-based agent capable of specific binding to a cell receptor, such as a natural ligand for the cell receptor. In various embodiments, the cell receptor is found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In some embodiments, the cell receptor is found on megakaryocytes, thrombocytes, erythrocytes, mast cells, basophils, neutrophils, eosinophils, or subsets thereof.

In some embodiments, the targeting moiety is a natural ligand such as a chemokine. Exemplary chemokines that may be included in the Fc-based chimeric protein complex of the invention include, but are not limited to, CCL1, CCL2, CCL4, CCL5, CCL6, CCL7, CCL8, CCL9, CCL10, CCL11, CCL12, CCL13, CCL14, CCL15, CCL16, CL17, CCL18, CCL19, CCL20, CCL21, CCL22, CCL23, CCL24, CLL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, HCC-4, and LDGF-PBP. In an illustrative embodiment, the targeting moiety may be XCL1 which is a chemokine that recognizes and binds to the dendritic cell receptor XCR1. In another illustrative embodiment, the targeting moiety is CCL1, which is a chemokine that recognizes and binds to CCR8. In another illustrative embodiment, the targeting moiety is CCL2, which is a chemokine that recognizes and binds to CCR2 or CCR9. In another illustrative embodiment, the targeting moiety is CCL3, which is a chemokine that recognizes and binds to CCR1, CCR5, or CCR9. In another illustrative embodiment, the targeting moiety is CCL4, which is a chemokine that recognizes and binds to CCR1 or CCR5 or CCR9. In another illustrative embodiment, the targeting moiety is CCL5, which is a chemokine that recognizes and binds to CCR1 or CCR3 or CCR4 or CCR5. In another illustrative embodiment, the targeting moiety is CCL6, which is a chemokine that recognizes and binds to CCR1. In another illustrative embodiment, the targeting moiety is CCL7, which is a chemokine that recognizes and binds to CCR2 or CCR9. In another illustrative embodiment, the targeting moiety is CCL8, which is a chemokine that recognizes and binds to CCR1 or CCR2 or CCR2B or CCR5 or CCR9. In another illustrative embodiment, the targeting moiety is CCL9, which is a chemokine that recognizes and binds to CCR1. In another illustrative embodiment, the targeting moiety is CCL10, which is a chemokine that recognizes and binds to CCR1. In another illustrative embodiment, the targeting moiety is CCL11, which is a chemokine that recognizes and binds to CCR2 or CCR3 or CCR5 or CCR9. In another illustrative embodiment, the targeting moiety is CCL13, which is a chemokine that recognizes and binds to CCR2 or CCR3 or CCR5 or CCR9. In another illustrative embodiment, the targeting moiety is CCL14, which is a chemokine that recognizes and binds to CCR1 or CCR9. In another illustrative embodiment, the targeting moiety is CCL15, which is a chemokine that recognizes and binds to CCR1 or CCR3. In another illustrative embodiment, the targeting moiety is CCL16, which is a chemokine that recognizes and binds to CCR1, CCR2, CCR5, or CCR8. In another illustrative embodiment, the targeting moiety is CCL17, which is a chemokine that recognizes and binds to CCR4. In another illustrative embodiment, the targeting moiety is CCL19, which is a chemokine that recognizes and binds to CCR7. In another illustrative embodiment, the targeting moiety is CCL20, which is a chemokine that recognizes and binds to CCR6. In another illustrative embodiment, the targeting moiety is CCL21, which is a chemokine that recognizes and binds to CCR7. In another illustrative embodiment, the targeting moiety is CCL22, which is a chemokine that recognizes and binds to CCR4. In another illustrative embodiment, the targeting moiety is CCL23, which is a chemokine that recognizes and binds to CCR1. In another illustrative embodiment, the targeting moiety is CCL24, which is a chemokine that recognizes and binds to CCR3. In another illustrative embodiment, the targeting moiety is CCL25, which is a chemokine that recognizes and binds to CCR9. In another illustrative embodiment, the targeting moiety is CCL26, which is a chemokine that recognizes and binds to CCR3. In another illustrative embodiment, the targeting moiety is CCL27, which is a chemokine that recognizes and binds to CCR10. In another illustrative embodiment, the targeting moiety is CCL28, which is a chemokine that recognizes and binds to CCR3 or CCR10.

In another illustrative embodiment, the targeting moiety is CXCL1, which is a chemokine that recognizes and binds to CXCR1 or CXCR2. In another illustrative embodiment, the targeting moiety is CXCL2, which is a chemokine that recognizes and binds to CXCR2. In another illustrative embodiment, the targeting moiety is CXCL3, which is a chemokine that recognizes and binds to CXCR2. In another illustrative embodiment, the targeting moiety is CXCL4, which is a chemokine that recognizes and binds to CXCR3B. In another illustrative embodiment, the targeting moiety is CXCL5, which is a chemokine that recognizes and binds to CXCR2. In another illustrative embodiment, the targeting moiety is CXCL6, which is a chemokine that recognizes and binds to CXCR1 or CXCR2.

In another illustrative embodiment, the targeting moiety is CXCL8, which is a chemokine that recognizes and binds to CXCR1 or CXCR2. In another illustrative embodiment, the targeting moiety is CXCL9, which is a chemokine that recognizes and binds to CXCR3. In another illustrative embodiment, the targeting moiety is CXCL10, which is a chemokine that recognizes and binds to CXCR3. In another illustrative embodiment, the targeting moiety is CXCL11, which is a chemokine that recognizes and binds to CXCR3 or CXCR7. In another illustrative embodiment, the targeting moiety is CXCL12, which is a chemokine that recognizes and binds to CXCR4 or CXCR7. In another illustrative embodiment, the targeting moiety is CXCL13, which is a chemokine that recognizes and binds to CXCR5. In another illustrative embodiment, the targeting moiety is CXCL16, which is a chemokine that recognizes and binds to CXCR6. In another illustrative embodiment, the targeting moiety is LDGF-PBP, which is a chemokine that recognizes and binds to CXCR2. In another illustrative embodiment, the targeting moiety is XCL2, which is a chemokine that recognizes and binds to XCR1. In another illustrative embodiment, the targeting moiety is CX3CL1, which is a chemokine that recognizes and binds to CX3CR1.

In some embodiments, the targeting moiety is a natural ligand such as Flt3 or a truncated region thereof. In some embodiments, the targeting moiety is an extracellular domain of Flt3, or a functional portion thereof (e.g. one that is still able to bind the cognate ligand or receptor).

Functional equivalent of extracellular domains of natural ligands encompass N-terminal and/or C-terminally shortened versions that retain the binding capacitiy of the full-length extracellular domains.

In some embodiments, the targeting moiety is a NGR peptide or a truncated region thereof.

By way of non-limiting example, in various embodiments, the present Fc-based chimeric protein complex has a targeting moiety directed against a checkpoint marker expressed on a T cell, e.g. one or more of PD-1, CD28, CTLA4, ICOS, BTLA, KIR, LAG3, CD137, OX40, CD27, CD40L, TIM3, and A2aR.

In some embodiments, the targeting moiety is an extracellular domain of PD-1, PD-L1, or PD-L2, or a functional portion thereof (e.g. one that is still able to bind the cognate ligand or receptor).

For example, in some embodiments, the recognition domains specifically bind to a target (e.g. antigen, receptor) associated with B cells. In some embodiments, the recognition domains directly or indirectly recruit B cells, e.g., in some embodiments, to a therapeutic site (e.g. a locus with one or more disease cell or cell to be modulated for a therapeutic effect). Illustrative B cell antigens of interest include, for example, CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, CDw150, and B-cell maturation antigen (BCMA). In various embodiments, a targeting moiety of the Fc-based chimeric protein complex binds one or more of these illustrative B cell antigens.

By way of further example, in some embodiments, the recognition domains specifically bind to a target (e.g. antigen, receptor) associated with Natural Killer cells. In some embodiments, the recognition domains directly or indirectly recruit Natural Killer cells, e.g., in some embodiments, to a therapeutic site (e.g. a locus with one or more disease cell or cell to be modulated for a therapeutic effect). Illustrative Natural Killer cell antigens of interest include, for example TIGIT, 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAM F6, Rae-1, Rae-1 α, Rae-1 β, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d and ULBP-3. In various embodiments, a targeting moiety of the Fc-based chimeric protein complex binds one or more of these illustrative NK cell antigens.

Also, in some embodiments, the recognition domains specifically bind to a target (e.g. antigen, receptor) associated with macrophages/monocytes. In some embodiments, the recognition domains directly or indirectly recruit macrophages/monocytes, e.g., in some embodiments, to a therapeutic site (e.g. a locus with one or more disease cell or cell to be modulated for a therapeutic effect). Illustrative macrophages/monocyte antigens of interest include, for example SIRP1a, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR1/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-γ R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, LMIR1/CD300A, CD97, LMIR2/CD300c, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-1, IL-6 R, TREM-2, CXCRI/IL-8 RA, TREM-3 and TREMLI/TLT-1. In various embodiments, a targeting moiety of the Fc-based chimeric protein complex binds one or more of these illustrative macrophage/monocyte antigens.

Also, in some embodiments, the recognition domains specifically bind to a target (e.g. antigen, receptor) associated with dendritic cells. In some embodiments, the recognition domains directly or indirectly recruit dendritic cells, e.g., in some embodiments, to a therapeutic site (e.g. a locus with one or more disease cell or cell to be modulated for a therapeutic effect). Illustrative dendritic cell antigens of interest include, for example, CLEC9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, lutegrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, CLEC-8, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102 and Vanilloid R1. In various embodiments, a targeting moiety of the Fc-based chimeric protein complex binds one or more of these illustrative DC antigens.

In some embodiments, the recognition domains specifically bind to a target (e.g. antigen, receptor) on immune cells selected from, but not limited to, megakaryocytes, thrombocytes, erythrocytes, mast cells, basophils, neutrophils, eosinophils, or subsets thereof. In some embodiments, the recognition domains directly or indirectly recruit megakaryocytes, thrombocytes, erythrocytes, mast cells, basophils, neutrophils, eosinophils, or subsets thereof, e.g., in some embodiments, to a therapeutic site (e.g. a locus with one or more disease cell or cell to be modulated for a therapeutic effect).

In some embodiments, the recognition domains specifically bind to a target (e.g. antigen, receptor) associated with megakaryocytes and/or thrombocytes. Illustrative megakaryocyte and/or thrombocyte antigens of interest include, for example, GP IIb/IIIa, GPIb, vWF, PF4, and TSP. In various embodiments, a targeting moiety of the Fc-based chimeric protein complex binds one or more of these illustrative megakaryocyte and/or thrombocyte antigens.

In some embodiments, the recognition domains specifically bind to a target (e.g. antigen, receptor) associated with erythrocytes. Illustrative erythrocyte antigens of interest include, for example, CD34, CD36, CD38, CD41a (platelet glycoprotein IIb/IIIa), CD41b (GPIIb), CD71 (transferrin receptor), CD105, glycophorin A, glycophorin C, c-kit, HLA-DR, H2 (MHC-II), and Rhesus antigens. In various embodiments, a targeting moiety of the Fc-based chimeric protein complex binds one or more of these illustrative erythrocyte antigens.

In some embodiments, the recognition domains specifically bind to a target (e.g. antigen, receptor) associated with mast cells. Illustrative mast cells antigens of interest include, for example, SCFR/CD117, Fc_{ε}RI, CD2, CD25, CD35, CD88, CD203c, C5R1, CMAI, FCERIA, FCER2, TPSABI. In various embodiments, a targeting moiety of the Fc-based chimeric protein complex binds one or more of these mast cell antigens.

In some embodiments, the recognition domains specifically bind to a target (e.g. antigen, receptor) associated with basophils. Illustrative basophils antigens of interest include, for example, Fc_{ε}RI, CD203c, CD123, CD13, CD107a, CD107b, and CD164. In various embodiments, a targeting moiety of the Fc-based chimeric protein complex binds one or more of these basophil antigens.

In some embodiments, the recognition domains specifically bind to a target (e.g. antigen, receptor) associated with neutrophils. Illustrative neutrophils antigens of interest include, for example, 7D5, CD10/CALLA, CD13, CD16 (FcRIII), CD18 proteins (LFA-1, CR3, and p150, 95), CD45, CD67, and CD177. In various embodiments, a targeting moiety of the Fc-based chimeric protein complex binds one or more of these neutrophil antigens.

In some embodiments, the recognition domains specifically bind to a target (e.g. antigen, receptor) associated with eosinophils. Illustrative eosinophils antigens of interest include, for example, CD35, CD44 and CD69. In various embodiments, a targeting moiety of the Fc-based chimeric protein complex binds one or more of these eosinophil antigens.

In various embodiments, the recognition domain may bind to any appropriate target, antigen, receptor, or cell surface markers known by the skilled artisan. In some embodiments, the antigen or cell surface marker is a tissue-specific marker. Illustrative tissue-specific markers include, but are not limited to, endothelial cell surface markers such as ACE, CD14, CD34, CDH5, ENG, ICAM2, MCAM, NOS3, PECAMI, PROCR, SELF, SELP, TEK, THBD, VCAMI, VWF; smooth muscle cell surface markers such as ACTA2, MYHIO, MYHI 1, MYH9, MYOCD; fibroblast (stromal) cell surface markers such as ALCAM, CD34, COLIAI, COL1A2, COL3A1, FAP, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (α_{V}β₃), E-selectin; and adipocyte surface markers such as ADIPOQ, FABP4, and RETN. In various embodiments, a targeting moiety of the Fc-based chimeric protein complex binds one or more of these antigens. In various embodiments, a targeting moiety of the Fc-based chimeric protein complex binds one or more of cells having these antigens.

In some embodiments, the recognition domains specifically bind to a target (e.g. antigen, receptor) associated with tumor cells. In some embodiments, the recognition domains directly or indirectly recruit tumor cells. For instance, in some embodiments, the direct or indirect recruitment of the tumor cell is to one or more effector cell (e.g. an immune cell as described herein) that can kill and/or suppress the tumor cell.

Tumor cells or cancer cells refer to an uncontrolled growth of cells or tissues and/or an abnormal increase in cell survival and/or inhibition of apoptosis which interferes with the normal functioning of bodily organs and systems.

For example, tumor cells include benign and malignant cancers, polyps, hyperplasia, as well as dormant tumors or micrometastases. Illustrative tumor cells include, but are not limited to cells of: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Tumor cells, or cancer cells also include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative tumor antigens include, but are not limited to, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD56, CD70, CD74, CD138, AGS16, MUC1, GPNMB, Ep-CAM, PD-L1, PD-L2, PMSA, and BCMA (TNFRSF17). In various embodiments, a targeting moiety of the Fc-based chimeric protein complex binds one or more of these tumor antigens. In an embodiment, the Fc-based chimeric protein complex binds to HER2.

In another embodiment, the Fc-based chimeric protein complex binds to PD-L2.

In various embodiments, the recognition domain of the present Fc-based chimeric protein complex binds but does not functionally modulate the target (e.g. antigen, receptor) of interest, e.g. the recognition domain is, or is akin to, a binding antibody. For instance, in various embodiments, the recognition domain simply targets the antigen or receptor but does not substantially inhibit, reduce or functionally modulate a biological effect that the antigen or receptor has. For example, some of the smaller antibody formats described above (e.g. as compared to, for example, full antibodies) have the ability to target hard to access epitopes and provide a larger spectrum of specific binding locales. In various embodiments, the recognition domain binds an epitope that is physically separate from an antigen or receptor site that is important for its biological activity (e.g. the antigen's active site).

Such non-neutralizing binding finds use in various embodiments of the present invention, including methods in which the present Fc-based chimeric protein complex is used to directly or indirectly recruit active immune cells to a site of need via an effector antigen, such as any of those described herein. For example, in various embodiments, the present Fc-based chimeric protein complex may be used to directly or indirectly recruit cytotoxic T cells via CD8 to a tumor cell in a method of reducing or eliminating a tumor (e.g. the Fc-based chimeric protein complex may comprise an anti-CD8 recognition domain and a recognition domain directed against a tumor antigen). In such embodiments, it is desirable to directly or indirectly recruit CD8-expressing cytotoxic T cells but not to functionally modulate the CD8 activity. On the contrary, in these embodiments, CD8 signaling is an important piece of the tumor reducing or eliminating effect. By way of further example, in various methods of reducing or eliminating tumors, the present Fc-based chimeric protein complex is used to directly or indirectly recruit dendritic cells (DCs) via CLEC9A (e.g. the Fc-based chimeric protein complex may comprise an anti-CLEC9A recognition domain and a recognition domain directed against a tumor antigen). In such embodiments, it is desirable to directly or indirectly recruit CLEC9A-expressing DCs but not to functionally modulate the CLEC9A activity. On the contrary, in these embodiments, CLEC9A signaling is an important piece of the tumor reducing or eliminating effect.

In various embodiments, the recognition domain of the present Fc-based chimeric protein complex binds to an immune modulatory antigen (e.g. immune stimulatory or immune inhibitory). In various embodiments, the immune modulatory antigen is one or more of 4-1BB, OX-40, HVEM, GITR, CD27, CD28, CD30, CD40, ICOS ligand; OX-40 ligand, LIGHT (CD258), GITR ligand, CD70, B7-1, B7-2, CD30 ligand, CD40 ligand, ICOS, ICOS ligand, CD137 ligand and TL1A. In various embodiments, such immune stimulatory antigens are expressed on a tumor cell. In various embodiments, the recognition domain of the present Fc-based chimeric protein complex binds but does not functionally modulate such immune stimulatory antigens and therefore allows recruitment of cells expressing these antigens without the reduction or loss of their potential tumor reducing or eliminating capacity.

In various embodiments, the recognition domain of the present Fc-based chimeric protein complex may be in the context of Fc-based chimeric protein complex that comprises two recognition domains that have neutralizing activity, or comprises two recognition domains that have non-neutralizing (e.g. binding) activity, or comprises one recognition domain that has neutralizing activity and one recognition domain that has non-neutralizing (e.g. binding) activity.

### Clec9A Targeting Moieties

In some embodiments, the targeting moiety is a Clec9A targeting moiety that is a protein-based agent capable of specific binding to Clec9A. In some embodiments, the Clec9A targeting moiety is a protein-based agent capable of specific binding to Clec9A without functional modulation (e.g., partial or full neutralization) of Clec9A. Clec9A is a group V C-type lectin-like receptor (CTLR) expressed on the surface of a subset of dendritic cells (i.e., BDCA₃+ dendritic cells) specialized for the uptake and processing of materials from dead cells. Clec9A recognizes a conserved component within nucleated and nonnucleated cells, exposed when cell membranes are damaged. Clec9A is expressed at the cell surface as a glycosylated dimer and can mediate endocytosis, but not phagocytosis. Clec9A possesses a cytoplasmic immunoreceptor tyrosine-based activation-like motif that can recruit Syk kinase and induce proinflammatory cytokine production (see Huysamen et al. (2008), JBC, 283:16693-701).

In various embodiments, the Clec9A targeting moiety comprises an antigen recognition domain that recognizes an epitope present on Clec9A. In an embodiment, the antigen-recognition domain recognizes one or more linear epitopes present on Clec9A. In some embodiments, a linear epitope refers to any continuous sequence of amino acids present on Clec9A. In another embodiment, the antigen-recognition domain recognizes one or more conformational epitopes present on Clec9A. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the Clec9A targeting moiety can bind to the full-length and/or mature forms and/or isoforms and/or splice variants and/or fragments and/or any other naturally occurring or synthetic analogs, variants, or mutants of human Clec9A. In various embodiments, the Clec9A targeting moiety can bind to any forms of the human Clec9A, including monomeric, dimeric, heterodimeric, multimeric and associated forms. In an embodiment, the Clec9A binding agent binds to the monomeric form of Clec9A. In another embodiment, the Clec9A targeting moiety binds to a dimeric form of Clec9A. In a further embodiment, the Clec9A targeting moiety binds to glycosylated form of Clec9A, which may be either monomeric or dimeric.

In an embodiment, the Clec9A targeting moiety an antigen recognition domain that recognizes one or more epitopes present on human Clec9A. In an embodiment, the human Clec9A comprises the amino acid sequence of:

(SEQ ID NO: 26)
MHEEEIYTSLQWDSPAPDTYQKCLSSNKCSGACCLVMVISCVFCMGLLTA
SIFLGVKLLQVSTIAMQQQEKLIQQERALLNFTEWKRSCALQMKYCQAFM
QNSLSSAHNSSPCPNNWIQNRESCYYVSEIWSIWHTSQENCLKEGSTLLQ
IESKEEMDFITGSLRKIKGSYDYWVGLSQDGHSGRWLWQDGSSPSPGLLP
AERSQSANQVCGYVKSNSLLSSNCSTWKYFICEKYALRSSV.

In various embodiments, the Clec9A targeting moiety is capable of specific binding. In various embodiments, the Clec9A targeting moiety comprises an antigen recognition domain such as an antibody or derivatives thereof.

In some embodiments, the Clec9A targeting moiety comprises an antibody derivative or format. In some embodiments, the Clec9A targeting moiety comprises a targeting moiety which is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an alphabody; a bicyclic peptide; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the Clec9A targeting moiety is a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3).

In an embodiment, the Clec9A targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO2016/113555 and WO2016/113557, the entire disclosure of which is incorporated by reference.

In some embodiments, the Clec9A targeting moiety is a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In various embodiments, the Clec9A targeting moiety comprises a VHH having a variable domain comprising at least one CDR1, CDR2, and/or CDR3 sequences. In various embodiments, the Clec9A targeting moiety comprises a VHH having a variable region comprising at least one FR1, FR2, FR3, and FR4 sequences.

In some embodiments, the CDR1 sequence is selected from SEQ ID Nos: 27-112.

In some embodiments, the CDR2 sequence is selected from SEQ ID Nos: 113-200.

In some embodiments, the CDR3 sequence is selected from SEQ ID Nos: 201-287, LGR, and VIK.

In an exemplary embodiment, the Clec9A targeting moiety comprises SEQ ID NO: 27, SEQ ID NO: 113, and SEQ ID NO: 201.

In an exemplary embodiment, the Clec9A targeting moiety comprises SEQ ID NO: 28, SEQ ID NO: 114, and SEQ ID NO: 202.

In an exemplary embodiment, the Clec9A targeting moiety comprises SEQ ID NO: 29, SEQ ID NO: 115, and SEQ ID NO: 202.

In an exemplary embodiment, the Clec9A targeting moiety comprises SEQ ID NO: 27, SEQ ID NO: 116, and SEQ ID NO: 203.

In an exemplary embodiment, the Clec9A targeting moiety comprises SEQ ID NO: 30, SEQ ID NO: 117, and SEQ ID NO: 205.

In an exemplary embodiment, the Clec9A targeting moiety comprises SEQ ID NO: 31, SEQ ID NO: 118, and SEQ ID NO: 205.

In an exemplary embodiment, the Clec9A targeting moiety comprises SEQ ID NO: 32, SEQ ID NO: 119, and SEQ ID NO: 206.

In an exemplary embodiment, the Clec9A targeting moiety comprises SEQ ID NO: 33, SEQ ID NO: 120, and SEQ ID NO: 207.

In an exemplary embodiment, the Clec9A targeting moiety comprises SEQ ID NO: 33, SEQ ID NO: 120, and SEQ ID NO: 208.

In an exemplary embodiment, the Clec9A targeting moiety comprises SEQ ID NO: 33, SEQ ID NO: 120, and SEQ ID NO: 209.

In an exemplary embodiment, the Clec9A targeting moiety comprises SEQ ID NO: 34, SEQ ID NO: 121, and SEQ ID NO: 210.

In an exemplary embodiment, the Clec9A targeting moiety comprises SEQ ID NO: 35, SEQ ID NO: 122, and SEQ ID NO: 211.

In an exemplary embodiment, the Clec9A targeting moiety comprises SEQ ID NO:35, SEQ ID NO: 122, and SEQ ID NO: 212.

In an exemplary embodiment, the Clec9A targeting moiety comprises SEQ ID NO: 36, SEQ ID NO: 123, and SEQ ID NO: 213.

In an exemplary embodiment, the Clec9A targeting moiety comprises SEQ ID NO: 37, SEQ ID NO: 124, and SEQ ID NO: 214.

In an exemplary embodiment, the Clec9A targeting moiety comprises SEQ ID NO: 38, SEQ ID NO: 125, and SEQ ID NO: 214.

In an exemplary embodiment, the Clec9A targeting moiety comprises SEQ ID NO: 39, SEQ ID NO: 126, and SEQ ID NO: 214.

In an exemplary embodiment, the Clec9A targeting moiety comprises SEQ ID NO: 40, SEQ ID NO: 127, and SEQ ID NO: 214.

In an exemplary embodiment, the Clec9A targeting moiety comprises SEQ ID NO: 41, SEQ ID NO: 128, and SEQ ID NO: 214.

In an exemplary embodiment, the Clec9A targeting moiety comprises SEQ ID NO: 42, SEQ ID NO: 128, and SEQ ID NO: 214.

In an exemplary embodiment, the Clec9A targeting moiety comprises SEQ ID NO: 43, SEQ ID NO: 129, and SEQ ID NO: 215.

In an exemplary embodiment, the Clec9A targeting moiety comprises SEQ ID NO: 44, SEQ ID NO: 130, and LGR.

In an exemplary embodiment, the Clec9A targeting moiety comprises SEQ ID NO: 44, SEQ ID NO: 131, and LGR.

In an exemplary embodiment, the Clec9A targeting moiety comprises SEQ ID NO: 44, SEQ ID NO: 132, and LGR.

In an exemplary embodiment, the Clec9A targeting moiety comprises SEQ ID NO: 45, SEQ ID NO: 133, and LGR.

In an exemplary embodiment, the Clec9A targeting moiety comprises SEQ ID NO: 46, SEQ ID NO: 134, and VIK.

By way of example, in some embodiments, the Clec9A targeting moiety comprises an amino acid sequence selected from the following sequences:

R2CHCL8 (SEQ ID NO: 288); R1CHCL50 (SEQ ID NO: 289); R1CHCL21 (SEQ ID NO: 290); R2CHCL87 (SEQ ID NO: 291); R2CHCL24 (SEQ ID NO: 292); R2CHCL38 (SEQ ID NO: 293); R1CHCL16 (SEQ ID NO: 294); R2CHCL10 (SEQ ID NO: 295); R1CHCL34 (SEQ ID NO: 296); R1CHCL82 (SEQ ID NO: 297); R2CHCL3 (SEQ ID NO: 298); R2CHCL69 (SEQ ID NO:299); R1CHCL56 (SEQ ID NO: 300); R2CHCL32 (SEQ ID NO: 301); R2CHCL49 (SEQ ID NO: 302); R2CHCL53 (SEQ ID NO: 303); R2CHCL22 (SEQ ID NO: 304); R2CHCL25 (SEQ ID NO: 305); R2CHCL18 (SEQ ID NO: 306); R1CHCL23 (SEQ ID NO: 307); R1CHCL27 (SEQ ID NO: 308); R2CHCL13 (SEQ ID NO: 309); R2CHCL14 (SEQ ID NO: 310); R2CHCL42 (SEQ ID NO: 311); R2CHCL41 (SEQ ID NO: 312); R2CHCL94 (SEQ ID NO: 313); or R2CHCL27 (SEQ ID NO: 314).

By way of example, in some embodiments, the Clec9A targeting moiety comprises an amino acid sequence selected from the following sequences:

1LEC 7 (SEQ ID NO: 315); 1LEC 9 (SEQ ID NO: 316); 1LEC 26 (SEQ ID NO: 317); 1LEC 27 (SEQ ID NO: 318); 1LEC 28 (SEQ ID NO: 319); 1LEC 30 (SEQ ID NO: 320); 1LEC 38 (SEQ ID NO: 333); 1LEC 42 (SEQ ID NO: 334); 1LEC 51 (SEQ ID NO: 335); 1LEC 61 (SEQ ID NO: 336); 1LEC 62 (SEQ ID NO: 337); 1LEC 63 (SEQ ID NO: 338); 1LEC 64 (SEQ ID NO: 339); 1LEC 70 (SEQ ID NO: 340); 1LEC 84 (SEQ ID NO: 341); 1LEC 88 (SEQ ID NO: 342); 1LEC 91 (SEQ ID NO: 343); 1LEC 92 (SEQ ID NO: 344); 1LEC 94 (SEQ ID NO: 345); 2LEC 6 (SEQ ID NO: 346); 2LEC 13 (SEQ ID NO: 347); 2LEC 16 (SEQ ID NO: 348); 2LEC 20 (SEQ ID NO: 349); 2LEC 23 (SEQ ID NO: 350); 2LEC 24 (SEQ ID NO: 351); 2LEC 26 (SEQ ID NO: 352); 2LEC 38 (SEQ ID NO: 353); 2LEC 48 (SEQ ID NO: 354); 2LEC 53 (SEQ ID NO: 355); 2LEC 54 (SEQ ID NO: 356); 2LEC 55 (SEQ ID NO: 357); 2LEC 59 (SEQ ID NO: 358); 2LEC 60 (SEQ ID NO: 359); 2LEC 61 (SEQ ID NO: 360); 2LEC 62 (SEQ ID NO: 361); 2LEC 63 (SEQ ID NO: 362); 2LEC 67 (SEQ ID NO: 363); 2LEC 68 (SEQ ID NO: 364); 2LEC 76 (SEQ ID NO: 365); 2LEC 83 (SEQ ID NO: 366); 2LEC 88 (SEQ ID NO: 367); 2LEC 89 (SEQ ID NO: 368); 2LEC 90 (SEQ ID NO: 369); 2LEC 93 (SEQ ID NO: 370); 2LEC 95 (SEQ ID NO: 371); 3LEC 4 (SEQ ID NO: 372); 3LEC 6 (SEQ ID NO: 373); 3LEC 9 (SEQ ID NO: 374); 3LEC 11 (SEQ ID NO: 375); 3LEC 13 (SEQ ID NO: 376); 3LEC 15 (SEQ ID NO: 377); 3LEC 22 (SEQ ID NO: 378); 3LEC 23 (SEQ ID NO: 379); 3LEC 27 (SEQ ID NO: 380); 3LEC 30 (SEQ ID NO: 381); 3LEC 36 (SEQ ID NO: 382); 3LEC 55 (SEQ ID NO: 383); 3LEC 57 (SEQ ID NO: 384); 3LEC 61 (SEQ ID NO: 385); 3LEC 62 (SEQ ID NO: 386); 3LEC 66 (SEQ ID NO: 387); 3LEC 69 (SEQ ID NO: 388); 3LEC 76 (SEQ ID NO: 389); 3LEC 82 (SEQ ID NO: 390); 3LEC 89 (SEQ ID NO: 391); or 3LEC 94 (SEQ ID NO: 392).

In some embodiments, the Clec9A targeting moiety comprises an amino acid sequence selected from SEQ ID Nos: 315-320 and 333-392 (provided above) without the terminal histidine tag sequence (i.e., HHHHHH; SEQ ID NO: 393).

In some embodiments, the Clec9A targeting moiety comprises an amino acid sequence selected from SEQ ID Nos: 315-320 and 333-392 (provided above) without the HA tag (i.e., YPYDVPDYGS; SEQ ID NO: 394).

In some embodiments, the Clec9A targeting moiety comprises an amino acid sequence selected from SEQ ID Nos: 315-320 and 333-392 (provided above) without the AAA linker (i.e., MA).

In some embodiments, the Clec9A targeting moiety comprises an amino acid sequence selected from SEQ ID Nos: 315-320 and 333-392 (provided above) without the AAA linker, HA tag, and terminal histidine tag sequence (i.e., AAAYPYDVPDYGSHHHHHH; SEQ ID NO: 395).

In an embodiment, the Clec9A targeting moiety comprises the anti-Clec9A antibody as disclosed in Tullett et al., JCI Insight. 2016; 1(7):e87102, the entire disclosures of which are hereby incorporated by reference.

In some embodiments, the present technology contemplates the use of any natural or synthetic analogs, mutants, variants, alleles, homologs and orthologs (herein collectively referred to as “analogs”) of the Clec9A targeting moieties described herein. In various embodiments, the amino acid sequence of the Clec9A targeting moiety further includes an amino acid analog, an amino acid derivative, or other non-classical amino acids.

In various embodiments, the Clec9A targeting moiety comprising a sequence that is at least 60% identical to any one of the sequences disclosed herein. For example, the Clec9A targeting moiety may comprise a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the Clec9A sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the Clec9A sequences disclosed herein).

In various embodiments, the Clec9A targeting moiety comprising an amino acid sequence having one or more amino acid mutations with respect to any one of the sequences disclosed herein. In various embodiments, the Clec9A targeting moiety comprises an amino acid sequence having one, or two, or three, or four, or five, or six, or seen, or eight, or nine, or ten, or fifteen, or twenty amino acid mutations with respect to any one of the sequences disclosed herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids. Exemplary non-classical amino acids include, but are not limited to, selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the present Clec9A binding agent's capability to specifically bind to Clec9A. In various embodiments, the mutations do not substantially reduce the present Clec9A binding agent's capability to specifically bind to Clec9A and without functionally modulating (e.g., partially or fully neutralizing) Clec9A.

In various embodiments, the binding affinity of the Clec9A targeting moiety for the full-length and/or mature forms and/or isoforms and/or splice variants and/or fragments and/or monomeric and/or dimeric forms and/or any other naturally occurring or synthetic analogs, variants, or mutants (including monomeric and/or dimeric forms) of human Clec9A may be described by the equilibrium dissociation constant (K_{D}). In various embodiments, the Clec9A targeting moiety binds to the full-length and/or mature forms and/or isoforms and/or splice variants and/or fragments and/or any other naturally occurring or synthetic analogs, variants, or mutants (including monomeric and/or dimeric forms) of human Clec9A with a K_{D} of less than about 1 uM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In various embodiments, the Clec9A targeting moiety binds but does not functionally modulate (e.g., partially or fully neutralize) the antigen of interest, i.e., Clec9A. For instance, in various embodiments, the Clec9A targeting moiety simply targets the antigen but does not substantially functionally modulate (e.g. partially or fully inhibit, reduce or neutralize) a biological effect that the antigen has. In various embodiments, the Clec9A targeting moiety binds an epitope that is physically separate from an antigen site that is important for its biological activity (e.g. an antigen's active site).

Such binding without significant function modulation finds use in various embodiments of the present invention, including methods in which the Clec9A targeting moiety is used to directly or indirectly recruit active immune cells to a site of need via an effector antigen. For example, in various embodiments, the Clec9A targeting moiety may be used to directly or indirectly recruit dendritic cells via Clec9A to a tumor cell in a method of reducing or eliminating a tumor (e.g. the Clec9A binding agent may comprise a targeting moiety having an anti-Clec9A antigen recognition domain and a targeting moiety having a recognition domain (e.g. antigen recognition domain) directed against a tumor antigen or receptor). In such embodiments, it is desirable to directly or indirectly recruit dendritic cells but not to functionally modulate or neutralize the Clec9A activity. In these embodiments, Clec9A signaling is an important piece of the tumor reducing or eliminating effect.

In some embodiments, the Clec9A targeting moiety enhances antigen-presentation by dendritic cells. For example, in various embodiments, the Clec9A targeting moiety can directly or indirectly recruit dendritic cells via Clec9A to a tumor cell, where tumor antigens are subsequently endocytosed and presented on the dendritic cell for induction of potent humoral and cytotoxic T cell responses.

In other embodiments (for example, related to treating autoimmune or neurodegenerative disease), the Clec9A targeting moiety binds and neutralizes the antigen of interest, i.e., Clec9A. For instance, in various embodiments, the present methods may inhibit or reduce Clec9A signaling or expression, e.g. to cause a reduction in an immune response.

### CD8 Targeting Moieties

In various embodiments, the targeting moiety is a CD8 targeting moiety that is a protein-based agent capable of specific binding to CD8. In various embodiments, the CD8 targeting moiety is a protein-based agent capable of specific binding to CD8 without functionally modulating (e.g. partial or complete neutralization) CD8.

CD8 is a heterodimeric type I transmembrane glycoprotein, whose α and β chains are both comprised of an immunoglobulin (Ig)-like extracellular domain connected by an extended 0-glycosylated stalk to a single-pass transmembrane domain and a short cytoplasmic tail. The cytoplasmic region of the CD8 α-chain contains two cysteine motifs that serve as a docking site for src tyrosine kinase p56lck (Lck). In contrast, this Lck binding domain appears to be absent from the CD8 β chain, suggesting that the β chain is not involved in downstream signaling. CD8 functions as a co-receptor for the T-cell receptor with its principle role being the recruitment of Lck to the TCR-pMHC complex following co-receptor binding to MHC. The increase in the local concentration of this kinase activates a signaling cascade that recruits and activates ζ-chain-associated protein kinase 70 (ZAP-70), subsequently leading to the amplification of T-cell activation signals.

In some embodiments, the CD8 targeting moiety comprises an antigen recognition domain that recognizes an epitope present on the CD8 α and/or β chains. In an embodiment, the antigen-recognition domain recognizes one or more linear epitopes on the CD8 α and/or β chains. In some embodiment, a linear epitope refers to any continuous sequence of amino acids present on the CD8 α and/or β chains. In another embodiment, the antigen-recognition domain recognizes one or more conformational epitopes present on the CD8 α and/or β chains. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the CD8 targeting moiety may bind to the full-length and/or mature forms and/or isoforms and/or splice variants and/or fragments and/or any other naturally occurring or synthetic analogs, variants, or mutants of human CD8 α and/or β chains. In various embodiments, the CD8 targeting moiety may bind to any forms of the human CD8 α and/or β chains, including monomeric, dimeric, heterodimeric, multimeric and associated forms. In an embodiment, the CD8 binding agent binds to the monomeric form of CD8 α chain or CD8 β chain. In another embodiment, the CD8 targeting moiety binds to a homodimeric form comprised of two CD8 α chains or two CD8 β chains. In a further embodiment, the CD8 binding agent binds to a heterodimeric form comprised of one CD8 α chain and one CD8 β chain.

In an embodiment, the CD8 targeting moiety comprises an antigen recognition domain that recognizes one or more epitopes present on the human CD8 α chain. In an embodiment, the human CD8 α chain comprises the amino acid sequence of Isoform 1 (SEQ ID NO: 396).

In an embodiment, the human CD8 α chain comprises the amino acid sequence of Isoform 2 (SEQ ID NO: 397).

In an embodiment, the human CD8 α chain comprises the amino acid sequence of Isoform 3 (SEQ ID NO: 398).

In an embodiment, the CD8 targeting moiety comprises an antigen recognition domain that recognizes one or more epitopes present on the human CD8 β chain. In an embodiment, the human CD8 β chain comprises the amino acid sequence of Isoform 1 (SEQ ID NO: 399).

In an embodiment, the human CD8 β chain comprises the amino acid sequence of Isoform 2 (SEQ ID NO: 400).

In an embodiment, the human CD8 β chain comprises the amino acid sequence of Isoform 3 (SEQ ID NO: 401).

In an embodiment, the human CD8 β chain comprises the amino acid sequence of Isoform 4 (SEQ ID NO: 402).

In an embodiment, the human CD8 β chain comprises the amino acid sequence of Isoform 5 (SEQ ID NO: 403).

In an embodiment, the human CD8 β chain comprises the amino acid sequence of Isoform 6 (SEQ ID NO: 404).

In an embodiment, the human CD8 β chain comprises the amino acid sequence of Isoform 7 (SEQ ID NO: 405).

In an embodiment, the human CD8 β chain comprises the amino acid sequence of Isoform 8 (SEQ ID NO: 406).

In some embodiments, the CD8 targeting moiety is capable of specific binding. In various embodiments, the CD8 targeting moiety comprises an antigen recognition domain such as an antibody or derivatives thereof.

In some embodiments, the CD8 targeting moiety comprise an antibody derivative or format. In some embodiments, the CD8 targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an alphabody; a bicyclic peptide; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the CD8 targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3).

In an embodiment, the CD8 targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. a HUMABODIES are described in, for example, WO2016/113555 and WO2016/113557, the entire disclosure of which is incorporated by reference.

In some embodiments, the CD8 targeting moiety comprises a VHH comprising a single amino acid chain having four “framework regions” or FRs and three “complementary determining regions” or CDRs. As used herein, “framework region” or “FR” refers to a region in the variable domain that is located between the CDRs. As used herein, “complementary determining region” or “CDR” refers to variable regions in VHHs that contains the amino acid sequences capable of specifically binding to antigenic targets.

In various embodiments, the CD8 targeting moiety comprises a VHH having a variable domain comprising at least one CDR1, CDR2, and/or CDR3 sequences.

In some embodiments, the CDR1 sequence is selected from SEQ ID Nos: 407-477.

In some embodiments, the CDR2 sequence is selected from SEQ ID Nos: 478-548.

In some embodiments, the CDR3 sequence is selected from SEQ ID Nos: 549-620.

In various embodiments, the CD8 targeting moiety comprises SEQ ID NO: 407, SEQ ID NO: 478, and SEQ ID NO: 549.

In various embodiments, the CD8 targeting moiety comprises SEQ ID NO: 407, SEQ ID NO: 478, and SEQ ID NO: 550.

In various embodiments, the CD8 targeting moiety comprises SEQ ID NO: 407, SEQ ID NO: 478, and SEQ ID NO: 551.

In various embodiments, the CD8 targeting moiety comprises SEQ ID NO: 407, SEQ ID NO: 479, and SEQ ID NO: 549.

In various embodiments, the CD8 targeting moiety comprises SEQ ID NO: 407, SEQ ID NO: 479, and SEQ ID NO: 550.

In various embodiments, the CD8 targeting moiety comprises SEQ ID NO: 407, SEQ ID NO: 479, and SEQ ID NO: 551.

In various embodiments, the CD8 targeting moiety comprises SEQ ID NO: 408, SEQ ID NO: 478, and SEQ ID NO: 549.

In various embodiments, the CD8 targeting moiety comprises SEQ ID NO: 408, SEQ ID NO: 478, and SEQ ID NO: 550.

In various embodiments, the CD8 targeting moiety comprises SEQ ID NO: 408, SEQ ID NO: 478, and SEQ ID NO: 551.

In various embodiments, the CD8 targeting moiety comprises SEQ ID NO: 408, SEQ ID NO: 479, and SEQ ID NO: 549.

In various embodiments, the CD8 targeting moiety comprises SEQ ID NO: 408, SEQ ID NO: 479, and SEQ ID NO: 550.

In various embodiments, the CD8 targeting moiety comprises SEQ ID NO: 408, SEQ ID NO: 479, and SEQ ID NO: 551.

By way of example, in some embodiments, the CD8 targeting moiety comprises an amino acid sequence selected from the following sequences: R3HCD27 (SEQ ID NO: 621); R3HCD129 (SEQ ID NO: 622); or R2HCD26 (SEQ ID NO: 623).

In various embodiments, the CD8 targeting moiety comprises an amino acid sequence selected from the following sequences: 1CDA 7 (SEQ ID NO: 624); 1CDA 12 (SEQ ID NO: 625); 1CDA 14 (SEQ ID NO: 626); 1CDA 15 (SEQ ID NO: 627); 1CDA 17 (SEQ ID NO: 628); 1CDA 18 (SEQ ID NO: 629); 1CDA 19 (SEQ ID NO: 630); 1CDA 24 (SEQ ID NO: 631); 1CDA 26 (SEQ ID NO: 632); 1CDA 28 (SEQ ID NO: 633); 1CDA 37 (SEQ ID NO: 634); 1CDA 43 (SEQ ID NO: 635); 1CDA 45 (SEQ ID NO: 636); 1CDA 47 (SEQ ID NO: 637); 1CDA 48 (SEQ ID NO: 638); 1CDA 58 (SEQ ID NO: 639); 1CDA 65 (SEQ ID NO: 640); 1CDA 68 (SEQ ID NO: 641); 1CDA 73 (SEQ ID NO: 642); 1CDA 75 (SEQ ID NO: 643); 1CDA 86 (SEQ ID NO: 644); 1CDA 87 (SEQ ID NO: 645); 1CDA 88 (SEQ ID NO: 646); 1CDA 89 (SEQ ID NO: 647); 1CDA 92 (SEQ ID NO: 648); 1CDA 93 (SEQ ID NO: 649); 2CDA 1 (SEQ ID NO: 650); 2CDA 5 (SEQ ID NO: 651); 2CDA 22 (SEQ ID NO: 652); 2CDA 28 (SEQ ID NO: 653); 2CDA 62 (SEQ ID NO: 654); 2CDA 68 (SEQ ID NO: 655); 2CDA 73 (SEQ ID NO: 656); 2CDA 74 (SEQ ID NO: 657); 2CDA 75 (SEQ ID NO: 658); 2CDA 77 (SEQ ID NO: 659); 2CDA 81 (SEQ ID NO: 660); 2CDA 87 (SEQ ID NO: 661); 2CDA 88 (SEQ ID NO: 662); 2CDA 89 (SEQ ID NO: 663); 2CDA 91 (SEQ ID NO: 664); 2CDA 92 (SEQ ID NO: 665); 2CDA 93 (SEQ ID NO: 666); 2CDA 94 (SEQ ID NO: 667); 2CDA 95 (SEQ ID NO: 668); 3CDA 3 (SEQ ID NO: 669); 3CDA 8 (SEQ ID NO: 670); 3CDA 11 (SEQ ID NO: 671); 3CDA 18 (SEQ ID NO: 672); 3CDA 19 (SEQ ID NO: 673); 3CDA 21 (SEQ ID NO: 674); 3CDA 24 (SEQ ID NO: 675); 3CDA 28 (SEQ ID NO: 676); 3CDA 29 (SEQ ID NO: 677); 3CDA 31 (SEQ ID NO: 678); 3CDA 32 (SEQ ID NO: 679); 3CDA 33 (SEQ ID NO: 680); 3CDA 37 (SEQ ID NO: 681); 3CDA 40 (SEQ ID NO: 682); 3CDA 41 (SEQ ID NO:683); 3CDA 48 (SEQ ID NO: 684); 3CDA 57 (SEQ ID NO: 685); 3CDA 65 (SEQ ID NO: 686); 3CDA 70 (SEQ ID NO: 687); 3CDA 73 (SEQ ID NO: 688); 3CDA 83 (SEQ ID NO: 689); 3CDA 86 (SEQ ID NO: 690); 3CDA 88 (SEQ ID NO: 691); or 3CDA 90 (SEQ ID NO: 692).

In some embodiments, the CD8 targeting moiety comprises an amino acid sequence selected from SEQ ID NOs: 624-692 (provided above) without the terminal histidine tag sequence (i.e., HHHHHH; SEQ ID NO: 393).

In some embodiments, the CD8 targeting moiety comprises an amino acid sequence selected from SEQ ID NOs: 621-692 (provided above) without the HA tag (i.e., YPYDVPDYGS; SEQ ID NO: 394).

In some embodiments, the CD8 targeting moiety comprises an amino acid sequence selected from SEQ ID NOs: 621-692 (provided above) without the AAA linker (i.e., MA).

In some embodiments, the CD8 targeting moiety comprises an amino acid sequence selected from SEQ ID NOs: 621-623 (provided above) without the AAA linker and HA tag.

In some embodiments, the CD8 targeting moiety comprises an amino acid sequence selected from SEQ ID NOs: 624-692 (provided above) without the AAA linker, HA tag, and terminal histidine tag sequence (i.e., AAAYPYDVPDYGSHHHHHH; SEQ ID NO: 395).

In some embodiments, the CD8 targeting moiety comprises an amino acid sequence described in US Patent Publication No. 2014/0271462, the entire contents of which are incorporated by reference. In various embodiments, the CD8 binding agent comprises an amino acid sequence described in Table 0.1, Table 0.2, Table 0.3, and/or FIGS. 1A-12I of US Patent Publication No. 2014/0271462, the entire contents of which are incorporated by reference. In various embodiments, the CD8 binding agent comprises a HCDR1 of SEQ ID NO: 693 or 694 and/or a HCDR2 of SEQ ID NO: 693 or 694 and/or a HCDR3 of SEQ ID NO: 693 or 694 and/or a LCDR1 of SEQ ID NO: 695 and/or a LCDR2 of SEQ ID NO: 695 and/or a LCDR3 of SEQ ID NO: 695.

In some embodiments, the present technology contemplates the use of any natural or synthetic analogs, mutants, variants, alleles, homologs and orthologs (herein collectively referred to as “analogs”) of the CD8 targeting moiety described herein. In some embodiments, the amino acid sequence of the CD8 targeting moiety further includes an amino acid analog, an amino acid derivative, or other non-classical amino acids.

In some embodiments, the CD8 targeting moiety comprises a targeting moiety comprising a sequence that is at least 60% identical to any one of the CD8 sequences disclosed herein. For example, the CD8 targeting moiety may comprise a targeting moiety comprising a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any one of the CD8 sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the CD8 sequences disclosed herein).

In various embodiments, the CD8 targeting moiety comprises an amino acid sequence having one or more amino acid mutations with respect to any one of the CD8 sequences disclosed herein. In various embodiments, the CD8 binding agent comprises a targeting moiety comprising an amino acid sequence having one, or two, or three, or four, or five, or six, or seen, or eight, or nine, or ten, or fifteen, or twenty amino acid mutations with respect to any one of the CD8 sequences disclosed herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the CD8 targeting moiety's capability to specifically bind to CD8. In various embodiments, the mutations do not substantially reduce the CD8 targeting moiety's capability to specifically bind to CD8 without functionally modulating CD8.

In various embodiments, the binding affinity of the CD8 targeting moiety for the full-length and/or mature forms and/or isoforms and/or splice variants and/or fragments and/or any other naturally occurring or synthetic analogs, variants, or mutants (including monomeric, dimeric, heterodimeric, multimeric and/or associated forms) of human CD8 α and/or β chains may be described by the equilibrium dissociation constant (K_{D}). In various embodiments, the CD8 targeting moiety binds to the full-length and/or mature forms and/or isoforms and/or splice variants and/or fragments and/or any other naturally occurring or synthetic analogs, variants, or mutants (including monomeric, dimeric, heterodimeric, multimeric and/or associated forms) of human CD8 α and/or β chains with a K_{D} of less than about 1 uM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In various embodiments, the CD8 targeting moiety binds but does not functionally modulate the antigen of interest, i.e., CD8. For instance, in various embodiments, the CD8 targeting moiety simply targets the antigen but does not substantially functionally modulate the antigen, e.g. it does not substantially inhibit, reduce or neutralize a biological effect that the antigen has. In various embodiments, the CD8 targeting moiety binds an epitope that is physically separate from an antigen site that is important for its biological activity (e.g. an antigen's active site).

Such non-functionally modulating (e.g. non-neutralizing) binding finds use in various embodiments of the present invention, including methods in which the CD8 targeting moiety is used to directly or indirectly recruit active immune cells to a site of need via an effector antigen. For example, in various embodiments, the CD8 targeting moiety may be used to directly or indirectly recruit cytotoxic T cells via CD8 to a tumor cell in a method of reducing or eliminating a tumor (e.g. the CD8 binding agent may comprise a targeting moiety having an anti-CD8 antigen recognition domain and a targeting moiety having a recognition domain (e.g. an antigen recognition domain) directed against a tumor antigen or receptor). In such embodiments, it is desirable to directly or indirectly recruit CD8-expressing cytotoxic T cells but not to neutralize the CD8 activity. In these embodiments, CD8 signaling is an important piece of the tumor reducing or eliminating effect.

### PD-1, PD-L1, or PD-L2 Targeting Moieties

In some embodiments, the targeting moiety is a PD-1, PD-L1, or PD-L2 targeting moiety that is a protein-based agent capable of specific binding to PD-1, PD-L1, or PD-L2. In some embodiments, the PD-1, PD-L1, or PD-L2 targeting moiety binds but does not functionally modulate (e.g., partially or fully neutralize) the antigen of interest, i.e., PD-1, PD-L1, or PD-L2. For instance, in various embodiments, the PD-1, PD-L1, or PD-L2 targeting moiety simply targets the antigen but does not substantially functionally modulate (e.g. partially or fully inhibit, reduce or neutralize) a biological effect that the antigen has. In various embodiments, the PD-1, PD-L1, or PD-L2 targeting moiety binds an epitope that is physically separate from an antigen site that is important for its biological activity (e.g. an antigen's active site).

PD-1 Targeting Moieties Programmed cell death protein 1, also known as PD-1 and cluster of differentiation 279 (CD279), is a cell surface receptor that is primarily expressed on activated T cells, B cells, and macrophages. PD-1 has been shown to negatively regulate antigen receptor signaling upon engagement of its ligands (i.e., PD-L1 and/or PD-L2). PD-1 plays an important role in down-regulating the immune system and promoting self tolerance by suppressing T cell inflammatory activity. PD-1 is a type I transmembrane glycoprotein containing an Ig Variable-type (V-type) domain responsible for ligand binding and a cytoplasmic tail that is responsible for the binding of signaling molecules. The cytoplasmic tail of PD-1 contains two tyrosine-based signaling motifs, an ITIM (immunoreceptor tyrosine-based inhibition motif) and an ITSM (immunoreceptor tyrosine-based switch motif).

In some embodiments, the PD-1 targeting moiety comprises an antigen recognition domain that recognizes an epitope present on PD-1. In an embodiment, the antigen-recognition domain recognizes one or more linear epitopes present on PD-1. In some embodiments, a linear epitope refers to any continuous sequence of amino acids present on PD-1. In another embodiment, the antigen-recognition domain recognizes one or more conformational epitopes present on PD-1. As used herein, a conformation epitope refers to one or more sections of amino acids (which may be discontinuous) which form a three-dimensional surface with features and/or shapes and/or tertiary structures capable of being recognized by an antigen recognition domain.

In some embodiments, the PD-1 targeting moiety may bind to the full-length and/or mature forms and/or isoforms and/or splice variants and/or fragments and/or any other naturally occurring or synthetic analogs, variants, or mutants of human PD-1. In various embodiments, the PD-1 targeting moiety may bind to any forms of the human PD-1. In an embodiment, the PD-1 targeting moiety binds to a phosphorylated form of PD-1.

In an embodiment, the PD-1 targeting moiety comprises an antigen recognition domain that recognizes one or more epitopes present on human PD-1. In an embodiment, the human PD-1 comprises the amino acid sequence of (signal peptide underlined):

(SEQ ID NO: 696)
MQIPQAPWPVVWAVLQLGWRPGWFLDSPDRPWNPPTFSPALLVVTEGD
NATFTCSFSNTSESFVLNWYRMSPSNQTDKLAAFPEDRSQPGQDCRFR
VTQLPNGRDFHMSVVRARRNDSGTYLCGAISLAPKAQIKESLRAELRV
TERRAEVPTAHPSPSPRPAGQFQTLVVGVVGGLLGSLVLLVWVLAVIC
SRAARGTIGARRTGQPLKEDPSAVPVFSVDYGELDFQWREKTPEPPVP
CVPEQTEYATIVFPSGMGTSSPARRGSADGPRSAQPLRPEDGHCSWPL.

In another embodiment, the human PD-1 comprises the amino acid sequence of SEQ ID NO: 696 without the amino-terminal signal peptide.

In some embodiments, the PD-1 targeting moiety is capable of specific binding. In various embodiments, the PD-1 targeting moiety comprises an antigen recognition domain such as an antibody or derivatives thereof.

In some embodiments, the PD-1 targeting moiety comprises an antibody derivative or format. In some embodiments, the PD-1 targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an alphabody; a bicyclic peptide; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the PD-1 targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3).

In an embodiment, the PD-1 targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO2016/113555 and WO2016/113557, the entire disclosure of which is incorporated by reference.

In some embodiments, the PD-1 targeting moiety comprises a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In various embodiments, the PD-1 targeting moiety comprises a VHH having a variable domain comprising at least one CDR1, CDR2, and/or CDR3 sequences. In various embodiments, the PD-1 binding agent comprises a VHH having a variable region comprising at least one FR1, FR2, FR3, and FR4 sequences.

In some embodiments, the CDR1 sequence is selected from SEQ ID Nos.: 697-710.

In some embodiments, the CDR2 sequence is selected from SEQ ID Nos.: 711-724.

In some embodiments, the CDR3 sequence is selected from SEQ ID Nos.: 725-738.

In various exemplary embodiments, the PD-1 targeting moiety comprises an amino acid sequence selected from the following sequences:

2PD23 (SEQ ID NO: 739); 2PD26 (SEQ ID NO: 740); 2PD90 (SEQ ID NO: 741); 2PD-106 (SEQ ID NO: 742); 2PD-16 (SEQ ID NO: 743); 2PD71 (SEQ ID NO: 744); 2PD-152 (SEQ ID NO: 745); 2PD-12 (SEQ ID NO: 746); 3PD55 (SEQ ID NO: 747); 3PD82 (SEQ ID NO: 748); 2PD8 (SEQ ID NO: 749); 2PD27 (SEQ ID NO: 750); 2PD82 (SEQ ID NO: 751); or 3PD36 (SEQ ID NO: 752).

In some embodiments, the PD-1 targeting moiety comprises an amino acid sequence selected from SEQ ID NOs: 739-752 (provided above) without the terminal histidine tag sequence (i.e., HHHHHH; SEQ ID NO: 393).

In some embodiments, the PD-1 targeting moiety comprises an amino acid sequence selected from SEQ ID NOs: 739-752 (provided above) without the HA tag (i.e., YPYDVPDYGS; SEQ ID NO: 394).

In some embodiments, the PD-1 targeting moiety comprises an amino acid sequence selected from SEQ ID NOs: 739-752 (provided above) without the AAA linker (i.e., MA).

In some embodiments, the PD-1 targeting moiety comprises an amino acid sequence selected from SEQ ID NOs: 739-752 (provided above) without the AAA linker, HA tag, and terminal histidine tag sequence (i.e., AAAYPYDVPDYGSHHHHHH; SEQ ID NO: 395).

In some embodiments, the present technology contemplates the use of any natural or synthetic analogs, mutants, variants, alleles, homologs and orthologs (herein collectively referred to as “analogs”) of the PD-1 targeting moiety described herein. In some embodiments, the amino acid sequence of the PD1 targeting moiety further includes an amino acid analog, an amino acid derivative, or other non-classical amino acids.

In some embodiments, the PD-1 targeting moiety comprises the anti-PD-1 antibody pembrolizumab (aka MK-3475, KEYTRUDA), or fragments thereof. Pembrolizumab and other humanized anti-PD-1 antibodies are disclosed in Hamid, et al. (2013) New England Journal of Medicine 369 (2): 134-44, U.S. Pat. No. 8,354,509, and WO 2009/114335, the entire disclosures of which are hereby incorporated by reference. In illustrative embodiments, pembrolizumab or an antigen-binding fragment thereof for use in the methods provided herein comprises a heavy chain comprising the amino acid sequence of SEQ ID NO: 753; and/or a light chain comprising the amino acid sequence of (SEQ ID NO: 754).

In an embodiment, the PD-1 targeting moiety comprises the anti-PD-1 antibody, nivolumab (aka BMS-936558, MDX-1106, ONO-4538, OPDIVO), or fragments thereof. Nivolumab (clone 5C4) and other human monoclonal antibodies that specifically bind to PD-1 are disclosed in U.S. Pat. No. 8,008,449 and WO 2006/121168, the entire disclosures of which are hereby incorporated by reference. In illustrative embodiments, nivolumab or an antigen-binding fragment thereof comprises a heavy chain comprising the amino acid sequence of SEQ ID NO: 755; and/or a light chain comprising the amino acid sequence of (SEQ ID NO: 756).

In an embodiment, the PD-1 targeting moiety comprises the anti-PD-1 antibody pidilizumab (aka CT-011, hBAT or hBAT-1), or fragments thereof. Pidilizumab and other humanized anti-PD-I monoclonal antibodies are disclosed in US 2008/0025980 and WO 2009/101611, the entire disclosures of which are hereby incorporated by reference. In illustrative embodiments, the anti-PD-1 antibody or an antigen-binding fragment thereof for use in the methods provided herein comprises a light chain variable regions comprising an amino acid sequence selected from SEQ ID NOS: 15-18 of US 2008/0025980 (SEQ ID Nos: 757-760 of this application); and/or a heavy chain comprising an amino acid sequence selected from SEQ ID NOS: 20-24 of US 2008/0025980 (SEQ ID Nos: 761-765 of this application).

In an embodiment, the targeting moiety comprises a light chain comprising SEQ ID NO: 18 of US 2008/0025980 (SEQ ID NO: 760) and a heavy chain comprising SEQ ID NO: 22 of US 2008/0025980 (SEQ ID NO: 763).

In an embodiment, the PD-1 targeting moiety comprises AMP-514 (aka MEDI-0680).

In an embodiment, the PD-1 targeting moiety comprises the PD-L2-Fc fusion protein AMP-224, which is disclosed in WO2010/027827 and WO 2011/066342, the entire disclosures of which are hereby incorporated by reference.

In such an embodiment, the targeting moiety may include a targeting domain which comprises SEQ ID NO: 4 of WO2010/027827 (SEQ ID NO: 766 of this application) and/or the B7-DC fusion protein which comprises SEQ ID NO:83 of WO2010/027827 (SEQ ID NO: 767 of this application).

In an embodiment, the PD-1 targeting moiety comprises the peptide AUNP 12 or any of the other peptides disclosed in US 2011/0318373 or U.S. Pat. No. 8,907,053. For example, the targeting moiety may comprise AUNP 12 (i.e., Compound 8 or SEQ ID NO:49 of US 2011/0318373) which has the sequence of:

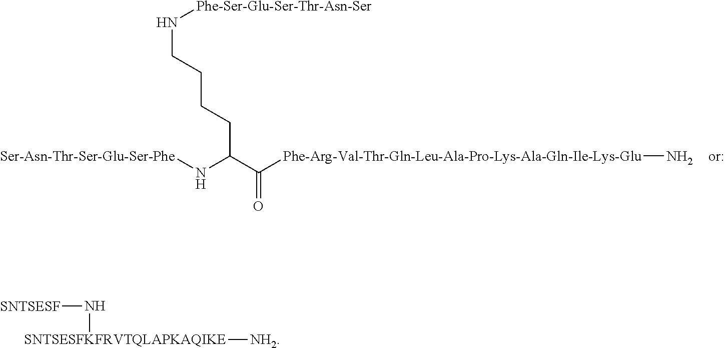

In an embodiment, the PD-1 targeting moiety comprises the anti-PD-1 antibody 1E3, or fragments thereof, as disclosed in US 2014/0044738, the entire disclosures of which are hereby incorporated by reference. In illustrative embodiments, 1E3 or an antigen-binding fragment thereof for use in the methods provided herein comprises a heavy chain variable region comprising the amino acid sequence of SEQ ID NO: 768; and/or a light chain variable region comprising the amino acid sequence of SEQ ID NO: 769.

In an embodiment, the PD-1 targeting moiety comprises the anti-PD-1 antibody 1E8, or fragments thereof, as disclosed in US 2014/0044738, the entire disclosures of which are hereby incorporated by reference. In illustrative embodiments, 1E8 or an antigen-binding fragment thereof for use in the methods provided herein comprises a heavy chain variable region comprising the amino acid sequence of SEQ ID NO: 770; and/or a light chain variable region comprising the amino acid sequence of SEQ ID NO: 771.

In an embodiment, the PD-1 targeting moiety comprises the anti-PD-1 antibody 1H3, or fragments thereof, as disclosed in US 2014/0044738, the entire disclosures of which are hereby incorporated by reference. In illustrative embodiments, 1H3 or an antigen-binding fragment thereof for use in the methods provided herein comprises a heavy chain variable region comprising the amino acid sequence of SEQ ID NO: 772; and/or light chain variable region comprising the amino acid sequence of SEQ ID NO: 773.

In an embodiment, the PD-1 targeting moiety comprises a VHH directed against PD-1 as disclosed, for example, in U.S. Pat. No. 8,907,065 and WO 2008/071447, the entire disclosures of which are hereby incorporated by reference. In illustrative embodiments, the VHHs against PD-1 comprise SEQ ID NOS: 347-351 of U.S. Pat. No. 8,907,065 (SEQ ID Nos: 774-778).

In an embodiment, the PD-1 targeting moiety comprises any one of the anti-PD-1 antibodies, or fragments thereof, as disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID NOS: 25-29 of US2011/0271358 (SEQ ID Nos: 779-783 of this application); and/or a light chain comprising an amino acid sequence selected from SEQ ID NOS: 30-33 of US2011/0271358 (SEQ ID Nos: 784-787 of this application).

In some embodiments, the PD-1 targeting moiety is an antibody directed against PD-1, or an antibody fragment thereof, selected from TSR-042 (Tesaro, Inc.), REGN2810 (Regeneron Pharmaceuticals, Inc.), PDR001 (Novartis Pharmaceuticals), and BGB-A317 (BeiGene Ltd.)

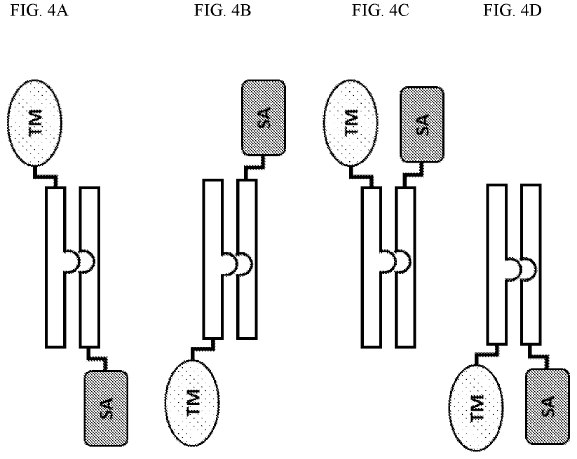

In one embodiment, the targeting moiety binds to PD-1 and the signaling moiety is a wild type IFNα or a modified IFNα. In some embodiments, the Fc chimeric protein is in one of the configurations shown in FIGS. 4A to 4D, where the targeting moiety binds to PD-1 and the signaling moiety is a wild type IFNα. In some embodiments, the targeting moiety binds to PD-1 (e.g., scFv) and the signaling moiety is a wild type IFNα2.

PD-L1 Targeting Moieties

In some embodiments, the targeting moiety is a PD-L1 targeting moiety. Programmed death-ligand 1 (PD-L1) also known as cluster of differentiation 274 (CD274) or B7 homolog 1 (B7-H1) is a type 1 transmembrane protein that has been speculated to play a major role in suppressing the immune system. PD-L1 is upregulated on macrophages and dendritic cells (DC) in response to LPS and GM-CSF treatment, and on T cells and B cells upon TCR and B cell receptor signaling.

In various embodiments, the PD-L1 targeting moiety comprises an antigen recognition domain that recognizes an epitope present on PD-L1. In an embodiment, the antigen-recognition domain recognizes one or more linear epitopes present on PD-L1. In some embodiment, a linear epitope refers to any continuous sequence of amino acids present on PD-L1. In another embodiment, the antigen-recognition domain recognizes one or more conformational epitopes present on PD-L1. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the PD-L1 targeting moiety may bind to the full-length and/or mature forms and/or isoforms and/or splice variants and/or fragments and/or any other naturally occurring or synthetic analogs, variants, or mutants of human PD-L1. In various embodiments, the PD-L1 targeting moiety may bind to any forms of the human PD-L1. In an embodiment, the PD-L1 targeting moiety binds to a phosphorylated form of PD-L1. In an embodiment, the PD-L1 targeting moiety binds to an acetylated form of PD-L1.

In an embodiment, the PD-L1 targeting moiety comprises an antigen recognition domain that recognizes one or more epitopes present on human PD-L1. In an embodiment, the human PD-L1 comprises the amino acid sequence of (signal peptide underlined):

Isoform 1:
(SEQ ID NO: 788)
MRIFAVFIFMTYWHLLNAFTVTVPKDLYWEYGSNMTIECKFPVEKQLDLA
ALIVYWEMEDKNIIQFVHGEEDLKVQHSSYRQRARLLKDQLSLGNAALQI
TDVKLQDAGVYRCMISYGGADYKRITVKVNAPYNKINQRILVVDPVTSEH
ELTCQAEGYPKAEVIWTSSDHQVLSGKTTTTNSKREEKLFNVTSTLRINT
TTNEIFYCTFRRLDPEENHTAELVIPELPLAHPPNERTHLVILGAILLCL
GVALTFIFRLRKGRMMDVKKCGIQDTNSKKQSDTHLEET;
Isoform 2:
(SEQ ID NO: 789)
MRIFAVFIFMTYWHLLNAPYNKINQRILVVDPVTSEHELTCQAEGYPKAE
VIWTSSDHQVLSGKTTTTNSKREEKLFNVTSTLRINTTTNEIFYCTFRRL
DPEENHTAELVIPELPLAHPPNERTHLVILGAILLCLGVALTFIFRLRKG
RMMDVKKCGIQDTNSKKQSDTHLEET; or
Isoform 3:
(SEQ ID NO: 790)
MRIFAVFIFMTYWHLLNAFTVTVPKDLYWEYGSNMTIECKFPVEKQLDLA
ALIVYWEMEDKNIIQFVHGEEDLKVQHSSYRQRARLLKDQLSLGNAALQI
TDVKLQDAGVYRCMISYGGADYKRITVKVNAPYNKINQRILVVDPVTSEH
ELTCQAEGYPKAEVIWTSSDHQVLSGD.

In various embodiments, the PD-L1 targeting moiety is capable of specific binding. In various embodiments, the PD-L1 targeting moiety comprises an antigen recognition domain such as an antibody or derivatives thereof. In an embodiment, the PD-L1 targeting moiety comprises an antibody.

In some embodiments, the PD-L1 targeting moiety comprises an antibody derivative or format. In some embodiments, the PD-L1 targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an alphabody; a bicyclic peptide; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)2, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the PD-L1 targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3).

In an embodiment, the PD-L1 targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO2016/113555 and WO2016/113557, the entire disclosure of which is incorporated by reference.

In some embodiments, the PD-L1 targeting moiety comprises a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In various embodiments, the PD-L1 targeting moiety comprises a VHH having a variable domain comprising at least one CDR1, CDR2, and/or CDR3 sequences. In various embodiments, the PD-L1 targeting moiety comprises a VHH having a variable region comprising at least one FR1, FR2, FR3, and FR4 sequences.

In some embodiments, the CDR1 sequence is selected from SEQ ID Nos.: 791-821.

In some embodiments, the CDR2 sequence is selected from SEQ ID Nos.: 822-852.

In some embodiments, the CDR3 sequence is selected from SEQ ID Nos.: 853-883.

In various exemplary embodiments, the PD-L1 targeting moiety comprises an amino acid sequence selected from the following sequences: 2LIG2 (SEQ ID NO: 884); 2LIG3 (SEQ ID NO: 885); 2LIG16 (SEQ ID NO: 886); 2LIG22 (SEQ ID NO: 887); 2LIG27 (SEQ ID NO: 888); 2LIG29 (SEQ ID NO: 889); 2LIG30 (SEQ ID NO: 890); 2LIG34 (SEQ ID NO: 891); 2LIG35 (SEQ ID NO: 892); 2LIG48 (SEQ ID NO: 893); 2LIG65 (SEQ ID NO: 894); 2LIG85 (SEQ ID NO: 895); 2LIG86 (SEQ ID NO: 896); 2LIG89 (SEQ ID NO: 897); 2LIG97 (SEQ ID NO: 898); 2LIG99 (SEQ ID NO: 899); 2LIG109 (SEQ ID NO: 900); 2LIG127 (SEQ ID NO: 901); 2LIG139 (SEQ ID NO: 902); 2LIG176 (SEQ ID NO: 903); 2LIG189 (SEQ ID NO: 904); 3LIG3 (SEQ ID NO: 905); 3LIG7 (SEQ ID NO: 906); 3LIG8 (SEQ ID NO: 907); 3LIG9 (SEQ ID NO: 908); 3LIG18 (SEQ ID NO: 909); 3LIG20 (SEQ ID NO: 910); 3LIG28 (SEQ ID NO: 911); 3LIG29 (SEQ ID NO: 912); 3LIG30 (SEQ ID NO: 913); or 3LIG33 (SEQ ID NO: 914).

In some embodiments, the PD-L1 targeting moiety comprises an amino acid sequence selected from SEQ ID NOs: 884-914 (provided above) without the terminal histidine tag sequence (i.e., HHHHHH; SEQ ID NO: 393).

In some embodiments, the PD-L1 targeting moiety comprises an amino acid sequence selected from SEQ ID NOs: 884-914 (provided above) without the HA tag (i.e., YPYDVPDYGS; SEQ ID NO: 394).

In some embodiments, the PD-L1 targeting moiety comprises an amino acid sequence selected from SEQ ID NOs: 884-914 (provided above) without the AAA linker (i.e., MA).

In some embodiments, the PD-L1 targeting moiety comprises an amino acid sequence selected from SEQ ID NOs: 884-914 (provided above) without the AAA linker, HA tag, and terminal histidine tag sequence (i.e., AAAYPYDVPDYGSHHHHHH; SEQ ID NO: 395).

In an embodiment, the PD-L1 targeting moiety comprises the anti-PD-L1 antibody MED14736 (aka durvalumab), or fragments thereof. MED14736 is selective for PD-L1 and blocks the binding of PD-L1 to the PD-1 and CD80 receptors. MED14736 and antigen-binding fragments thereof for use in the methods provided herein comprises a heavy chain and a light chain or a heavy chain variable region and a light chain variable region. The sequence of MED14736 is disclosed in WO/2016/06272, the entire contents of which are hereby incorporated by reference. In illustrative embodiments, MED14736 or an antigen-binding fragment thereof for use in the methods provided herein comprises a heavy chain comprising the amino acid sequence of SEQ ID NO: 915; and/or a light chain comprising the amino acid sequence of SEQ ID NO: 916.

In illustrative embodiments, the MED14736 or an antigen-binding fragment thereof for use in the methods provided herein comprises a heavy chain variable region comprising the amino acid sequence of SEQ ID NO: 4 of WO/2016/06272 (SEQ ID NO: 917); and/or a light chain variable region comprising the amino acid sequence of SEQ ID NO: 3 of WO/2016/06272 (SEQ ID NO: 918).

In an embodiment, the PD-L1 targeting moiety comprises the anti-PD-L1 antibody atezolizumab (aka MPDL3280A, RG7446), or fragments thereof. In illustrative embodiments, atezolizumab or an antigen-binding fragment thereof for use in the methods provided herein comprises a heavy chain comprising the amino acid sequence of SEQ ID NO: 919; and/or a light chain comprising the amino acid sequence of SEQ ID NO: 920.

In an embodiment, the PD-L1 targeting moiety comprises the anti-PD-L1 antibody avelumab (aka MSB0010718C), or fragments thereof. In illustrative embodiments, avelumab or an antigen-binding fragment thereof for use in the methods provided herein comprises a heavy chain comprising the amino acid sequence of SEQ ID NO: 921; and/or a light chain comprising the amino acid sequence of SEQ ID NO: 922.

In an embodiment, the PD-L1 targeting moiety comprises the anti-PD-L1 antibody BMS-936559 (aka 12A4, MDX-1105), or fragments thereof, as disclosed in US 2013/0309250 and WO2007/005874, the entire disclosures of which are hereby incorporated by reference. In illustrative embodiments, BMS-936559 or an antigen-binding fragment thereof for use in the methods provided herein comprises a heavy chain variable region comprising the amino acid sequence of SEQ ID NO: 923; and/or a light chain variable region comprising the amino acid sequence of SEQ ID NO: 924.

In an embodiment, the PD-L1 targeting moiety comprises the anti-PD-L1 antibody 3G10, or fragments thereof, as disclosed in US 2013/0309250 and WO2007/005874, the entire disclosures of which are hereby incorporated by reference. In illustrative embodiments, 3G10 or an antigen-binding fragment thereof for use in the methods provided herein comprises a heavy chain variable region comprising the amino acid sequence of SEQ ID NO: 925; and/or a light chain variable region comprising the amino acid sequence of SEQ ID NO: 926.

In an embodiment, the PD-L1 targeting moiety comprises the anti-PD-L1 antibody 10A5, or fragments thereof, as disclosed in US 2013/0309250 and WO2007/005874, the entire disclosures of which are hereby incorporated by reference. In illustrative embodiments, 10A5 or an antigen-binding fragment thereof for use in the methods provided herein comprises a heavy chain variable region comprising the amino acid sequence of SEQ ID NO: 927; and/or a light chain variable region comprising the amino acid sequence of SEQ ID NO: 928.

In an embodiment, the PD-L1 targeting moiety comprises the anti-PD-L1 antibody 5F8, or fragments thereof, as disclosed in US 2013/0309250 and WO2007/005874, the entire disclosures of which are hereby incorporated by reference. In illustrative embodiments, 5F8 or an antigen-binding fragment thereof for use in the methods provided herein comprises a heavy chain variable region comprising the amino acid sequence of SEQ ID NO: 929; and/or a light chain variable region comprising the amino acid sequence of SEQ ID NO: 930.

In an embodiment, the PD-L1 targeting moiety comprises the anti-PD-L1 antibody 10H10, or fragments thereof, as disclosed in US 2013/0309250 and WO2007/005874, the entire disclosures of which are hereby incorporated by reference. In illustrative embodiments, 10H10 or an antigen-binding fragment thereof for use in the methods provided herein comprises a heavy chain variable region comprising the amino acid sequence of SEQ ID NO: 931; and/or a light chain variable region comprising the amino acid sequence of SEQ ID NO: 932.

In an embodiment, PD-L1 the targeting moiety comprises the anti-PD-L1 antibody 1B12, or fragments thereof, as disclosed in US 2013/0309250 and WO2007/005874, the entire disclosures of which are hereby incorporated by reference. In illustrative embodiments, 1B12 or an antigen-binding fragment thereof for use in the methods provided herein comprises a heavy chain variable region comprising the amino acid sequence of SEQ ID NO: 933; and/or a light chain variable region comprising the amino acid sequence of SEQ ID NO: 934.

In an embodiment, the PD-L1 targeting moiety comprises the anti-PD-L1 antibody 7H1, or fragments thereof, as disclosed in US 2013/0309250 and WO2007/005874, the entire disclosures of which are hereby incorporated by reference. In illustrative embodiments, 7H1 or an antigen-binding fragment thereof for use in the methods provided herein comprises a heavy chain variable region comprising the amino acid sequence of SEQ ID NO: 935; and/or a light chain variable region comprising the amino acid sequence of SEQ ID NO: 936.

In an embodiment, the PD-L1 targeting moiety comprises the anti-PD-L1 antibody 11E6, or fragments thereof, as disclosed in US 2013/0309250 and WO2007/005874, the entire disclosures of which are hereby incorporated by reference. In illustrative embodiments, 11E6 or an antigen-binding fragment thereof for use in the methods provided herein comprises a heavy chain variable region comprising the amino acid sequence of SEQ ID NO: 937; and/or a light chain variable region comprising the amino acid sequence of SEQ ID NO: 938.

In an embodiment, the PD-L1 targeting moiety comprises the anti-PD-L1 antibody 12B7, or fragments thereof, as disclosed in US 2013/0309250 and WO2007/005874, the entire disclosures of which are hereby incorporated by reference. In illustrative embodiments, 12B7 or an antigen-binding fragment thereof for use in the methods provided herein comprises a heavy chain variable region comprising the amino acid sequence of SEQ ID NO: 939; and/or a light chain variable region comprising the amino acid sequence of SEQ ID NO: 940.

In an embodiment, the PD-L1 targeting moiety comprises the anti-PD-L1 antibody 13G4, or fragments thereof, as disclosed in US 2013/0309250 and WO2007/005874, the entire disclosures of which are hereby incorporated by reference. In illustrative embodiments, 13G4 or an antigen-binding fragment thereof for use in the methods provided herein comprises a heavy chain variable region comprising the amino acid sequence of SEQ ID NO: 941; and/or a light chain variable region comprising the amino acid sequence of SEQ ID NO: 942.

In an embodiment, the PD-L1 targeting moiety comprises the anti-PD-L1 antibody 1E12, or fragments thereof, as disclosed in US 2014/0044738, the entire disclosures of which are hereby incorporated by reference. In illustrative embodiments, 1E12 or an antigen-binding fragment thereof for use in the methods provided herein comprises a heavy chain variable region comprising the amino acid sequence of SEQ ID NO: 943; and/or a light chain variable region comprising the amino acid sequence of SEQ ID NO: 944.

In an embodiment, the PD-L1 targeting moiety comprises the anti-PD-L1 antibody 1F4, or fragments thereof, as disclosed in US 2014/0044738, the entire disclosures of which are hereby incorporated by reference. In illustrative embodiments, 1F4 or an antigen-binding fragment thereof for use in the methods provided herein comprises a heavy chain variable region comprising the amino acid sequence of SEQ ID NO: 945; and/or a light chain variable region comprising the amino acid sequence of SEQ ID NO: 946.

In an embodiment, the PD-L1 targeting moiety comprises the anti-PD-L1 antibody 2G11, or fragments thereof, as disclosed in US 2014/0044738, the entire disclosures of which are hereby incorporated by reference. In illustrative embodiments, 2G11 or an antigen-binding fragment thereof for use in the methods provided herein comprises a heavy chain variable region comprising the amino acid sequence of SEQ ID NO: 947; and/or a light chain variable region comprising the amino acid sequence of SEQ ID NO: 948.

In an embodiment, the PD-L1 targeting moiety comprises the anti-PD-L1 antibody 3B6, or fragments thereof, as disclosed in US 2014/0044738, the entire disclosures of which are hereby incorporated by reference. In illustrative embodiments, 3B6 or an antigen-binding fragment thereof for use in the methods provided herein comprises a heavy chain variable region comprising the amino acid sequence of SEQ ID NO: 949; and/or a light chain variable region comprising the amino acid sequence of SEQ ID NO: 950.

In an embodiment, the PD-L1 targeting moiety comprises the anti-PD-L1 antibody 3D10, or fragments thereof, as disclosed in US 2014/0044738 and WO2012/145493, the entire disclosures of which are hereby incorporated by reference. In illustrative embodiments, 3D10 or an antigen-binding fragment thereof for use in the methods provided herein comprises a heavy chain variable region comprising the amino acid sequence of SEQ ID NO: 951; and/or a light chain variable region comprising the amino acid sequence of SEQ ID NO: 952.

In an embodiment, the PD-L1 targeting moiety comprises any one of the anti-PD-L1 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 34-38 of US2011/0271358 (SEQ ID Nos.: 953-957) and/or a light chain comprising an amino acid sequence selected from SEQ ID Nos: 39-42 of US2011/0271358 (SEQ ID Nos.: 958-961).

In an embodiment, the PD-L1 targeting moiety comprises the anti-PD-L1 antibody 2.7A4, or fragments thereof, as disclosed in WO 2011/066389, U.S. Pat. No. 8,779,108, and US2014/0356353, the entire disclosures of which are hereby incorporated by reference. In illustrative embodiments, 2.7A4 or an antigen-binding fragment thereof for use in the methods provided herein comprises a heavy chain variable region comprising the amino acid sequence of SEQ ID No: 2 of WO 2011/066389 (SEQ ID NO: 962); and/or a light chain variable region comprising the amino acid sequence of SEQ ID No: 7 of WO 2011/066389 (SEQ ID NO: 963).

In an embodiment, the PD-L1 targeting moiety comprises the anti-PD-L1 antibody 2.9D10, or fragments thereof, as disclosed in WO 2011/066389, U.S. Pat. No. 8,779,108, and US2014/0356353, the entire disclosures of which are hereby incorporated by reference. In illustrative embodiments, 2.9D10 or an antigen-binding fragment thereof for use in the methods provided herein comprises a heavy chain variable region comprising the amino acid sequence of SEQ ID No: 12 of WO 2011/066389 (SEQ ID NO: 964); and/or a light chain variable region comprising the amino acid sequence of SEQ ID No: 17 of WO 2011/066389 (SEQ ID NO: 965).

In an embodiment, the PD-L1 targeting moiety comprises the anti-PD-L1 antibody 2.14H9, or fragments thereof, as disclosed in WO 2011/066389, U.S. Pat. No. 8,779,108, and US2014/0356353, the entire disclosures of which are hereby incorporated by reference. In illustrative embodiments, 2.14H9 or an antigen-binding fragment thereof for use in the methods provided herein comprises a heavy chain variable region comprising the amino acid sequence of SEQ ID No: 22 of WO 2011/066389 (SEQ ID NO: 966); and/or a light chain variable region comprising the amino acid sequence of SEQ ID No: 27 of WO 2011/066389 (SEQ ID NO: 967).

In an embodiment, the PD-L1 targeting moiety comprises the anti-PD-L1 antibody 2.20A8, or fragments thereof, as disclosed in WO 2011/066389, U.S. Pat. No. 8,779,108, and US2014/0356353, the entire disclosures of which are hereby incorporated by reference. In illustrative embodiments, 2.20A8 or an antigen-binding fragment thereof for use in the methods provided herein comprises a heavy chain variable region comprising the amino acid sequence of SEQ ID No: 32 of WO 2011/066389 (SEQ ID NO: 968); and/or a light chain variable region comprising the amino acid sequence of SEQ ID No: 37 of WO 2011/066389 (SEQ ID NO: 969).

In an embodiment, the PD-L1 targeting moiety comprises the anti-PD-L1 antibody 3.15G8, or fragments thereof, as disclosed in WO 2011/066389, U.S. Pat. No. 8,779,108, and US2014/0356353, the entire disclosures of which are hereby incorporated by reference. In illustrative embodiments, 3.15G8 or an antigen-binding fragment thereof for use in the methods provided herein comprises a heavy chain variable region comprising the amino acid sequence of SEQ ID No: 42 of WO 2011/066389 (SEQ ID NO: 970); and/or a light chain variable region comprising the amino acid sequence of SEQ ID No: 47 of WO 2011/066389 (SEQ ID NO: 971).

In an embodiment, the PD-L1 targeting moiety comprises the anti-PD-L1 antibody 3.18G1, or fragments thereof, as disclosed in WO 2011/066389, U.S. Pat. No. 8,779,108, and US2014/0356353, the entire disclosures of which are hereby incorporated by reference. In illustrative embodiments, 3.18G1 or an antigen-binding fragment thereof for use in the methods provided herein comprises a heavy chain variable region comprising the amino acid sequence of SEQ ID No: 52 of WO 2011/066389 (SEQ ID NO: 972); and/or a light chain variable region comprising the amino acid sequence of SEQ ID No: 57 of WO 2011/066389 (SEQ ID NO: 973).

In an embodiment, the PD-L1 targeting moiety comprises the anti-PD-L1 antibody 2.7A4OPT, or fragments thereof, as disclosed in WO 2011/066389, U.S. Pat. No. 8,779,108, and US2014/0356353, and US2014/0356353, the entire disclosures of which are hereby incorporated by reference. In illustrative embodiments, 2.7A4OPT or an antigen-binding fragment thereof for use in the methods provided herein comprises a heavy chain variable region comprising the amino acid sequence of SEQ ID No: 62 of WO 2011/066389 (SEQ ID NO: 974); and/or a light chain variable region comprising the amino acid sequence of SEQ ID No: 67 of WO 2011/066389 (SEQ ID NO: 975).

In an embodiment, the PD-L1 targeting moiety comprises the anti-PD-L1 antibody 2.14H9OPT, or fragments thereof, as disclosed in WO 2011/066389, U.S. Pat. No. 8,779,108, and US2014/0356353, the entire disclosures of which are hereby incorporated by reference. In illustrative embodiments, 2.14H9OPT or an antigen-binding fragment thereof for use in the methods provided herein comprises a heavy chain variable region comprising the amino acid sequence of SEQ ID No: 72 of WO 2011/066389 (SEQ ID NO: 976); and/or a light chain variable region comprising the amino acid sequence of SEQ ID No: 77 of WO 2011/066389 (SEQ ID NO: 977).

In an embodiment, the PD-L1 targeting moiety comprises any one of the anti-PD-L1 antibodies disclosed in WO2016/061142, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 18, 30, 38, 46, 50, 54, 62, 70, and 78 of WO2016/061142 (SEQ ID Nos.: 978, 979, 980, 981, 982, 983, 984, 985, and 986, respectively); and/or a light chain comprising an amino acid sequence selected from SEQ ID Nos: 22, 26, 34, 42, 58, 66, 74, 82, and 86 of WO2016/061142 (SEQ ID Nos.: 987, 988, 989, 990, 991, 992, 993, 994, and 995, respectively).

In an embodiment, the PD-L1 targeting moiety comprises any one of the anti-PD-L1 antibodies disclosed in WO2016/022630, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 6, 10, 14, 18, 22, 26, 30, 34, 38, 42, and 46 of WO2016/022630 (SEQ ID Nos.: 996, 997, 998, 999, 1000, 1001, 1002, 1003, 1004, 1005, 1006, and 1007, respectively); and/or a light chain comprising an amino acid sequence selected from SEQ ID Nos: 4, 8, 12, 16, 20, 24, 28, 32, 36, 40, 44, and 48 of WO2016/022630 (SEQ ID Nos: 1008, 1009, 1010, 1011, 1012, 1013, 1014, 1015, 1016, 1017, 1018, and 1019, respectively).

In an embodiment, the PD-L1 targeting moiety comprises any one of the anti-PD-L1 antibodies disclosed in WO2015/112900, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 38, 50, 82, and 86 of WO 2015/112900 (SEQ ID Nos.: 1020, 1021, 1022, and 1023, respectively); and/or a light chain comprising an amino acid sequence selected from SEQ ID Nos: 42, 46, 54, 58, 62, 66, 70, 74, and 78 of WO 2015/112900 (SEQ ID Nos: 1024, 1025, 1026, 1027, 1028, 1029, 1030, 1031, and 1032, respectively).

In an embodiment, the PD-L1 targeting moiety comprises any one of the anti-PD-L1 antibodies disclosed in WO 2010/077634 and U.S. Pat. No. 8,217,149, the entire disclosures of which are hereby incorporated by reference. In illustrative embodiments, the anti-PD-L1 antibody or an antigen-binding fragment thereof for use in the methods provided herein comprises a heavy chain region comprising the amino acid sequence of SEQ ID No: 20 of WO 2010/077634 (SEQ ID NO: 1033); and/or a light chain variable region comprising the amino acid sequence of SEQ ID No: 21 of WO 2010/077634 (SEQ ID NO: 1034). In an embodiment, the PD-L1 targeting moiety comprises any one of the anti-PD-L1 antibodies obtainable from the hybridoma accessible under CNCM deposit numbers CNCM I-4122, CNCM I-4080 and CNCM I-4081 as disclosed in US 20120039906, the entire disclosures of which are hereby incorporated by reference.

In an embodiment, the PD-L1 targeting moiety comprises a VHH directed against PD-L1 as disclosed, for example, in U.S. Pat. No. 8,907,065 and WO 2008/071447, the entire disclosures of which are hereby incorporated by reference. In illustrative embodiments, the VHHs against PD-L1 comprise SEQ ID NOS: 394-399 of U.S. Pat. No. 8,907,065 (SEQ ID NOS: 1035-1040, respectively).

In some embodiments, the targeting moiety binds to PD-L1 and the signaling moiety is a wild type IFNα or a modified IFNα. In some embodiments, the Fc chimeric protein is in one of the forms shown in FIGS. 4A to 4D, where the targeting moiety binds to PD-L1 and the signaling moiety is a wild type IFNα. In some embodiments, the targeting moiety binds to PD-L1 (e.g., scFv) and the signaling moiety is a wild type IFNα2.

PD-L2 Targeting Moieties

In some embodiments, the targeting moiety is directed against PD-L2. In some embodiments, the targeting moiety selectively binds a PD-L2 polypeptide. In some embodiments, the PD-L2 targeting moiety comprises an antibody, an antibody derivative or format, a peptide or polypeptide, or a fusion protein that selectively binds a PD-L2 polypeptide.

In an embodiment, the PD-L2 targeting moiety comprises a VHH directed against PD-L2 as disclosed, for example, in U.S. Pat. No. 8,907,065 and WO 2008/071447, the entire disclosures of which are hereby incorporated by reference. In illustrative embodiments, the VHHs against PD-L2 comprise SEQ ID Nos: 449-455 of U.S. Pat. No. 8,907,065 (SEQ ID Nos: 1041-1047, respectively).

In an embodiment, the PD-L2 targeting moiety comprises any one of the anti-PD-L2 antibodies disclosed in US2011/0271358 and WO2010/036959, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 43-47 of US2011/0271358 (SEQ ID Nos: 1048-1052, respectively); and/or a light chain comprising an amino acid sequence selected from SEQ ID Nos: 48-51 of US2011/0271358 (SEQ ID Nos: 1053-1056, respectively).

In some embodiments, the present technology contemplates the use of any natural or synthetic analogs, mutants, variants, alleles, homologs and orthologs (herein collectively referred to as “analogs”) of the PD-1, PD-L1, or PD-L2 targeting moieties described herein. In some embodiments, the amino acid sequence of the PD-1, PD-L1, or PD-L2 targeting moiety further includes an amino acid analog, an amino acid derivative, or other non-classical amino acids.

In various embodiments, the PD-1, PD-L1, or PD-L2 targeting moieties disclosed herein comprise a sequence that targets PD-1, PD-L1, or PD-L2 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the PD-1, PD-L1, and/or PD-L2 sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the PD-1, PD-L1, and/or PD-L2 sequences disclosed herein).

In various embodiments, the PD-1, PD-L1, or PD-L2 targeting moiety comprises a binding agent comprising an amino acid sequence having one or more amino acid mutations with respect to any one of the PD-1, PD-L1, or PD-L2 sequences disclosed herein. In various embodiments, the PD-1, PD-L1, or PD-L2 targeting moiety comprises a binding agent comprising an amino acid sequence having one, or two, or three, or four, or five, or six, or seen, or eight, or nine, or ten, or fifteen, or twenty amino acid mutations with respect to any one of the sequences disclosed herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids. Exemplary non-classical amino acids include, but are not limited to, selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the present PD-1, PD-L1, or PD-L2 targeting moiety's capability to specifically bind to PD-1, PD-L1, or PD-L2. In various embodiments, the mutations do not substantially reduce the PD-1, PD-L1, or PD-L2 targeting moiety's capability to specifically bind to PD-1, PD-L1, or PD-L2 and without functionally modulating (e.g., partially or fully neutralizing) PD-1, PD-L1, or PD-L2.

In various embodiments, the binding affinity of the PD-1, PD-L1, or PD-L2 targeting moiety for the full-length and/or mature forms and/or isoforms and/or splice variants and/or fragments and/or monomeric and/or dimeric forms and/or any other naturally occurring or synthetic analogs, variants, or mutants (including monomeric and/or dimeric forms) of human PD-1, PD-L1, or PD-L2 may be described by the equilibrium dissociation constant (K_{D}). In various embodiments, the PD-1, PD-L1, or PD-L2 targeting moiety binds to the full-length and/or mature forms and/or isoforms and/or splice variants and/or fragments and/or any other naturally occurring or synthetic analogs, variants, or mutants (including monomeric and/or dimeric forms) of human PD-1, PD-L1, or PD-L2 with a K_{D} of less than about 1 uM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In some embodiments, the PD-1, PD-L1, and/or PD-L2 targeting moieties disclosed herein may comprise any combination of heavy chain, light chain, heavy chain variable region, light chain variable region, complementarity determining region (CDR), and framework region sequences that target PD-1, PD-L1, and/or PD-L2 as disclosed herein.

Additional antibodies, antibody derivatives or formats, peptides or polypeptides, or fusion proteins that selectively bind or target PD-1, PD-L1 and/or PD-L2 are disclosed in WO 2011/066389, US 2008/0025980, US 2013/0034559, U.S. Pat. No. 8,779,108, US 2014/0356353, U.S. Pat. No. 8,609,089, US 2010/028330, US 2012/0114649, WO 2010/027827, WO 2011/066342, U.S. Pat. No. 8,907,065, WO 2016/062722, WO 2009/101611, WO2010/027827, WO 2011/066342, WO 2007/005874, WO 2001/014556, US2011/0271358, WO 2010/036959, WO 2010/077634, U.S. Pat. No. 8,217,149, US 2012/0039906, WO 2012/145493, US 2011/0318373, U.S. Pat. No. 8,779,108, US 20140044738, WO 2009/089149, WO 2007/00587, WO 2016061142, WO 2016,02263, WO 2010/077634, and WO 2015/112900, the entire disclosures of which are hereby incorporated by reference.

### SIRP1α Targeting Moieties

In some embodiments, the targeting moiety binds a signal regulatory protein α-1 (SIRP1α). SIRP1α (also known as SIRPα) belongs to a family of cell immune receptors encompassing inhibitory (SIRPα), activating (SIRPβ), nonsignaling (SIRPγ) and soluble (SIRPδ) members. SIRP1α is expressed primarily on myeloid cells, including macrophages, granulocytes, myeloid dendritic cells (DCs), mast cells, and their precursors, including hematopoietic stem cells. SIRP1α acts as an inhibitory receptor that interacts with a broadly expressed transmembrane glycoprotein CD47 to regulate phagocytosis. In particular, the binding of SIRP1α on macrophages by CD47 expressed on target cells, generates an inhibitory signal that negatively regulates phagocytosis of the target cell.

In some embodiments, the SIRP1α targeting moiety specifically recognizes and binds SIRP1α on macrophages.

In some embodiments, the SIRP1α targeting moiety specifically recognizes and binds SIRP1α on monocytes.

In some embodiments, the SIRP1α targeting moiety specifically recognizes and binds SIRP1α on TAMs (Tumor Associated Macrophages).

In some embodiments, the SIRP1α targeting moiety specifically recognizes and binds SIRP1α on dendritic cells, including without limitation cDC2 and pDC

In some embodiments, the SIRP1α targeting moiety recognizes one or more linear epitopes present on SIRP1α.

In some embodiments, a linear epitope refers to any continuous sequence of amino acids present on SIRP1α. In another embodiment, the recognition domain recognizes one or more conformational epitopes present on SIRP1α. As used herein, a conformation epitope refers to one or more sections of amino acids (which may be discontinuous) which form a three-dimensional surface with features and/or shapes and/or tertiary structures capable of being recognized by an antigen recognition domain.

In some embodiments, the SIRP1α targeting moiety binds to the full-length and/or mature forms and/or isoforms and/or splice variants and/or fragments and/or any other naturally occurring or synthetic analogs, variants, or mutants of SIRP1α. In an embodiment, the SIRP1α is human SIRP1α. In various embodiments, the SIRP1α targeting moiety may bind to any forms of the human SIRP1α, including monomeric, dimeric, heterodimeric, multimeric and associated forms. In an embodiment, the SIRP1α targeting moiety binds to the monomeric form of SIRP1α. In another embodiment, the SIRP1α targeting moiety binds to a dimeric form of SIRP1α.

In some embodiments, the SIRP1α targeting moiety comprises a recognition domain that recognizes one or more epitopes present on human SIRP1α. In an embodiment, the SIRP1α targeting moiety comprises a recognition domain that recognizes human SIRP1α with a signal peptide sequence. An exemplary human SIRP1α polypeptide with a signal peptide sequence is SEQ ID NO:1057.

In some embodiments, the SIRP1α targeting moiety comprises a recognition domain that recognizes human SIRP1α without a signal peptide sequence. An exemplary human SIRP1α polypeptide without a signal peptide sequence is SEQ ID NO: 1058.

In some embodiments, the SIRP1α targeting moiety comprises a recognition domain that recognizes a polypeptide encoding human SIRP1α isoform 2 (SEQ ID NO: 1059).

In some embodiment, the SIRP1α targeting moiety comprises a recognition domain that recognizes a polypeptide encoding human SIRP1α isoform 4 (SEQ ID NO:1060).

In some embodiments, the SIRP1α targeting moiety may be any protein-based agent capable of specific binding, such as an antibody or derivatives thereof.

In some embodiments, the SIRP1α targeting moiety comprises antibody derivatives or formats. In some embodiments, the SIRP1α targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an alphabody; a bicyclic peptide; an Affilin; a Microbody; a peptide aptamer; an alterase; a plastic antibodies; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; Affimers, a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the SIRP1α targeting moiety comprises a single-domain antibody, such as VHH from, for example, an organism that produces VHH antibody such as a camelid, a shark, or a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3).

In some embodiments, the SIRP1α targeting moiety comprises a VHH. In some embodiments, the VHH is a humanized VHH or camelized VHH.

In some embodiments, the VHH comprises a fully human V_{H} domain, e.g. a HUMABODY (Crescendo Biologics, Cambridge, UK). In some embodiments, fully human V_{H} domain, e.g. a HUMABODY is monovalent, bivalent, or trivalent. In some embodiments, the fully human V_{H} domain, e.g. a HUMABODY is mono- or multi-specific such as monospecific, bispecific, or trispecific. Illustrative fully human VH domains, e.g. HUMABODIES are described in, for example, WO 2016/113555 and WO2016/113557, the entire disclosure of which is incorporated by reference.

For example, in some embodiments, the SIRP1α targeting moiety comprises one or more antibodies, antibody derivatives or formats, peptides or polypeptides, VHHs, or fusion proteins that selectively bind SIRP1α. In some embodiments, the SIRP1α targeting moiety comprises an antibody or derivative thereof that specifically binds to SIRP1α. In some embodiments, the SIRP1α targeting moiety comprises a camelid heavy chain antibody (VHH) that specifically binds to SIRP1α.

In some embodiments, the SIRP1α targeting moiety is a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets. In various embodiments, the present Fc-based chimeric protein complex comprises a VHH having a variable domain comprising at least one CDR1, CDR2, and/or CDR3 sequences.

In some embodiments, the SIRP1α targeting moiety may comprise any combination of heavy chain, light chain, heavy chain variable region, light chain variable region, complementarity determining region (CDR), and framework region sequences that is known to recognize and bind to SIRP1α.

In some embodiments, the present technology contemplates the use of any natural or synthetic analogs, mutants, variants, alleles, homologs and orthologs (herein collectively referred to as “analogs”) of the SIRP1α targeting moieties described herein. In various embodiments, the amino acid sequence of the SIRP1α targeting moiety further includes an amino acid analog, an amino acid derivative, or other non-classical amino acids.

In some embodiments, the SIRP1α targeting moiety comprises a sequence that is at least 60% identical to any one of the SIRP1α sequences disclosed herein. For example, in some embodiments, the SIRP1α targeting moiety comprises a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the SIRP1α sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the SIRP1α sequences disclosed herein).

In some embodiments, the SIRP1α targeting moiety comprises an amino acid sequence having one or more amino acid mutations with respect to any targeting moiety sequence that is known to recognize and bind to SIRP1α. In various embodiments, the SIRP1α targeting moiety comprises an amino acid sequence having one, or two, or three, or four, or five, or six, or seen, or eight, or nine, or ten, or fifteen, twenty, thirty, forty, or fifty amino acid mutations with respect to any targeting moiety sequence that is known to recognize and bind to SIRP1α. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids. Exemplary non-classical amino acids include, but are not limited to, selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the SIRP1α targeting moiety's capability to specifically recognize and bind to SIRP1α. In various embodiments, the mutations do not substantially reduce the SIRP1α targeting moiety's ability to bind specifically to SIRP1α and without functionally modulating (e.g., partially or fully neutralizing) SIRP1α.

In various embodiments, the binding affinity of the SIRP1α targeting moiety for the full-length and/or mature forms and/or isoforms and/or splice variants and/or fragments and/or monomeric and/or dimeric forms and/or any other naturally occurring or synthetic analogs, variants, or mutants of SIRP1α may be described by the equilibrium dissociation constant (K_{D}). In various embodiments, the SIRP1α targeting moiety that binds to the full-length and/or mature forms and/or isoforms and/or splice variants and/or fragments and/or any other naturally occurring or synthetic analogs, variants, or mutants (including monomeric and/or dimeric forms) of SIRP1α with a K_{D} of less than about 1 uM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In various embodiments, the SIRP1α targeting moiety binds but does not functionally modulate the antigen of interest, i.e., SIRP1α. For example, in some embodiments, the SIRP1α targeting moiety simply targets the antigen but does not substantially functionally modulate (e.g. substantially inhibit, reduce or neutralize) a biological effect that the antigen has. In various embodiments, the targeting moiety of the present Fc-based chimeric protein complex binds an epitope that is physically separate from an antigen site that is important for its biological activity (e.g. an antigen's active site).

In some embodiments, the SIRP1α targeting moiety binds but functionally modulates the antigen of interest, i.e., SIRP1α. For example, in some embodiments, the SIRP1α targeting moiety targets the antigen, i.e., SIRP1α, and functionally modulates (e.g. inhibit, reduce or neutralize) a biological effect that the antigen has. Such binding along with functional modulation may find use in various embodiments of the present invention including methods in which the present Fc-based chimeric protein complex is used to directly or indirectly recruit active immune cells to a site of need via an effector antigen.

In some embodiments, the SIRP1α targeting moiety may be used to directly or indirectly recruit macrophages via SIRP1α to a tumor cell in a method of reducing or eliminating a tumor (e.g. the present Fc-based chimeric protein complex may comprise a targeting moiety having an anti-SIRP1α antigen recognition domain and a targeting moiety having a recognition domain (e.g. antigen recognition domain) directed against a tumor antigen or receptor). Evidence indicates that tumor cells frequently upregulate CD47 which engages SIRP1α so as to evade phagocytosis. Accordingly, in various embodiments, it may be desirable to directly or indirectly recruit macrophages to tumor cells and functionally inhibit, reduce, or neutralize the inhibitory activity of SIRP1α thereby resulting in phagocytosis of the tumor cells by the macrophages. In various embodiments, the present Fc-based chimeric protein complex enhances phagocytosis of tumor cells or any other undesirable cells by macrophages.

SIRP alpha targeting moieties may comprise CDRs of antibodies as described in WO200140307A1, WO2013056352A1, WO2015138600A2, WO2017178653A2, WO2018057669A1, WO2018107058A1, WO2018190719A2, WO2019023347A1, the contents of which are hereby incorporated by reference in their entireties.

### FAP Targeting Moieties

Fibroblast activation protein (FAP) is a 170 kDa melanoma membrane-bound gelatinase that belongs to the serine protease family. FAP is selectively expressed in reactive stromal fibroblasts of epithelial cancers, granulation tissue of healing wounds, and malignant cells of bone and soft tissue sarcomas. FAP is believed to be involved in the control of fibroblast growth or epithelial-mesenchymal interactions during development, tissue repair, and epithelial carcinogenesis.

In some embodiments, the targeting moiety is a FAP targeting moiety that is a protein-based agent capable of specific binding to FAP. In some embodiments, the FAP targeting moiety is a protein-based agent capable of specific binding to FAP without functional modulation (e.g., partial or full neutralization) of FAP.

In some embodiments, the fibroblast targeting moiety targets F2 fibroblasts. In some embodiments, the fibroblast targeting moiety directly or indirectly alters the microenvironment of the F2 fibroblasts. In some embodiments, the fibroblast binding agent directly or indirectly polarizes the F2 fibroblast into F1 fibroblast.

F2 fibroblast(s) refers to pro-tumorigenic (or tumor promoting) cancer-associated fibroblasts (CAFs) (a/k/a Type II-CAF). F1 fibroblast(s) refers to tumor suppressive CAFs (a/k/a Type I-CAF). Polarization refers to changing the phenotype of cell, e.g. changing a tumorigenic F2 fibroblast to a tumor suppressive F1 fibroblast.

In some embodiments, the FAP targeting moiety targets a FAP marker.

In some embodiments, the FAP targeting moiety comprises a binding agent having an antigen recognition domain that recognizes an epitope present on FAP. In some embodiments, the antigen-recognition domain recognizes one or more linear epitopes present on FAP. In some embodiments, a linear epitope refers to any continuous sequence of amino acids present on FAP. In another embodiment, the antigen-recognition domain recognizes one or more conformational epitopes present on FAP. As used herein, a conformation epitope refers to one or more sections of amino acids (which may be discontinuous), which form a three-dimensional surface with features and/or shapes and/or tertiary structures capable of being recognized by an antigen recognition domain.

In some embodiments, the FAP targeting moiety can bind to the full-length and/or mature forms and/or isoforms and/or splice variants and/or fragments and/or any other naturally occurring or synthetic analogs, variants, or mutants of human FAP. In some embodiments, the FAP targeting moiety can bind to any forms of the human FAP, including monomeric, dimeric, heterodimeric, multimeric and associated forms. In an embodiment, the FAP targeting moiety binds to the monomeric form of FAP. In another embodiment, the FAP targeting moiety binds to a dimeric form of FAP. In a further embodiment, the FAP targeting moiety binds to glycosylated form of FAP, which may be either monomeric or dimeric.

In an embodiment, the FAP targeting moiety comprises an antigen recognition domain that recognizes one or more epitopes present on human FAP. In some embodiments, the human FAP comprises the amino acid sequence of SEQ ID NO: 1061.

In some embodiments, the FAP targeting moiety is capable of specific binding. In some embodiments, the FAP targeting moiety comprises an antigen recognition domain such as an antibody or derivatives thereof.

In some embodiments, the FAP targeting moiety comprises an antibody derivative or format. In some embodiments, the FAP targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an alphabody; a bicyclic peptide; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)2, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the FAP targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (VHH) and two constant domains (CH2 and CH3).

In an embodiment, the FAP targeting moiety comprises a VHH. In some embodiments, the VHH is a humanized VHH or camelized VHH.

In some embodiments, the VHH comprises a fully human VH domain, e.g. a HUMABODY (Crescendo Biologics, Cambridge, UK). In some embodiments, fully human VH domain, e.g. a HUMABODY is monovalent, bivalent, or trivalent. In some embodiments, the fully human VH domain, e.g. a HUMABODY is mono- or multi-specific such as monospecific, bispecific, or trispecific. Illustrative fully human VH domains, e.g. HUMABODIES are described in, for example, WO 2016/113555 and WO 2016/113557, the entire disclosures of which are incorporated by reference.

By way of example, but not by way of limitation, in some embodiments, a human VHH FAP targeting moiety comprises an amino acid sequence selected from the following sequences: 2HFA44 (SEQ ID NO: 1062); 2HFA52 (SEQ ID NO: 1063); 2HFA11 (SEQ ID NO: 1064); 2HFA4 (SEQ ID NO: 1065); 2HFA46 (SEQ ID NO: 1066); 2HFA10 (SEQ ID NO: 1067); 2HFA38 (SEQ ID NO: 1068); 2HFA20 (SEQ ID NO: 1069); 2HFA5 (SEQ ID NO: 1070); 2HFA19 (SEQ ID NO: 1071); 2HFA2 (SEQ ID NO: 1072); 2HFA41 (SEQ ID NO: 1073); 2HFA42 (SEQ ID NO: 1074); 2HFA12 (SEQ ID NO: 1075); 2HFA24 (SEQ ID NO: 1076); 2HFA67 (SEQ ID NO: 1077); 2HFA29 (SEQ ID NO: 1078); 2HFA51 (SEQ ID NO: 1079); 2HFA63 (SEQ ID NO: 1080); 2HFA62 (SEQ ID NO: 1081); 2HFA26 (SEQ ID NO: 1082); 2HFA25 (SEQ ID NO: 1083); 2HFA1 (SEQ ID NO: 1084); 2HFA3 (SEQ ID NO: 1085); 2HFA7 (SEQ ID NO: 1086); 2HFA31 (SEQ ID NO: 1087); 2HFA6 (SEQ ID NO: 1088); 2HFA53 (SEQ ID NO: 1089); 2HFA9 (SEQ ID NO: 1090); 2HFA73 (SEQ ID NO: 1091); 2HFA55 (SEQ ID NO: 1092); 2HFA71 (SEQ ID NO: 1093); 2HFA60 (SEQ ID NO: 1094); 2HFA65 (SEQ ID NO: 1095); 2HFA49 (SEQ ID NO: 1096); 2HFA57 (SEQ ID NO: 1097); 2HFA23 (SEQ ID NO: 1098); 2HFA36 (SEQ ID NO: 1099); 2HFA14 (SEQ ID NO: 1100); 2HFA43 (SEQ ID NO: 1101); and 2HFA50 (SEQ ID NO: 1102).

In some embodiments, the FAP targeting moiety comprises an amino acid sequence selected from SEQ ID NOs: 1062-1102 (provided above) without the terminal histidine tag sequence (i.e., HHHHHH; SEQ ID NO: 393).

In some embodiments, the FAP targeting moiety comprises an amino acid sequence selected from SEQ ID NOs: 1062-1102 (provided above) without the HA tag (i.e., YPYDVPDYGS; SEQ ID NO: 394).

In some embodiments, the FAP targeting moiety comprises an amino acid sequence selected from SEQ ID NOs: 1062-1102 (provided above) without the AAA linker (i.e., AAA).

In some embodiments, the FAP targeting moiety comprises an amino acid sequence selected from SEQ ID NOs: 1062-1102 (provided above) without the AAA linker, HA tag, and terminal histidine tag sequence (i.e., AAAYPYDVPDYGSHHHHHH; SEQ ID NO: 395).

By way of example, but not by way of limitation, in some embodiments, a human VHH FAP targeting moiety comprises an amino acid sequence selected from the following sequences: 2HFA44 (SEQ ID NO: 1103); 2HFA52 (SEQ ID NO: 1104); 2HFA11 (SEQ ID NO: 1105); 2HFA4 (SEQ ID NO: 1106); 2HFA46 (SEQ ID NO: 1107); 2HFA10 (SEQ ID NO: 1108); 2HFA38 (SEQ ID NO: 1109); 2HFA20 (SEQ ID NO: 1110); 2HFA5 (SEQ ID NO: 1111); 2HFA19 (SEQ ID NO: 1112); 2HFA2 (SEQ ID NO: 1113); 2HFA41 (SEQ ID NO: 1114); 2HFA42 (SEQ ID NO: 1115); 2HFA12 (SEQ ID NO: 1116); 2HFA24 (SEQ ID NO: 1117); 2HFA67 (SEQ ID NO: 1118); 2HFA29 (SEQ ID NO: 1119); 2HFA51 (SEQ ID NO: 1120); 2HFA63 (SEQ ID NO: 1121); 2HFA62 (SEQ ID NO: 1122); 2HFA26 (SEQ ID NO: 1123); 2HFA25 (SEQ ID NO: 1124); 2HFA1 (SEQ ID NO: 1125); 2HFA3 (SEQ ID NO: 1126); 2HFA7 (SEQ ID NO: 1127); 2HFA31 (SEQ ID NO: 1128); 2HFA6 (SEQ ID NO: 1129); 2HFA53 (SEQ ID NO: 1130); 2HFA9 (SEQ ID NO: 1131); 2HFA73 (SEQ ID NO: 1132); 2HFA55 (SEQ ID NO: 1133); 2HFA71 (SEQ ID NO: 1134); 2HFA60 (SEQ ID NO: 1135); 2HFA65 (SEQ ID NO: 1136); 2HFA49 (SEQ ID NO: 1137); 2HFA57 (SEQ ID NO: 1138); 2HFA23 (SEQ ID NO: 1139); 2HFA36 (SEQ ID NO: 1140); 2HFA14 (SEQ ID NO: 1141); 2HFA43 (SEQ ID NO: 1142); and 2HFA50 (SEQ ID NO: 1143).

In some embodiments, the FAP targeting moiety comprises a binding agent that is a VHH comprising a single amino acid chain having four “framework regions” or FRs and three “complementary determining regions” or CDRs.

As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In some embodiments, the FAP targeting moiety comprises a VHH having a variable domain comprising at least one CDR1, CDR2, and/or CDR3 sequences. In some embodiments, the FAP targeting moiety comprises a VHH having a variable region comprising at least one FR1, FR2, FR3, and FR4 sequences.

In some embodiments, a human FAP targeting moiety comprises a CDR1 sequence selected from SEQ ID Nos: 1144-1172. In some embodiments, a human FAP targeting moiety comprises a CDR2 sequence selected from SEQ ID Nos: 1173-1201. In some embodiments, a human FAP targeting moiety comprises a CDR3 sequence selected from SEQ ID Nos.: 1202-1232.

In some embodiments, the FAP targeting moiety has at least 90% identity with any FAP amino acid sequence selected disclosed herein. In some embodiments, the FAP targeting moiety has about 90%, about 91%, about 92%, about 93%, about 94%, about 95%, about 96%, about 97%, about 98%, about 99% identity with any FAP amino acid sequence selected disclosed herein.

In various illustrative embodiments, the murine FAP targeting moiety has at least 90% identity with the amino acid sequence of sibrotuzumab.

In some embodiments, the present technology contemplates the use of any natural or synthetic analogs, mutants, variants, alleles, homologs and orthologs (herein collectively referred to as “analogs”) of the FAP targeting moieties as described herein. In some embodiments, the amino acid sequence of the FAP targeting moiety further includes an amino acid analog, an amino acid derivative, or other non-classical amino acids.

In some embodiments, the FAP targeting moiety comprises a sequence that is at least 60% identical to any one of the FAP sequences disclosed herein. For example, the FAP targeting moiety may comprise a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the FAP sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the FAP sequences disclosed herein).

In some embodiments, the FAP targeting moiety comprises an amino acid sequence having one or more amino acid mutations with respect to any one of the sequences disclosed herein. In some embodiments, the FAP targeting moiety comprises an amino acid sequence having one, or two, or three, or four, or five, or six, or seen, or eight, or nine, or ten, or fifteen, or twenty amino acid mutations with respect to any one of the sequences disclosed herein.

In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In some embodiments, the substitutions include non-classical amino acids. Illustrative non-classical amino acids include, but are not limited to, selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In some embodiments, one or more amino acid mutations are in the CDRs of the FAP targeting moiety (e.g., the CDR1, CDR2 or CDR3 regions). In another embodiment, one or more amino acid mutations are in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In some embodiments, the mutations do not substantially reduce the FAP targeting moiety's capability to specifically bind to FAP. In some embodiments, the mutations do not substantially reduce the present FAP targeting moiety's capability to specifically bind to FAP and without functionally modulating (e.g., partially or fully neutralizing) FAP.

In some embodiments, the binding affinity of the FAP targeting moiety for the full-length and/or mature forms and/or isoforms and/or splice variants and/or fragments and/or monomeric and/or dimeric forms and/or any other naturally occurring or synthetic analogs, variants, or mutants (including monomeric and/or dimeric forms) of human FAP may be described by the equilibrium dissociation constant (K_{D}). In some embodiments, the FAP targeting moiety binds to the full-length and/or mature forms and/or isoforms and/or splice variants and/or fragments and/or any other naturally occurring or synthetic analogs, variants, or mutants (including monomeric and/or dimeric forms) of human FAP with a K_{D} of less than about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In some embodiments, the FAP targeting moiety binds but does not functionally modulate (e.g., partially or fully neutralize) the antigen of interest, i.e., FAP. For instance, in some embodiments, the FAP targeting moiety simply targets the antigen but does not substantially functionally modulate (e.g. partially or fully inhibit, reduce or neutralize) a biological effect that the antigen has. In some embodiments, the FAP targeting moiety binds an epitope that is physically separate from an antigen site that is important for its biological activity (e.g. an antigen's active site).

Such binding without significant function modulation finds use in some embodiments of the present technology, including methods in which the FAP targeting moiety is used to directly or indirectly recruit active immune cells to a site of need via an effector antigen. For example, in some embodiments, the FAP targeting moiety can be used to directly or indirectly recruit dendritic cells via FAP to a tumor cell in a method of reducing or eliminating a tumor (e.g. the FAP targeting moiety may comprise a binding agent having an anti-FAP antigen recognition domain and a targeting moiety having a recognition domain (e.g. antigen recognition domain) directed against a tumor antigen or receptor). In such embodiments, it is desirable to directly or indirectly recruit dendritic cells but not to functionally modulate or neutralize the FAP activity. In these embodiments, FAP signaling is an important piece of the tumor reducing or eliminating effect.

In some embodiments, the FAP targeting moiety enhances antigen-presentation by dendritic cells. For example, in some embodiments, the FAP targeting moiety directly or indirectly recruits dendritic cells via FAP to a tumor cell, where tumor antigens are subsequently endocytosed and presented on the dendritic cell for induction of potent humoral and cytotoxic T cell responses.

In other embodiments (for example, related to treating cancer, autoimmune, or neurodegenerative disease), the FAP targeting moiety comprises a binding agent that binds and neutralizes the antigen of interest, i.e., FAP. For instance, in some embodiments, the present methods may inhibit or reduce FAP signaling or expression, e.g. to cause a reduction in an immune response.

### XCR1 Targeting Moieties

In some embodiments, the targeting moiety is an XCR1 targeting moiety that is capable of specific binding to XCR1. In various embodiments, the XCR1 targeting moiety is a protein-based agent capable of specific binding to XCR1 without functional modulation (e.g., partial or full neutralization) of XCR1. XCR1 is a chemokine receptor belonging to the G protein-coupled receptor superfamily. The family members are characterized by the presence of 7 transmembrane domains and numerous conserved amino acids. XCR1 is most closely related to RBS11 and the MIP1-alpha/RANTES receptor. XCR1 transduces a signal by increasing the intracellular calcium ions level. XCR1 is the receptor for XCL1 and XCL2 (or lymphotactin-1 and -2).

In some embodiments, the XCR1 targeting moiety comprises an antigen recognition domain that recognizes an epitope present on XCR1. In some embodiments, the antigen-recognition domain recognizes one or more linear epitopes present on XCR1. In some embodiment, a linear epitope refers to any continuous sequence of amino acids present on XCR1. In another embodiment, the antigen-recognition domain recognizes one or more conformational epitopes present on XCR1. As used herein, a conformation epitope refers to one or more sections of amino acids (which may be discontinuous) which form a three-dimensional surface with features and/or shapes and/or tertiary structures capable of being recognized by an antigen recognition domain.

In some embodiments, the XCR1 targeting moiety can bind to the full-length and/or mature forms and/or isoforms and/or splice variants and/or fragments and/or any other naturally occurring or synthetic analogs, variants, or mutants of human XCR1. In various embodiments, the XCR1 targeting moiety can bind to any forms of the human XCR1, including monomeric, dimeric, heterodimeric, multimeric and associated forms. In an embodiment, the Fc-based chimeric protein complex binds to the monomeric form of XCR1. In another embodiment, the XCR1 targeting moiety binds to a dimeric form of XCR1. In a further embodiment, the XCR1 targeting moiety binds to glycosylated form of XCR1, which may be either monomeric or dimeric.

In an embodiment, the XCR1 targeting moiety comprises an antigen recognition domain that recognizes one or more epitopes present on human XCR1. In an embodiment, the human XCR1 comprises the amino acid sequence of SEQ ID NO: 1233.

In various embodiments, the XCR1 targeting moiety is capable of specific binding. In various embodiments, the XCR1 targeting moiety comprises an antigen recognition domain such as an antibody or derivatives thereof.

In some embodiments, the XCR1 targeting moiety comprises an antibody derivative or format. In some embodiments, the XCR1 targeting moiety comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; an Affimer, a Transbody; an Anticalin; an AdNectin; an alphabody; a bicyclic peptide; an Affilin; a Microbody; a peptide aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)2, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the XCR1 targeting moiety comprises a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3). In an embodiment, the Fc-based chimeric protein complex comprises a VHH.

In some embodiments, the XCR1 targeting moiety comprises a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In some embodiments, the XCR1 targeting moiety comprises a VHH having a variable domain comprising at least one CDR1, CDR2, and/or CDR3 sequences. In various embodiments, the XCR1 targeting moiety comprises a VHH having a variable region comprising at least one FR1, FR2, FR3, and FR4 sequences.

In some embodiments, the present invention contemplates the use of any natural or synthetic analogs, mutants, variants, alleles, homologs and orthologs (herein collectively referred to as “analogs”) of the XCR1 targeting moieties described herein. In various embodiments, the amino acid sequence of the XCR1 targeting moiety further includes an amino acid analog, an amino acid derivative, or other non-classical amino acids.

In some embodiments, the XCR1 targeting moiety comprises a sequence that is at least 60% identical to any one of the XCR1sequences disclosed herein. For example, the XCR1 targeting moiety may comprise a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the XCR1 sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the XCR1 sequences disclosed herein).

In some embodiments, the XCR1 targeting moiety comprises an amino acid sequence having one or more amino acid mutations with respect to any one of the sequences disclosed herein. In various embodiments, the XCR1 targeting moiety comprises an amino acid sequence having one, or two, or three, or four, or five, or six, or seen, or eight, or nine, or ten, or fifteen, or twenty amino acid mutations with respect to any one of the sequences disclosed herein. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids. Exemplary non-classical amino acids include, but are not limited to, selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general.

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the XCR1 targeting moiety's capability to specifically bind to XCR1. In various embodiments, the mutations do not substantially reduce the XCR1 targeting moiety's capability to specifically bind to XCR1 and without functionally modulating (e.g., partially or fully neutralizing) XCR1.

In various embodiments, the binding affinity of the XCR1 targeting moiety for the full-length and/or mature forms and/or isoforms and/or splice variants and/or fragments and/or monomeric and/or dimeric forms and/or any other naturally occurring or synthetic analogs, variants, or mutants (including monomeric and/or dimeric forms) of human XCR1 may be described by the equilibrium dissociation constant (K_{D}). In various embodiments, the Fc-based chimeric protein complex comprises a targeting moiety that binds to the full-length and/or mature forms and/or isoforms and/or splice variants and/or fragments and/or any other naturally occurring or synthetic analogs, variants, or mutants (including monomeric and/or dimeric forms) of human XCR1 with a K_{D} of less than about 1 uM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 1 nM.

In various embodiments, the XCR1 targeting moiety binds but does not functionally modulate (e.g., partially or fully neutralize) the antigen of interest, i.e., XCR1. For instance, in various embodiments, the XCR1 targeting moiety simply targets the antigen but does not substantially functionally modulate (e.g. partially or fully inhibit, reduce or neutralize) a biological effect that the antigen has. In various embodiments, the XCR1 targeting moiety binds an epitope that is physically separate from an antigen site that is important for its biological activity (e.g. an antigen's active site).

Such binding without significant function modulation finds use in various embodiments of the present invention, including methods in which the XCR1 targeting moiety is used to directly or indirectly recruit active immune cells to a site of need via an effector antigen. For example, in various embodiments, the XCR1 targeting moiety can be used to directly or indirectly recruit dendritic cells via XCR1 to a tumor cell in a method of reducing or eliminating a tumor (e.g. the XCR1 targeting moiety can comprise a binding agent having an anti-XCR1 antigen recognition domain and a targeting moiety having a recognition domain (e.g. antigen recognition domain) directed against a tumor antigen or receptor). In such embodiments, it is desirable to directly or indirectly recruit dendritic cells but not to functionally modulate or neutralize the XCR1 activity. In these embodiments, XCR1 signaling is an important piece of the tumor reducing or eliminating effect.

In some embodiments, the XCR1 targeting moiety enhances antigen-presentation by dendritic cells. For example, in various embodiments, the XCR1 targeting moiety directly or indirectly recruits dendritic cells via XCR1 to a tumor cell, where tumor antigens are subsequently endocytosed and presented on the dendritic cell for induction of potent humoral and cytotoxic T cell responses.

In other embodiments (for example, related to treating autoimmune or neurodegenerative disease), the XCR1 targeting moiety comprises a binding agent that binds and neutralizes the antigen of interest, i.e., XCR1. For instance, in various embodiments, the present methods may inhibit or reduce XCR1 signaling or expression, e.g. to cause a reduction in an immune response.

### Non-Cellular Structure Targeting Moieties

In some embodiments, the targeting moiety's target (e.g. antigen or receptor) is part of a non-cellular structure. In some embodiments, the antigen or receptor is not an integral component of an intact cell or cellular structure. In some embodiments, the antigen or receptor is an extracellular antigen or receptor. In some embodiments, the target is a non-proteinaceous, non-cellular marker, including, without limitation, nucleic acids, inclusive of DNA or RNA, such as, for example, DNA released from necrotic tumor cells or extracellular deposits such as cholesterol.

In some embodiments, the target of interest (e.g. antigen, receptor) is part of the non-cellular component of the stroma or the extracellular matrix (ECM) or the markers associated therewith. As used herein, stroma refers to the connective and supportive framework of a tissue or organ. Stroma may include a compilation of cells such as fibroblasts/myofibroblasts, glial, epithelia, fat, immune, vascular, smooth muscle, and immune cells along with the extracellular matrix (ECM) and extracellular molecules. In various embodiments, the target (e.g. antigen, receptor) of interest is part of the non-cellular component of the stroma such as the extracellular matrix and extracellular molecules. As used herein, the ECM refers to the non-cellular components present within all tissues and organs. The ECM is composed of a large collection of biochemically distinct components including, without limitation, proteins, glycoproteins, proteoglycans, and polysaccharides. These components of the ECM are usually produced by adjacent cells and secreted into the ECM via exocytosis. Once secreted, the ECM components often aggregate to form a complex network of macromolecules. In various embodiments, the Fc-based chimeric protein complex of the invention comprises a targeting moiety that recognizes a target (e.g., an antigen or receptor or non-proteinaceous molecule) located on any component of the ECM. Illustrative components of the ECM include, without limitation, the proteoglycans, the non-proteoglycan polysaccharides, fibers, and other ECM proteins or ECM non-proteins, e.g. polysaccharides and/or lipids, or ECM associated molecules (e.g. proteins or non-proteins, e.g. polysaccharides, nucleic acids and/or lipids).

In some embodiments, the targeting moiety recognizes a target (e.g. antigen, receptor) on ECM proteoglycans. Proteoglycans are glycosylated proteins. The basic proteoglycan unit includes a core protein with one or more covalently attached glycosaminoglycan (GAG) chains. Proteoglycans have a net negative charge that attracts positively charged sodium ions (Na+), which attracts water molecules via osmosis, keeping the ECM and resident cells hydrated. Proteoglycans may also help to trap and store growth factors within the ECM. Illustrative proteoglycans that may be targeted by the Fc-based chimeric protein complexes of the invention include, but are not limited to, heparan sulfate, chondroitin sulfate, and keratan sulfate. In an embodiment, the targeting moiety recognizes a target (e.g. antigen, receptor) on non-proteoglycan polysaccharides such as hyaluronic acid.

In some embodiments, the targeting moiety recognizes a target (e.g. antigen, receptor) on ECM fibers. ECM fibers include collagen fibers and elastin fibers. In some embodiments, the targeting moiety recognizes one or more epitopes on collagens or collagen fibers. Collagens are the most abundant proteins in the ECM. Collagens are present in the ECM as fibrillar proteins and provide structural support to resident cells. In one or more embodiments, the targeting moiety recognizes and binds to various types of collagens present within the ECM including, without limitation, fibrillar collagens (types I, II, III, V, XI), facit collagens (types IX, XII, XIV), short chain collagens (types VIII, X), basement membrane collagens (type IV), and/or collagen types VI, VII, or XIII. Elastin fibers provide elasticity to tissues, allowing them to stretch when needed and then return to their original state. In some embodiments, the target moiety recognizes one or more epitopes on elastins or elastin fibers.

In some embodiments, the targeting moiety recognizes one or more ECM proteins including, but not limited to, a tenascin, a fibronectin, a fibrin, a laminin, or a nidogen/entactin.

In an embodiment, the targeting moiety recognizes and binds to tenascin. The tenascin (TN) family of glycoproteins includes at least four members, tenascin-C, tenascin-R, tenascin-X, and tenascin W. The primary structures of tenascin proteins include several common motifs ordered in the same consecutive sequence: amino-terminal heptad repeats, epidermal growth factor (EGF)-like repeats, fibronectin type III domain repeats, and a carboxyl-terminal fibrinogen-like globular domain. Each protein member is associated with typical variations in the number and nature of EGF-like and fibronectin type III repeats. Isoform variants also exist particularly with respect to tenascin-C. Over 27 splice variants and/or isoforms of tenascin-C are known. In a particular embodiment, the targeting moiety recognizes and binds to tenascin-CA1. Similarly, tenascin-R also has various splice variants and isoforms. Tenascin-R usually exists as dimers or trimers. Tenascin-X is the largest member of the tenascin family and is known to exist as trimers. Tenascin-W exists as trimers. In some embodiments, the targeting moiety recognizes one or more epitopes on a tenascin protein. In some embodiments, the targeting moiety recognizes the monomeric and/or the dimeric and/or the trimeric and/or the hexameric forms of a tenascin protein.

In some embodiments, the targeting moiety recognizes tenascin-CA1.

In some embodiments, the targeting moieties recognize and bind to fibronectin. Fibronectins are glycoproteins that connect cells with collagen fibers in the ECM, allowing cells to move through the ECM. Upon binding to integrins, fibronectins unfolds to form functional dimers. In some embodiments, the targeting moiety recognizes the monomeric and/or the dimeric forms of fibronectin. In some embodiments, the targeting moiety recognizes one or more epitopes on fibronectin. In illustrative embodiments, the targeting moiety recognizes fibronectin extracellular domain A (EDA) or fibronectin extracellular domain B (EDB). Elevated levels of EDA are associated with various diseases and disorders including psoriasis, rheumatoid arthritis, diabetes, and cancer. In some embodiments, the targeting moiety recognizes fibronectin that contains the EDA isoform and may be utilized to target the Fc-based chimeric protein complex to diseased cells including cancer cells. In some embodiments, the targeting moiety recognizes fibronectin that contains the EDB isoform. In various embodiments, such targeting moieties may be utilized to target the Fc-based chimeric protein complex to tumor cells including the tumor neovasculature.

In an embodiment, the targeting moiety recognizes and binds to fibrin. Fibrin is another protein substance often found in the matrix network of the ECM. Fibrin is formed by the action of the protease thrombin on fibrinogen which causes the fibrin to polymerize. In some embodiments, the targeting moiety recognizes one or more epitopes on fibrin. In some embodiments, the targeting moiety recognizes the monomeric as well as the polymerized forms of fibrin.

In an embodiment, the targeting moiety recognizes and binds to laminin. Laminin is a major component of the basal lamina, which is a protein network foundation for cells and organs. Laminins are heterotrimeric proteins that contain an α-chain, a β-chain, and a γ-chain. In some embodiments, the targeting moiety recognizes one or more epitopes on laminin. In some embodiments, the targeting moiety recognizes the monomeric, the dimeric as well as the trimeric forms of laminin.

In an embodiment, the targeting moiety recognizes and binds to a nidogen or entactin. Nidogens/entactins are a family of highly conserved, sulfated glycoproteins. They make up the major structural component of the basement membranes and function to link laminin and collagen IV networks in basement membranes. Members of this family include nidogen-1 and nidogen-2. In various embodiments, the targeting moiety recognizes an epitope on nidogen-1 and/or nidogen-2.

In various embodiments, the targeting moiety comprises an antigen recognition domain that recognizes an epitope present on any of the targets described herein. In an embodiment, the antigen-recognition domain recognizes one or more linear epitopes present on the protein. As used herein, a linear epitope refers to any continuous sequence of amino acids present on the protein. In another embodiment, the antigen-recognition domain recognizes one or more conformational epitopes present on the protein. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the targeting moiety may bind to the full-length and/or mature forms and/or isoforms and/or splice variants and/or fragments and/or any other naturally occurring or synthetic analogs, variants, or mutants of any of the targets described herein. In various embodiments, the targeting moiety may bind to any forms of the proteins described herein, including monomeric, dimeric, trimeric, tetrameric, heterodimeric, multimeric and associated forms. In various embodiments, the targeting moiety may bind to any post-translationally modified forms of the proteins described herein, such as glycosylated and/or phosphorylated forms.

In various embodiments, the targeting moiety comprises an antigen recognition domain that recognizes extracellular molecules such as DNA. In some embodiments, the targeting moiety comprises an antigen recognition domain that recognizes DNA. In an embodiment, the DNA is shed into the extracellular space from necrotic or apoptotic tumor cells or other diseased cells.

In some embodiments, the targeting moiety comprises an antigen recognition domain that recognizes one or more non-cellular structures associated with atherosclerotic plaques. Two types of atherosclerotic plaques are known. The fibro-lipid (fibro-fatty) plaque is characterized by an accumulation of lipid-laden cells underneath the intima of the arteries. Beneath the endothelium there is a fibrous cap covering the atheromatous core of the plaque. The core includes lipid-laden cells (macrophages and smooth muscle cells) with elevated tissue cholesterol and cholesterol ester content, fibrin, proteoglycans, collagen, elastin, and cellular debris. In advanced plaques, the central core of the plaque usually contains extracellular cholesterol deposits (released from dead cells), which form areas of cholesterol crystals with empty, needle-like clefts. At the periphery of the plaque are younger foamy cells and capillaries. A fibrous plaque is also localized under the intima, within the wall of the artery resulting in thickening and expansion of the wall and, sometimes, spotty localized narrowing of the lumen with some atrophy of the muscular layer. The fibrous plaque contains collagen fibers (eosinophilic), precipitates of calcium (hematoxylinophilic) and lipid-laden cells. In some embodiments, the targeting moiety recognizes and binds to one or more of the non-cellular components of these plaques such as the fibrin, proteoglycans, collagen, elastin, cellular debris, and calcium or other mineral deposits or precipitates. In some embodiments, the cellular debris is a nucleic acid, e.g. DNA or RNA, released from dead cells.

In various embodiments, the targeting moiety comprises an antigen recognition domain that recognizes one or more non-cellular structures found in the brain plaques associated with neurodegenerative diseases. In some embodiments, the targeting moiety recognizes and binds to one or more non-cellular structures located in the amyloid plaques found in the brains of patients with Alzheimer's disease. For example, the targeting moiety may recognize and bind to the peptide amyloid beta, which is a major component of the amyloid plaques. In some embodiments, the targeting moiety recognizes and binds to one or more non-cellular structures located in the brains plaques found in patients with Huntington's disease. In various embodiments, the targeting moiety recognizes and binds to one or more non-cellular structures found in plaques associated with other neurodegenerative or musculoskeletal diseases such as Lewy body dementia and inclusion body myositis

In some embodiments, the targeting moiety is a protein-based agent capable of specific binding, such as an antibody or derivatives thereof.

### CD3 Targeting Moieties

In some embodiments, the present Fc-based chimeric protein complex has one or more targeting moieties directed against CD3 expressed on T cells. In some embodiments, the Fc-based chimeric protein complex has one or more targeting moieties which selectively bind a CD3 polypeptide. In some embodiments, the Fc-based chimeric protein complex comprises one or more antibodies, antibody derivatives or formats, peptides or polypeptides, or fusion proteins that selectively bind a CD3 polypeptide.

In some embodiments, the targeting moiety comprises the anti-CD3 antibody muromonab-CD3 (aka Orthoclone OKT3), or fragments thereof. Muromonab-CD3 is disclosed in U.S. Pat. No. 4,361,549 and Wilde et al. (1996) 51:865-894, the entire disclosures of which are hereby incorporated by reference. In illustrative embodiments, muromonab-CD3 or an antigen-binding fragment thereof for use in the methods provided herein comprises a heavy chain comprising the amino acid sequence of SEQ ID NO: 1234; and/or a light chain comprising the amino acid sequence of SEQ ID NO: 1235.

In some embodiments, the targeting moiety comprises the anti-CD3 antibody otelixizumab, or fragments thereof. Otelixizumab is disclosed in U.S. Patent Publication No. 20160000916 and Chatenoud et al. (2012) 9:372-381, the entire disclosures of which are hereby incorporated by reference. In illustrative embodiments, otelixizumab or an antigen-binding fragment thereof for use in the methods provided herein comprises a heavy chain comprising the amino acid sequence of SEQ ID NO: 1236; and/or a light chain comprising the amino acid sequence of SEQ ID NO: 1237.

In some embodiments, the targeting moiety comprises the anti-CD3 antibody teplizumab (AKA MGA031 and hOKT3γ1(Ala-Ala)), or fragments thereof. Teplizumab is disclosed in Chatenoud et al. (2012) 9:372-381, the entire disclosures of which are hereby incorporated by reference. In illustrative embodiments, teplizumab or an antigen-binding fragment thereof for use in the methods provided herein comprises a heavy chain comprising the amino acid sequence of SEQ ID NO: 1238; and/or a light chain comprising the amino acid sequence of SEQ ID NO: 1239.

In some embodiments, the targeting moiety comprises the anti-CD3 antibody visilizumab (AKA Nuvion®; HuM291), or fragments thereof. Visilizumab is disclosed in U.S. Pat. No. 5,834,597 and WO2004052397, and Cole et al., Transplantation (1999) 68:563-571, the entire disclosures of which are hereby incorporated by reference. In illustrative embodiments, visilizumab or an antigen-binding fragment thereof for use in the methods provided herein comprises a heavy chain variable region comprising the amino acid sequence of SEQ ID NO: 1240; and/or a light chain variable region comprising the amino acid sequence of SEQ ID NO: 1241.

In some embodiments, the targeting moiety comprises the anti-CD3 antibody foralumab (aka NI-0401), or fragments thereof. In various embodiments, the targeting moiety comprises any one of the anti-CD3 antibodies disclosed in US20140193399, U.S. Pat. No. 7,728,114, US20100183554, and U.S. Pat. No. 8,551,478, the entire disclosures of which are hereby incorporated by reference.

In illustrative embodiments, the anti-CD3 antibody or an antigen-binding fragment thereof for use in the methods provided herein comprises a heavy chain variable region comprising the amino acid sequence of SEQ ID Nos: 2 and 6 of U.S. Pat. No. 7,728,114 (SEQ ID NO: 1242 and 1243, respectively) and/or a light chain variable region comprising the amino acid sequence of SEQ ID NOs 4 and 8 of U.S. Pat. No. 7,728,114 (SEQ ID NO: 1244 and 1245).

In an embodiment, the targeting moiety comprises a heavy chain variable region comprising the amino acid sequence of SEQ ID NO:2 of U.S. Pat. No. 7,728,114 and a light chain variable region comprising the amino acid sequence of SEQ ID NO:4 of U.S. Pat. No. 7,728,114. In an embodiment, the targeting moiety comprises any one of the anti-CD3 antibodies disclosed in US2016/0168247, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0168247 (SEQ ID Nos.: 1246-1249, respectively) and/or a light chain comprising an amino acid sequence selected from SEQ ID Nos: 10-12 of US2016/0168247 (SEQ ID Nos.: 1250-1252, respectively).

In an embodiment, the targeting moiety comprises any one of the anti-CD3 antibodies disclosed in US2015/0175699, the entire contents of which are hereby incorporated by reference. In illustrative embodiments, the antibody or an antigen-binding fragment thereof for use in the methods provided herein comprises a heavy chain comprising an amino acid sequence selected from SEQ ID No: 9 of US2015/0175699 (SEQ ID NO: 1253); and/or a light chain comprising an amino acid sequence selected from SEQ ID No: 10 of US2015/0175699 (SEQ ID NO: 1254).

In an embodiment, the targeting moiety comprises any one of the anti-CD3 antibodies disclosed in U.S. Pat. No. 8,784,821, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 2, 18, 34, 50, 66, 82, 98 and 114 of U.S. Pat. No. 8,784,821 (SEQ ID Nos.: 1255, 1256, 1257, 1258, 1259, 1260, 1261, and 1262, respectively); and/or a light chain comprising an amino acid sequence selected from SEQ ID Nos: 10, 26, 42, 58, 74, 90, 106 and 122 of U.S. Pat. No. 8,784,821 (SEQ ID No.: 1263, 1264, 1265, 1266, 1267, 1268, 1269, and 1270, respectively).

In an embodiment, the targeting moiety comprises any one of the anti-CD3 binding constructs disclosed in US20150118252, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6 and 86 of US20150118252 (SEQ ID NO: 1271 and 1272, respectively); and/or a light chain comprising an amino acid sequence selected from SEQ ID No: 3 of US2015/0175699 (SEQ ID NO: 1273).

In an embodiment, the targeting moiety comprises any one of the anti-CD3 binding proteins disclosed in US2016/0039934, the entire contents of which are hereby incorporated by reference. In illustrative embodiments, the antibody or an antigen-binding fragment thereof for use in the methods provided herein comprises a heavy chain comprising an amino acid sequence selected from SEQ ID Nos: 6-9 of US2016/0039934 (SEQ ID Nos.: 1274-1277); and/or a light chain comprising an amino acid sequence selected from SEQ ID Nos: 1-4 of US2016/0039934 (SEQ ID Nos.: 1278-1281).

In various embodiments, the targeting moieties of the invention may comprise a sequence that targets CD3 which is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any of the sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity with any of the sequences disclosed herein).

In various embodiments, the targeting moieties of the invention may comprise any combination of heavy chain, light chain, heavy chain variable region, light chain variable region, complementarity determining region (CDR), and framework region sequences that target CD3 as disclosed herein. In various embodiments, the targeting moieties of the invention may comprise any heavy chain, light chain, heavy chain variable region, light chain variable region, complementarity determining region (CDR), and framework region sequences of the CD3-specific antibodies including, but not limited to, X35-3, VIT3, BMA030 (BW264/56), CLB-T3/3, CRIS7, YTH12.5, FI 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, WT31 and F101.01. These CD3-specific antibodies are well known in the art and, inter a/ia, described in Tunnacliffe (1989), Int. Immunol. 1, 546-550, the entire disclosures of which are hereby incorporated by reference.

Additional antibodies, antibody derivatives or formats, peptides or polypeptides, or fusion proteins that selectively bind or target CD3 are disclosed in US Patent Publication No. 2016/0000916, U.S. Pat. Nos. 4,361,549, 5,834,597, 6,491,916, 6,406,696, 6,143,297, 6,750,325 and International Publication No. WO 2004/052397, the entire disclosures of which are hereby incorporated by reference.

### CD20 Targeting Moieties

In various embodiments, the CD20 targeting moiety is a protein-based agent capable of specific binding to CD20. In various embodiments, the CD20 targeting moiety is a protein-based agent capable of specific binding to CD20 without neutralization of CD20. CD20 is a non-glycosylated member of the membrane-spanning 4-A (MS4A) family. It functions as a B cell specific differentiation antigen in both mouse and human. In particular, human CD20 cDNA encodes a transmembrane protein consisting of four hydrophobic membrane-spanning domains, two extracellular loops and intracellular N- and C-terminal regions.

In various embodiments, the CD20 targeting moiety comprises a targeting moiety having an antigen recognition domain that recognizes an epitope present on CD20. In an embodiment, the antigen-recognition domain recognizes one or more linear epitopes present on CD20. In some embodiments, a linear epitope refers to any continuous sequence of amino acids present on CD20. In another embodiment, the antigen-recognition domain recognizes one or more conformational epitopes present on CD20. As used herein, a conformation epitope refers to one or more sections of amino acids (which may be discontinuous) which form a three-dimensional surface with features and/or shapes and/or tertiary structures capable of being recognized by an antigen recognition domain.

In various embodiments, the CD20 targeting moiety may bind to the full-length and/or mature forms and/or isoforms and/or splice variants and/or fragments and/or any other naturally occurring or synthetic analogs, variants, or mutants of CD20 (e.g., human CD20). In various embodiments, the CD20 targeting moiety may bind to any forms of CD20 (e.g., human CD20), including monomeric, dimeric, trimeric, tetrameric, heterodimeric, multimeric and associated forms. In an embodiment, the CD20 targeting moiety binds to the monomeric form of CD20. In another embodiment, the CD20 targeting moiety binds to a dimeric form of CD20. In another embodiment, the CD20 targeting moiety binds to a tetrameric form of CD20. In a further embodiment, the CD20 targeting moiety to phosphorylated form of CD20, which may be either monomeric, dimeric, or tetrameric.

In an embodiment, the CD20 targeting moiety comprises a targeting moiety with an antigen recognition domain that recognizes one or more epitopes present on human CD20. In an embodiment, the human CD20 comprises the amino acid sequence of SEQ ID NO: 1346.

In various embodiments, the CD20 targeting moiety comprises a targeting moiety capable of specific binding. In various embodiments, the CD20 targeting moiety comprises a targeting moiety having an antigen recognition domain such as an antibody or derivatives thereof.

In some embodiments, the CD20 targeting moiety comprises a targeting moiety which is an antibody derivative or format. In some embodiments, the CD20 targeting moiety comprises a targeting moiety that is a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; an Affimer, a Transbody; an Anticalin; an AdNectin; an Affilin; a Microbody; a peptide aptamer; an alterases; a plastic antibodies; a phylomer; a stradobodies; a maxibodies; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a small (e.g. synthetic or natural) molecule, e.g. without limitation, as described in Patent Publication Nos. or U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In some embodiments, the CD20 targeting moiety comprises a targeting moiety that is a single-domain antibody, such as a VHH. The VHH may be derived from, for example, an organism that produces VHH antibody such as a camelid, a shark, or the VHH may be a designed VHH. VHHs are antibody-derived therapeutic proteins that contain the unique structural and functional properties of naturally-occurring heavy-chain antibodies. VHH technology is based on fully functional antibodies from camelids that lack light chains. These heavy-chain antibodies contain a single variable domain (V_{H}H) and two constant domains (CH2 and CH3). VHHs are commercially available under the trademark of NANOBODIES. In an embodiment, the CD20 targeting moiety comprises a Nanobody. In some embodiments, the single domain antibody as described herein is an immunoglobulin single variable domain or ISVD.

In some embodiments, the CD20 targeting moiety comprises a targeting moiety which is a VHH comprising a single amino acid chain having four “framework regions” or FRs and three “complementary determining regions” or CDRs. As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets.

In various embodiments, the CD20 targeting moiety comprises a VHH having a variable domain comprising at least one CDR1, CDR2, and/or CDR3 sequences.

In some embodiments, the CDR1 sequence is selected from SEQ ID Nos.: 1347-1366. In some embodiments, the CDR2 sequence is selected from SEQ ID Nos.: 1367-1383. In some embodiments, the CDR3 sequence is selected from SEQ ID Nos.: 1384-1396.

In various embodiments, the CD20 targeting moiety comprises a CDR1 comprising the amino acid sequence of SEQ ID NO: 1347, a CDR2 comprising the amino acid sequence of SEQ ID NO: 1367, and a CDR3 comprising the amino acid sequence of SEQ ID NO: 1384.

In various embodiments, the CD20 targeting moiety comprises a CDR1 comprising the amino acid sequence of SEQ ID NO: 1347, a CDR2 comprising the amino acid sequence of SEQ ID NO: 1368, and a CDR3 comprising the amino acid sequence of SEQ ID NO: 1384.

In various embodiments, the CD20 targeting moiety comprises a CDR1 comprising the amino acid sequence of SEQ ID NO: 1348, a CDR2 comprising the amino acid sequence of SEQ ID NO: 1367, and a CDR3 comprising the amino acid sequence of SEQ ID NO: 1384.

In various embodiments, the CD20 targeting moiety comprises a CDR1 comprising the amino acid sequence of SEQ ID NO: 1349, a CDR2 comprising the amino acid sequence of SEQ ID NO: 1367, and a CDR3 comprising the amino acid sequence of SEQ ID NO: 1384.

In various embodiments, the CD20 targeting moiety comprises a CDR1 comprising the amino acid sequence of SEQ ID NO: 1350, a CDR2 comprising the amino acid sequence of SEQ ID NO: 1369, and a CDR3 comprising the amino acid sequence of SEQ ID NO: 1385.

In various embodiments, the CD20 targeting moiety comprises a CDR1 comprising the amino acid sequence of SEQ ID NO: 1351, a CDR2 comprising the amino acid sequence of SEQ ID NO: 1370, and a CDR3 comprising the amino acid sequence of SEQ ID NO: 1386.

In various embodiments, the CD20 targeting moiety comprises a CDR1 comprising the amino acid sequence of SEQ ID NO: 1352, a CDR2 comprising the amino acid sequence of SEQ ID NO: 1371, and a CDR3 comprising the amino acid sequence of SEQ ID NO: 1387.

In various embodiments, the CD20 targeting moiety comprises a CDR1 comprising the amino acid sequence of SEQ ID NO: 1353, a CDR2 comprising the amino acid sequence of SEQ ID NO: 1371, and a CDR3 comprising the amino acid sequence of SEQ ID NO: 1388.

In various embodiments, the CD20 targeting moiety comprises a CDR1 comprising the amino acid sequence of SEQ ID NO: 1354, a CDR2 comprising the amino acid sequence of SEQ ID NO: 1372, and a CDR3 comprising the amino acid sequence of SEQ ID NO: 1389.

In various embodiments, the CD20 targeting moiety comprises a CDR1 comprising the amino acid sequence of SEQ ID NO: 1355, a CDR2 comprising the amino acid sequence of SEQ ID NO: 1373, and a CDR3 comprising the amino acid sequence of SEQ ID NO: 1390.

In various embodiments, the CD20 targeting moiety comprises a CDR1 comprising the amino acid sequence of SEQ ID NO: 1355, a CDR2 comprising the amino acid sequence of SEQ ID NO: 1374, and a CDR3 comprising the amino acid sequence of SEQ ID NO: 1390.

In various embodiments, the CD20 targeting moiety comprises a CDR1 comprising the amino acid sequence of SEQ ID NO: 1355, a CDR2 comprising the amino acid sequence of SEQ ID NO: 1375, and a CDR3 comprising the amino acid sequence of SEQ ID NO: 1390.

In various embodiments, the CD20 targeting moiety comprises a CDR1 comprising the amino acid sequence of SEQ ID NO: 1356, a CDR2 comprising the amino acid sequence of SEQ ID NO: 1374, and a CDR3 comprising the amino acid sequence of SEQ ID NO: 1390.

In various embodiments, the CD20 targeting moiety comprises a CDR1 comprising the amino acid sequence of SEQ ID NO: 1357, a CDR2 comprising the amino acid sequence of SEQ ID NO: 1376, and a CDR3 comprising the amino acid sequence of SEQ ID NO: 1391.

In various embodiments, the CD20 targeting moiety comprises a CDR1 comprising the amino acid sequence of SEQ ID NO: 1358, a CDR2 comprising the amino acid sequence of SEQ ID NO: 1377, and a CDR3 comprising the amino acid sequence of SEQ ID NO: 1392.

In various embodiments, the CD20 targeting moiety comprises a CDR1 comprising the amino acid sequence of SEQ ID NO: 1359, a CDR2 comprising the amino acid sequence of SEQ ID NO: 1377, and a CDR3 comprising the amino acid sequence of SEQ ID NO: 1392.

In various embodiments, the CD20 targeting moiety comprises a CDR1 comprising the amino acid sequence of SEQ ID NO: 1360, a CDR2 comprising the amino acid sequence of SEQ ID NO: 1377, and a CDR3 comprising the amino acid sequence of SEQ ID NO: 1392.

In various embodiments, the CD20 targeting moiety comprises a CDR1 comprising the amino acid sequence of SEQ ID NO: 1361, a CDR2 comprising the amino acid sequence of SEQ ID NO: 1378, and a CDR3 comprising the amino acid sequence of SEQ ID NO: 1392.

In various embodiments, the CD20 targeting moiety comprises a CDR1 comprising the amino acid sequence of SEQ ID NO: 1362, a CDR2 comprising the amino acid sequence of SEQ ID NO: 1379, and a CDR3 comprising the amino acid sequence of SEQ ID NO: 1392.

In various embodiments, the CD20 targeting moiety comprises a CDR1 comprising the amino acid sequence of SEQ ID NO: 1363, a CDR2 comprising the amino acid sequence of SEQ ID NO: 1377, and a CDR3 comprising the amino acid sequence of SEQ ID NO: 1392.

In various embodiments, the CD20 targeting moiety comprises a CDR1 comprising the amino acid sequence of SEQ ID NO: 1364, a CDR2 comprising the amino acid sequence of SEQ ID NO: 1380, and a CDR3 comprising the amino acid sequence of SEQ ID NO: 1393.

In various embodiments, the CD20 targeting moiety comprises a CDR1 comprising the amino acid sequence of SEQ ID NO: 1365, a CDR2 comprising the amino acid sequence of SEQ ID NO: 1381, and a CDR3 comprising the amino acid sequence of SEQ ID NO: 1394.

In various embodiments, the CD20 targeting moiety comprises a CDR1 comprising the amino acid sequence of SEQ ID NO: 1366, a CDR2 comprising the amino acid sequence of SEQ ID NO: 1382, and a CDR3 comprising the amino acid sequence of SEQ ID NO: 1395.

In various embodiments, the CD20 targeting moiety comprises a CDR1 comprising the amino acid sequence of SEQ ID NO: 1366, a CDR2 comprising the amino acid sequence of SEQ ID NO: 1383, and a CDR3 comprising the amino acid sequence of SEQ ID NO: 1396.

In various embodiments, the CD20 targeting moiety comprises an amino acid sequence selected from the following sequences: 2HCD16 (SEQ ID NO: 1397); 2HCD22 (SEQ ID NO: 1398);

2HCD35 (SEQ ID NO: 1399); 2HCD42 (SEQ ID NO: 1400); 2HCD73 (SEQ ID NO: 1401); 2HCD81 (SEQ ID NO: 1402); R3CD105 (SEQ ID NO: 1403); R3CD18 (SEQ ID NO: 1404); R3CD7 (SEQ ID NO: 1405); 2HCD25 (SEQ ID NO: 1406); 2HCD78 (SEQ ID NO: 1407); 2HCD17 (SEQ ID NO: 1408); 2HCD40 (SEQ ID NO: 1409); 2HCD88 (SEQ ID NO: 1410); 2HCD59 (SEQ ID NO: 1411); 2HCD68 (SEQ ID NO: 1412); 2HCD43 (SEQ ID NO: 1413); 2MC57 (SEQ ID NO: 1414); R2MUC70 (SEQ ID NO: 1415); R3MUC17 (SEQ ID NO: 1416); R3MUC56 (SEQ ID NO: 1417); R3MUC57 (SEQ ID NO: 1418); R3MUC58 (SEQ ID NO: 1419); R2MUC85 (SEQ ID NO: 1420); R3MUC66 (SEQ ID NO: 1421); R2MUC21 (SEQ ID NO: 1422); 2MC52 (SEQ ID NO: 1423); R3MUC22 (SEQ ID NO: 1424); R3MUC75 (SEQ ID NO: 1425); 2MC39 (SEQ ID NO: 1426); 2MC51 (SEQ ID NO: 1427); 2MC38 (SEQ ID NO: 1428); 2MC82 (SEQ ID NO: 1429); 2MC20 (SEQ ID NO: 1430); 2MC42 (SEQ ID NO:1431); R2MUC36 (SEQ ID NO: 1432); R3MCD137 (SEQ ID NO: 1433); or R3MCD22 (SEQ ID NO: 1434).

In some embodiments, the CD20 targeting moiety comprises an amino acid sequence selected from SEQ ID NOs: 1397-1434 (provided above) without the terminal histidine tag sequence (i.e., HHHHHH; SEQ ID NO: 393).

In some embodiments, the CD20 targeting moiety comprises an amino acid sequence selected from SEQ ID NOs: 1397-1434 (provided above) without the HA tag (i.e., YPYDVPDYGS; SEQ ID NO: 394).

In some embodiments, the CD20 targeting moiety comprises an amino acid sequence selected from SEQ ID NOs: 1397-1434 (provided above) without the AAA linker (i.e., AAA).

In some embodiments, the CD20 targeting moiety comprises an amino acid sequence selected from SEQ ID NOs: 1397-1434 (provided above) without the AAA linker and HA tag.

In some embodiments, the CD20 targeting moiety comprises an amino acid sequence selected from SEQ ID NOs: 1397-1434 (provided above) without the AAA linker, HA tag, and terminal histidine tag sequence (i.e., AAAYPYDVPDYGSHHHHHH; SEQ ID NO: 395).

In various embodiments, the present technology contemplates the use of any natural or synthetic analogs, mutants, variants, alleles, homologs and orthologs (herein collectively referred to as “analogs”) of the CD20 targeting moiety as described herein. In various embodiments, the amino acid sequence of the CD20 targeting moiety further includes an amino acid analog, an amino acid derivative, or other non-classical amino acids.

In various embodiments, the CD20 targeting moiety comprises a targeting moiety comprising a sequence that is at least 60% identical to any one of the CD20 sequences disclosed above. In various embodiments, the CD20 targeting moiety comprises a sequence that is at least 60% identical to any one of the CD20 sequences disclosed above minus the linker sequence, the HA tag and/or the HIS₆ tag. For example, the CD20 targeting moiety may comprise a sequence that is at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identical to any one of the CD20 sequences disclosed herein (e.g. about 60%, or about 61%, or about 62%, or about 63%, or about 64%, or about 65%, or about 66%, or about 67%, or about 68%, or about 69%, or about 70%, or about 71%, or about 72%, or about 73%, or about 74%, or about 75%, or about 76%, or about 77%, or about 78%, or about 79%, or about 80%, or about 81%, or about 82%, or about 83%, or about 84%, or about 85%, or about 86%, or about 87%, or about 88%, or about 89%, or about 90%, or about 91%, or about 92%, or about 93%, or about 94%, or about 95%, or about 96%, or about 97%, or about 98%, about 99% or about 100% sequence identity to any one of the CD20 sequences disclosed herein).

In various embodiments, the CD20 targeting moiety comprises an amino acid sequence having one or more amino acid mutations. In various embodiments, the CD20 targeting moiety comprises an amino acid sequence having one, or two, or three, or four, or five, or six, or seen, or eight, or nine, or ten, or fifteen, or twenty amino acid mutations with respect to any one of the CD20 sequences disclosed above. In some embodiments, the one or more amino acid mutations may be independently selected from substitutions, insertions, deletions, and truncations.

In some embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices.

As used herein, “non-conservative substitutions” are defined as exchanges of an amino acid by another amino acid listed in a different group of the six standard amino acid groups (1) to (6) shown above.

In various embodiments, the substitutions may also include non-classical amino acids (e.g. selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

In various embodiments, the amino acid mutation may be in the CDRs of the targeting moiety (e.g., the CDR1, CDR2 or CDR3 regions). In another embodiment, amino acid alteration may be in the framework regions (FRs) of the targeting moiety (e.g., the FR1, FR2, FR3, or FR4 regions).

Modification of the amino acid sequences may be achieved using any known technique in the art e.g., site-directed mutagenesis or PCR based mutagenesis. Such techniques are described, for example, in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989.

In various embodiments, the mutations do not substantially reduce the CD20 targeting moiety's capability to specifically bind to CD20. In various embodiments, the mutations do not substantially reduce the CD20 targeting moiety's capability to specifically bind to CD20 without neutralizing CD20.

In various embodiments, the binding affinity of the CD20 targeting moiety for the full-length and/or mature forms and/or isoforms and/or splice variants and/or fragments and/or monomeric and/or dimeric and/or tetrameric forms and/or any other naturally occurring or synthetic analogs, variants, or mutants (including monomeric and/or dimeric and/or tetrameric forms) of human CD20 may be described by the equilibrium dissociation constant (K_{D}). In various embodiments, the CD20 targeting moiety comprises a targeting moiety that binds to the full-length and/or mature forms and/or isoforms and/or splice variants and/or fragments and/or any other naturally occurring or synthetic analogs, variants, or mutants (including monomeric and/or dimeric and/or tetrameric forms) of human CD20 with a K_{D} of less than about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, or about 5 nM, or about 4.5 nM, or about 1 nM.

In various embodiments, the CD20 targeting moiety comprises a targeting moiety that binds but does not functionally modulate the antigen of interest, i.e., CD20. For instance, in various embodiments, the targeting moiety of the CD20 targeting moiety simply targets the antigen but does not substantially functionally modulate (e.g. substantially inhibit, reduce or neutralize) a biological effect that the antigen has. In various embodiments, the CD20 targeting moiety binds an epitope that is physically separate from an antigen site that is important for its biological activity (e.g. an antigen's active site).

Such binding without significant function modulation finds use in various embodiments of the present application. In various embodiments, the CD20 targeting moiety binds to CD20 positive cells and induces the death of such cells. In some embodiments, the CD20 targeting moiety induces cell death as mediated by one or more of apoptosis or direct cell death, complement-dependent cytotoxicity (CDC), antibody-dependent cellular cytotoxicity (ADCC), and/or or antibody-dependent cellular phagocytosis (ADCP). In some embodiments, the present CD20 targeting moiety induces translocation of CD20 into large lipid microdomains or ‘lipid rafts’ within the plasma membrane upon binding. This clustering process enhances the activation of complement and exerts strong complement-dependent cytotoxicity (CDC). In other embodiments, the CD20 targeting moiety induces direct cell death. In alternative embodiments, the therapeutic efficacy of the CD20 targeting moiety is not dependent on B cell depletion.

In various embodiments, the CD20 targeting moiety may be used to directly or indirectly recruit active immune cells to a site of need via an effector antigen. For example, in various embodiments, the CD20 targeting moiety may be used to directly or indirectly recruit an immune cell to a cancer or tumor cell in a method of reducing or eliminating a cancer or tumor (e.g. the CD20 targeting moiety may comprise an anti-CD20 antigen recognition domain and a targeting moiety having a recognition domain (e.g. antigen recognition domain) directed against Clec9A, which is an antigen expressed on dendritic cells). In these embodiments, CD20 signaling is an important piece of the cancer reducing or eliminating effect. In various embodiments, the CD20 targeting moiety may recruit a T cell, a B cell, a dendritic cell, a macrophage, and a natural killer (NK) cell.

### Bispecific and Multispecific Targeting Moiety Formats

In some embodiments, the Fc-based chimeric protein complexes of the present technology comprise one or more targeting moieties disclosed herein. In various embodiments, the Fc-based chimeric protein complexes have targeting moieties that target two different cells (e.g. to make a synapse) or the same cell (e.g. to get a more concentrated signaling agent effect). In various embodiments, the Fc-based chimeric protein complexes have two or more copies of the same targeting moiety (multivalency), e.g. to increase the affinity of target binding.

In various embodiments, the Fc-based chimeric protein complexes of the present technology are multi-specific, i.e., the Fc-based chimeric protein complex comprises two or more targeting moieties having recognition domains (e.g. antigen recognition domains) that recognize and bind two or more targets (e.g. antigens, or receptors, or epitopes). In such embodiments, the Fc-based chimeric protein complexes may comprise two more targeting moieties having recognition domains that recognize and bind two or more epitopes on the same antigen or on different antigens or on different receptors. In various embodiments, such multi-specific Fc-based chimeric protein complexes exhibit advantageous properties such as increased avidity and/or improved selectivity. In some embodiments, the Fc-based chimeric protein complex comprises two targeting moieties and is bispecific, i.e., binds and recognizes two epitopes on the same antigen or on different antigens or different receptors. Accordingly, in various embodiments, the Fc-based chimeric protein complex encompasses such multi-specific Fc-based chimeric protein complexes comprising two or more targeting moieties.

In various embodiments, the multi-specific Fc-based chimeric protein complexes comprises two or more targeting moieties with each targeting moiety being an antibody or an antibody derivative as described herein. In an embodiment, the multi-specific Fc-based chimeric protein complex comprises at least one VHH comprising an antigen recognition domain against one target and one antibody or antibody derivative comprising a recognition domain against a tumor antigen and/or an immune cell marker.

In various embodiments, the present multi-specific Fc-based chimeric protein complexes have two or more targeting moieties that target different antigens or receptors, and one targeting moiety may be attenuated for its antigen or receptor, e.g. the targeting moiety binds its antigen or receptor with a low affinity or avidity (including, for example, at an affinity or avidity that is less than the affinity or avidity the other targeting moiety has for its for its antigen or receptor, for instance the difference between the binding affinities may be about 10-fold, or 25-fold, or 50-fold, or 100-fold, or 300-fold, or 500-fold, or 1000-fold, or 5000-fold; for instance the lower affinity or avidity targeting moiety may bind its antigen or receptor at a K_{D} in the mid- to high-nM or low- to mid-μM range while the higher affinity or avidity targeting moiety may bind its antigen or receptor at a K_{D} in the mid- to high-μM or low- to mid-nM range). For instance, in some embodiments, the present multi-specific Fc-based chimeric protein complex comprises an attenuated targeting moiety that is directed against a promiscuous antigen or receptor, which may improve targeting to a cell of interest (e.g. via the other targeting moiety) and prevent effects across multiple types of cells, including those not being targeted for therapy (e.g. by binding promiscuous antigen or receptor at a higher affinity than what is provided in these embodiments).

The multi-specific Fc-based chimeric protein complexes may be constructed using methods known in the art, see for example, U.S. Pat. No. 9,067,991, U.S. Patent Publication No. 20110262348 and WO 2004/041862, the entire contents of which are hereby incorporated by reference. In an illustrative embodiment, the multi-specific Fc-based chimeric protein complex comprising two or more targeting moieties may be constructed by chemical crosslinking, for example, by reacting amino acid residues with an organic derivatizing agent as described by Blattler et al., Biochemistry 24, 1517-1524 and EP294703, the entire contents of which are hereby incorporated by reference. In another illustrative embodiment, the multi-specific Fc-based chimeric protein complex comprising two or more targeting moieties is constructed by genetic fusion, i.e., constructing a single polypeptide which includes the polypeptides of the individual targeting moieties. For example, a single polypeptide construct may be formed which encodes a first VHH with an antigen recognition domain against a first target and a second antibody or antibody derivative with an antigen recognition domain against e.g., a tumor antigen or a checkpoint inhibitor. A method for producing bivalent or multivalent VHH polypeptide constructs is disclosed in PCT patent application WO 96/34103, the entire contents of which is hereby incorporated by reference. In a further illustrative embodiment, the multi-specific Fc-based chimeric protein complex may be constructed by using linkers. For example, the carboxy-terminus of a first VHH with an antigen recognition domain against a first target may be linked to the amino-terminus of a second antibody or antibody derivative with an antigen recognition domain against e.g., a tumor antigen or a checkpoint inhibitor (or vice versa). Illustrative linkers that may be used are described herein. In some embodiments, the components of the multi-specific Fc-based chimeric protein complex are directly linked to each other without the use of linkers.

In various embodiments, the multi-specific Fc-based chimeric protein complex recognizes and binds to a target (e.g., XCR1, Clec9A, FAP, PD-1, PD-L1, PD-L2, SIRP1α, or CD8) and one or more antigens found on one or more immune cells, which can include, without limitation, megakaryocytes, thrombocytes, erythrocytes, mast cells, basophils, neutrophils, eosinophils, monocytes, macrophages, natural killer cells, T lymphocytes (e.g., cytotoxic T lymphocytes, T helper cells, natural killer T cells), B lymphocytes, plasma cells, dendritic cells, or subsets thereof. In some embodiments, the Fc-based chimeric protein complex specifically binds to an antigen of interest and effectively directly or indirectly recruits one of more immune cells.

In various embodiments, the multi-specific Fc-based chimeric protein complex recognizes and binds to target (e.g., XCR1, Clec9A, FAP, PD-1, PD-L1, PD-L2, SIRP1α, or CD8) and one or more antigens found on tumor cells. In these embodiments, the present Fc-based chimeric protein complex may directly or indirectly recruit an immune cell to a tumor cell or the tumor microenvironment. In some embodiments, the present Fc-based chimeric protein complex may directly or indirectly recruit an immune cell, e.g. an immune cell that can kill and/or suppress a tumor cell (e.g., a CTL), to a site of action (such as, by way of non-limiting example, the tumor microenvironment). In some embodiments, the present Fc-based chimeric protein complex enhances antigen presentation (e.g. tumor antigen presentation) by dendritic cells for the induction of a potent humoral and cytotoxic T cell response.

In some embodiments, the Fc-based chimeric protein complex may have two or more targeting moieties that bind to non-cellular structures. In some embodiments, there are two targeting moieties and one targets a cell while the other targets a non-cellular structure.

In some embodiments, the present Fc-based chimeric protein complex has (i) one or more of the targeting moieties which is directed against an immune cell selected from a T cell, a B cell, a dendritic cell, a macrophage, a NK cell, or subsets thereof and (ii) one or more of the targeting moieties which is directed against a tumor cell, along with any of the signaling agents described herein. In one embodiment, the Fc-based chimeric protein complex has (i) a targeting moiety directed against a T cell (including, without limitation an effector T cell) and (ii) a targeting moiety is directed against a tumor cell, along with any of the signaling agents described herein. In one embodiment, the present Fc-based chimeric protein complex has (i) a targeting moiety directed against a B cell and (ii) a targeting moiety is directed against a tumor cell, along with any of the signaling agents described herein. In one embodiment, the present Fc-based chimeric protein complex has (i) a targeting moiety directed against a dendritic cell and (ii) a targeting moiety is directed against a tumor cell, along with any of the signaling agents described herein. In one embodiment, the present Fc-based chimeric protein complex has (i) a targeting moiety directed against a macrophage and (ii) a targeting moiety is directed against a tumor cell, along with any of the signaling agents described herein. In one embodiment, the present Fc-based chimeric protein complex has (i) a targeting moiety directed against a NK cell and (ii) a targeting moiety is directed against a tumor cell, along with any of the signaling agents described herein.

By way of non-limiting example, in various embodiments, the present Fc-based chimeric protein complex has (i) a targeting moiety directed against a T cell, for example, mediated by targeting to CD8, SLAMF4, IL-2 R α, 4-1BB/TNFRSF9, IL-2 R β, ALCAM, B7-1, IL-4 R, B7-H3, BLAME/SLAMFS, CEACAM1, IL-6 R, CCR3, IL-7 Rα, CCR4, CXCRI/IL-S RA, CCR5, CCR6, IL-10R α, CCR 7, IL-I 0 R β, CCRS, IL-12 R β1, CCR9, IL-12 R β2, CD2, IL-13 R α 1, IL-13, CD3, CD4, ILT2/CDS5j, ILT3/CDS5k, ILT4/CDS5d, ILT5/CDS5a, lutegrin α 4/CD49d, CDS, Integrin α E/CD103, CD6, Integrin α M/CD 11 b, CDS, Integrin α X/CD11c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIR1, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β 1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, TNFR1/TNFRSF1A, Granulysin, TNF RIII/TNFRSF1B, TRAIL RI/TNFRSFIOA, ICAM-1/CD54, TRAIL R2/TNFRSF10B, ICAM-2/CD102, TRAILR3/TNFRSF10C, IFN-γR1, TRAILR4/TNFRSF10D, IFN-γ R2, TSLP, IL-1 R1, or TSLP R; and (ii) a targeting moiety is directed against a tumor cell, along with any of the signaling agents described herein.

By way of non-limiting example, in various embodiments, the present Fc-based chimeric protein complex has a targeting moiety directed against (i) a checkpoint marker expressed on a T cell, e.g. one or more of PD-1, CD28, CTLA4, ICOS, BTLA, KIR, LAG3, CD137, OX40, CD27, CD40L, TIM3, and A2aR and (ii) a targeting moiety is directed against a tumor cell, along with any of the signaling agents described herein.

In one embodiment, the present Fc-based chimeric protein complex has (i) a targeting moiety directed against a T cell, for example, mediated by targeting to CD8 and (ii) a targeting moiety is directed against a tumor cell, along with any of the signaling agents described herein. In an embodiment, the present Fc-based chimeric protein complex has a targeting moiety directed against CD8 on T cells and a second targeting moiety directed against PD-L1 or PD-L2 on tumor cells.

In one embodiment, the present Fc-based chimeric protein complex has (i) a targeting moiety directed against a T cell, for example, mediated by targeting to CD4 and (ii) a targeting moiety is directed against a tumor cell, along with any of the signaling agents described herein. In an embodiment, the present Fc-based chimeric protein complex has a targeting moiety directed against CD4 on T cells and a second targeting moiety directed against PD-L1 or PD-L2 on tumor cells.

In one embodiment, the present Fc-based chimeric protein complex has (i) a targeting moiety directed against a T cell, for example, mediated by targeting to CD3, CXCR3, CCR4, CCR9, CD70, CD103, or one or more immune checkpoint markers and (ii) a targeting moiety is directed against a tumor cell, along with any of the signaling agents described herein. In an embodiment, the present Fc-based chimeric protein complex has a targeting moiety directed against CD3 on T cells and a second targeting moiety directed against PD-L1 or PD-L2 on tumor cells.

In one embodiment, the present Fc-based chimeric protein complex has (i) a targeting moiety directed against a T cell, for example, mediated by targeting to PD-1 and (ii) a targeting moiety is directed against a tumor cell, along with any of the signaling agents described herein.

By way of non-limiting example, in various embodiments, the present Fc-based chimeric protein complex has (i) a targeting moiety directed against a B cell, for example, mediated by targeting to CD10, CD19, CD20, CD21, CD22, CD23, CD24, CD37, CD38, CD39, CD40, CD70, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138, or CDw150; and (ii) a targeting moiety is directed against a tumor cell, along with any of the signaling agents described herein. In an embodiment, the present Fc-based chimeric protein complex has a targeting moiety directed against CD20.

In one embodiment, the present Fc-based chimeric protein complex has (i) a targeting moiety directed against a B cell, for example, mediated by targeting to CD19, CD20 or CD70 and (ii) a targeting moiety is directed against a tumor cell, along with any of the signaling agents described herein.

In one embodiment, the present Fc-based chimeric protein complex has (i) a targeting moiety directed against a B cell, for example, mediated by targeting to CD20 and (ii) a targeting moiety is directed against a tumor cell, along with any of the signaling agents described herein. In an embodiment, the present has a targeting moiety directed against CD20 on B cells and a second targeting moiety directed against PD-L1 or PD-L2 on tumor cells.

By way of non-limiting example, in various embodiments, the present Fc-based chimeric protein complex has (i) a targeting moiety directed against a NK cell, for example, mediated by targeting to 2B4/SLAMF4, KIR2DS4, CD155/PVR, KIR3DL1, CD94, LMIR1/CD300A, CD69, LMIR2/CD300c, CRACC/SLAMF7, LMIR3/CD300LF, DNAM-1, LMIR5/CD300LB, Fc-epsilon RII, LMIR6/CD300LE, Fc-γ RI/CD64, MICA, Fc-γ RIIB/CD32b, MICB, Fc-γ RIIC/CD32c, MULT-1, Fc-γ RIIA/CD32a, Nectin-2/CD112, Fc-γ RIII/CD16, NKG2A, FcRH1/IRTA5, NKG2C, FcRH2/IRTA4, NKG2D, FcRH4/IRTA1, NKp30, FcRH5/IRTA2, NKp44, Fc-Receptor-like 3/CD16-2, NKp46/NCR1, NKp80/KLRF1, NTB-A/SLAMF6, Rae-1, Rae-1 α, Rae-1 ρ, Rae-1 delta, H60, Rae-1 epsilon, ILT2/CD85j, Rae-1 γ, ILT3/CD85k, TREM-1, ILT4/CD85d, TREM-2, ILT5/CD85a, TREM-3, KIR/CD158, TREML1/TLT-1, KIR2DL1, ULBP-1, KIR2DL3, ULBP-2, KIR2DL4/CD158d, or ULBP-3; and (ii) a targeting moiety is directed against a tumor cell, along with any of the signaling agents described herein.

In one embodiment, the present Fc-based chimeric protein complex has (i) a targeting moiety directed against a NK cell, for example, mediated by targeting to Kir1alpha, DNAM-1 or CD64 and (ii) a targeting moiety is directed against a tumor cell, along with any of the signaling agents described herein.

In one embodiment, the present Fc-based chimeric protein complex has (i) a targeting moiety directed against a NK cell, for example, mediated by targeting to KIR1 and (ii) a targeting moiety is directed against a tumor cell, along with any of the signaling agents described herein. In an embodiment, the present Fc-based chimeric protein complex has a targeting moiety directed against KIR1 on NK cells and a second targeting moiety directed against PD-L1 or PD-L2 on tumor cells.

In one embodiment, the present Fc-based chimeric protein complex has (i) a targeting moiety directed against a NK cell, for example, mediated by targeting to TIGIT or KIR1 and (ii) a targeting moiety is directed against a tumor cell, along with any of the signaling agents described herein. In an embodiment, the present Fc-based chimeric protein complex has a targeting moiety directed against TIGIT on NK cells and a second targeting moiety directed against PD-L1 or PD-L2 on tumor cells.

By way of non-limiting example, in various embodiments, the present Fc-based chimeric protein complex has (i) a targeting moiety directed against a dendritic cell, for example, mediated by targeting to CLEC-9A, XCR1, RANK, CD36/SRB3, LOX-1/SR-E1, CD68, MARCO, CD163, SR-A1/MSR, CD5L, SREC-1, CL-PI/COLEC12, SREC-II, LIMPIIISRB2, RP105, TLR4, TLR1, TLR5, TLR2, TLR6, TLR3, TLR9, 4-IBB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, lutegrin α 4/CD49d, Aag, Integrin β 2/CD18, AMICA, Langerin, B7-2/CD86, Leukotriene B4 RI, B7-H3, LMIR1/CD300A, BLAME/SLAMF8, LMIR2/CD300c, Clq R1/CD93, LMIR3/CD300LF, CCR6, LMIR5/CD300LB CCR7, LMIR6/CD300LE, CD40/TNFRSF5, MAG/Siglec-4-a, CD43, MCAM, CD45, MD-1, CD68, MD-2, CD83, MDL-1/CLEC5A, CD84/SLAMF5, MMR, CD97, NCAMLI, CD2F-10/SLAMF9, Osteoactivin GPNMB, Chern 23, PD-L2, CLEC-1, RP105, CLEC-2, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc-γ R1/CD64, TLR3, Fc-γ RIIB/CD32b, TREM-1, Fc-γ RIIC/CD32c, TREM-2, Fc-γ RIIA/CD32a, TREM-3, Fc-γ RIII/CD16, TREML1/TLT-1, ICAM-2/CD102, or Vanilloid R1; and (ii) a targeting moiety is directed against a tumor cell, along with any of the signaling agents described herein.

In one embodiment, the present Fc-based chimeric protein complex has (i) a targeting moiety directed against a dendritic cell, for example, mediated by targeting to CLEC-9A, DC-SIGN, CD64, CLEC4A, or DEC205 and (ii) a targeting moiety is directed against a tumor cell, along with any of the signaling agents described herein. In an embodiment, the present Fc-based chimeric protein complex has a targeting moiety directed against CLEC9A on dendritic cells and a second targeting moiety directed against PD-L1 or PD-L2 on tumor cells.

In one embodiment, the present Fc-based chimeric protein complex has (i) a targeting moiety directed against a dendritic cell, for example, mediated by targeting to CLEC9A and (ii) a targeting moiety is directed against a tumor cell, along with any of the signaling agents described herein. In an embodiment, the present Fc-based chimeric protein complex has a targeting moiety directed against CLEC9A on dendritic cells and a second targeting moiety directed against PD-L1 or PD-L2 on tumor cells.

In one embodiment, the present Fc-based chimeric protein complex has (i) a targeting moiety directed against a dendritic cell, for example, mediated by targeting to XCR1 and (ii) a targeting moiety is directed against a tumor cell, along with any of the signaling agents described herein. In an embodiment, the present Fc-based chimeric protein complex has a targeting moiety directed against XCR1 on dendritic cells and a second targeting moiety directed against PD-L1 or PD-L2 on tumor cells.

In one embodiment, the present Fc-based chimeric protein complex has (i) a targeting moiety directed against a dendritic cell, for example, mediated by targeting to RANK and (ii) a targeting moiety is directed against a tumor cell, along with any of the signaling agents described herein. In an embodiment, the present Fc-based chimeric protein complex has a targeting moiety directed against RANK on dendritic cells and a second targeting moiety directed against PD-L1 or PD-L2 on tumor cells.

By way of non-limiting example, in various embodiments, the present Fc-based chimeric protein complex has (i) a targeting moiety directed against a monocyte/macrophage, for example, mediated by targeting to SIRP1α, B7-1/CD80, ILT4/CD85d, B7-H1, ILT5/CD85a, Common β Chain, Integrin α 4/CD49d, BLAME/SLAMF8, Integrin α X/CDIIc, CCL6/C10, Integrin β 2/CD18, CD155/PVR, Integrin β 3/CD61, CD31/PECAM-1, Latexin, CD36/SR-B3, Leukotriene B4 R1, CD40/TNFRSF5, LIMPIIISR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc-γ RI/CD64, Osteopontin, Fc-γ RIIB/CD32b, PD-L2, Fc-γ RIIC/CD32c, Siglec-3/CD33, Fc-γ RIIA/CD32a, SIGNR1/CD209, Fc-γ RIII/CD16, SLAM, GM-CSF R α, TCCR/WSX-1, ICAM-2/CD102, TLR3, IFN-γ RI, TLR4, IFN-γ R2, TREM-I, IL-I RII, TREM-2, ILT2/CD85j, TREM-3, ILT3/CD85k, TREML1/TLT-1, 2B4/SLAMF 4, IL-10 R α, ALCAM, IL-10 R β, AminopeptidaseN/ANPEP, ILT2/CD85j, Common β Chain, ILT3/CD85k, Clq R1/CD93, ILT4/CD85d, CCR1, ILT5/CD85a, CCR2, CD206, Integrin α 4/CD49d, CCR5, Integrin α M/CDII b, CCR8, Integrin α X/CDIIc, CD155/PVR, Integrin β 2/CD18, CD14, Integrin β 3/CD61, CD36/SR-B3, LAIR1, CD43, LAIR2, CD45, Leukotriene B4-R1, CD68, LIMPIIISR-B2, CD84/SLAMF5, LMIR1/CD300A, CD97, LMIR2/CD300c, CD163, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc-γ RI/CD64, PSGL-1, Fc-γ RIIIICD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-1, IL-6 R, TREM-2, CXCRI/IL-8 RA, TREM-3, or TREMLI/TLT-1; and (ii) a targeting moiety is directed against a tumor cell, along with any of the signaling agents described herein.

In one embodiment, the present Fc-based chimeric protein complex has (i) a targeting moiety directed against a monocyte/macrophage, for example, mediated by targeting to B7-H1, CD31/PECAM-1, CD163, CCR2, or Macrophage Mannose Receptor CD206 and (ii) a targeting moiety is directed against a tumor cell, along with any of the signaling agents described herein.

In one embodiment, the present Fc-based chimeric protein complex has (i) a targeting moiety directed against a monocyte/macrophage, for example, mediated by targeting to SIRP1α and (ii) a targeting moiety is directed against a tumor cell, along with any of the signaling agents described herein. In an embodiment, the present Fc-based chimeric protein complex has a targeting moiety directed against SIRP1α on macrophage cells and a second targeting moiety directed against PD-L1 or PD-L2 on tumor cells.

In various embodiments, the present Fc-based chimeric protein complex has one or more targeting moieties directed against a checkpoint marker, e.g. one or more of PD-1/PD-L1 or PD-L2, CD28/CD80 or CD86, CTLA4/CD80 or CD86, ICOS/ICOSL or B7RP1, BTLA/HVEM, KIR, LAG3, CD137/CD137L, OX40/OX40L, CD27, CD40L, TIM3/Gal9, and A2aR. In one embodiment, the present Fc-based chimeric protein complex has (i) a targeting moiety directed against a checkpoint marker on a T cell, for example, PD-1 and (ii) a targeting moiety directed against a tumor cell, for example, PD-L1 or PD-L2, along with any of the signaling agents described herein. In an embodiment, the present Fc-based chimeric protein complex has a targeting moiety directed against PD-1 on T cells and a second targeting moiety directed against PD-L1 on tumor cells. In another embodiment, the present Fc-based chimeric protein complex has a targeting moiety directed against PD-1 on T cells and a second targeting moiety directed against PD-L2 on tumor cells.

In some embodiments, the present Fc-based chimeric protein complex comprises two or more targeting moieties directed to the same or different immune cells. In some embodiments, the present Fc-based chimeric protein complex has (i) one or more targeting moieties directed against an immune cell selected from a T cell, a B cell, a dendritic cell, a macrophage, a NK cell, or subsets thereof and (ii) one or more targeting moieties directed against either the same or another immune cell selected from a T cell, a B cell, a dendritic cell, a macrophage, a NK cell, or subsets thereof, along with any of the signaling agents described herein.

In one embodiment, the present Fc-based chimeric protein complex comprises one or more targeting moieties directed against a T cell and one or more targeting moieties directed against the same or another T cell. In one embodiment, the present Fc-based chimeric protein complex comprises one or more targeting moieties directed against a T cell and one or more targeting moieties directed against a B cell. In one embodiment, the present Fc-based chimeric protein complex comprises one or more targeting moieties directed against a T cell and one or more targeting moieties directed against a dendritic cell. In one embodiment, the present Fc-based chimeric protein complex comprises one or more targeting moieties against a T cell and one or more targeting moieties directed against a macrophage. In one embodiment, the present Fc-based chimeric protein complex comprises one or more targeting moieties against a T cell and one or more targeting moieties directed against a NK cell. For example, in an illustrative embodiment, the Fc-based chimeric protein complex may include a targeting moiety against CD8 and a targeting moiety against Clec9A. In another illustrative embodiment, the Fc-based chimeric protein complex may include a targeting moiety against CD8 and a targeting moiety against CD3. In another illustrative embodiment, the Fc-based chimeric protein complex may include a targeting moiety against CD8 and a targeting moiety against PD-1.

In one embodiment, the present Fc-based chimeric protein complex comprises one or more targeting moieties directed against a B cell and one or more targeting moieties directed against the same or another B cell. In one embodiment, the present Fc-based chimeric protein complex comprises one or more targeting moieties directed against a B cell and one or more targeting moieties directed against a T cell. In one embodiment, the present Fc-based chimeric protein complex comprises one or more targeting moieties directed against a B cell and one or more targeting moieties directed against a dendritic cell. In one embodiment, the present Fc-based chimeric protein complex comprises one or more targeting moieties against a B cell and one or more targeting moieties directed against a macrophage. In one embodiment, the present Fc-based chimeric protein complex comprises one or more targeting moieties against a B cell and one or more targeting moieties directed against a NK cell.

In one embodiment, the present Fc-based chimeric protein complex comprises one or more targeting moieties directed against a dendritic cell and one or more targeting moieties directed against the same or another dendritic cell. In one embodiment, the present Fc-based chimeric protein complex comprises one or more targeting moieties directed against a dendritic cell and one or more targeting moieties directed against a T cell. In one embodiment, the present Fc-based chimeric protein complex comprises one or more targeting moieties directed against a dendritic cell and one or more targeting moieties directed against a B cell. In one embodiment, the present Fc-based chimeric protein complex comprises one or more targeting moieties against a dendritic cell and one or more targeting moieties directed against a macrophage. In one embodiment, the present Fc-based chimeric protein complex comprises one or more targeting moieties against a dendritic cell and one or more targeting moieties directed against a NK cell.

In one embodiment, the present Fc-based chimeric protein complex comprises one or more targeting moieties directed against a macrophage and one or more targeting moieties directed against the same or another macrophage. In one embodiment, the present Fc-based chimeric protein complex comprises one or more targeting moieties directed against a macrophage and one or more targeting moieties directed against a T cell. In one embodiment, the present Fc-based chimeric protein complex comprises one or more targeting moieties directed against a macrophage and one or more targeting moieties directed against a B cell. In one embodiment, the present Fc-based chimeric protein complex comprises one or more targeting moieties against a macrophage and one or more targeting moieties directed against a dendritic cell. In one embodiment, the present Fc-based chimeric protein complex comprises one or more targeting moieties against a macrophage and one or more targeting moieties directed against a NK cell.

In one embodiment, the present Fc-based chimeric protein complex comprises one or more targeting moieties directed against an NK cell and one or more targeting moieties directed against the same or another NK cell. In one embodiment, the present Fc-based chimeric protein complex comprises one or more targeting moieties directed against an NK cell and one or more targeting moieties directed against a T cell. In one embodiment, the present Fc-based chimeric protein complex comprises one or more targeting moieties directed against an NK cell and one or more targeting moieties directed against a B cell. In one embodiment, the present Fc-based chimeric protein complex comprises one or more targeting moieties against an NK cell and one or more targeting moieties directed against a macrophage. In one embodiment, the present Fc-based chimeric protein complex comprises one or more targeting moieties against an NK cell and one or more targeting moieties directed against a dendritic cell.

In one embodiment, the present Fc-based chimeric protein complex comprises a targeting moiety directed against a tumor cell and a second targeting moiety directed against the same or a different tumor cell. In such embodiments, the targeting moieties may bind to any of the tumor antigens described herein.

In some embodiments, the Fc-based chimeric protein complex of the invention comprises one or more targeting moieties having recognition domains that bind to a target (e.g. antigen, receptor) of interest including those found on one or more cells selected from adipocytes (e.g., white fat cell, brown fat cell), liver lipocytes, hepatic cells, kidney cells (e.g., kidney parietal cell, kidney salivary gland, mammary gland, etc.), duct cells (of seminal vesicle, prostate gland, etc.), intestinal brush border cells (with microvilli), exocrine gland striated duct cells, gall bladder epithelial cells, ductulus efferens nonciliated cells, epididymal principal cells, epididymal basal cells, endothelial cells, ameloblast epithelial cells (tooth enamel secretion), planum semilunatum epithelial cells of vestibular system of ear (proteoglycan secretion), organ of Corti interdental epithelial cells (secreting tectorial membrane covering hair cells), loose connective tissue fibroblasts, corneal fibroblasts (corneal keratocytes), tendon fibroblasts, bone marrow reticular tissue fibroblasts, nonepithelial fibroblasts, pericytes, nucleus pulposus cells of intervertebral disc, cementoblasts/cementocytes (tooth root bonelike ewan cell secretion), odontoblasts/odontocytes (tooth dentin secretion), hyaline cartilage chondrocytes, fibrocartilage chondrocytes, elastic cartilage chondrocytes, osteoblasts/osteocytes, osteoprogenitor cells (stem cell of osteoblasts), hyalocytes of vitreous body of eye, stellate cells of perilymphatic space of ear, hepatic stellate cells (Ito cell), pancreatic stelle cells, skeletal muscle cells, satellite cells, heart muscle cells, smooth muscle cells, myoepithelial cells of iris, myoepithelial cells of exocrine glands, exocrine secretory epithelial cells (e.g., salivary gland cells, mammary gland cells, lacrimal gland cells, sweat gland cells, sebaceious gland cells, prostate gland cells, gastric glad cells, pancreatic acinar cells, pneumocytes), a hormone secreting cells (e.g., pituitary cells, neurosecretory cells, gut and respiratory tract cells, thyroid gland cells, parathyroid glad cells, adrenal gland cells, Leydig cells of testes, pancreatic islet cells), keratinizing epithelial cells, wet stratified barrier epithelial cells, neuronal cells (e.g., sensory transducer cells, autonomic neuron cells, sense organ and peripheral neuron supporting cells, and central nervous system neurons and glial cells such as interneurons, principal cells, astrocytes, oligodendrocytes, and ependymal cells).

### Fc-Based Chimeric Protein Complexes

In embodiments. the Fc-based chimeric protein complexes of the present technology comprise at least one Fc domain disclosed herein, at least one signaling agent (SA) disclosed herein, and at least one targeting moiety (TM) disclosed herein.

It is understood that, the present Fc-based chimeric protein complexes may encompass a complex of two fusion proteins.

In some embodiments, the Fc-based chimeric protein complex is heterodimeric. In some embodiments, the heterodimeric Fc-based chimeric protein complex has a trans orientation/configuration. In some embodiments, the heterodimeric Fc-based chimeric protein complex has a cis orientation/configuration. In some embodiments, the heterodimeric Fc-based chimeric protein complex does not comprise the signaling agent and targeting moiety on a single polypeptide. In some embodiments, the signaling agent and targeting moiety are on the same end (N-terminus or C-terminus) of the Fc domain or the Fc chains thereof. In some embodiments, the signaling agent and targeting moiety are on different ends (N-terminus or C-terminus) of the Fc domain or the Fc chains thereof.

In some embodiments, the Fc-based chimeric protein has an improved in vivo half-life relative to a chimeric protein lacking an Fc or a chimeric protein which is not a heterodimeric complex. In some embodiments, the Fc-based chimeric protein has an improved solubility, stability and other pharmacological properties relative to a chimeric protein lacking an Fc or a chimeric protein which is not a heterodimeric complex.

Heterodimeric Fc-based chimeric protein complexes are composed of two different polypeptides. In embodiments described herein, the targeting domain is on a different polypeptide than the signaling agent and accordingly, proteins that contain only one targeting domain copy, and also only one signaling agent copy can be made (this provides a configuration in which potential interference with desired properties can be controlled). Further, in embodiments, one targeting domain (e.g. VHH) only can avoid cross-linking of the antigen on the cell surface (which could elicit undesired effects in some cases) Further, in embodiments, one signaling agent may alleviate molecular “crowding” and potential interference with avidity mediated restoration of effector function in dependence of the targeting domain. Further, in embodiments, heterodimeric Fc-based chimeric protein complexes can have two targeting moieties and these can be placed on the two different polypeptides. For instance, in embodiments, the C-terminus of both targeting moieties (e.g. VHHs) can be masked to avoid potential autoantibodies or pre-existing antibodies (e.g. VHH autoantibodies or pre-existing antibodies). Further, in embodiments, heterodimeric Fc-based chimeric protein complexes, e.g. with the targeting domain on a different polypeptide than the signaling agent (e.g. wild type signaling agent), may favor “cross-linking” of two cell types (e.g. a tumor cell and an immune cell). Further, in embodiments, heterodimeric Fc-based chimeric protein complexes can have two signaling agents, each on different polypeptides to allow more complex effector responses (e.g. with any two of the signaling agents described herein, by way of illustration IFN alpha2 and TNF).

Further, in embodiments, heterodimeric Fc-based chimeric protein complexes, e.g. with the targeting domain on a different polypeptide than the signaling agent, combinatorial diversity of targeting moiety and signaling agent is provided in a practical manner. For instance, in embodiments, polypeptides with any of the targeting moieties described herein can be combined “off the shelf” with polypeptides with any of the signaling agents described herein to allow rapid generation of various combinations of targeting moieties and signaling agents in single Fc-based chimeric protein complexes.

In some embodiments, the Fc-based chimeric protein complex comprises one or more linkers. In some embodiments, the Fc-based chimeric protein complex includes a linker that connects the Fc domain, signaling agent(s) and targeting moiety(ies). In some embodiments, the Fc-based chimeric protein complex includes a linker that connects each signaling agent and targeting moiety (or, if more than one targeting moiety, a signaling agent to one of the targeting moieties). In some embodiments, the Fc-based chimeric protein complex includes a linker that connects each signaling agent to the Fc domain. In some embodiments, the Fc-based chimeric protein complex includes a linker that connects each targeting moiety to the Fc domain. In some embodiments, the Fc-based chimeric protein complex includes a linker that connects a targeting moiety to another targeting moiety. In some embodiments, the Fc-based chimeric protein complex includes a linker that connects a signaling agent to another signaling agent.

In some embodiments, an Fc-based chimeric protein complex comprises two or more targeting moieties. In such embodiments, the targeting moieties can be the same targeting moiety or they can be different targeting moieties.

In some embodiments, an Fc-based chimeric protein complex comprises two or more signaling agents. In such embodiments, the signaling agents can be the same targeting moiety or they can be different targeting moieties.

By way of example, in some embodiments, the Fc-based chimeric protein complex comprise an Fc domain, at least two signaling agents (SA), and at least two targeting moieties (TM), wherein the Fc domain, signaling agents, and targeting moieties are selected from any of the Fc domains, signaling agents, and targeting moieties disclosed herein. In some embodiments, the Fc domain is homodimeric.

In various embodiments, the Fc-based chimeric protein complex takes the form of any of the schematics of FIGS. 1A-F.

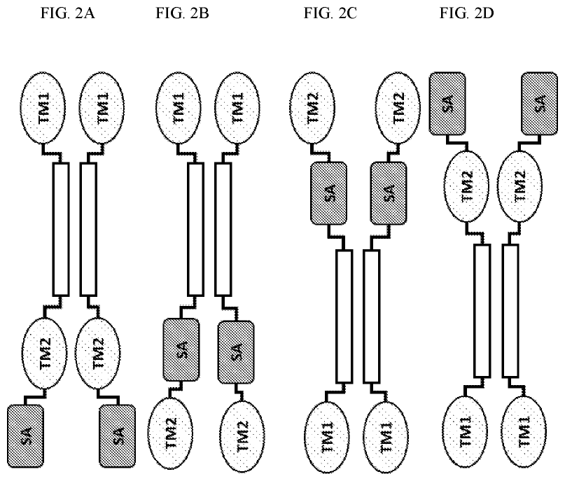

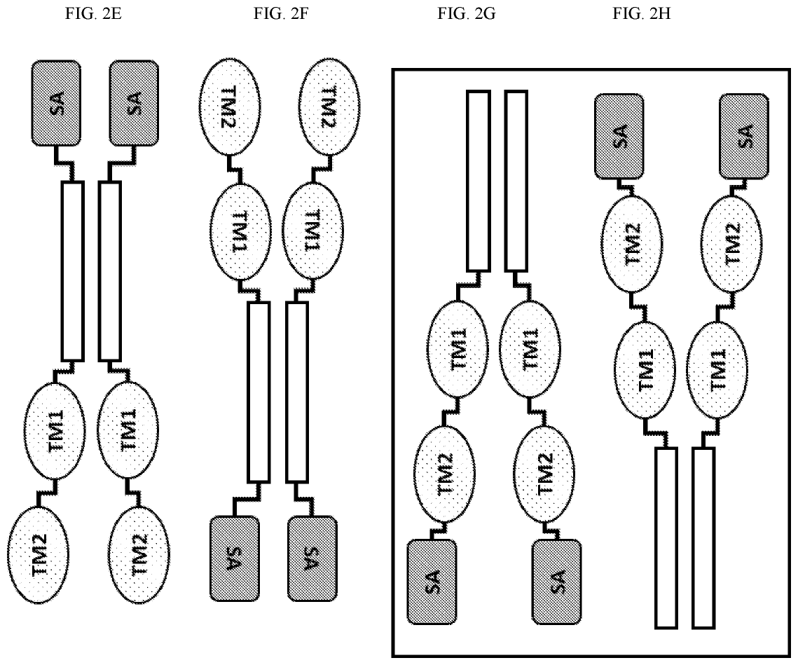

In various embodiments, the Fc-based chimeric protein complex takes the form of any of the schematics of FIGS. 2A-H.

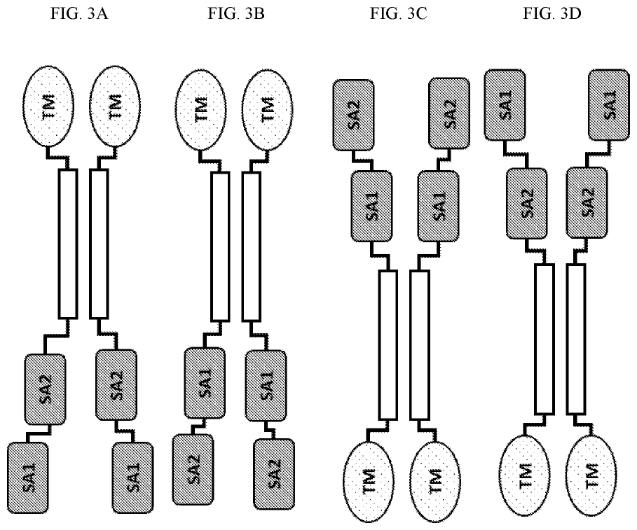

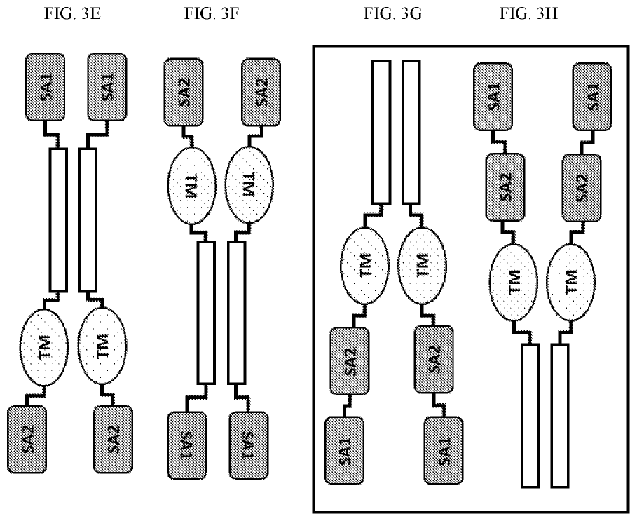

In various embodiments, the Fc-based chimeric protein complex takes the form of any of the schematics of FIGS. 3A-H.

In various embodiments, the Fc-based chimeric protein complex takes the form of any of the schematics of FIGS. 4A-D.

In various embodiments, the Fc-based chimeric protein complex takes the form of any of the schematics of FIGS. 5A-F.

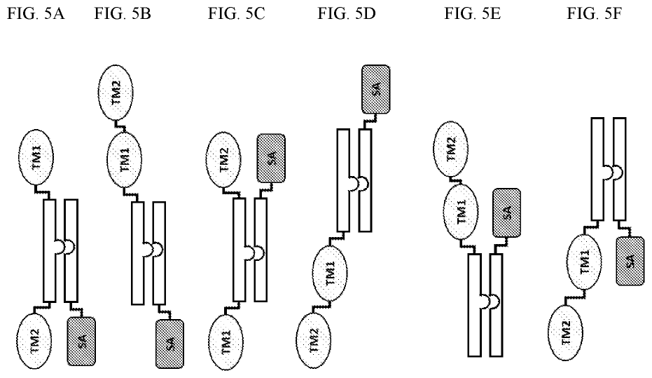

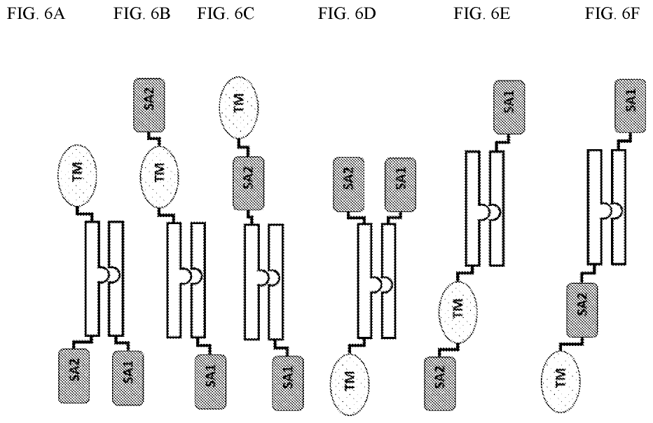

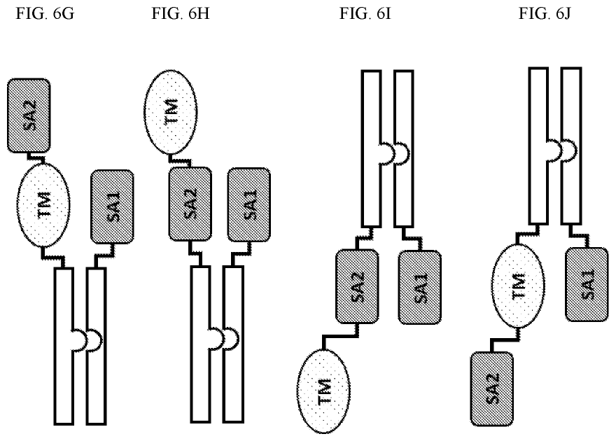

In various embodiments, the Fc-based chimeric protein complex takes the form of any of the schematics of FIGS. 6A-J.

In various embodiments, the Fc-based chimeric protein complex takes the form of any of the schematics of FIGS. 7A-D.

In various embodiments, the Fc-based chimeric protein complex takes the form of any of the schematics of FIG. 7B.

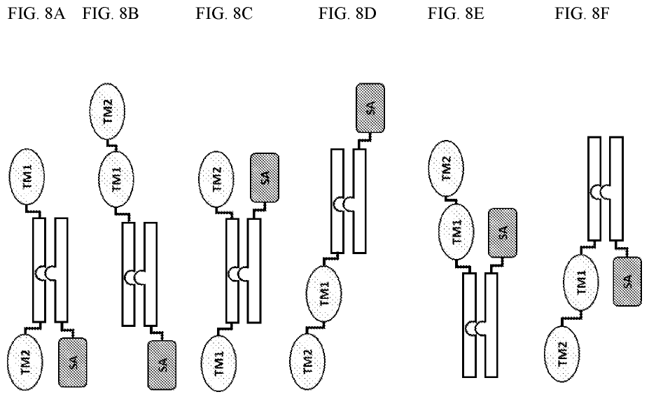

In various embodiments, the Fc-based chimeric protein complex takes the form of any of the schematics of FIGS. 8A-F.

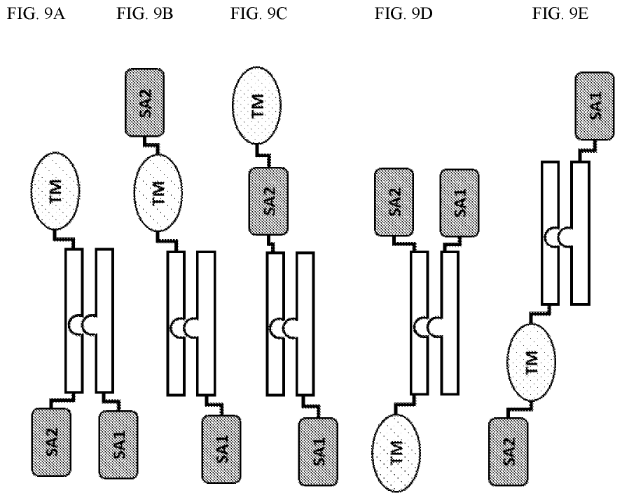

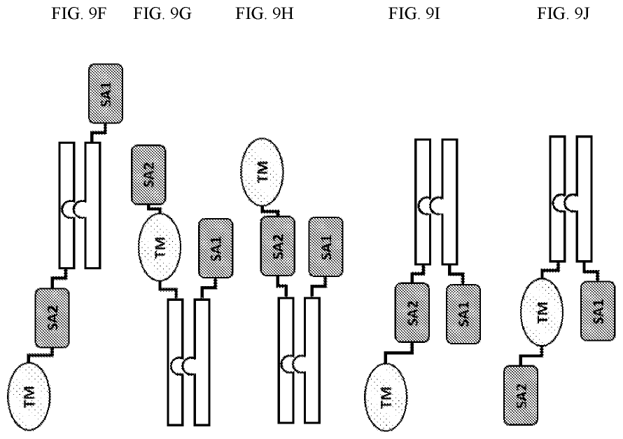

In various embodiments, the Fc-based chimeric protein complex takes the form of any of the schematics of FIGS. 9A-J.

In various embodiments, the Fc-based chimeric protein complex takes the form of any of the schematics of FIGS. 10A-F.

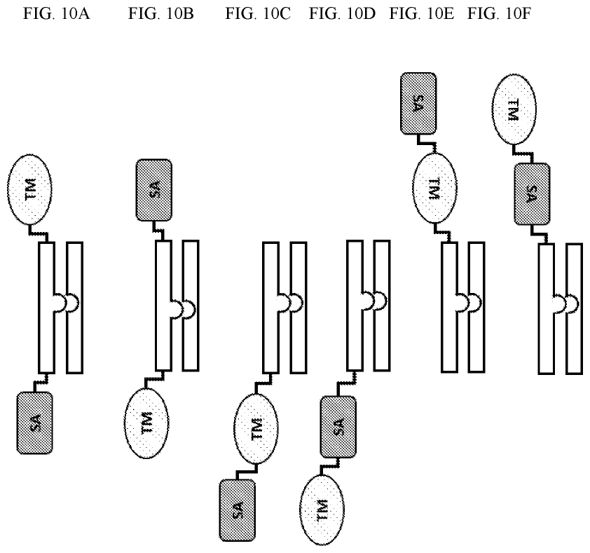

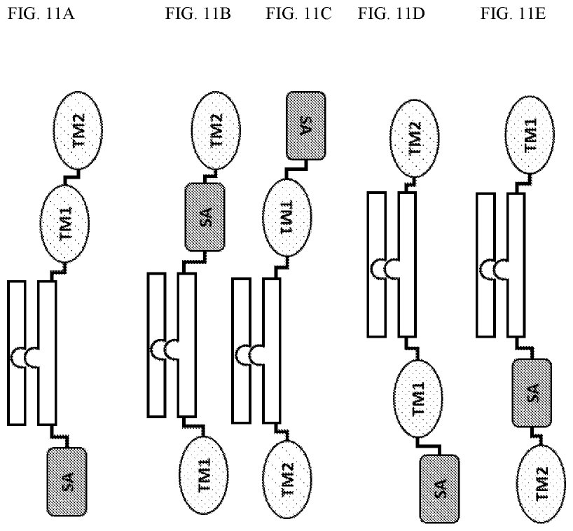

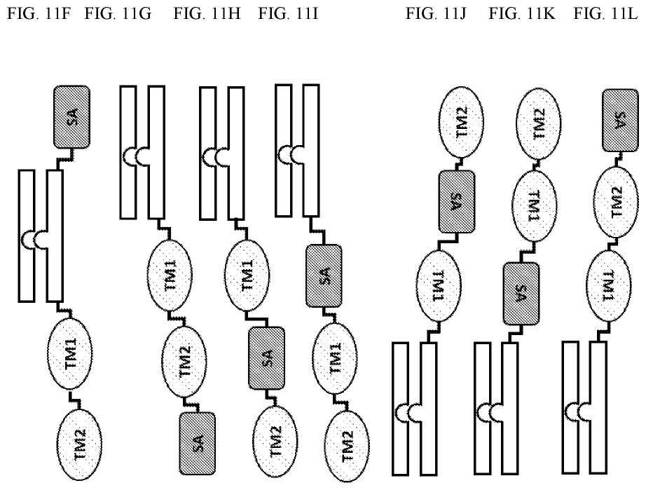

In various embodiments, the Fc-based chimeric protein complex takes the form of any of the schematics of FIGS. 11A-L.

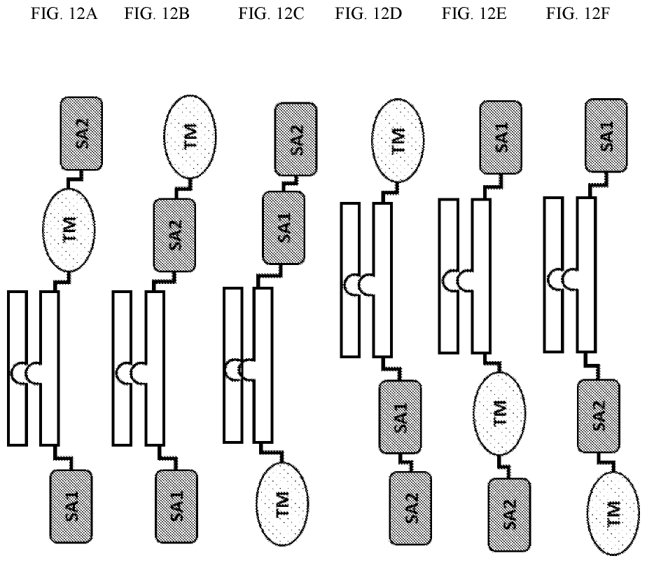

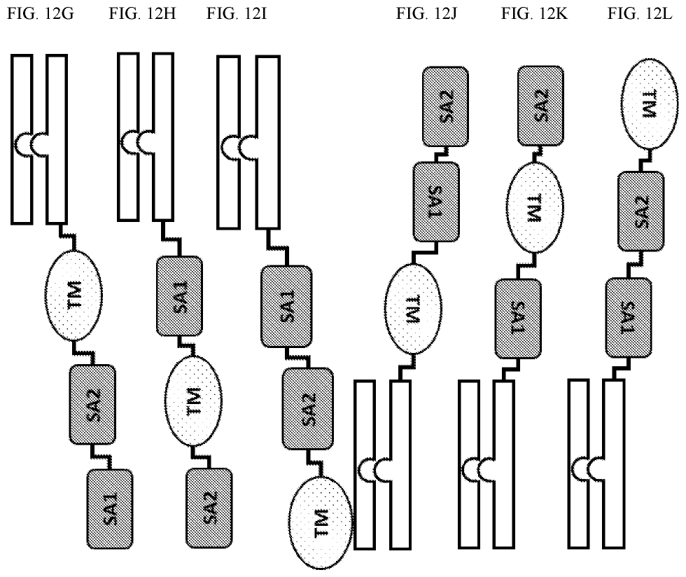

In various embodiments, the Fc-based chimeric protein complex takes the form of any of the schematics of FIGS. 12A-L.

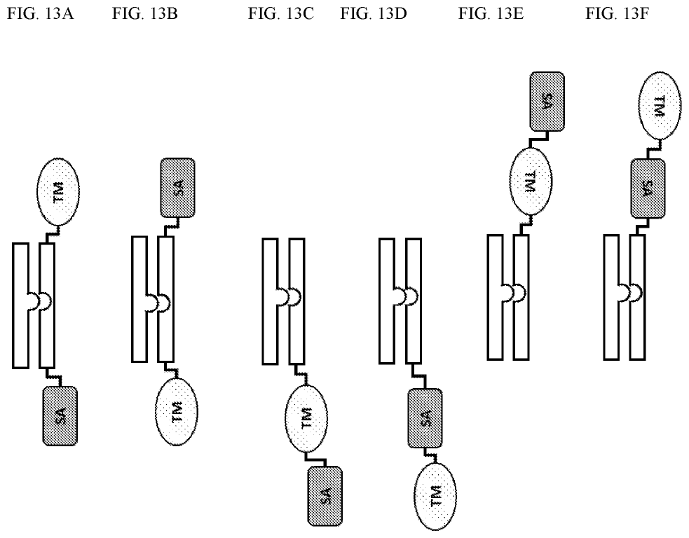

In various embodiments, the Fc-based chimeric protein complex takes the form of any of the schematics of FIGS. 13A-F.

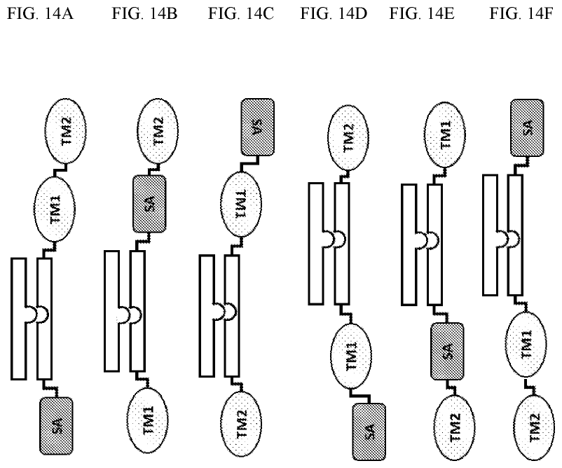

In various embodiments, the Fc-based chimeric protein complex takes the form of any of the schematics of FIGS. 14A-L.

In various embodiments, the Fc-based chimeric protein complex takes the form of any of the schematics of FIGS. 15A-L.

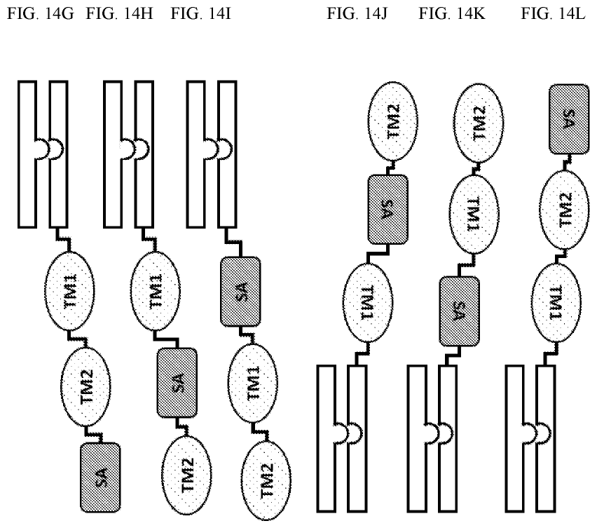

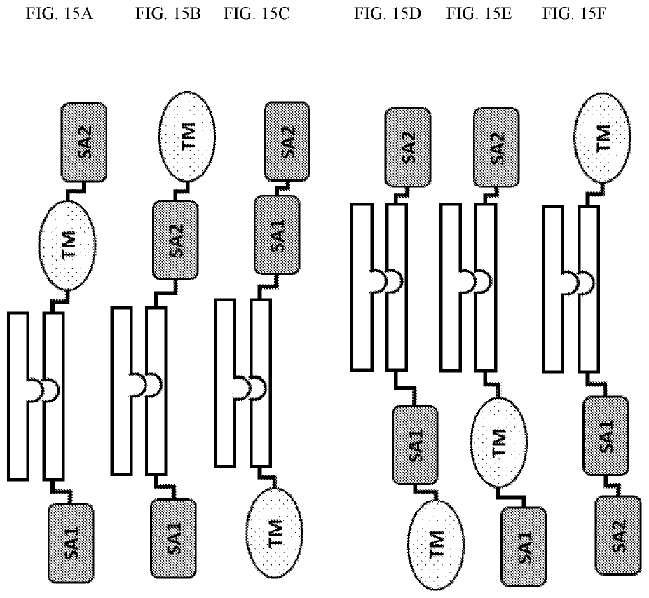

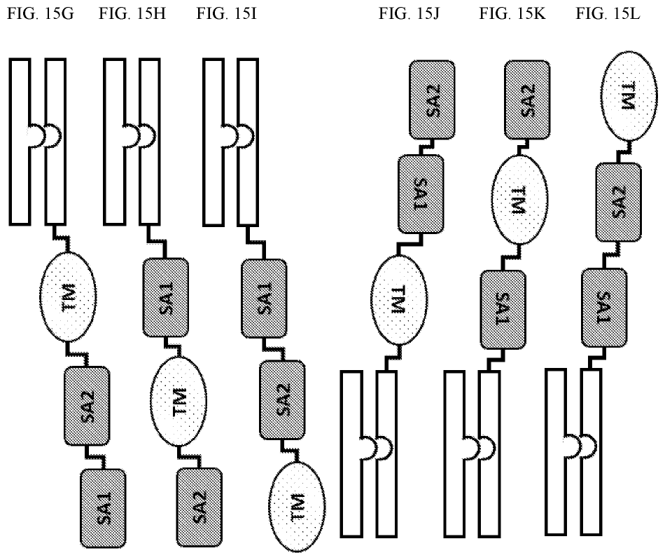

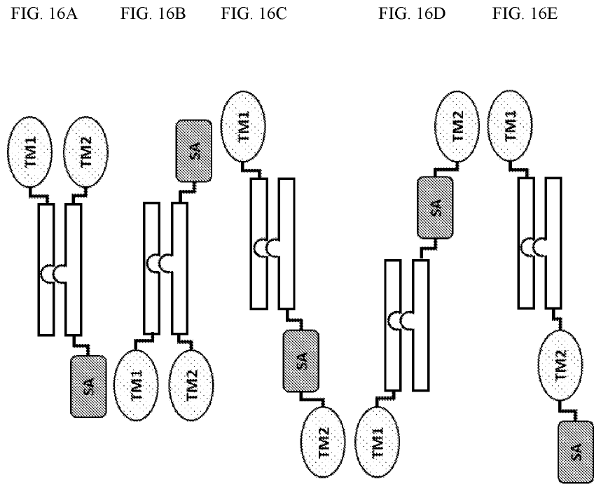

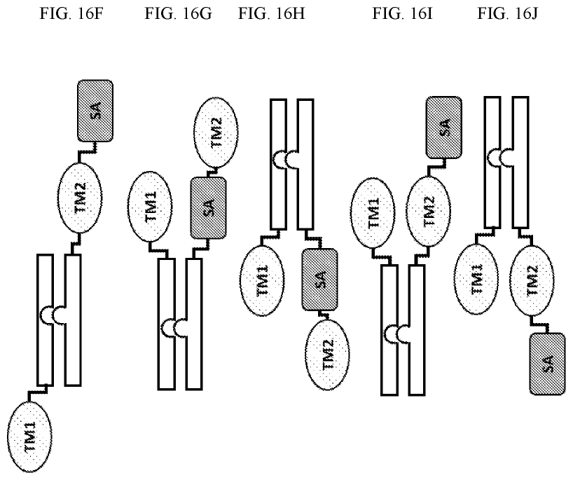

In various embodiments, the Fc-based chimeric protein complex takes the form of any of the schematics of FIGS. 16A-J.

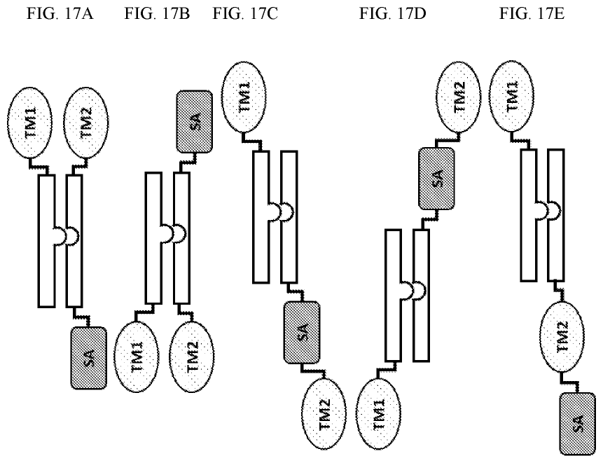

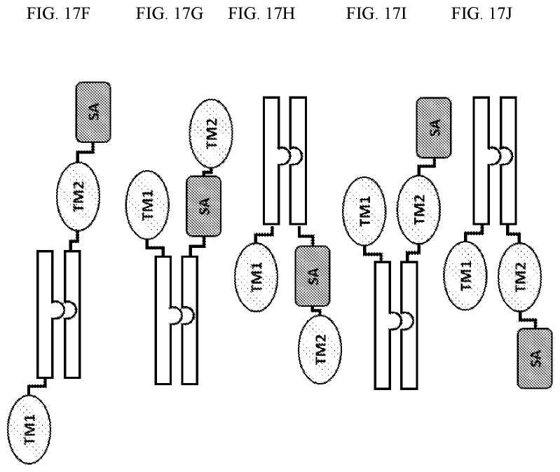

In various embodiments, the Fc-based chimeric protein complex takes the form of any of the schematics of FIGS. 17A-J.

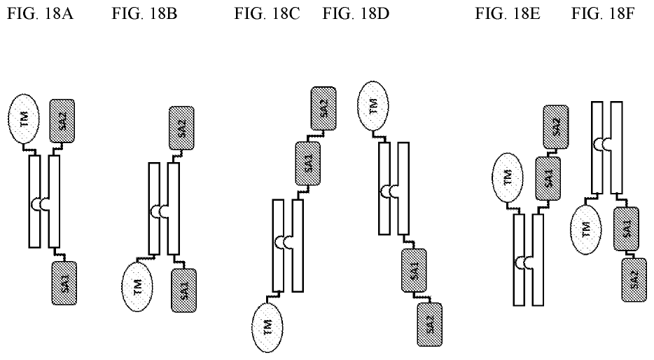

In various embodiments, the Fc-based chimeric protein complex takes the form of any of the schematics of FIGS. 18A-F.

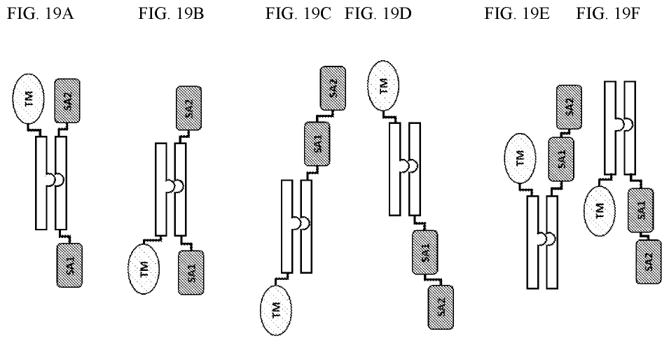

In various embodiments, the Fc-based chimeric protein complex takes the form of any of the schematics of FIGS. 19A-F.

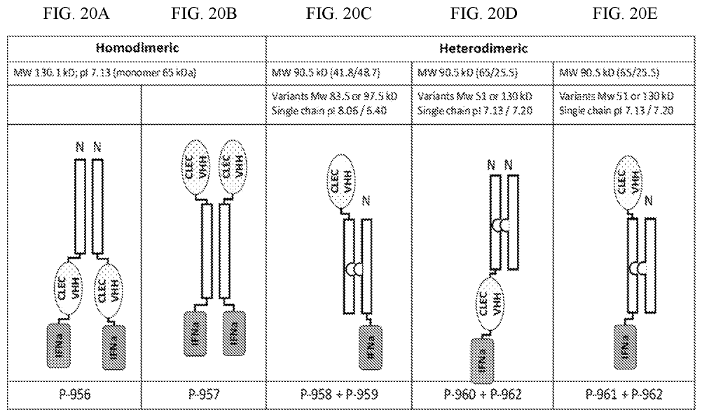

In various embodiments, the Fc-based chimeric protein complex takes the form of any of the schematics of FIGS. 20A-E.

In some embodiments, the signaling agents are linked to the targeting moieties and the targeting moieties are linked to the Fc domain on the same terminus (see FIGS. 1A-F). In some embodiments, the Fc domain is homodimeric.

In some embodiments, the signaling agents and targeting moieties are linked to the Fc domain, wherein the targeting moieties and signaling agents are linked on the same terminus (see FIGS. 1A-F). In some embodiments, the Fc domain is homodimeric.

In some embodiments, the targeting moieties are linked to signaling agents and the signaling agents are linked to the Fc domain on the same terminus (see FIGS. 1A-F). In some embodiments, the Fc domain is homodimeric.

In some embodiments, the homodimeric Fc-based chimeric protein complex has two or more targeting moieties.

In some embodiments, there are four targeting moieties and two signaling agents, the targeting moieties are linked to the Fc domain and the signaling agents are linked to targeting moieties on the same terminus (see FIGS. 2A-H). In some embodiments, the Fc domain is homodimeric. In some embodiments, where there are four targeting moieties and two signaling agents, two targeting moieties are linked to the Fc domain and two targeting moieties are linked to the signaling agents, which are linked to the Fc domain on the same terminus (see FIGS. 2A-H). In some embodiments, the Fc domain is homodimeric. In some embodiments, where there are four targeting moieties and two signaling agents, two targeting moieties are linked to each other and one of the targeting moieties of from each pair is linked to the Fc domain on the same terminus and the signaling agents are linked to the Fc domain on the same terminus (see FIGS. 2A-H). In some embodiments, the Fc domain is homodimeric. In some embodiments, where there are four targeting moieties and two signaling agents, two targeting moieties are linked to each other, wherein one of the targeting moieties of from each pair is linked to a signaling agent and the other targeting moiety of the pair is linked the Fc domain, wherein the targeting moieties linked to the Fc domain are linked on the same terminus (see FIGS. 2A-H). In some embodiments, the Fc domain is homodimeric.

In some embodiments, the homodimeric Fc-based chimeric protein complex has two or more signaling agents. In some embodiments, where there are four signaling agents and two targeting moieties, two signaling agents are linked to each other and one of the signaling agents of from pair is linked to the Fc domain on the same terminus and the targeting moieties are linked to the Fc domain on the same terminus (see FIGS. 3A-H). In some embodiments, the Fc domain is homodimeric. In some embodiments, where there are four signaling agents and two targeting moieties, two signaling agents are linked to the Fc domain one the same terminus and two of the signaling agents are each linked to a targeting moiety, wherein the targeting moieties are linked to the Fc domain at the same terminus (see FIGS. 3A-H). In some embodiments, the Fc domain is homodimeric. In some embodiments, where there are four signaling agents and two targeting moieties, two signaling agents are linked to each other and one of the signaling agents of from pair is linked to a targeting moiety and the targeting moieties are linked to the Fc domain on the same terminus (see FIGS. 3A-H). In some embodiments, the Fc domain is homodimeric.

By way of example, in some embodiments, the Fc-based chimeric protein complex comprise an Fc domain, wherein the Fc domain comprises ionic pairing mutation(s) and/or knob-in-hole mutation(s), at least one signaling agent, and at least one targeting moiety, wherein the ionic pairing motif and/or a knob-in-hole motif, signaling agent, and targeting moiety are selected from any of the ionic pairing motif and/or a knob-in-hole motif, signaling agents, and targeting moieties disclosed herein. In some embodiments, the Fc domain is heterodimeric. In some embodiments, the Fc domain comprises a mutation that reduces or eliminates its effector function.

In some embodiments, the signaling agent is linked to the targeting moiety, which is linked to the Fc domain (see FIGS. 10A-F and 13A-F). In some embodiments, the targeting moiety is linked to the signaling agent, which is linked to the Fc domain (see FIGS. 10A-F and 13A-F). In some embodiments, the Fc domain is heterodimeric. In some embodiments, the Fc domain comprises a mutation that reduces or eliminates its effector function.

In some embodiments, the signaling agent and targeting moiety are linked to the Fc domain (see FIGS. 4A-D, 7A-D, 10A-F, and 13A-F). In some embodiments, the targeting moiety and the signaling agent are linked to different Fc chains on the same terminus (see FIGS. 4A-D and 7A-D). In some embodiments, the targeting moiety and the signaling agent are linked to different Fc chains on different termini (see FIGS. 4A-D and 7A-D). In some embodiments, the targeting moiety and the signaling agent are linked to the same Fc chain (see FIGS. 10A-F and 13A-F). In some embodiments, the Fc domain is heterodimeric. In some embodiments, the Fc domain comprises a mutation that reduces or eliminates its effector function.

In some embodiments, where there are one signaling agent and two targeting moieties, the signaling agent is linked to the Fc domain and two targeting moieties can be: 1) linked to each other with one of the targeting moieties linked to the Fc domain; or 2) each linked to the Fc domain (see FIGS. 5A-F, 8A-F, 11A-L, 14A-L, 16A-J, and 17A-J). In some embodiments, the targeting moieties are linked on one Fc chain and the signaling agent is on the other Fc chain (see FIGS. 5A-F and 8A-F). In some embodiments, the paired targeting moieties and the signaling agent are linked to the same Fc chain (see FIGS. 11A-L and 14A-L). In some embodiments, a targeting moiety is linked to the Fc domain and the other targeting moiety is linked to the signaling agent, and the paired targeting moiety is linked to the Fc domain (see FIGS. 11A-L, 14A-L, 16A-J, and 17A-J). In some embodiments, the unpaired targeting moiety and paired targeting moiety are linked to the same Fc chain (see FIGS. 11A-L and 14A-L). In some embodiments, the unpaired targeting moiety and paired targeting moiety are linked to different Fc chains (see FIGS. 16A-J and 17A-J). In some embodiments, the unpaired targeting moiety and paired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent and two targeting moieties, a targeting moiety is linked to the signaling agent, which is linked to the Fc domain, and the unpaired targeting moiety is linked the Fc domain (see FIGS. 11A-L, 14A-L, 16A-J, and 17A-J). In some embodiments, the paired signaling agent and unpaired targeting moiety are linked to the same Fc chain (see FIGS. 11A-L and 14A-L). In some embodiments, the paired signaling agent and unpaired targeting moiety are linked to different Fc chains (see FIGS. 16A-J and 17A-J). In some embodiments, the paired signaling agent and unpaired targeting moiety are linked on the same terminus (see FIGS. 16A-J and 17A-J). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the targeting moiety not linked to the signaling agent is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties are linked together and the signaling agent is linked to one of the paired targeting moieties, wherein the signaling agent is linked to the Fc domain (see FIGS. 11A-L and 14A-L). In some embodiments, the Fc domain is heterodimeric.

In some embodiments, the Fc domain comprises a mutation that reduces or eliminates its effector function.

In some embodiments, where there are one signaling agent and two targeting moieties, the targeting moieties are both linked to the signaling agent, wherein one of the targeting moieties is linked to the Fc domain (see FIGS. 11A-L and 14A-L). In some embodiments, the Fc domain is heterodimeric. In some embodiments, the Fc domain comprises a mutation that reduces or eliminates its effector function.

In some embodiments, where there are one signaling agent and two targeting moieties, the targeting moieties and the signaling agent are linked to the Fc domain (see FIGS. 16A-J and 17A-J). In some embodiments, the targeting moieties are linked on the terminus (see FIGS. 16A-J and 17A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked to the Fc domain on the same terminus and the targeting moiety is linked to the Fc domain (see FIGS. 6A-J and 9A-J). In some embodiments, the signaling agents are linked to the Fc domain on the same Fc chain and the targeting moiety is linked on the other Fc chain (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, a signaling agent is linked to the targeting moiety, which is linked to the Fc domain and the other signaling agent is linked to the Fc domain (see FIGS. 6A-J, 9A-J, 12A-L, and 15A-L). In some embodiments, the targeting moiety and the unpaired signaling agent are linked to different Fc chains (see FIGS. 6A-J and 9A-J). In some embodiments, the targeting moiety and the unpaired signaling agent are linked to different Fc chains on the same terminus (see FIGS. 6A-J and 9A-J). In some embodiments, the targeting moiety and the unpaired signaling agent are linked to different Fc chains on different termini (see FIGS. 6A-J and 9A-J). In some embodiments, the targeting moiety and the unpaired signaling agent are linked to the same Fc chains (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the targeting moiety is linked to a signaling agent, which is linked to the Fc domain and the other signaling agent is linked to the Fc domain (see FIGS. 6A-J and 9A-J). In some embodiments, the paired signaling agent and the unpaired signaling agent are linked to different Fc chains (see FIGS. 6A-J and 9A-J). In some embodiments, the paired signaling agent and the unpaired signaling agent are linked to different Fc chains on the same terminus (see FIGS. 6A-J and 9A-J). In some embodiments, the paired signaling agent and the unpaired signaling agent are linked to different Fc chains on different termini (see FIGS. 6A-J and 9A-J). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and the targeting moiety is linked to one of the paired signaling agents, wherein the targeting moiety is linked to the Fc domain (see FIGS. 12A-L and 15A-L). In some embodiments, the Fc domain is heterodimeric.

In some embodiments, the Fc domain comprises a mutation that reduces or eliminates its effector function.

In some embodiments, where there are two signaling agents and one targeting moiety, the signaling agents are linked together and one of the signaling agents is linked to the Fc domain and the targeting moiety is linked to the Fc domain (see FIGS. 12A-L, 15A-L, 18A-F, and 19A-F). In some embodiments, the paired signaling agents and targeting moiety are linked to the same Fc chain (see FIGS. 12A-L and 15A-L). In some embodiments, the paired signaling agents and targeting moiety are linked to different Fc chains (see FIGS. 18A-F and 19A-F). In some embodiments, the paired signaling agents and targeting moiety are linked to different Fc chains on the same terminus (see FIGS. 18A-F and 19A-F). In some embodiments, the Fc domain is heterodimeric. In some embodiments, the Fc domain comprises a mutation that reduces or eliminates its effector function.

In some embodiments, where there are two signaling agents and one targeting moiety, the signaling agents are both linked to the targeting moiety, wherein one of the signaling agents is linked to the Fc domain (see FIGS. 12A-L and 15A-L). In some embodiments, the Fc domain is heterodimeric. In some embodiments, the Fc domain comprises a mutation that reduces or eliminates its effector function.

In some embodiments, where there are two signaling agents and one targeting moiety, the signaling agents are linked together and one of the signaling agents is linked to the targeting moiety and the other signaling agent is linked to the Fc domain (see FIGS. 12A-L and 15A-L).

In some embodiments, where there are two signaling agents and one targeting moiety, each signaling agent is linked to the Fc domain and the targeting moiety is linked to one of the signaling agents (see FIGS. 12A-L and 15A-L). In some embodiments, the signaling agents are linked to the same Fc chain (see FIGS. 12A-L and 15A-L).

In some embodiments, a targeting moiety or signaling agent is linked to the Fc domain, comprising one or both of C_{H}2 and C_{H}3 domains, and optionally a hinge region. For example, vectors encoding the targeting moiety, signaling agent, or combination thereof, linked as a single nucleotide sequence to an Fc domain can be used to prepare such polypeptides.

In some embodiments, the linker may be utilized to link various functional groups, residues, or moieties as described herein to the Fc-based chimeric protein complex. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid.

In some embodiments, the Fc-based chimeric protein complex comprises a linker connecting a targeting moiety and the signaling agent. In some embodiments, the Fc-based chimeric protein complex comprises a linker within the signaling agent (e.g. in the case of single chain TNF, which can comprise two linkers to yield a trimer or in the case of IFN gamma, which can comprise a linkers to yield a dimer).

The present technology contemplates the use of a variety of linker sequences. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et al., (2000), Protein Eng. 13(5):309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the Fc-based chimeric protein complex

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In some embodiments, the linker length allows for efficient binding of a targeting moiety, a signaling agent, and/or an Fc domain to their targets (e.g., receptors). For instance, in some embodiments, the linker length allows for efficient binding of one of the targeting moieties and the signaling agent to receptors on the same cell as well as the efficient binding of the other targeting moiety to another cell. Illustrative pairs of cells are provided elsewhere herein.

In some embodiments the linker length is at least equal to the minimum distance between the binding sites of a targeting moiety, a signaling agent, and/or an Fc domain targets (e.g., receptors) on the same cell. In some embodiments the linker length is at least twice, or three times, or four times, or five times, or ten times, or twenty times, or 25 times, or 50 times, or one hundred times, or more the minimum distance between the binding sites of a targeting moiety, a signaling agent, and/or an Fc domain targets on the same cell.

In some embodiments, a linker connects the two targeting moieties to each other and this linker has a short length and a linker connects a targeting moiety and a signaling agent this linker is longer than the linker connecting the two targeting moieties. For example, the difference in amino acid length between the linker connecting the two targeting moieties and the linker connecting a targeting moiety and a signaling agent may be about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids.

In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NO: 1283-SEQ ID NO: 1290, respectively). In an embodiment, the linker sequence is GGSGGSGGGGSGGGGS (SEQ ID NO: 1291). Additional illustrative linkers include, but are not limited to, linkers having the sequence LE, GGGGS (SEQ ID NO: 1283), (GGGGS)ₙ (n=1-7) (SEQ ID NO: 1283-SEQ ID NO: 1289), (Gly)₈ (SEQ ID NO: 1292), (Gly)₆ (SEQ ID NO: 1293), (EAAAK)ₙ (n=1-3) (SEQ ID NO: 1294-SEQ ID NO: 1296), A(EAAAK)ₙA (n=2-5) (SEQ ID NO: 1297-SEQ ID NO: 1300), AEAAAKEAAAKA (SEQ ID NO: 1297), A(EAAAK)₄ALEA(EAAAK)₄A (SEQ ID NO: 1301), PAPAP (SEQ ID NO: 1302), KESGSVSSEQLAQFRSLD (SEQ ID NO: 1303), EGKSSGSGSESKST (SEQ ID NO: 1304), GSAGSAAGSGEF (SEQ ID NO: 1305), and (XP)ₙ, with X designating any amino acid, e.g., Ala, Lys, or Glu. In various embodiments, the linker is GGS or (GGS)ₙ, (n=2-20) (SEQ ID NO: 1306-SEQ ID NO: 1324). In some embodiments, the linker is G. In some embodiments, the linker is MA. In some embodiments, the linker is (GGGGS)ₙ (n=9-20) (SEQ ID NO: 1325-SEQ ID NO: 1336).

In some embodiments, the linker is one or more of GGGSE (SEQ ID NO: 1337), GSESG (SEQ ID NO: 1338), GSEGS (SEQ ID NO: 1339), GEGGSGEGSSGEGSSSEGGGSEGGGSEGGGSEGGS (SEQ ID NO: 1340), and a linker of randomly placed G, S, and E every 4 amino acid intervals.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of wild-type human IgG1 contains the sequence Cys-Pro-Pro-Cys (SEQ ID NO: 1341), which when dimerized by disulfide bond formation, results in a cyclic octapeptide believed to act as a pivot, thus conferring flexibility. In various embodiments, the linker comprises, one, or two, or three of the upper hinge region, the core region, and the lower hinge region of any antibody (e.g., of IgG, IgA, IgD, and IgE, inclusive of subclasses (e.g. IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker of the present invention comprises one or more glycosylation sites. In various embodiments, the linker is a hinge-CH2-CH3 domain of a human IgG4 antibody.

In some embodiments, the linker is a synthetic linker such as PEG.

In various embodiments, the linker may be functional. For example, without limitation, the linker may function to improve the folding and/or stability, improve the expression, improve the pharmacokinetics, and/or improve the bioactivity of the Fc-based chimeric protein complex. In another example, the linker may function to target the Fc-based chimeric protein complex to a particular cell type or location.

### Functional Groups

In some embodiments, the Fc-based chimeric protein complex of the present technology includes one or more functional groups, residues, or moieties. In various embodiments, the one or more functional groups, residues, or moieties are attached or genetically fused to any of the Fc-proteins, the signaling agents, and the targeting moieties described herein. In some embodiments, such functional groups, residues or moieties confer one or more desired properties or functionalities to the Fc-based chimeric protein complex of the present technology. Examples of such functional groups and of techniques for introducing them into the Fc-based chimeric protein complex are known in the art, for example, see Remington's Pharmaceutical Sciences, 16th ed., Mack Publishing Co., Easton, Pa. (1980).

In various embodiments, the Fc-based chimeric protein complex may by conjugated and/or fused with another agent to extend half-life or otherwise improve pharmacodynamic and pharmacokinetic properties. For example, in some embodiments, the Fc-based chimeric protein complex may be fused or conjugated with one or more of PEG, XTEN (e.g., as rPEG), polysialic acid (POLYXEN), albumin (e.g., human serum albumin or HSA), elastin-like protein (ELP), PAS, HAP, GLK, CTP, transferrin, and the like.

In some embodiments, the functional groups, residues, or moieties comprise a suitable pharmacologically acceptable polymer, such as poly(ethyleneglycol) (PEG) or derivatives thereof (such as methoxypoly(ethyleneglycol) or mPEG). In some embodiments, attachment of the PEG moiety increases the half-life and/or reduces the immunogenecity of the Fc-based chimeric protein complex. Generally, any suitable form of pegylation can be used, such as the pegylation used in the art for antibodies and antibody fragments (including but not limited to single domain antibodies such as VHHs); see, for example, Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO04060965, the entire contents of which are hereby incorporated by reference. Various reagents for pegylation of proteins are also commercially available, for example, from Nektar Therapeutics, USA. In some embodiments, site-directed pegylation is used, in particular via a cysteine-residue (see, for example, Yang et al., Protein Engineering, 16, 10, 761-770 (2003), the entire contents of which is hereby incorporated by reference). For example, for this purpose, PEG may be attached to a cysteine residue that naturally occurs in the Fc-based chimeric protein complex. In some embodiments, the Fc-based chimeric protein complex is modified so as to suitably introduce one or more cysteine residues for attachment of PEG, or an amino acid sequence comprising one or more cysteine residues for attachment of PEG may be fused to the amino- and/or carboxy-terminus of the Fc-based chimeric protein complex, using techniques known in the art.

In some embodiments, the functional groups, residues, or moieties comprise N-linked or O-linked glycosylation. In some embodiments, the N-linked or O-linked glycosylation is introduced as part of a co-translational and/or post-translational modification.

In some embodiments, the functional groups, residues, or moieties comprise one or more detectable labels or other signal-generating groups or moieties. Suitable labels and techniques for attaching, using and detecting them are known in the art and, include, but are not limited to, fluorescent labels (such as fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes, metals, metals chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels include moieties that can be detected using NMR or ESR spectroscopy. Such labeled VHHs and polypeptides of the invention may, for example, be used for in vitro, in vivo or in situ assays (including immunoassays known per se such as ELISA, RIA, EIA and other “sandwich assays,” etc.) as well as in vivo diagnostic and imaging purposes, depending on the choice of the specific label.

In some embodiments, the functional groups, residues, or moieties comprise a tag that is attached or genetically fused to the Fc-based chimeric protein complex. In some embodiments, the Fc-based chimeric protein complex may include a single tag or multiple tags. The tag for example is a peptide, sugar, or DNA molecule that does not inhibit or prevent binding of the Fc-based chimeric protein complex to at target of interest or any other antigen of interest, such as, e.g., tumor antigens. In various embodiments, the tag is at least about: three to five amino acids long, five to eight amino acids long, eight to twelve amino acids long, twelve to fifteen amino acids long, or fifteen to twenty amino acids long. Illustrative tags are described for example, in U.S. Patent Publication No. US2013/0058962. In some embodiment, the tag is an affinity tag such as glutathione-S-transferase (GST) and histidine (His) tag. In an embodiment, the Fc-based chimeric protein complex comprises a His tag.

In some embodiments, the functional groups, residues, or moieties comprise a chelating group, for example, to chelate one of the metals or metallic cations. Suitable chelating groups, for example, include, without limitation, diethyl-enetriaminepentaacetic acid (DTPA) or ethylenediaminetetraacetic acid (EDTA).

In some embodiments, the functional groups, residues, or moieties comprise a functional group that is one part of a specific binding pair, such as the biotin-(strept)avidin binding pair. Such a functional group may be used to link the Fc-based chimeric protein complex to another protein, polypeptide or chemical compound that is bound to the other half of the binding pair, i.e., through formation of the binding pair. For example, an Fc-based chimeric protein complex may be conjugated to biotin, and linked to another protein, polypeptide, compound or carrier conjugated to avidin or streptavidin. For example, such a conjugated Fc-based chimeric protein complex may be used as a reporter, for example, in a diagnostic system where a detectable signal-producing agent is conjugated to avidin or streptavidin. Such binding pairs may, for example, also be used to bind the Fc-based chimeric protein complex to a carrier, including carriers suitable for pharmaceutical purposes. One non-limiting example are the liposomal formulations described by Cao and Suresh, Journal of Drug Targeting, 8, 4, 257 (2000). Such binding pairs may also be used to link a therapeutically active agent to the Fc-based chimeric protein complex.

### Modifications and Production of Fc-Based Chimeric Protein Complex

In various embodiments, the Fc-based chimeric protein complex comprises a targeting moiety that is a VHH. In various embodiments, the VHH is not limited to a specific biological source or to a specific method of preparation. For example, the VHH can generally be obtained: (1) by isolating the V_{H}H domain of a naturally occurring heavy chain antibody; (2) by expression of a nucleotide sequence encoding a naturally occurring V_{H}H domain; (3) by “humanization” of a naturally occurring V_{H}H domain or by expression of a nucleic acid encoding a such humanized V_{H}H domain; (4) by “camelization” of a naturally occurring VH domain from any animal species, such as from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known in the art; (7) by preparing a nucleic acid encoding a VHH using techniques for nucleic acid synthesis known in the art, followed by expression of the nucleic acid thus obtained; and/or (8) by any combination of one or more of the foregoing.

In an embodiment, the Fc-based chimeric protein complex comprises a VHH that corresponds to the VHH domains of naturally occurring heavy chain antibodies directed against a target of interest. In some embodiments, such VHH sequences can generally be generated or obtained by suitably immunizing a species of Camelid with a molecule of based on the target of interest (e.g., XCR1, Clec9A, CD8, SIRP1α, FAP, etc.) (i.e., so as to raise an immune response and/or heavy chain antibodies directed against the target of interest), by obtaining a suitable biological sample from the Camelid (such as a blood sample, or any sample of B-cells), and by generating V_{H}H sequences directed against the target of interest, starting from the sample, using any suitable known techniques. In some embodiments, naturally occurring V_{H}H domains against the target of interest can be obtained from naive libraries of Camelid VHH sequences, for example, by screening such a library using the target of interest or at least one part, fragment, antigenic determinant or epitope thereof using one or more screening techniques known in the art. Such libraries and techniques are, for example, described in WO9937681, WO0190190, WO03025020 and WO03035694, the entire contents of which are hereby incorporated by reference. In some embodiments, improved synthetic or semi-synthetic libraries derived from naive V_{H}H libraries may be used, such as V_{H}H libraries obtained from naive V_{H}H libraries by techniques such as random mutagenesis and/or CDR shuffling, as for example, described in WO0043507, the entire contents of which are hereby incorporated by reference. In some embodiments, another technique for obtaining VHH sequences directed against a target of interest involves suitably immunizing a transgenic mammal that is capable of expressing heavy chain antibodies (i.e., so as to raise an immune response and/or heavy chain antibodies directed against the target of interest), obtaining a suitable biological sample from the transgenic mammal (such as a blood sample, or any sample of B-cells), and then generating V_{H}H sequences directed against XCR1 starting from the sample, using any suitable known techniques. For example, for this purpose, the heavy chain antibody-expressing mice and the further methods and techniques described in WO02085945 and in WO04049794 (the entire contents of which are hereby incorporated by reference) can be used.

In an embodiment, the Fc-based chimeric protein complex comprises a VHH that has been “humanized” i.e., by replacing one or more amino acid residues in the amino acid sequence of the naturally occurring V_{H}H sequence (and in particular in the framework sequences) by one or more of the amino acid residues that occur at the corresponding position(s) in a VH domain from a conventional 4-chain antibody from a human being. This can be performed using humanization techniques known in the art. In some embodiments, possible humanizing substitutions or combinations of humanizing substitutions may be determined by methods known in the art, for example, by a comparison between the sequence of a VHH and the sequence of a naturally occurring human VH domain. In some embodiments, the humanizing substitutions are chosen such that the resulting humanized VHHs still retain advantageous functional properties. Generally, as a result of humanization, the VHHs of the invention may become more “human-like,” while still retaining favorable properties such as a reduced immunogenicity, compared to the corresponding naturally occurring VHH domains. In various embodiments, the humanized VHHs of the invention can be obtained in any suitable manner known in the art and thus are not strictly limited to polypeptides that have been obtained using a polypeptide that comprises a naturally occurring V_{H}H domain as a starting material.

In an embodiment, the Fc-based chimeric protein complex comprises a VHH that has been “camelized,” i.e., by replacing one or more amino acid residues in the amino acid sequence of a naturally occurring VH domain from a conventional 4-chain antibody by one or more of the amino acid residues that occur at the corresponding position(s) in a V_{H}H domain of a heavy chain antibody of a camelid. In some embodiments, such “camelizing” substitutions are inserted at amino acid positions that form and/or are present at the VH-VL interface, and/or at the so-called Camelidae hallmark residues (see, for example, WO9404678, the entire contents of which are hereby incorporated by reference). In some embodiments, the VH sequence that is used as a starting material or starting point for generating or designing the camelized VHH is a VH sequence from a mammal, for example, the VH sequence of a human being, such as a VH3 sequence. In various embodiments, the camelized VHHs can be obtained in any suitable manner known in the art (i.e., as indicated under points (1)-(8) above) and thus are not strictly limited to polypeptides that have been obtained using a polypeptide that comprises a naturally occurring VH domain as a starting material.

In various embodiments, both “humanization” and “camelization” can be performed by providing a nucleotide sequence that encodes a naturally occurring V_{H}H domain or VH domain, respectively, and then changing, in a manner known in the art, one or more codons in the nucleotide sequence in such a way that the new nucleotide sequence encodes a “humanized” or “camelized” VHH, respectively. This nucleic acid can then be expressed in a manner known in the art, so as to provide the desired VHH of the invention. Alternatively, based on the amino acid sequence of a naturally occurring V_{H}H domain or VH domain, respectively, the amino acid sequence of the desired humanized or camelized VHH of the invention, respectively, can be designed and then synthesized de novo using techniques for peptide synthesis known in the art. Also, based on the amino acid sequence or nucleotide sequence of a naturally occurring V_{H}H domain or VH domain, respectively, a nucleotide sequence encoding the desired humanized or camelized VHH, respectively, can be designed and then synthesized de novo using techniques for nucleic acid synthesis known in the art, after which the nucleic acid thus obtained can be expressed in a manner known in the art, so as to provide the desired VHH of the invention. Other suitable methods and techniques for obtaining the VHHs of the invention and/or nucleic acids encoding the same, starting from naturally occurring VH sequences or V_{H}H sequences, are known in the art, and may, for example, comprise combining one or more parts of one or more naturally occurring VH sequences (such as one or more FR sequences and/or CDR sequences), one or more parts of one or more naturally occurring VHH sequences (such as one or more FR sequences or CDR sequences), and/or one or more synthetic or semi-synthetic sequences, in a suitable manner, so as to provide a VHH of the invention or a nucleotide sequence or nucleic acid encoding the same.

Methods for producing the Fc-based chimeric protein complex of the present technology are described herein. For example, DNA sequences encoding the Fc-based chimeric protein complex of the present technology can be chemically synthesized using methods known in the art. Synthetic DNA sequences can be ligated to other appropriate nucleotide sequences, including, e.g., expression control sequences, to produce gene expression constructs encoding the desired Fc-based chimeric protein complex of the present technology. Accordingly, in various embodiments, the present invention provides for isolated nucleic acids comprising a nucleotide sequence encoding the Fc-based chimeric protein complex of the present technology.

Nucleic acids encoding the Fc-based chimeric protein complex of the present technology can be incorporated (ligated) into expression vectors, which can be introduced into host cells through transfection, transformation, or transduction techniques. For example, nucleic acids encoding the Fc-based chimeric protein complex of the present technology invention can be introduced into host cells by retroviral transduction. Illustrative host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the Fc-based chimeric protein complex of the present technology. Accordingly, in various embodiments, the present invention provides expression vectors comprising nucleic acids that encode the Fc-based chimeric protein complex of the present technology. In various embodiments, the present invention additional provides host cells comprising such expression vectors.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. In another example, if the engineered gene is to be expressed in eukaryotic host cells, e.g., CHO cells, it is first inserted into an expression vector containing for example, a suitable eukaryotic promoter, a secretion signal, enhancers, and various introns. The gene construct can be introduced into the host cells using transfection, transformation, or transduction techniques.

The Fc-based chimeric protein complex of the present technology can be produced by growing a host cell transfected with an expression vector encoding the Fc-based chimeric protein complex under conditions that permit expression of the protein. Following expression, the protein can be harvested and purified using techniques well known in the art, e.g., affinity tags such as glutathione-S-transferase (GST) and histidine (His) tags or by chromatography. In an embodiment, the Fc-based chimeric protein complex comprises a His tag. In an embodiment, the Fc-based chimeric protein complex comprises a His tag and a proteolytic site to allow cleavage of the His tag.

Accordingly, in various embodiments, the present invention provides for a nucleic acid encoding an Fc-based chimeric protein complex of the present invention. In various embodiments, the present invention provides for a host cell comprising a nucleic acid encoding an Fc-based chimeric protein complex of the present invention.

In various embodiments, the methods of modifying and producing the Fc-based chimeric protein complex as described herein can be easily adapted for the modification and production of any multi-specific Fc-based chimeric protein complex comprising two or more targeting moieties and/or signaling agents.

In various embodiments, the present Fc-based chimeric protein complex may be expressed in vivo, for instance, in a patient. For example, in various embodiments, the present Fc-based chimeric protein complex may be administered in the form of nucleic acid which encodes the present Fc-based chimeric protein complex. In various embodiments, the nucleic acid is DNA or RNA. In some embodiments, the present Fc-based chimeric protein complex is encoded by a modified mRNA, i.e. an mRNA comprising one or more modified nucleotides. In some embodiments, the modified mRNA comprises one or modifications found in U.S. Pat. No. 8,278,036, the entire contents of which are hereby incorporated by reference. In some embodiments, the modified mRNA comprises one or more of m5C, m5U, m6A, s2U, Ψ, and 2′-O-methyl-U. In some embodiments, the present invention relates to administering a modified mRNA encoding one or more of the present Fc-based chimeric protein complexes. In some embodiments, the present invention relates to gene therapy vectors comprising the same. In some embodiments, the present invention relates to gene therapy methods comprising the same. In various embodiments, the nucleic acid is in the form of an oncolytic virus, e.g. an adenovirus, reovirus, measles, herpes simplex, Newcastle disease virus or vaccinia.

### Pharmaceutically Acceptable Salts and Excipients

The Fc-based chimeric protein complex described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, cam phorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

In some embodiments, the compositions described herein are in the form of a pharmaceutically acceptable salt.

### Pharmaceutical Compositions and Formulations

In various embodiments, the present invention pertains to pharmaceutical compositions comprising the Fc-based chimeric protein complex described herein and a pharmaceutically acceptable carrier or excipient. In some embodiments, the present invention pertains to pharmaceutical compositions comprising the present Fc-based chimeric protein complex. In a further embodiment, the present invention pertains to pharmaceutical compositions comprising a combination of the present Fc-based chimeric protein complex and any other therapeutic agents described herein. Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for proper administration.

In various embodiments, pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

The present invention includes the described pharmaceutical compositions (and/or additional therapeutic agents) in various formulations. Any inventive pharmaceutical composition (and/or additional therapeutic agents) described herein can take the form of solutions, suspensions, emulsion, drops, tablets, pills, pellets, capsules, capsules containing liquids, gelatin capsules, powders, sustained-release formulations, suppositories, emulsions, aerosols, sprays, suspensions, lyophilized powder, frozen suspension, desiccated powder, or any other form suitable for use. In one embodiment, the composition is in the form of a capsule. In another embodiment, the composition is in the form of a tablet. In yet another embodiment, the pharmaceutical composition is formulated in the form of a soft-gel capsule. In a further embodiment, the pharmaceutical composition is formulated in the form of a gelatin capsule. In yet another embodiment, the pharmaceutical composition is formulated as a liquid.

Where necessary, the inventive pharmaceutical compositions (and/or additional agents) can also include a solubilizing agent. Also, the agents can be delivered with a suitable vehicle or delivery device as known in the art. Combination therapies outlined herein can be co-delivered in a single delivery vehicle or delivery device.

The formulations comprising the inventive pharmaceutical compositions (and/or additional agents) of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art).

In various embodiments, any pharmaceutical compositions (and/or additional agents) described herein is formulated in accordance with routine procedures as a composition adapted for a mode of administration described herein.

Routes of administration include, for example: oral, intradermal, intramuscular, intraperitoneal, intravenous, subcutaneous, intranasal, epidural, sublingual, intranasal, intracerebral, intravaginal, transdermal, rectally, by inhalation, or topically. Administration can be local or systemic. In some embodiments, the administering is effected orally. In another embodiment, the administration is by parenteral injection. The mode of administration can be left to the discretion of the practitioner, and depends in-part upon the site of the medical condition. In most instances, administration results in the release of any agent described herein into the bloodstream.

In one embodiment, the Fc-based chimeric protein complex described herein is formulated in accordance with routine procedures as a composition adapted for oral administration. Compositions for oral delivery can be in the form of tablets, lozenges, aqueous or oily suspensions, granules, powders, emulsions, capsules, syrups, or elixirs, for example. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving any Fc-based chimeric protein complex described herein are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

The compositions provided herein, alone or in combination with other suitable components, can be made into aerosol formulations (i.e., “nebulized”) to be administered via inhalation. Aerosol formulations can be placed into pressurized acceptable propellants, such as dichlorodifluoromethane, propane, nitrogen, and the like.

Any inventive pharmaceutical compositions (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropyl cellulose, hydropropylmethyl cellulose, polyvinylpyrrolidone, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the agents described herein. The invention thus provides single unit dosage forms suitable for oral administration such as, but not limited to, tablets, capsules, gelcaps, and caplets that are adapted for controlled- or sustained-release.

Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### Administration and Dosage

It will be appreciated that the actual dose of the Fc-based chimeric protein complex described herein to be administered according to the present invention will vary according to the particular dosage form, and the mode of administration. Many factors that may modify the action of the Fc-based chimeric protein complex (e.g., body weight, gender, diet, time of administration, route of administration, rate of excretion, condition of the subject, drug combinations, genetic disposition and reaction sensitivities) can be taken into account by those skilled in the art. Administration can be carried out continuously or in one or more discrete doses within the maximum tolerated dose. Optimal administration rates for a given set of conditions can be ascertained by those skilled in the art using conventional dosage administration tests.

In some embodiments, a suitable dosage of the Fc-based chimeric protein complex described herein is in a range of about 0.01 mg/kg to about 10 g/kg of body weight of the subject, about 0.01 mg/kg to about 1 g/kg of body weight of the subject, about 0.01 mg/kg to about 100 mg/kg of body weight of the subject, about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, about 100 mg/kg body weight, about 1 g/kg of body weight, about 10 g/kg of body weight, inclusive of all values and ranges there between.

Individual doses of the Fc-based chimeric protein complex described herein can be administered in unit dosage forms containing, for example, from about 0.01 mg to about 100 g, from about 0.01 mg to about 75 g, from about 0.01 mg to about 50 g, from about 0.01 mg to about 25 g, about 0.01 mg to about 10 g, about 0.01 mg to about 7.5 g, about 0.01 mg to about 5 g, about 0.01 mg to about 2.5 g, about 0.01 mg to about 1 g, about 0.01 mg to about 100 mg, from about 0.1 mg to about 100 mg, from about 0.1 mg to about 90 mg, from about 0.1 mg to about 80 mg, from about 0.1 mg to about 70 mg, from about 0.1 mg to about 60 mg, from about 0.1 mg to about 50 mg, from about 0.1 mg to about 40 mg active ingredient, from about 0.1 mg to about 30 mg, from about 0.1 mg to about 20 mg, from about 0.1 mg to about 10 mg, from about 0.1 mg to about 5 mg, from about 0.1 mg to about 3 mg, from about 0.1 mg to about 1 mg per unit dosage form, or from about 5 mg to about 80 mg per unit dosage form. For example, a unit dosage form can be about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 500 mg, about 1 g, about 2.5 g, about 5 g, about 10 g, about 25 g, about 50 g, about 75 g, about 100 g, inclusive of all values and ranges there between.

In one embodiment, the Fc-based chimeric protein complex described herein are administered at an amount of from about 0.01 mg to about 100 g daily, from about 0.01 mg to about 75 g daily, from about 0.01 mg to about 50 g daily, from about 0.01 mg to about 25 g daily, from about 0.01 mg to about 10 g daily, from about 0.01 mg to about 7.5 g daily, from about 0.01 mg to about 5 g daily, from about 0.01 mg to about 2.5 g daily, from about 0.01 mg to about 1 g daily, from about 0.01 mg to about 100 mg daily, from about 0.1 mg to about 100 mg daily, from about 0.1 mg to about 95 mg daily, from about 0.1 mg to about 90 mg daily, from about 0.1 mg to about 85 mg daily, from about 0.1 mg to about 80 mg daily, from about 0.1 mg to about 75 mg daily, from about 0.1 mg to about 70 mg daily, from about 0.1 mg to about 65 mg daily, from about 0.1 mg to about 60 mg daily, from about 0.1 mg to about 55 mg daily, from about 0.1 mg to about 50 mg daily, from about 0.1 mg to about 45 mg daily, from about 0.1 mg to about 40 mg daily, from about 0.1 mg to about 35 mg daily, from about 0.1 mg to about 30 mg daily, from about 0.1 mg to about 25 mg daily, from about 0.1 mg to about 20 mg daily, from about 0.1 mg to about 15 mg daily, from about 0.1 mg to about 10 mg daily, from about 0.1 mg to about 5 mg daily, from about 0.1 mg to about 3 mg daily, from about 0.1 mg to about 1 mg daily, or from about 5 mg to about 80 mg daily. In various embodiments, the Fc-based chimeric protein complex is administered at a daily dose of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 3 mg, about 4 mg, about 5 mg, about 6 mg, about 7 mg, about 8 mg, about 9 mg about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 200 mg, about 500 mg, about 1 g, about 2.5 g, about 5 g, about 7.5 g, about 10 g, about 25 g, about 50 g, about 75 g, about 100 g, inclusive of all values and ranges there between.

In accordance with certain embodiments of the invention, the pharmaceutical composition comprising the Fc-based chimeric protein complex described herein may be administered, for example, more than once daily (e.g., about two times, about three times, about four times, about five times, about six times, about seven times, about eight times, about nine times, or about ten times daily), about once per day, about every other day, about every third day, about once a week, about once every two weeks, about once every month, about once every two months, about once every three months, about once every six months, or about once every year.

### Combination Therapy and Additional Therapeutic Agents

In various embodiments, the pharmaceutical composition of the present invention is co-administered in conjunction with additional therapeutic agent(s). Co-administration can be simultaneous or sequential.

In one embodiment, the additional therapeutic agent and the Fc-based chimeric protein complex are administered to a subject simultaneously. The term “simultaneously” as used herein, means that the additional therapeutic agent and the Fc-based chimeric protein complex are administered with a time separation of no more than about 60 minutes, such as no more than about 30 minutes, no more than about 20 minutes, no more than about 10 minutes, no more than about 5 minutes, or no more than about 1 minute. Administration of the additional therapeutic agent and the Fc-based chimeric protein complex be by simultaneous administration of a single formulation (e.g., a formulation comprising the additional therapeutic agent and the Fc-based chimeric protein complex) or of separate formulations (e.g., a first formulation including the additional therapeutic agent and a second formulation including the Fc-based chimeric protein complex).

Co-administration does not require the therapeutic agents to be administered simultaneously, if the timing of their administration is such that the pharmacological activities of the additional therapeutic agent and the Fc-based chimeric protein complex overlap in time, thereby exerting a combined therapeutic effect. For example, the additional therapeutic agent and the Fc-based chimeric protein complex can be administered sequentially. The term “sequentially” as used herein means that the additional therapeutic agent and the Fc-based chimeric protein complex are administered with a time separation of more than about 60 minutes. For example, the time between the sequential administration of the additional therapeutic agent and the Fc-based chimeric protein complex can be more than about 60 minutes, more than about 2 hours, more than about 5 hours, more than about 10 hours, more than about 1 day, more than about 2 days, more than about 3 days, more than about 1 week, or more than about 2 weeks, or more than about one month apart. The optimal administration times will depend on the rates of metabolism, excretion, and/or the pharmacodynamic activity of the additional therapeutic agent and the Fc-based chimeric protein complex being administered. Either the additional therapeutic agent or the Fc-based chimeric protein complex may be administered first.

Co-administration also does not require the therapeutic agents to be administered to the subject by the same route of administration. Rather, each therapeutic agent can be administered by any appropriate route, for example, parenterally or non-parenterally.

In some embodiments, the Fc-based chimeric protein complex described herein acts synergistically when co-administered with another therapeutic agent. In such embodiments, the Fc-based chimeric protein complex and the additional therapeutic agent may be administered at doses that are lower than the doses employed when the agents are used in the context of monotherapy.

In some embodiments, the present invention pertains to chemotherapeutic agents as additional therapeutic agents. For example, without limitation, such combination of the present Fc-based chimeric protein complex and chemotherapeutic agent find use in the treatment of cancers, as described elsewhere herein. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

Accordingly, in some embodiments, the present invention relates to combination therapies using the Fc-based chimeric protein complex and a chemotherapeutic agent. In some embodiments, the present invention relates to administration of the Fc-based chimeric protein complex to a patient undergoing treatment with a chemotherapeutic agent. In some embodiments, the chemotherapeutic agent is a DNA-intercalating agent such as, without limitation, doxorubicin, cisplatin, daunorubicin, and epirubicin. In an embodiment, the DNA-intercalating agent is doxorubicin.

In illustrative embodiments, the Fc-based chimeric protein complex acts synergistically when co-administered with doxorubicin. In an illustrative embodiment, the Fc-based chimeric protein complex acts synergistically when co-administered with doxorubicin for use in treating tumor or cancer. For example, co-administration of the Fc-based chimeric protein complex and doxorubicin may act synergistically to reduce or eliminate the tumor or cancer, or slow the growth and/or progression and/or metastasis of the tumor or cancer. In illustrative embodiments, the combination of the Fc-based chimeric protein complex and doxorubicin may exhibit improved safety profiles when compared to the agents used alone in the context of monotherapy. In illustrative embodiments, the Fc-based chimeric protein complex and doxorubicin may be administered at doses that are lower than the doses employed when the agents are used in the context of monotherapy. In some embodiments, the Fc-based chimeric protein complex comprises a mutated interferon such as a mutated IFNα2. In illustrative embodiments, the mutated IFNα2 comprises one or more mutations at positions 148, 149, and 153 with reference to SEQ ID NO: 1 or SEQ ID NO: 2, such as the substitutions M148A, R149A, and L153A.

In some embodiments, the present invention relates to combination therapy with one or more immune-modulating agents, for example, without limitation, agents that modulate immune checkpoint. In various embodiments, the immune-modulating agent targets one or more of PD-1, PD-L1, and PD-L2. In various embodiments, the immune-modulating agent is PD-1 inhibitor. In various embodiments, the immune-modulating agent is an antibody specific for one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL3280A (ROCHE). In some embodiments, the immune-modulating agent targets one or more of CD137 or CD137L. In various embodiments, the immune-modulating agent is an antibody specific for one or more of CD137 or CD137L. For instance, in some embodiments, the immune-modulating agent is an antibody such as, by way of non-limitation, urelumab (also known as BMS-663513 and anti-4-1BB antibody). In some embodiments, the present Fc-based chimeric protein complex is combined with urelumab (optionally with one or more of nivolumab, lirilumab, and urelumab) for the treatment of solid tumors and/or B-cell non-Hodgkins lymphoma and/or head and neck cancer and/or multiple myeloma. In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the present Fc-based chimeric protein complexis combined with ipilimumab (optionally with bavituximab) for the treatment of one or more of melanoma, prostate cancer, and lung cancer. In various embodiments, the immune-modulating agent targets CD20. In various embodiments, the immune-modulating agent is an antibody specific CD20. For instance, in some embodiments, the immune-modulating agent is an antibody such as, by way of non-limitation, Ofatumumab (GENMAB), obinutuzumab (GAZYVA), AME-133v (APPLIED MOLECULAR EVOLUTION), Ocrelizumab (GENENTECH), TRU-015 (TRUBION/EMERGENT), veltuzumab (IMMU-106).

In some embodiments, the present invention relates to combination therapy using the Fc-based chimeric protein complex and a checkpoint inhibitor. In some embodiments, the present invention relates to administration of the Fc-based chimeric protein complex to a patient undergoing treatment with a checkpoint inhibitor. In some embodiments, the checkpoint inhibitor is an agent that targets one or more of PD-1, PD-L1, PD-L2, and CTLA-4 (including any of the anti-PD-1, anti-PD-L1, anti-PD-L2, and anti-CTLA-4 agents described herein). In some embodiment, the checkpoint inhibitor is one or more of nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL3280A (ROCHE), ipilimumab (MDX-010, MDX-101, Yervoy, BMS) and tremelimumab (Pfizer). In an embodiment, the checkpoint inhibitor is an antibody against PD-L1.

In illustrative embodiments, the Fc-based chimeric protein complex acts synergistically when co-administered with the anti-PD-L1 antibody. In an illustrative embodiment, the Fc-based chimeric protein complex acts synergistically when co-administered with the anti-PD-L1 antibody for use in treating tumor or cancer. For example, co-administration of the Fc-based chimeric protein complex and the anti-PD-L1 antibody may act synergistically to reduce or eliminate the tumor or cancer, or slow the growth and/or progression and/or metastasis of the tumor or cancer. In some embodiments, the combination of the Fc-based chimeric protein complex and the anti-PD-L1 antibody may exhibit improved safety profiles when compared to the agents used alone in the context of monotherapy. In some embodiments, the Fc-based chimeric protein complex and the anti-PD-L1 antibody may be administered at doses that are lower than the doses employed when the agents are used in the context of monotherapy. In some embodiments, the Fc-based chimeric protein complex comprises a mutated interferon such as a mutated IFNα2. In illustrative embodiments, the mutated IFNα2 comprises one or more mutations at positions 148, 149, and 153 with reference to SEQ ID NO: 1 or SEQ ID NO: 2, such as the substitutions M148A, R149A, and L153A.

In some embodiments, the present invention relates to combination therapies using the Fc-based chimeric protein complex and an immunosuppressive agent. In some embodiments, the present invention relates to administration of the Fc-based chimeric protein complex to a patient undergoing treatment with an immunosuppressive agent. In an embodiment, the immunosuppressive agent is TNF.

In illustrative embodiments, the Fc-based chimeric protein complex acts synergistically when co-administered with TNF. In an illustrative embodiment, the Fc-based chimeric protein complex acts synergistically when co-administered with TNF for use in treating tumor or cancer. For example, co-administration of the Fc-based chimeric protein complex and TNF may act synergistically to reduce or eliminate the tumor or cancer, or slow the growth and/or progression and/or metastasis of the tumor or cancer. In some embodiments, the combination of the Fc-based chimeric protein complex and TNF may exhibit improved safety profiles when compared to the agents used alone in the context of monotherapy. In some embodiments, the Fc-based chimeric protein complex and TNF may be administered at doses that are lower than the doses employed when the agents are used in the context of monotherapy. In some embodiments, the Fc-based chimeric protein complex comprises a mutated interferon such as a mutated IFNα2. In illustrative embodiments, the mutated IFNα2 comprises one or more mutations at positions 148, 149, and 153 with reference to SEQ ID NO: 1 or SEQ ID NO: 2, such as the substitutions M148A, R149A, and L153A.

In some embodiments, the Fc-based chimeric protein complex acts synergistically when used in combination with Chimeric Antigen Receptor (CAR) T-cell therapy. In an illustrative embodiment, the Fc-based chimeric protein complex acts synergistically when used in combination with CAR T-cell therapy in treating tumor or cancer. In an embodiment, the Fc-based chimeric protein complex acts synergistically when used in combination with CAR T-cell therapy in treating blood-based tumors. In an embodiment, the Fc-based chimeric protein complex acts synergistically when used in combination with CAR T-cell therapy in treating solid tumors. For example, use of the Fc-based chimeric protein complex and CAR T-cells may act synergistically to reduce or eliminate the tumor or cancer, or slow the growth and/or progression and/or metastasis of the tumor or cancer. In various embodiments, the Fc-based chimeric protein complex of the invention induces CAR T-cell division. In various embodiments, the Fc-based chimeric protein complex of the invention induces CAR T-cell proliferation. In various embodiments, the Fc-based chimeric protein complex of the invention prevents anergy of the CAR T cells.

In various embodiments, the CAR T-cell therapy comprises CAR T cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Rα2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-α), GD2, human epidermal growth factor receptor 2 (HER2), mesothelin, EGFRvIII, fibroblast activation protein (FAP), carcinoembryonic antigen (CEA), and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Exemplary CAR T-cell therapy include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In some embodiments, the Fc-based chimeric protein complex is used in a method of treating multiple sclerosis (MS) in combination with one or more MS therapeutics including, but not limited to, 3-interferons, glatiramer acetate, T-interferon, IFN-ß-2 (U.S. Patent Publication No. 2002/0025304), spirogermaniums (e.g., N-(3-dimethyl aminopropyl)-2-aza-8,8-dimethyl-8-germ anspiro [4:5] decane, N-(3-dimethylaminopropyl)-2-aza-8,8-diethyl-8-germaspiro [4:5] decane, N-(3-dimethylaminopropyl)-2-aza-8,8-dipropyl-8-germaspiro [4:5] decane, and N-(3-dimethylaminopropyl)-2-aza-8, 8-dibutyl-8-germaspiro [4:5] decane), vitamin D analogs (e.g., 1,25 (OH) 2D3, (see, e.g., U.S. Pat. No. 5,716,946)), prostaglandins (e.g., latanoprost, brimonidine, PGE1, PGE2 and PGE3, see, e.g., U.S. Patent Publication No. 2002/0004525), tetracycline and derivatives (e.g., minocycline and doxycycline, see, e.g., U.S. Patent Publication No. 20020022608), a VLA-4 binding antibody (see, e.g., U.S. Patent Publication No. 2009/0202527), adrenocorticotrophic hormone, corticosteroid, prednisone, methylprednisone, 2-chlorodeoxyadenosine, mitoxantrone, sulphasalazine, methotrexate, azathioprine, cyclophosphamide, cyclosporin, fumarate, anti-CD20 antibody (e.g., rituximab), and tizanidine hydrochloride.

In some embodiments, the Fc-based chimeric protein complex is used in combination with one or more therapeutic agents that treat one or more symptoms or side effects of MS. Such agents include, but are not limited to, amantadine, baclofen, papaverine, meclizine, hydroxyzine, sulfamethoxazole, ciprofloxacin, docusate, pemoline, dantrolene, desmopressin, dexamethasone, tolterodine, phenyloin, oxybutynin, bisacodyl, venlafaxine, amitriptyline, methenamine, clonazepam, isoniazid, vardenafil, nitrofurantoin, psyllium hydrophilic mucilloid, alprostadil, gabapentin, nortriptyline, paroxetine, propantheline bromide, modafinil, fluoxetine, phenazopyridine, methylprednisolone, carbamazepine, imipramine, diazepam, sildenafil, bupropion, and sertraline.

In some embodiments, the Fc-based chimeric protein complex is used in a method of treating multiple sclerosis in combination with one or more of the disease modifying therapies (DMTs) described herein (e.g. the agents of Table A). In some embodiments, the present invention provides an improved therapeutic effect as compared to use of one or more of the DMTs described herein (e.g. the agents listed in the Table below) without the one or more disclosed binding agent. In an embodiment, the combination of the Fc-based chimeric protein complex and the one or more DMTs produces synergistic therapeutic effects.

Illustrative Disease Modifying Therapies

| Generic Name | Branded Name/Company | Frequency/Route of Delivery/Usual Dose |
|---|---|---|
| teriflunomide | AUBAGIO (GENZYME) | Every day; pill taken orally; 7 mg or 14 mg. |
| interferon beta-la | AVONEX | Once a week; intramuscular |
| | (BIOGEN IDEC) | (into the muscle) injection; 30 mcg |
| interferon beta-1b | BETASERON (BAYER | Every other day; subcutaneous |
| | HEALTHCARE | (under the skin) injection; 250 mcg. |
| | PHARMACEUTICALS, | |
| | INC.) | |
| glatiramer acetate | COPAXONE (TEVA | Every day; subcutaneous (under the skin) |
| | NEUROSCIENCE) | injection; 20 mg (20,000 mcg) OR Three times a |
| | | week; subcutaneous (under the skin) |
| | | injection; 40 mg (40,000 mcg) |
| interferon beta-1b | EXTAVIA (NOVARTIS | Every other day; subcutaneous |
| | PHARMACEUTICALS | (under the skin) injection; 250 mcg. |
| | CORP.) | |
| fingolimod | GILENYA (NOVARTIS | Every day; capsule taken orally; 0.5 mg. |
| | PHARMACEUTICALS | |
| | CORP.) | |
| Alemtuzumab (anti-CD52 | LEMTRADA | Intravenous infusion on five consecutive days, |
| monoclonal antibody) | (GENZYME) | followed by intravenous infusion on three |
| | | consecutive days one year later (12 mg) |
| mitoxantrone | NOVANTRONE | Four times a year by IV infusion in a medical |
| | (EMD SERONO) | facility. Lifetime cumulative dose limit of |
| | | approximately 8-12 doses |
| | | over 2-3 years (140 mg/m2). |
| pegylated interferon beta-la | PLEGRIDY | Every 14 days; subcutaneous |
| | (BIOGEN IDEC) | (under the skin) injection; 125 mcg |
| interferon beta-la | REBIF | Three times a week; subcutaneous |
| | (EMD SERONO, INC.) | (under the skin) injection; 44 mcg |
| dimethyl fumarate (BG-12) | TECFIDERA | Twice a day; capsule taken orally; |
| | (BIOGEN IDEC) | 120 mg for one week and 240 mg therafter |
| Natalizumab (humanized | TYSABRI | Every four weeks by IV infusion in a registered |
| monoclonal antibody VLA-4 | (BIOGEN IDEC) | infusion facility; 300 mg |
| antagonist) | | |

DMTs in Development

| | | |
|---|---|---|
| Amiloride (targets Acid- | PAR | Oral |
| sensing ion channel-1 | PHARMACEUTICAL, | |
| Epithelial sodium channel | PERRIGO | |
| Na+/H+exchanger) | COMPANY, | |
| | SIGMAPHARM | |
| | LABORATORIES | |
| ATX-MS-1467 (targets Major | APITOPE/ | Intradermal Subcutaneous |
| histocompatibility complex | MERCK | |
| class II T cell responses to | SERONO | |
| myelin basic protein) | | |
| BAF312 (targets | NOVARTIS | Oral |
| Sphingosine 1-phosphate | PHARMA | |
| (S1P) receptor subtypes | | |
| S1P1 and S1P5 B cell | | |
| distrubution T cell | | |
| distribution) | | |
| BGC20-0134 (targets | BTG PLC | Oral |
| Proinflammatory and anti- | | |
| inflammatory cytokines) | | |
| BIIB033 (targets LINGO-1 | BIOGEN | Intravenous infusion used |
| (“leucine-rich repeat and | | in Phase I and Phase II |
| immunoglobulin-like domain- | | trials Subcutaneous |
| containing, Nogo receptor- | | injection used in Phase I trial |
| interacting protein”)) | | |
| Cladribine (targets CD4+ | MERCK SERONO | Oral |
| T cells DNA synthesis and | | |
| repair E-selectin Intracellular | | |
| adhesion molecule-1 Pro- | | |
| inflammatory cytokines | | |
| interleukin 2 and interleukin | | |
| 2R Pro-inflammatory | | |
| cytokines interleukin 8 and | | |
| RANTES Cytokine secretion | | |
| Monocyte and lymphocyte | | |
| migration) | | |
| Cyclophosphamide (targets | BAXTER | Oral, monthly intravenous pulses |
| T cells, particularly CD4+ | HEALTHCARE | |
| helper T cells B cells) | CORPORATION | |
| Daclizumab (humanized | BIOGEN IDEC/ | Projected to be IM injection once monthly |
| monoclonal antibody | ABBVIE | |
| targeting CD25 Immune | BIOTHERAPEUTICS | |
| modulator of T cells) | | |
| Dalfampridine (targets | ACORDA | One tablet every 12 hours |
| Voltage-gated potassium | THERAPEUTICS/ | (extended release), 10 mg twice a day |
| channels | BIOGEN IDEC | |
| Degenerin/epithelial sodium | | |
| channels L-type calcium | | |
| channels that contain | | |
| subunit Cavbeta3) | | |
| Dronabinol (targets | ABBVIE INC. | Oral |
| Cannabinoid receptor CB1 | | |
| Cannabinoid receptor CB2) | | |
| Firategrast (targets | GLAXOSMITHKLINE | Oral |
| Alpha4beta1 integrin) | | |
| GNbAC1MSRV-Env (targets | GENEURO SA/ | Intravenous infusion |
| envelope protein of the MS- | SERVIER | |
| associated retrovirus) | | |
| Idebenone (targets Reactive | SANTHERA | Oral Dose in clinical trial for PPMS is 2250 mg per |
| oxygen species) | PHARMACEUTICALS | day (750 mg dose, 3 times per day) |
| Imilecleucel-T (targets | OPEXA | Subcutaneous Given 5 times per year, according |
| Myelin-specific, autoreactive | THERAPEUTICS/ | to information from the manufacturer |
| T cells) | MERCK | |
| | SERONO | |
| Laquinimod | TEVA | Projected to be 0.6 mg or 1.2 mg |
| | | oral tablet taken daily |
| Masitinib (targets KIT (a | AB SCIENCE | Oral |
| stem cell factor, also called | | |
| c-KIT) receptor as well as | | |
| select other tyrosine kinases | | |
| Mast cells) | | |
| MEDI-551 (targets CD19, a | MEDIMMUNE | Intravenous Subcutaneous |
| B cell-specific antigen that is | | |
| part of the B cell receptor | | |
| complex and that functions | | |
| in determining the threshold | | |
| for B cell activation B cells | | |
| Plasmablasts, B cells that | | |
| express CD19 (but not | | |
| CD20) and that secrete large | | |
| quantities of antibodies; | | |
| depletion of plasmablasts | | |
| may be useful in | | |
| autoimmune diseases | | |
| involving pathogenic | | |
| autoantibodies) | | |
| Minocycline (targets T cells | VARIOUS | Oral Available as pellet-filled |
| Microglia Leukocyte | | capsules and an oral suspension |
| migration Matrix | | |
| metalloproteinases) | | |
| MIS416 (targets Innate | INNATE | Intravenous |
| immune system Pathogen- | IMMUNOTHERAPEUTICS | |
| associated molecular pattern | | |
| recognition receptors of the | | |
| innate immune system | | |
| Myeloid cells of the innate | | |
| immune system, which might | | |
| be able to remodel the | | |
| deregulated immune system | | |
| activity that occurs in SPMS) | | |
| Mycophenolate mofetil | MANUFACTURED BY | Oral |
| (targets Purine synthesis) | GENENTECH | |
| Naltrexone (targets Opioid | VARIOUS | Given at low doses (3 to 4.5 mg per day) in oral |
| receptors Toll-like receptor 4) | | form as “Low-dose naltrexone” (or “LDN”) |
| Ocrelizumab and | ROCHE/GSK | Projected to be IV infusion |
| Ofatumumab (humanized | | |
| monoclonal antibodies | | |
| targeting CD20 B cell | | |
| suppression | | |
| ONO-4641 (targets | ONO | Oral |
| Sphingosine 1-phosphate | PHARMACEUTICAL | |
| receptor) | CO. | |
| Phenytoin (targets Sodium | PFIZER | Intravenous Intramuscular |
| channels) | | (less favored option) Oral |
| Ponesimod | ACTELION | To be determined |
| Raltegravir (targets | MERCK | Oral 400 mg tablet twice daily, according to |
| Retroviral integrase | | information from the manufacturer |
| Herpesvirus DNA packaging | | |
| terminase) | | |
| RHB-104 | REDHILL | 95 mg clarithromycin, 45 mg |
| | BIOPHARMA | rifabutin, and 10 mg clofazimine |
| | LIMITED | |
| Riluzole (targets | COVIS | Oral |
| Glutamatergic | PHARMA/ | |
| neurotransmission | SANOFI | |
| Glutamate uptake and | | |
| release Voltage-gated | | |
| sodium channels Protein | | |
| kinase C) | | |

MS disease progression may be most intensive, and most damaging, at the earliest stages of disease progression. Accordingly, counter to many reimbursement policies and physician practice in light of, for example, costs and side effect mitigation, it may be most beneficial for a patient's long term disease status to begin treatment with the most intensive DMTs, for instance so-called second-line therapies. In some embodiments, a patient is treated with a regimen of the Fc-based chimeric protein complex in combination with a second-line therapy. Such a combination is used to reduce the side effect profile of one or more second-line therapies. In some embodiments, the combination is used to reduce dose of frequency of administration of one or more second-line therapies. For example, the doses of agents listed in the Table provided above may be reduced by about 50%, or about 40%, or about 30%, or about 25% in the context of the combination and the/or the frequency of dosing may be decreased to be half as often, or a third as often or may be reduced from, for example, daily to every other day or weekly, every other day to weekly or bi-weekly, weekly to bi-weekly or monthly, etc. Accordingly, in some embodiments, the Fc-based chimeric protein complex increase patient adherence by allowing for more convenient treatment regimens. Further, some DMTs have a suggested lifetime dose limitation e.g. for mitoxantrone, the lifetime cumulative dose should be strictly limited to 140 mg/m², or 2 to 3 years of therapy. In some embodiments, supplementation with the Fc-based chimeric protein complex preserves patient's access to mitoxantrone by allowing for lower or less frequent dosing with this DMT.

In some embodiments, the patient is a naive patient, who has not received treatment with one or more DMTs, and the Fc-based chimeric protein complex is used to buffer the side effects of a second-line therapy. Accordingly, the naive patient is able to benefit from the long-term benefits of a second-line therapy at disease outset. In some embodiments, the Fc-based chimeric protein complex is used as an entry therapy that precedes the use of a second-line therapy. For example, the Fc-based chimeric protein complex may be administered for an initial treatment period of about 3 months to stabilize disease and then the patient may be transitioned to a maintenance therapy of a second line agent.

It is generally believed that naive patients are more likely to respond to therapy as compared to patients that have received, and perhaps failed one or more DMT. In some embodiments, the Fc-based chimeric protein complex finds use in patients that have received, and perhaps failed one or more DMT. For example, in some embodiments, the Fc-based chimeric protein complex increases the therapeutic effect in patients that have received, and perhaps failed one or more DMT and may allow these patients to respond like naive patients.

In some embodiments, the patient has received or is receiving treatment with one or more DMTs and is not responding well. For example, the patient may be refractory or poorly responsive to one or more DMTs. In some embodiments, the patient is refractory, or poorly responsive to one or more of teriflunomide (AUBAGIO (GENZYME)); interferon beta-1a (AVONEX (BIOGEN IDEC); interferon beta-1b (BETASERON (BAYER HEALTHCARE PHARMACEUTICALS, INC.); glatiramer acetate (COPAXONE (TEVA NEUROSCIENCE); interferon beta-1b (EXTAVIA (NOVARTIS PHARMACEUTICALS CORP.); fingolimod (GILENYA (NOVARTIS PHARMACEUTICALS CORP.); alemtuzumab (LEMTRADA (GENZYME); mitoxantrone (NOVANTRONE (EMD SERONO); pegylated interferon beta-1a (PLEGRIDY (BIOGEN IDEC); interferon beta-1a (REBIF (EMD SERONO, INC.); dimethyl fumarate (BG-12) (TECFIDERA (BIOGEN IDEC); and natalizumab (TYSABRI (BIOGEN IDEC). In some embodiments, the one or more disclosed binding agent results in a therapeutic benefit of one or more DMTs in the patient and therefore reduces or eliminates the non-responsiveness to the DMT. For instance, this may spare the patient therapy with one or more DMTs at a higher dosing or frequency.

In patients with more aggressive disease, one approach is an induction treatment model, where a therapy with strong efficacy but strong safety concerns would be given first, followed by a maintenance therapy. An example of such a model might include initial treatment with alemtuzumab, followed by IFN-β, GA, or BG-12. In some embodiments, the one or more disclosed binding agent is used to prevent the need to switch therapies for maintenance. In some embodiments, the one or more disclosed binding agent is used to as maintenance therapy to one or more DMTs, including second line therapies. In some embodiments, the one or more disclosed binding agent is used to as first therapy in an induction, followed by another DMT as a maintenance therapy—such as, for example, a first line therapy.

In some embodiments, the one or more disclosed binding agent may be administered for an initial treatment period of about 3 months to stabilize disease and then the patient may be transitioned to a maintenance therapy of a first line agent.

In various embodiments, the one or more disclosed binding agent is used to reduce one or more side effects of a DMT, including without limitation any agent disclosed herein. For example, the one or more disclosed binding agent may be used in a regimen that allows dose sparing for one or more DMTs and therefore results in fewer side effects. For example, in some embodiments, the one or more disclosed binding agent may reduce one or more side effects of AUBAGIO or related agents, which may include hair thinning, diarrhea, flu, nausea, abnormal liver tests and unusual numbness or tingling in the hands or feet (paresthesias), levels of white blood cells, which can increase the risk of infections; increase in blood pressure; and severe liver damage. In some embodiments, the one or more disclosed binding agent may reduce one or more side effects of AVONEX or related agents which include flu-like symptoms following injection, depression, mild anemia, liver abnormalities, allergic reactions, and heart problems. In some embodiments, the one or more disclosed binding agent may reduce one or more side effects of BETASERON or related agents which include flu-like symptoms following injection, injection site reactions, allergic reactions, depression, liver abnormalities, and low white blood cell counts. In some embodiments, the one or more disclosed binding agent may reduce one or more side effects of COPAXONE or related agents which include injection site reactions, vasodilation (dilation of blood vessels); chest pain; a reaction immediately after injection, which includes anxiety, chest pain, palpitations, shortness of breath, and flushing. In some embodiments, the one or more disclosed binding agent may reduce one or more side effects of EXTAVIA or related agents which include flu-like symptoms following injection, injection site reactions, allergic reactions, depression, liver abnormalities, and low white blood cell counts. In some embodiments, the one or more disclosed binding agent may reduce one or more side effects of GILENYA or related agents which include headache, flu, diarrhea, back pain, liver enzyme elevations, cough, slowed heart rate following first dose, infections, and swelling in the eye. In some embodiments, the one or more disclosed binding agent may reduce one or more side effects of LEMTRADA or related agents which include rash, headache, fever, nasal congestion, nausea, urinary tract infection, fatigue, insomnia, upper respiratory tract infection, hives, itching, thyroid gland disorders, fungal Infection, pain in joints, extremities and back, diarrhea, vomiting, flushing, and infusion reactions (including nausea, hives, itching, insomnia, chills, flushing, fatigue, shortness of breath, changes in the sense of taste, indigestion, dizziness, pain). In some embodiments, the one or more disclosed binding agent may reduce one or more side effects of NOVANTRONE or related agents which include blue-green urine 24 hours after administration; infections, bone marrow suppression (fatigue, bruising, low blood cell counts), nausea, hair thinning, bladder infections, mouth sores, and serious liver and heart damage. In some embodiments, the one or more disclosed binding agent may reduce one or more side effects of PLEGRIDY or related agents which include flu-like symptoms following injection, injection site reactions, depression, mild anemia, liver abnormalities, allergic reactions, and heart problems. In some embodiments, the one or more disclosed binding agent may reduce one or more side effects of REBIF or related agents which include flu-like symptoms following injection, injection site reactions, liver abnormalities, depression, allergic reactions, and low red or white blood cell counts. In some embodiments, one or more disclosed binding agent may reduce one or more side effects of TECFIDERA or related agents which include flushing (sensation of heat or itching and a blush on the skin), gastrointestinal issues (nausea, diarrhea, abdominal pain), rash, protein in the urine, elevated liver enzymes; and reduction in blood lymphocyte (white blood cell) counts. In some embodiments, the one or more disclosed binding agent may reduce one or more side effects of TYSABRI or related agents which include headache, fatigue, urinary tract infections, depression, respiratory tract infections, joint pain, upset stomach, abdominal discomfort, diarrhea, vaginitis, pain in the arms or legs, rash, allergic or hypersensitivity reactions within two hours of infusion (dizziness, fever, rash, itching, nausea, flushing, low blood pressure, difficulty breathing, chest pain).

In some embodiments, the present invention relates to combination therapy with one or more chimeric agents described in WO 2013/10779, WO 2015/007536, WO 2015/007520, WO 2015/007542, and WO 2015/007903, the entire contents of which are hereby incorporated by reference in their entireties.

In some embodiments, inclusive of, without limitation, infectious disease applications, the present invention pertains to anti-infectives as additional therapeutic agents. In some embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In some embodiments, inclusive, without limitation, of autoimmmune applications, the additional therapeutic agent is an immunosuppressive agent. In some embodiments, the immunosuppressive agent is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In some embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin). Additional anti-inflammatory agents are described, for example, in U.S. Pat. No. 4,537,776, the entire contents of which is incorporated by reference herein.

In some embodiments, the Fc-based chimeric protein complex described herein, include derivatives that are modified, i.e., by the covalent attachment of any type of molecule to the composition such that covalent attachment does not prevent the activity of the composition. For example, but not by way of limitation, derivatives include composition that have been modified by, inter a/ia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of tunicamycin, etc.

In still other embodiments, the Fc-based chimeric protein complex described herein further comprise a cytotoxic agent, comprising, in illustrative embodiments, a toxin, a chemotherapeutic agent, a radioisotope, and an agent that causes apoptosis, necrosis or any other form of cell death. Such agents may be conjugated to a composition described herein.

The Fc-based chimeric protein complex described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Illustrative cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine, bleomycin, VEGF antagonists, EGFR antagonists, platins, taxols, irinotecan, 5-fluorouracil, gemcytabine, leucovorine, steroids, cyclophosphamide, melphalan, vinca alkaloids (e.g., vinblastine, vincristine, vindesine and vinorelbine), mustines, tyrosine kinase inhibitors, radiotherapy, sex hormone antagonists, selective androgen receptor modulators, selective estrogen receptor modulators, PDGF antagonists, TNF antagonists, IL-1β antagonists, interleukins (e.g. IL-12 or IL-2), IL-12R antagonists, Toxin conjugated monoclonal antibodies, tumor antigen specific monoclonal antibodies, Erbitux, Avastin, Pertuzumab, anti-CD20 antibodies, Rituxan, ocrelizumab, ofatumumab, DXL625, HERCEPTIN®, or any combination thereof. Toxic enzymes from plants and bacteria such as ricin, diphtheria toxin and Pseudomonas toxin may be conjugated to the therapeutic agents (e.g. antibodies) to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the Fc-based chimeric protein complex, with or without the use of a complex-forming agent. Such radionuclides include beta-emitters such as Phosphorus-32, Scandium-47, Copper-67, Gallium-67, Yttrium-88, Yttrium-90, Iodine-125, Iodine-131, Samarium-153, Lutetium-177, Rhenium-186 or Rhenium-188, and alpha-emitters such as Astatine-211, Lead-212, Bismuth-212, Bismuth-213 or Actinium-225.

Illustrative detectable moieties further include, but are not limited to, horseradish peroxidase, acetylcholinesterase, alkaline phosphatase, beta-galactosidase and luciferase. Further illustrative fluorescent materials include, but are not limited to, rhodamine, fluorescein, fluorescein isothiocyanate, umbelliferone, dichlorotriazinylamine, phycoerythrin and dansyl chloride. Further illustrative chemiluminescent moieties include, but are not limited to, luminol. Further illustrative bioluminescent materials include, but are not limited to, luciferin and aequorin. Further illustrative radioactive materials include, but are not limited to, Iodine-125, Carbon-14, Sulfur-35, Tritium and Phosphorus-32.

### Methods of Treatment

Methods and compositions described herein have application to treating various diseases and disorders, including, but not limited to cancer, infections, immune disorders, and inflammatory diseases or conditions.

Further, any of the present agents may be for use in the treating, or the manufacture of a medicament for treating, various diseases and disorders, including, but not limited to cancer, infections, immune disorders, inflammatory diseases or conditions, and autoimmune diseases.

In some embodiments, the present invention relates to the treatment of, or a patient having cancer. As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); acute myeloid leukemia (AML); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In various embodiments, the present invention provides Fc-based chimeric protein complexes which comprise wild type or modified signaling agents for the treatment of cancer. In some embodiments, the Fc-based chimeric protein complexes of the invention significantly reduce and/or eliminate tumors. In some embodiments, the present Fc-based chimeric protein complexes significant reduce and/or eliminate tumors when administered to a subject in combination with other anti-cancer agents such as chemotherapeutic agents, checkpoint inhibitors, and immunosuppressive agents. In various embodiments, the combination of Fc-based chimeric protein complexes and other anti-cancer agents synergistically reduced tumor size and/or eliminated tumor cells.

In various embodiments, the present invention relates to cancer combination therapies with an Fc-based chimeric protein complex comprising one or more targeting moieties and one or more wild type or modified signaling agents. Accordingly, the present invention provides for an Fc-based chimeric protein complex that include, for example, a targeting moiety and one or more signaling agents and uses thereof in combination with anti-cancer agents.

For instance, in various embodiments, the present invention pertains to combination therapies for cancer involving Fc-based chimeric protein complex and a wild type or modified signaling agent, including, without limitation a mutated human interferon, such as IFN alpha, including human interferon alpha 2.

In other embodiments, the present Fc-based chimeric protein complex comprises multiple targeting moieties and therefore be present in bispecific or trispecific formats. For instance, in various embodiments, the present invention pertains to combination therapies for cancer involving an Fc-based chimeric protein complex and a checkpoint inhibitor binding agent (e.g. anti-PD-L1, anti-PD-1, anti-PD-L2, or anti-CTLA) described herein and a modified signaling agent, including, without limitation a mutated human interferon, such as IFN alpha, including human interferon alpha 2.

In various embodiments, the signaling agent is wild type or modified to have reduced affinity or activity for one or more of its receptors, which allows for attenuation of activity (inclusive of agonism or antagonism) and/or prevents non-specific signaling or undesirable sequestration of the chimeric protein. In some embodiments, the reduced affinity or activity at the receptor is restorable by inclusion in the present complex having one or more of the targeting moieties as described herein.

In some embodiments, the present invention relates to the treatment of, or a patient having a microbial infection and/or chronic infection. Illustrative infections include, but are not limited to, HIV/AIDS, tuberculosis, osteomyelitis, hepatitis B, hepatitis C, Epstein-Barr virus or parvovirus, T cell leukemia virus, bacterial overgrowth syndrome, fungal or parasitic infections.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, cancer, heart failure, autoimmune disease, sickle cell disease, thalassemia, blood loss, transfusion reaction, diabetes, vitamin B12 deficiency, collagen vascular disease, Shwachman syndrome, thrombocytopenic purpura, Celiac disease, endocrine deficiency state such as hypothyroidism or Addison's disease, autoimmune disease such as Crohn's Disease, systemic lupus erythematosis, rheumatoid arthritis or juvenile rheumatoid arthritis, ulcerative colitis immune disorders such as eosinophilic fasciitis, hypoimmunoglobulinemia, or thymoma/thymic carcinoma, graft versus host disease, preleukemia, Nonhematologic syndrome (e.g. Down's, Dubowwitz, Seckel), Felty syndrome, hemolytic uremic syndrome, myelodysplasic syndrome, nocturnal paroxysmal hemoglobinuria, osteomyelofibrosis, pancytopenia, pure red-cell aplasia, Schoenlein-Henoch purpura, malaria, protein starvation, menorrhagia, systemic sclerosis, liver cirrhosis, hypometabolic states, and congestive heart failure.

In some embodiments, the present invention relates to the treatment of, or a patient having one or more of chronic granulomatous disease, osteopetrosis, idiopathic pulmonary fibrosis, Friedreich's ataxia, atopic dermatitis, Chagas disease, mycobacterial infections, cancer, scleroderma, hepatitis, hepatitis C, septic shock, and rheumatoid arthritis.

In various embodiments, the present compositions are used to treat or prevent one or more inflammatory diseases or conditions, such as inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconioses.

In various embodiments, the present invention has application to treating autoimmune and/or neurodegenerative diseases.

In various embodiments, the present compositions are used to treat or prevent one or more conditions characterized by undesirable CTL activity, and/or a conditions characterized by high levels of cell death. For instance, in various embodiments, the present compositions are used to treat or prevent one or more conditions associated with uncontrolled or overactive immune response.

In various embodiments, the present compositions are used to treat or prevent one or more autoimmune and/or neurodegenerative diseases or conditions, such as MS, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, Grave's disease, and other autoimmune diseases.

In various embodiments, the present invention is used to treat or prevent various autoimmune and/or neurodegenerative diseases. In some embodiments, the autoimmune and/or neurodegenerative diseases selected from MS (including without limitation the subtypes described herein), Alzheimer's disease (including, without limitation, Early-onset Alzheimer's, Late-onset Alzheimer's, and Familial Alzheimer's disease (FAD), Parkinson's disease and parkinsonism (including, without limitation, Idiopathic Parkinson's disease, Vascular parkinsonism, Drug-induced parkinsonism, Dementia with Lewy bodies, Inherited Parkinson's, Juvenile Parkinson's), Huntington's disease, Amyotrophic lateral sclerosis (ALS, including, without limitation, Sporadic ALS, Familial ALS, Western Pacific ALS, Juvenile ALS, Hiramaya Disease).

In an embodiment, the present invention provides methods for the treatment or prevention of one or more liver disorders, selected from viral hepatitis, alcohol hepatitis, autoimmune hepatitis, alcohol liver disease, fatty liver disease, steatosis, steatohepatitis, non-alcohol fatty liver disease, drug-induced liver disease, cirrhosis, fibrosis, liver failure, drug induced liver failure, metabolic syndrome, hepatocellular carcinoma, cholangiocarcinoma, primary biliary cirrhosis (primary biliary cholangitis), bile capillaries, Gilbert's syndrome, jaundice, and any other liver toxicity-associated indication. In some embodiments, the present invention provides methods for the treatment or prevention of liver fibrosis. In some embodiments, the present invention provides methods for the treatment or prevention of primary sclerosing cholangitis (PSC), chronic liver disease, nonalcoholic fatty liver disease (NAFLD), nonalcoholic steatohepatitis (NASH), hepatitis C infection, alcoholic liver disease, liver damage, optionally due to progressive fibrosis and liver fibrosis. In some embodiments, the present invention provides methods for the treatment or prevention of nonalcoholic steatohepatitis (NASH). In some embodiments, the present invention provides methods that reduce or prevent fibrosis. In some embodiments, the present invention provides methods that reduce or prevent cirrhosis. In some embodiments, the present invention provides methods that reduce or prevent hepatocellular carcinoma.

In various embodiments, the present invention provides methods for the treatment or prevention of cardiovascular disease, such as a disease or condition affecting the heart and vasculature, including but not limited to, coronary heart disease (CHD), cerebrovascular disease (CVD), aortic stenosis, peripheral vascular disease, atherosclerosis, arteriosclerosis, myocardial infarction (heart attack), cerebrovascular diseases (stroke), transient ischaemic attacks (TIA), angina (stable and unstable), atrial fibrillation, arrhythmia, valvular disease, and/or congestive heart failure. In various embodiments, the present invention provides methods for the treatment or prevention of cardiovascular disease which involves inflammation.

In various embodiments, the present invention provides methods for the treatment or prevention of one or more respiratory diseases, such as asthma, chronic obstructive pulmonary disease (COPD), bronchiectasis, allergic rhinitis, sinusitis, pulmonary vasoconstriction, inflammation, allergies, impeded respiration, respiratory distress syndrome, cystic fibrosis, pulmonary hypertension, pulmonary vasoconstriction, emphysema, Hantavirus pulmonary syndrome (HPS), Loeffler's syndrome, Goodpasture's syndrome, Pleurisy, pneumonitis, pulmonary edema, pulmonary fibrosis, Sarcoidosis, complications associated with respiratory syncitial virus infection, and other respiratory diseases.

In various embodiments, the present invention is used to treat or prevent MS. In various embodiments, the Fc-based chimeric protein complexes as described herein are used to eliminate and reduce multiple MS symptoms. Illustrative symptoms associated with multiple sclerosis, which can be prevented or treated with the compositions and methods described herein, include: optic neuritis, diplopia, nystagmus, ocular dysmetria, internuclear ophthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l′hermitte's sign, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoff's symptom, gastroesophageal reflux, and sleeping disorders. Mitigation or amelioration or one more of these symptoms in a subject can be achieved by the one or more agent as described herein.

In various embodiments, the Fc-based chimeric protein complexes as described herein is used to treat or prevent clinically isolated syndrome (CIS). A clinically isolated syndrome (CIS) is a single monosymptomatic attack compatible with MS, such as optic neuritis, brain stem symptoms, and partial myelitis. Patients with CIS that experience a second clinical attack are generally considered to have clinically definite multiple sclerosis (CDMS). Over 80 percent of patients with CIS and MRI lesions go on to develop MS, while approximately 20 percent have a self-limited process. Patients who experience a single clinical attack consistent with MS may have at least one lesion consistent with multiple sclerosis prior to the development of clinically definite multiple sclerosis. In various embodiments, the presently described Fc-based chimeric protein complexes is used to treat CIS so it does not develop into MS, including, for example RRMS.

In various embodiments, the Fc-based chimeric protein complexes as described herein are used to treat or prevent radiologically isolated syndrome (RIS). In RIS, incidental imaging findings suggest inflammatory demyelination in the absence of clinical signs or symptoms. In various embodiments, the Fc-based chimeric protein complex is used to treat RIS so it does not develop into MS, including, for example RRMS.

In various embodiments, the Fc-based chimeric protein complexes as described herein are used to treat one or more of benign multiple sclerosis; relapsing-remitting multiple sclerosis (RRMS); secondary progressive multiple sclerosis (SPMS); progressive relapsing multiple sclerosis (PRMS); and primary progressive multiple sclerosis (PPMS).

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis. In various embodiments, the Fc-based chimeric protein complex is used to treat benign multiple sclerosis so it does not develop into MS.

Patients suffering from RRMS experience sporadic exacerbations or relapses, as well as periods of remission. Lesions and evidence of axonal loss may or may not be visible on MRI for patients with RRMS. In various embodiments, the Fc-based chimeric protein complexes as described herein are used to treat RRMS. In some embodiments, RRMS includes patients with RRMS; patients with SPMS and superimposed relapses; and patients with CIS who show lesion dissemination on subsequent MRI scans according to McDonald's criteria. A clinical relapse, which may also be used herein as “relapse,” “confirmed relapse,” or “clinically defined relapse,” is the appearance of one or more new neurological abnormalities or the reappearance of one or more previously observed neurological abnormalities. This change in clinical state must last at least 48 hours and be immediately preceded by a relatively stable or improving neurological state of at least 30 days. In some embodiments, an event is counted as a relapse when the subject's symptoms are accompanied by observed objective neurological changes, consistent with an increase of at least 1.00 in the Expanded Disability Status Scale (EDSS) score or one grade in the score of two or more of the seven FS or two grades in the score of one of FS as compared to the previous evaluation.

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS. In various embodiments, the Fc-based chimeric protein complexes as described herein is used to treat RRMS so it does not develop into SPMS.

PPMS is characterized by a steady progression of increasing neurological deficits without distinct attacks or remissions. Cerebral lesions, diffuse spinal cord damage and evidence of axonal loss are evident on the MRI of patients with PPMS. PPMS has periods of acute exacerbations while proceeding along a course of increasing neurological deficits without remissions. Lesions are evident on MRI of patients suffering from PRMS. In various embodiments, the Fc-based chimeric protein complex as described herein is used to treat RRMS and/or SPMS so it does not develop into PPMS.

In some embodiments, the Fc-based chimeric protein complexes as described herein are used in a method of treatment of relapsing forms of MS. In some embodiments, the Fc-based chimeric protein complex is used in a method of treatment of relapsing forms of MS to slow the accumulation of physical disability and/or reduce the frequency of clinical exacerbations, and, optionally, for patients who have experienced a first clinical episode and have MRI features consistent with MS. In some embodiments, the Fc-based chimeric protein complexes as described herein are used in a method of treatment of worsening relapsing-remitting MS, progressive-relapsing MS or secondary-progressive MS to reduce neurologic disability and/or the frequency of clinical exacerbations. In some embodiments, the Fc-based chimeric protein complexes reduce the frequency and/or severity of relapses.

In some embodiments, the Fc-based chimeric protein complexes are used in a method of treatment of relapsing forms of MS in patients who have had an inadequate response to (or are refractory to) one, or two, or three, or four, or five, or six, or seven, or eight, or nine, or ten or more disease modifying therapies (DMTs).

In various embodiments, the subject's symptoms may be assessed quantitatively, such as by EDSS, or decrease in the frequency of relapses, or increase in the time to sustained progression, or improvement in the magnetic resonance imaging (MRI) behavior in frequent, serial MRI studies and compare the patient's status measurement before and after treatment. In a successful treatment, the patient status will have improved (e.g., the EDSS measurement number or frequency of relapses will have decreased, or the time to sustained progression will have increased, or the MRI scans will show less pathology).

In some embodiments, the patient can be evaluated, e.g., before, during or after receiving the Fc-based chimeric protein complexes e.g., for indicia of responsiveness. Various clinical or other indicia of effectiveness of treatment, e.g., EDSS score; MRI scan; relapse number, rate, or severity; multiple sclerosis functional composite (MSFC); multiple sclerosis quality of life inventory (MSQLI); Paced Serial Addition Test (PASAT); symbol digit modalities test (SDMT); 25-foot walk test; 9-hole peg test; low contrast visual acuity; Modified Fatigue Impact Scale; expanded disability status score (EDSS); multiple sclerosis functional composite (MSFC); Beck Depression Inventory; and 7/24 Spatial Recall Test can be used. In various embodiments, the Fc-based chimeric protein complexes cause an improvement in one or more of these measures. Further, the patient can be monitored at various times during a regimen. In some embodiments, the Fc-based chimeric protein complexes cause a disease improvement as assessed by MacDonald dissemination in space and time. For example, for dissemination in space, lesion imaging, such as, by way of illustration, Barkhof-Tintore MR imaging criteria, may be used, including at least one gadolinium-enhancing lesion or 9 T2 hyperintense lesions; at least one infratentorial lesion; at least one juxtacortical lesion; at least about three periventricular lesions; and a spinal cord lesion. For dissemination in time, MRI can also be used; for example, if an MRI scan of the brain performed at months after an initial clinical event demonstrates a new gadolinium-enhancing lesion, this may indicate a new CNS inflammatory event, because the duration of gadolinium enhancement in MS is usually less than 6 weeks. If there are no gadolinium-enhancing lesions but a new T2 lesion (presuming an MRI at the time of the initial event), a repeat MR imaging scan after another 3 months may be needed with demonstration of a new T2 lesion or gadolinium-enhancing lesion.

In some embodiments, disease effects are assessed using any of the measures described in Lavery, et al. Multiple Sclerosis International, Vol 2014 (2014), Article ID 262350, the entire contents of which are hereby incorporated by reference.

In some embodiments, the Fc-based chimeric protein complex results in one or more of: (a) prevention of worsening in disability defined as deterioration by 1.0 point on EDSS, (b) increase in time to relapse, (c) reduction or stabilization of number and/or volume of gadolinium enhancing lesions, (d) decreased annualized relapse rate, (e) increased relapse duration and severity by NRS score, (f) decrease in disease activity as measured by MRI (annual rate of new or enlarging lesions), (g) lower average number of relapses at 1 year, or 2 years, (h) sustained disease progression as measured by the EDSS at 3 months, (i) prevention of conversion to CDMS, (j) no or few new or enhancing T2 lesions, (k) minimal change in hyperintense T2 lesion volume, (I) increased time to McDonald defined MS, (m) prevention of progression of disability as measured by sustained worsening of EDSS at 12 weeks, (n) reduction in time to relapse at 96 weeks, and (o) reduction or stabilization of brain atrophy (e.g. percentage change from baseline).

In one embodiment, the Fc-based chimeric protein complexes are administered and is effective to result in a decreased rate of relapse (e.g., at least 10%, 20%, 30%, 40%, 50%, 60%, 70%, 80% or greater reduction in rate of relapse) compared to the rate of relapse before administration (e.g., compared to the rate of relapse following administration for 12 months or for less than 12 months, e.g., about 10, or about 8, or about 4, or about 2 or less months) of treatment, or before commencement of treatment, when measured between 3-24 months (e.g., between 6-18 months, e.g., 12 months) after a previous relapse.

In one embodiment, the Fc-based chimeric protein complexes are administered and are effective to result in a prevention of an increase in EDSS score from a pre-treatment state. The Kurtzke Expanded Disability Status Scale (EDSS) is a method of quantifying disability in multiple sclerosis. The EDSS replaced the previous Disability Status Scales which used to bunch people with MS in the lower brackets. The EDSS quantifies disability in eight Functional Systems (FS) and allows neurologists to assign a Functional System Score (FSS) in each of these. The Functional Systems are: pyramidal, cerebellar, brainstem, sensory, bowel and bladder, visual and cerebral.

In one embodiment, the Fc-based chimeric protein complexes are administered and is effective to result in a decreased EDSS score (e.g., a decrease of 1, 1.5, 2, 2.5, 3 points or more, e.g., over at least three months, six months, one year, or longer) compared to the EDSS score following administration of the Fc-based chimeric protein complexes (e.g. for 12 months or for less than 12 months, e.g., less than 10, 8, 4 or less months, or before the commencement of treatment).

In one embodiment, the Fc-based chimeric protein complexes are administered and is effective to result in a decreased number of new lesions overall or of any one type (e.g., at least 10%, 20%, 30%, 40% decrease), compared to the number of new lesions following administration of the Fc-based chimeric protein complexes for 12 months or for less than 12 months, e.g., less than 10, 8, 4 or less months, or before commencement of treatment;

In one embodiment, the Fc-based chimeric protein complexes are administered and is effective to result in a decreased number of lesions overall or of any one type (e.g., at least 10%, 20%, 30%, 40% decrease), compared to the number of lesions following administration of the Fc-based chimeric protein complexes for 12 months or for less than 12 months, e.g., less than 10, 8, 4 or less months, or before commencement of treatment;

In one embodiment, the Fc-based chimeric protein complexes are administered and is effective to result in a reduced rate of appearance of new lesions overall or of any one type (e.g., at least 10%, 20%, 30%, 40% reduced rate), compared to the rate of appearance of new lesions following administration for 12 months or for less than 12 months, e.g., less than 10, 8, 4 or less months, or before commencement of treatment;

In one embodiment, the Fc-based chimeric protein complexes are administered and is effective to result in a reduced increase in lesion area overall or of any one type (e.g., at least 10%, 20%, 30%, 40% decreased increase), compared to an increase in lesion area following administration for 12 months or less than 12 months, e.g., less than 10, 8, 4 or less months, or before commencement of treatment.

In one embodiment, the Fc-based chimeric protein complexes are administered and is effective to result in a reduced incidence or symptom of optic neuritis (e.g., improved vision), compared to the incidence or symptom of optic neuritis following administration for 12 months or for less than 12 months, e.g., less than 10, 8, 4 or less months, or before commencement of treatment.

In various embodiments, methods of the invention are useful in treatment a human subject. In some embodiments, the human is a pediatric human. In other embodiments, the human is an adult human. In other embodiments, the human is a geriatric human. In other embodiments, the human may be referred to as a patient. In some embodiments, the human is a female. In some embodiments, the human is a male.

In certain embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old. In various embodiments, the human has an age of more than 30 years old.

### Immune Modulation

In various embodiments, the present compositions are capable of, or find use in methods of, immune modulation. For instance, in various embodiments, the present methods of treatment may involve the immune modulation described herein. In some embodiments, the immune modulation involves IFN signaling, including modified IFN signaling, in the context of a dendritic cell (DC).

In various embodiments, a multi-specific Fc-based chimeric protein complex is provided. In some embodiments, such multi-specific Fc-based chimeric protein complex of the invention recognizes and binds to a first target and one or more antigens found on one or more immune cells, which can include, without limitation, megakaryocytes, thrombocytes, erythrocytes, mast cells, basophils, neutrophils, eosinophils, monocytes, macrophages, natural killer cells, T lymphocytes (e.g., cytotoxic T lymphocytes, T helper cells, natural killer T cells), B lymphocytes, plasma cells, dendritic cells, or subsets thereof. In some embodiments, the Fc-based chimeric protein complex specifically binds to an antigen of interest and effectively directly or indirectly recruits one of more immune cells.

In some embodiments, the Fc-based chimeric protein complex specifically binds to an antigen of interest and effectively directly or indirectly recruits one of more immune cells to cause an immunosuppressive effect, e.g. the Fc-based chimeric protein complex directly or indirectly recruits an immunosuppressive immune cell. In some embodiments, the immunosuppressive immune cell is a regulatory T cell (or “Tregs” which, as used herein, refers to a subpopulation of T cells which modulate the immune system, abrogate autoimmune disease, maintain tolerance to self-antigens and thwart anti-tumor immune responses). Other immunosuppressive immune cells include myeloid suppressor cells (or “MSC,” which, as used herein, refers to a heterogeneous population of cells, defined by their myeloid origin, immature state, and ability to potently suppress T cell responses); tumor associated neutrophils (or “TANs” which, as used herein, refers to a subset of neutrophils that are capable of suppressing immune responses); tumor associated macrophages (or “TAMs” which, as used herein, refers to a subset of macrophages that may reduce an immune response), M2 macrophages, and/or tumor-inducing mast cells (which as used herein, refers to a subset of bone marrow-derived, long-lived, heterogeneous cellular population). Also, immunosuppressive immune cells include Th2 cells and Th17 cells. Additionally, immunosuppressive immune cells include immune cells, e.g., CD4+ and/or CD8+ T cells, expressing one or more checkpoint inhibitory receptors (e.g. receptors, including CTLA-4, B7-H3, B7-H4, TIM-3, expressed on immune cells that prevent or inhibit uncontrolled immune responses). See Stagg, J. et. al., Immunotherapeutic approach in triple-negative breast cancer. Ther Adv Med Oncol. (2013) 5(3):169-181).

In some embodiments, the Fc-based chimeric protein complex stimulates regulatory T cell (Treg) proliferation. Treg cells are characterized by the expression of the Foxp3 (Forkhead box p3) transcription factor. Most Treg cells are CD4+ and CD25+, and can be regarded as a subset of helper T cells, although a small population may be CD8+. Thus the immune response, which is to be modulated by a method of the invention, may comprise inducing proliferation of Treg cells, optionally in response to an antigen. Thus the method may comprise administering to the subject an Fc-based chimeric protein complex comprising the antigen. The antigen may be administered with an adjuvant which promotes proliferation of Treg cells.

Insofar as this method involves stimulating proliferation and differentiation of Treg cells in response to a specific antigen, it can be considered to be a method of stimulating an immune response. However, given that Treg cells may be capable of modulating the response of other cells of the immune system against an antigen in other ways, e.g. inhibiting or suppressing their activity, the effect on the immune system as a whole may be to modulate (e.g. suppress or inhibit) the response against that antigen. Thus the methods of this aspect of the invention can equally be referred to as methods of modulating (e.g. inhibiting or suppressing) an immune response against an antigen.

In some embodiments, the methods therapeutically or prophylactically inhibit or suppress an undesirable immune response against a particular antigen, even in a subject with pre-existing immunity or an on-going immune response to that antigen. This may be particularly useful, for example, in the treatment of autoimmune disease.

Under certain conditions, it may also be possible to tolerize a subject against a particular antigen by targeting the antigen to an antigen presenting cell expressing a target of the targeting moiety of the Fc-based chimeric protein complex. The invention thus provides a method for inducing tolerance in a subject towards an antigen, comprising administering to the subject a composition comprising the antigen, wherein the antigen is associated with a binding agent having affinity for the targeting moiety of the Fc-based chimeric protein complex and wherein the antigen is administered in the absence of an adjuvant. Tolerance in this context typically involves depletion of immune cells which would otherwise be capable of responding to that antigen, or inducing a lasting reduction in responsiveness to an antigen in such immune cells.

It may be particularly desirable to raise a Treg response against an antigen to which the subject exhibits, or is at risk of developing, an undesirable immune response. For example, it may be a self-antigen against which an immune response occurs in an autoimmune disease. Examples of autoimmune diseases in which specific antigens have been identified as potentially pathogenically significant include multiple sclerosis (myelin basic protein), insulin-dependent diabetes mellitus (glutamic acid decarboxylase), insulin-resistant diabetes mellitus (insulin receptor), celiac disease (gliadin), bullous pemphigoid (collagen type XVII), auto-immune haemolytic anaemia (Rh protein), auto-immune thrombocytopenia (GpIIb/IIIa), myaesthenia gravis (acetylcholine receptor), Graves' disease (thyroid-stimulating hormone receptor), glomerulonephritis, such as Goodpasture's disease (alpha3(IV)NC1 collagen), and pernicious anaemia (intrinsic factor). Alternatively, the target antigen may be an exogenous antigen which stimulates a response which also causes damage to host tissues. For example, acute rheumatic fever is caused by an antibody response to a Streptococcal antigen which cross-reacts with a cardiac muscle cell antigen. Thus these antigens, or particular fragments or epitopes thereof, may be suitable antigens for use in the present invention.

In various embodiments, the present agents, or methods using these agents, reduce or suppress autoreactive T cells. In some embodiments, the multi-specific Fc-based chimeric protein complex, optionally through an interferon signaling in the context of an Fc-based chimeric protein complex, causes this immunosuppression. In some embodiments, the multi-specific Fc-based chimeric protein complex stimulates PD-L1 or PD-L2 signaling and/or expression which may suppress autoreactive T cells. In some embodiments, the Fc-based chimeric protein complex, optionally through an interferon signaling in the context of an Fc-based chimeric protein complex, causes this immunosuppression. In some embodiments, the Fc-based chimeric protein complex stimulates PD-L1 or PD-L2 signaling and/or expression, which may suppress autoreactive T cells.

In various embodiments, the present methods comprise modulating the ratio of regulatory T cells to effector T cells in favor of immunosuppression, for instance, to treat autoimmune diseases. For instance, the present methods, in some embodiments, reduce and/or suppress one or more of cytotoxic T cells; effector memory T cells; central memory T cells; CD8⁺ stem cell memory effector cells; TH1 effector T-cells; TH2 effector T cells; TH9 effector T cells; TH17 effector T cells. For instance, the present methods, in some embodiments, increase and/or stimulate one or more of CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In some embodiments, the present methods favor immune inhibitory signals over immune stimulatory signals. In some embodiments, the present methods allow for reversing or suppressing immune activating or co-stimulatory signals. In some embodiments, the present methods allow for providing immune inhibitory signals. For instance, in some embodiments, the present agents and methods reduce the effects of an immune stimulatory signal, which, without limitation, is one or more of 4-1BB, OX-40, HVEM, GITR, CD27, CD28, CD30, CD40, ICOS ligand; OX-40 ligand, LIGHT (CD258), GITR ligand, CD70, B7-1, B7-2, CD30 ligand, CD40 ligand, ICOS, ICOS ligand, CD137 ligand and TL1A. Further, in some embodiments, the present agents and methods increase the effects of an immune inhibitory signal, which, without limitation, is one or more of CTLA-4, PD-L1, PD-L2, PD-1, BTLA, HVEM, TIM3, GAL9, LAG3, VISTA, KIR, 2B4, CD160 (also referred to as BY55), CGEN-15049, CHK 1 and CHK2 kinases, A2aR, CEACAM (e.g., CEACAM-1, CEACAM-3 and/or CEACAM-5), and various B-7 family ligands (including, but are not limited to, B7-1, B7-2, B7-DC, B7-H1, B7-H2, B7-H3, B7-H4, B7-H5, B7-H6 and B7-H7.

### Kits

The present invention also provides kits for the administration of any Fc-based chimeric protein complex described herein (e.g. with or without additional therapeutic agents). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to effect a desired therapeutic outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the 1050 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

In some Examples, two variants of the knob-in-hole technology are used: Ridgway (derived from Ridgway et al., Protein Engineering 1996; 9:617-621 and used e.g. in Examples 1-6) and Merchant (derived from Merchant et al., Nature Biotechnology 1998; 16:677-681 and used in most examples as of Example 7). Sequences are referred to as Fc1 and Fc2 (Ridgway hole and knob, respectively) and Fc3 and Fc4 (Merchant hole and knob, respectively). The ‘standard’ effector-mutation in the Ridgway constructs is LALA-PG (P329G), unless stated otherwise (see Example 4). For the Merchant constructs the LALA-KQ (K322Q) mutation is used (based on the data of Example 4) to this end.

Fc1: Ridgway hole: hIgG1 Fc_L234A_L235A_P329G_Y407T having the following amino acid sequence:
(SEQ ID NO: 1565)
DKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLTSKLTVDKSRWQQGNVFSC
SVMHEALHNHYTQKSLSLSPGK
Fc2: Ridgway knob: hIgG1 Fc_L234A_L235A_P329G_T366Y having the following amino acid sequence:
(SEQ ID NO: 1566)
DKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRDE
LTKNQVSLYCLVKGFYPSDIAVEWESNGQPENNYKTTPPVLDSDGSFFLYSKLTVDKSRWQQGNVFSC
SVMHEALHNHYTQKSLSLSPGK
Fc3: Merchant hole: hIgG1 Fc_L234A_L235A_K322Q_Y3490_T3665_L368A_Y407V having the following
amino acid sequence:
(SEQ ID NO: 1567)
DKTHTCPPCPAPEAAGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVCTLPPSRDE
LTKNQVSLSCAVKGFYPSDIAVEWESNGQPENNYKTTPPVLDSDGSFFLVSKLTVDKSRWQQGNVFS
CSVMHEALHNHYTQKSLSLSPGK
Fc4: Merchant knob: hIgG1 Fc_L234A_L235A_K322Q_53540_T366W having the following amino acid
sequence:
(SEQ ID NO: 1568)
DKTHTCPPCPAPEAAGGPSVFLFPPKPKDTLMISRTPEVTC\NVDVSHEDPEVKFNWYVDGVEVHNAK
TKPREEQYNSTYRVVSVLTVLHQDWLNGKEYKCKVSNKALPAPIEKTISKAKGQPREPQVYTLPPCRDE
LTKNQVSLWCLVKGFYPSDIAVEWESNGQPENNYKTTPPVLDSDGSFFLYSKLTVDKSRWQQGNVFS
CSVMHEALHNHYTQKSLSLSPGK

Throughout the Examples, two Clec9A VHHs are used: R1CHCL50 (SEQ ID NO: 289) and 3LEC89 (SEQ ID NO: 391). Both are used in the original sequence or in a mutated form as follows:

R1CHCL50_opt4: R1CHCL50_E1D-A74S-K83R-Q108L-H13Q-T64K (where E1D, A74S, K83R, Q108L,
H13Q, and T64K refer to mutations in R1CHCL50 VHH having the amino acid sequence of SEQ ID NO:
289). R1CHCL50_opt4 has the following amino acid sequence:
(SEQ ID NO: 1569)
DVQLVESGGGLVQPGGSLRLSCAASGSFSSINVMGWYRQAPGKERELVARITNLGLPNYADSVKGRF
TISRDNSKNTVYLQMNSLRPEDTAVYYCYLVALKAEYWGQGTLVTVSS
3LEC89_opt4: 3LE089_E1D-Q5V-A745-Q108L-G75K (where E1D, Q5V, A745, Q108L, and G75K refer
to mutations in 3LEC89 VHH having the amino acid sequence of SEQ ID NO: 391).
3LEC89_opt4 has the following amino acid sequence:
(SEQ ID NO: 1570)
DVQLVESGGGLVQPGGSLRLSCAASGRIFSVNAMGWYRQAPGKQRELVAAITNQGAPTYADSVKGRF
TISRDNSKNTVYLQMNSLRPEDTAVYYCKAFTRGDDYWGQGTLVTVSS

### Example 1: Fc-Based Chimeric Protein Complex Construction

Chimeric proteins with a targeting moiety and signaling agent were cloned into a human IgG1 Fc-fusion format mutated for reduced FcγR and C1q binding (LALA-PG mutations). A total of 5 combinations (see FIGS. 20A-E): two homodimeric and three heterodimeric (based on the knob-in-hole mutations; see Ridgway et al., Protein Engineering, Design and Selection, Volume 9, Issue 7, 1 Jul. 1996, Pages 617-621,) were constructed. In these fusion proteins the anti-human Clec9A VHH 3LEC89 was the targeting moiety and human IFNα2 with a R149A mutation was the signaling agent; these reagents were used for illustration of the general concept.

The relevant sequences (see FIGS. 20A-E for the identifiers) were:

P-956: pcDNA3.4-mouse light chain kappa-hIgG1-LALA-PG-20*GGS-3LEC89-20*GGS+G-IFNa2 R149A
(SEQ ID NO: 1435)
MKLPVRLLVLMFWIPASSSDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQ
VYTLPPSRDELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGGSGGSGGSGGSGGSGGSGGS
GGSGGSGGSGGSGGSGGSQVQLQESGGGLVQPGGSLRLSCAASGRIFSVNAMGWYRQAPGKQRELVAAITN
QGAPTYADSVKGRFTISRDNAGNTVYLQMNSLRPEDTAVYYCKAFTRGDDYWGQGTQVTVSSGGSGGSGGSG
GSGGSGGSGGSGGSGGSGGSGGSGGSGGSGGSGGSGGSGGSGGSGGSGGSGCDLPQTHSLGSRRTLMLL
AQMRKISLFSCLKDRHDFGFPQEEFGNQFQKAETIPVLHEMIQQIFNLFSTKDSSAAWDETLLDKFYTELYQQLN
DLEACVIGVGVTETPLMKEDSILAVRKYFRITLYLKEKKYSPCAWEVVRAEIMASFSLSTNLESLRSKE
P-957: pcDNA3.4-mouse Ig heavy chain-3LEC89-20*GGS-hIgG1-LALA-PG-20*GGS+G-IFNa2 R149A
(SEQ ID NO: 1436)
MGWSCIIFFLVATATGVHSQVQLQESGGGLVQPGGSLRLSCAASGRIFSVNAMGWYRQAPGKQRELVAAITNQ
GAPTYADSVKGRFTISRDNAGNTVYLQMNSLRPEDTAVYYCKAFTRGDDYWGQGTQVTVSSGGSGGSGGSGG
SGGSGGSGGSGGSGGSGGSGGSGGSGGSGGSGGSGGSGGSGGSGGSGGSDKTHTCPPCPAPEAAGGPS
VFLFPPKPKDILMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVYTLPPSRDELTKNQVSLICLVKGFYPSDIAVEWESNG
QPENNYKTTPPVLDSDGSFFLYSKLTVDKSRWQQGNVFSCSVMHEALHNHYTQKSLSLSPGKGGSGGSGGS
GGSGGSGGSGGSGGSGGSGGSGGSGGSGGSGGSGGSGGSGGSGGSGGSGGSGCDLPQTHSLGSRRTLML
LAQMRKISLFSCLKDRHDFGFPQEEFGNQFQKAETIPVLHEMIQQIFNLFSTKDSSAAWDETLLDKFYTELYQQLN
DLEACVIQGVGVTETPLMKEDSILAVRKYFQRITLYLKEKKYSPCAWEVVRAEIMASFSLSTNLQESLRSKE
P-958: pcDNA3.4-mouse Ig heavy chain-3LEC89-20*GGS-hIgG1-LALA-PG-YT (hole)
(SEQ ID NO: 1437)
MGWSCIIFFLVATATGVHSQVQLQESGGGLVQPGGSLRLSCAASGRIFSVNAMGWYRQAPGKQRELVAAITNQ
GAPTYADSVKGRFTISRDNAGNTVYLQMNSLRPEDTAVYYCKAFTRGDDYWGQGTQVTVSSGGSGGSGGSGG
SGGSGGSGGSGGSGGSGGSGGSGGSGGSGGSGGSGGSGGSGGSGGSGGSDKTHTCPPCPAPEAAGGPS
VFLFPPKPKDILMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVYTLPPSRDELTKNQVSLICLVKGFYPSDIAVEWESNG
QPENNYKTTPPVLDSDGSFFLTSKLTVDKSRWQQGNVFSCSVMHEALHNHYTQKSLSLSPGK
P-959: pcDNA3.4-mouse light chain kappa-hIgGl-LALA-PG-TY (knob)-20*GGS+G-IFNa2_R149A
(SEQ ID NO: 1438)
MKLPVRLLVLMFWIPASSSDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQ
VYTLPPSRDELTKNQVSLYCLVKGFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGGSGGSGGSGGSGGSGGSGGS
GGSGGSGGSGGSGGSGGSGCDLPQTHSLGSRRTLMLLAQMRKISLFSCLKDRHDFGFPQEEFGNQFQKAETIP
VLHEMIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
P-960: pcDNA3.4-mouse light chain kappa-hIgGl-LALA-PG-YT (hole)-20*GGS-3LEC89-20*GGS+G-
IFNa2_R149A
(SEQ ID NO: 1439)
MKLPVRLLVLMFWIPASSSDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQ
VYTLPPSRDELTKNQVSLTCLVKGFYPSDIAVEWESNGQPENNYKTTPPVLDSDGSFFLTSKLTVDKSRWQQG
NVFSCSVMHEALHNHYTQKSLSLSPGKGGSGGSGGSGGSGGSGGSGGSGGSGGSGGSGGSGGSGGSGGS
GGSGGSGGSGGSGGSGGSQVQLQESGGGLVQPGGSLRLSCAASGRIFSVNAMGWYRQAPGKQRELVAAITN
QGAPTYADSVKGRFTISRDNAGNTVYLQMNSLRPEDTAVYYCKAFTRGDDYWGQGTQVTVSSGGSGGSGGSG
GSGGSGGSGGSGGSGGSGGSGGSGGSGGSGGSGGSGGSGGSGGSGGSGGSGCDLPQTHSLGSRRTLMLL
AQMRKISLFSCLKDRHDFGFPQEEFGNQFQKAETIPVLHEMIQQIFNLFSTKDSSAAWDETLLDKFYTELYQQLN
DLEACVIQGVGVTETPLMKEDSILAVRKYFQRITLYLKEKKYSPCAWEVVRAEIMASFSLSTNLQESLRSKE
P-961: pcDNA3.4-mouse Ig heavy chain-3LEC89-20*GGS-hIgG1-LALA-PG-YT (hole)-20*GGS+G-
IFNa2_R149A
(SEQ ID NO: 1440)
MGWSCIIFFLVATATGVHSQVQLQESGGGLVQPGGSLRLSCAASGRIFSVNAMGWYRQAPGKQRELVAAITNQ
GAPTYADSVKGRFTISRDNAGNTVYLQMNSLRPEDTAVYYCKAFTRGDDYWGQGTQVTVSSGGSGGSGGSGG
SGGSGGSGGSGGSGGSGGSGGSGGSGGSGGSGGSGGSGGSGGSGGSGGSDKTHTCPPCPAPEAAGGPS
VFLFPPKPKDILMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVYTLPPSRDELTKNQVSLICLVKGFYPSDIAVEWESNG
QPENNYKTIPPVLDSDGSFFLTSKLTVDKSRWQQGNVFSCSVMHEALHNHYTQKSLSLSPGKGGSGGSGGS
GGSGGSGGSGGSGGSGGSGGSGGSGGSGGSGGSGGSGGSGGSGGSGGSGGSGCDLPQTHSLGSRRTLML
LAQMRKISLFSCLKDRHDFGFPQEEFGNQFQKAETIPVLHEMIQQIFLFSTKDSSAAWDETLLDKFYTELYQQLN
DLEACVIQGVGVTETPLMKEDSILAVRKYFQRITLYLKEKKYSPCAWEVVRAEIMASFSLSTNWESLRSKE
P-962: pcDNA3.4-mouse light chain kappa-hIgG1-LALA-PG-TY (knob)
(SEQ ID NO: 1441)
MKLPVRLLVLMFWIPASSSDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALGAPIEKTISKAKGQPREPQ
VYTLPPSRDELTKNQVSLYCLVKGFYPSDIAVEWESNGQPENNYKTTPPVLDSDGSFFLYSKLTVDKSRWQQG
NVFSCSVMHEALHNHYTQKSLSLSPGK

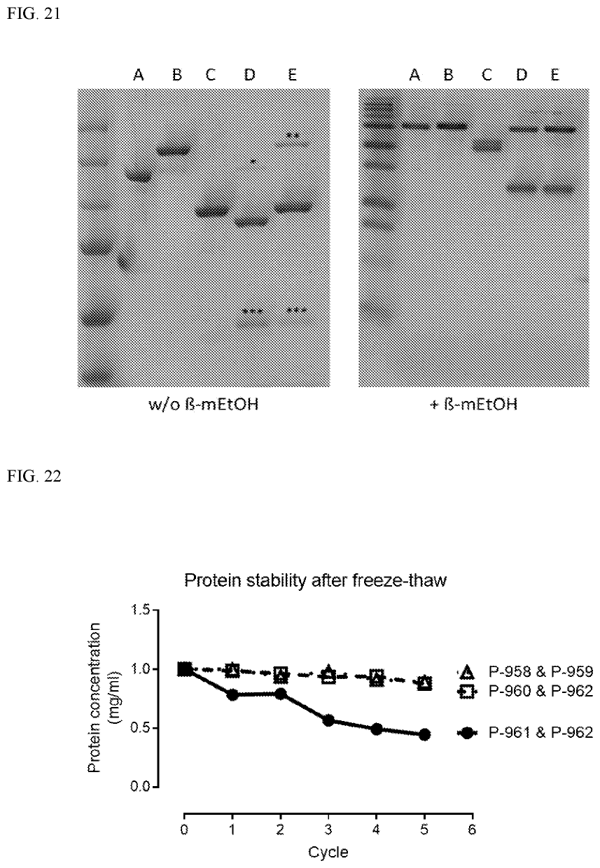

Different constructs were made by GeneArt (Thermo Fisher) and transiently expressed in the ExpiCHO expression system (Thermo Fisher) according to the manufacturer's guidelines. Ten days after transfection, supernatant was collected and cells removed by centrifugation. Recombinant proteins were purified from the medium using the rProtein A Sepharose Fast Flow resin (GE Healthcare) according to the manufacturer's guidelines. Average yields ranged from 70 to 200 mg per liter (see table below). SDS-PAGE under reducing (with added β-mercaptoethanol (β-mEtOH)) and non-reducing conditions (without β-mEtOH) (see FIG. 21, protein loaded in lanes A-E of the gels are described in table below) clearly showed that proteins are expressed as disulfide-linked complexes. Aberrant clustering (combination of two knob or two hole chains; extra bands on the gel, as shown by *, **, or *** in lanes D and E of gels without β-mEtOH) is observed in the heterodimeric Fc-based chimeric protein complexes D and E. Under reducing conditions, the two parts of the heterodimeric Fc-based chimeric protein complexes are nicely separated.

Heterodimeric Fc-based chimeric protein complexes appeared more soluble than the homodimeric proteins. In the latter case, even at concentrations below 1 mg/ml proteins tended to aggregate and precipitate. Protein melting analysis using SYPRO Orange (Sigma-Aldrich) on a LightCycler 480 System (Roche) illustrated that the melting temperatures of the different proteins were very comparable and ranged from 66 to 70° C.

| | | | | Yield (mg) per liter |
|---|---|---|---|---|
| Homodimeric | A | P-956 | Fc-3LEC89-IFNa2_R149A | 73, 2 |
| | B | P-957 | 3LEC89-Fc-IFNa2_R149A | 176, 8 |
| Heterodimeric | C | P-958 + P-959 | 3LEC89-Fc + Fc-IFNa2_R149A | 199, 2 |
| | D | P-960 + P-962 | Fc-3LEC89-IFNa2_R149A + Fc | 174, 8 |
| | E | P-961 + P-962 | 3LEC89-Fc-IFNa2_R149A + Fc | 156 |

To further estimate the stability of the proteins, the heterodimeric Fc-based chimeric protein complexes “C,” “D,” and “E” (see FIGS. 20A-E) were subjected to five cycles of freezing and thawing. Protein aggregates were removed by centrifugation just after thawing and protein concentrations were measured and plotted (see FIG. 22). Proteins C and D seemed more resistant to freezing-thawing than protein E.

### Example 2: Fc-Based Chimeric Protein Complex Characterization

Affinity of the heterodimeric Fc-based chimeric protein complexes for human Clec9A was determined using the bio-layer interferometry (BLI) technology on an Octet instrument (ForteBio). Recombinant Clec9A was biotinylated and trapped on Streptavidin sensors. Loaded tips were incubated with a serial dilution Fc-fusion. Shifts in the interference pattern were used to quantify the association and dissociation rates and thus the affinity. Data in the table below illustrates that the affinity is comparable in proteins C and E, while that in protein D was 10-fold lower.

| Sample ID | KD (M) | kon (1/Ms) | kdis (1/s) |
|---|---|---|---|
| 958 + 959 (C) | 2.61E−09 | 1.29E+05 | 3.37E−04 |
| 960 + 962 (D) | 3.16E−08 | 3.12E+04 | 9.88E−04 |
| 961 + 962 (E) | 3.88E−09 | 1.07E+05 | 4.16E−04 |

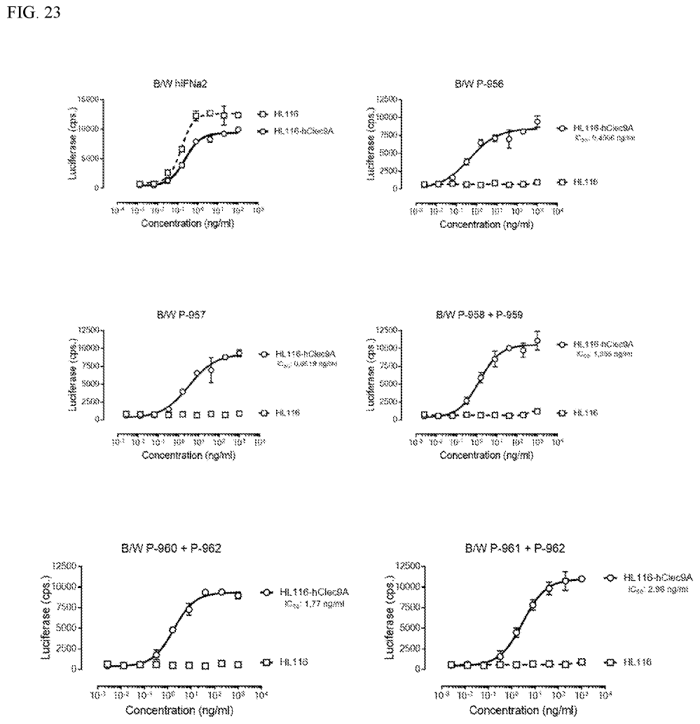

To study biological activity of the heterodimeric Fc-based chimeric protein complexes, 6-16 reporter activity was measured in parental and HL116-hClec9A cell-lines. The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well, before stimulation with a serial dilution of IFNα2 as a positive control and Fc AFNs for 6 hours. Luciferase activity was measured in cell lysates. Data in FIG. 23 illustrate that homodimeric Fc-based chimeric protein complexes are slightly more active than the heterodimeric Fc-based chimeric protein complexes. In all cases activity on targeted cells vs activity on parental cells was observed at over a 10,000-fold difference.

### Example 3: Pharmacokinetic Study in Mouse

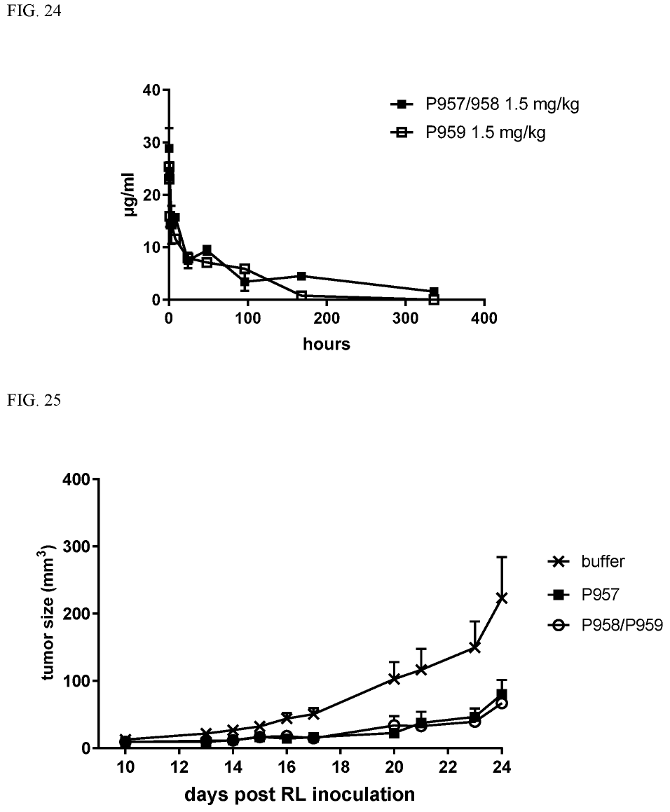

To evaluate the pharmacokinetic behavior of the Fc-based Actaferons (Fc-AFNs), the molecules P957 and P958/P959 (Example 1) were dosed intravenously at 1.5 mg/kg in 12 mice for each construct. K-EDTA blood was taken from a first group of 3 mice at 5 minutes, 8 hours and 2 days, from a second group of 3 mice at 15 minutes, 1 day and 4 days, from a third group of 3 mice at 1, 7 and 14 days and from a fourth group of 3 mice at 3 hours, 21 and 28 days. The concentration of intact Fc-AFN was measured by ELISA. In brief the MAXISORP Nunc Immune plates (Thermo Scientific) were coated overnight with anti-human interferon alpha mAb (clone MMHA-13; PBL Assay Science) at 0.5 μg/ml in PBS. After washing the plates four times with PBS+0.05% Tween-20, they were blocked with 0.1% Casein in PBS for at least 1 hour at room temperature. Subsequently, diluted samples and standards were incubated in 0.1% Casein in PBS for 2 hours at room temperature. After another wash cycle a custom made rabbit-anti-VHH (diluted 1/20000 in 0.1% Casein in PBS) was incubated for 2 hours at room temperature followed by an additional wash cycle and incubation with HRP-conjugated goat anti-rabbit (Jackson—111-035-144; 1:5000 in 0.1% Casein) for 1 hour at room temperature. After a final washing cycle, peroxidase activity was measured using KPL substrate (5120-0047; SeraCare) according to the manufacturer's instructions. Concentrations from samples were calculated using GraphPad Prism. Measured concentrations are plotted in FIG. 24. Terminal half-life was estimated on average at about 4.5 days P958/P959 and at about 3.25 days for P957.

### Example 4: Efficacy Study in Humanized Mouse

To evaluate the in vivo efficacy of Fc-based AFNs the molecules P957 and P958/P959 (Example 1; targeted to human CLEC9A, a highly specific conventional dendritic cell 1 (cDC1) marker) were tested in a tumor model in a humanized mouse. In brief, newborn NSG mice (1-2 days of age) were sublethal irradiated with 100 cGy prior to intrahepatic delivery of 1×10⁵CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of interferons (IFN)). Mice were treated daily intraperitoneally with 30 μg of human FMS-like tyrosine kinase 3 ligand (Flt3L) protein, from day 6 to day 17 after tumor inoculation. Weekly intravenous injection with buffer or Fc-AFN (25 μg) was initiated at day 10 after tumor inoculation, when a palpable tumor was visible (n=6 mice per group). Tumor size (caliper measurements), body weight and temperature were assessed daily. FIG. 25 shows the tumor growth until 7 days after the second treatment (mice received weekly injections with either PBS or AFN, the data show tumor growth up to 7 days after the second weekly treatment) and demonstrates that all constructs induced a similar level of tumor growth inhibition. Data on body weight and temperature did not show any major difference between buffer treatment and AFN treatment supporting that the AFN treatment was well tolerated.

### Example 5: Linker Lengths Heterodimeric Constructs

In the heterodimeric Fc AFN configuration of Examples 1 and 2, 20*GGS linkers between VHH and Fc as well as between Fc and IFN were used. The effect on different lengths of these linkers on biological activity (HL116-hClec9A reporter) or affinity for Clec9A in bio-layer interferometry (BLI) was evaluated.

In this and following Examples, various Fc variants are as follows (amino acids numbers refer to EU convention (PNAS, Edelman et al., 1969; 63 (1) 78-85)):
Fc: hIgG1 Fc_L234A_L235A_P329G (where L234A, L235A, and P329G are mutations in the hIgG1 Fc sequence)
Fc1: Ridgway hole: hIgG1 Fc_L234A_L235A_P329G_Y407T (where L234A, L235A, P329G, and Y407T are mutations in the hIgG1 Fc1 sequence)
Fc2: Ridgway knob: hIgG1 Fc_L234A_L235A_P329G_T366Y (where L234A, L235A, P329G and T366Y are mutations in the hIgG1 Fc sequence)

The leader sequences used for expression are identical as those in Example 1 and not further detailed in any of the constructs as of this Example.

The linkers used consist of a number of GGS repeats optionally followed by an additional single glycine residue.

They are referred to a nGGS where n represents the number of GGS repeats, the optional additional G is not separately indicated in the construct name.

### Constructs:

R1CHCL50_opt4-5*GGS-Fc1(P-1105)
(SEQ ID NO: 1442)
DVQLVESGGGLVQPGGSLRLSCAASGSFSSINVMGVVYRQAPGKERELVARITNLGLPNYADSVKGRFTIS
RDNSKNTVYLQMNSLRPEDTAVYYCYLVALKAEYWGQGTLVTVSSGGSGGSGGSGGSGGSDKTHTCPP
CPAPEAAGGPSVFLFPPKPKDTLMISRTPEVTCVVVDVSHEDPEVKFNVVYVDGVEVHNAKTKPREEQYNS
TYRVVSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
R1CHCL50_opt4-3*GGS-Fc1(P-1106)
(SEQ ID NO: 1443)
DVQLVESGGGLVQPGGSLRLSCAASGSFSSINVMGVVYRQAPGKERELVARITNLGLPNYADSVKGRFTIS
RDNSKNTVYLQMNSLRPEDTAVYYCYLVALKAEYWGQGTLVTVSSGGSGGSGGSDKTHTCPPCPAPEAA
GGPSVFLFPPKPKDTLMISRTPEVTCVVVDVSHEDPEVKFNVVYVDGVEVHNAKTKPREEQYNSTYRVVSV
LTVLHQDWLNGKEYKCKVSNKALGAPIEKTISKAKGQPREPQVYTLPPSRDELTKNQVSLTCLVKGFYPSDI
AVEWESNGQPENNYKTTPPVLDSDGSFFLTSKLTVDKSRWQQGNVFSCSVMHEALHNHYTQKSLSLSPG
K
3LEC89_opt4-5*GGS-Fc1(P-1107)
(SEQ ID NO: 1444)
DVQLVESGGGLVQPGGSLRLSCAASGRIFSVNAMGVVYRQAPGKQRELVAAITNQGAPTYADSVKGRFTIS
RDNSKNTVYLQMNSLRPEDTAVYYCKAFTRGDDYWGQGTLVTVSSGGSGGSGGSGGSGGSDKTHTCPP
CPAPEAAGGPSVFLFPPKPKDTLMISRTPEVTCVVVDVSHEDPEVKFNVVYVDGVEVHNAKTKPREEQYNS
TYRVVSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
3LEC89_opt4-3*GGS-Fc1(P-1108)
(SEQ ID NO: 1445)
DVQLVESGGGLVQPGGSLRLSCAASGRIFSVNAMGVVYRQAPGKQRELVAAITNQGAPTYADSVKGRFTIS
RDNSKNTVYLQMNSLRPEDTAVYYCKAFTRGDDYWGQGTLVTVSSGGSGGSGGSDKTHTCPPCPAPEA
AGGPSVFLFPPKPKDTLMISRTPEVTCVVVDVSHEDPEVKFNWYVDGVEVHNAKTKPREEQYNSTYRVVS
VLTVLHQDWLNGKEYKCKVSNKALGAPIEKTISKAKGQPREPQVYTLPPSRDELTKNQVSLTCLVKGFYPS
DIAVEWESNGQPENNYKTTPPVLDSDGSFFLTSKLTVDKSRWQQGNVFSCSVMHEALHNHYTQKSLSLSP
GK
Fc2-10*GGS-IFNa2_R149A(P-1109)
(SEQ ID NO: 1446)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRKISLFSCLKDRHDFGFPQEEFGNQFQKAETIPVLHEMIQQIFNLFSTKDSSAAWDETLLDKFYTELYQQL
NDLEACVIQGVGVTETPLMKEDSILAVRKYFQRITLYLKEKKYSPCAWEVVRAEIMASFSLSTNLQESLRSKE
Fc2-5*GGS-IFNa2_R149A(P-1110)
(SEQ ID NO: 1447)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGCDLPQTHSLGSRRTLMLLAQMRKISLFSCLKDRHDFG
FPQEEFGNQFQKAETIPVLHEMIQQIFNLFSTKDSSAAWDETLLDKFYTELYQQLNDLEACVIQGVGVTETP
LMKEDSILAVRKYFQRITLYLKEKKYSPCAWEVVRAEIMASFSLSTNLQESLRSKE
Fc2-3*GGS-IFNa2_R149A(P-1111)
(SEQ ID NO: 1448)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGCDLPQTHSLGSRRTLMLLAQMRKISLFSCLKDRHDFGFPQEEFG
NQFQKAETIPVLHEMIQQ1FNLFSTKDSSAAWDETLLDKFYTELYQQLNDLEACVIQGVGVTETPLMKEDSIL
AVRKYFQRITLYLKEKKYSPCAWEVVRAEIMASFSLSTNLQESLRSKE

### Production and Purification of Linker-Length Variants

Different constructs were made by GeneArt (Thermo Fisher) and combinations of hole (Fc1-based) and knob (Fc2-based) were combined to a heterodimeric configuration similar to the construct P958/959 of Example 1 as outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher). Variations in linker-lengths appeared not to affect the production (yields ranging from 100 to 300 mg per liter) and stability of the resulting proteins.

### Biological Activity on HL116 Reporter Cell Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well and subsequently stimulated with a serial dilution of Fc AFNs for 6 hours. Luciferase activity was measured in cell lysates. Data in the FIG. 26 and Table 6 illustrate that variations in linker-length had no major effects on biological activity in targeted cells (HL116-hClec9A cells). Of note the biological activity measured for any of the 3LEC89-5*GGS-Fc1 based constructs is also comparable to the biological activity measured for 3LEC89-20*GGS-Fc1/Fc2-20*GGS-IFNα2_R149A as measured in Example 2.

TABLE 6
Biological activity of linker-length variants on H L116 cells.
Summary of EC₅₀ (ng/ml) values of the biological
activity measured in FIG. 26.

| | EC₅₀ (ng/ml) | | | |
|---|---|---|---|---|
| | R1CHCL50- | R1CHCL50- | 3LEC89- | 3LEC89- |
| | 5*GGS-Fc1 | 3*GGS-Fc1 | 5*GGS-Fc1 | 3*GGS-Fc1 |
| Fc2-10*GGS- | 2.04 | 3.37 | 1.52 | 3.11 |
| IFNa2_R149A | | | | |
| Fc2-5*GGS | 1.89 | 4.33 | 1.14 | 3.22 |
| IFNa2_R149A | | | | |
| Fc2-3*GGS- | 2.42 | 3.63 | 1.15 | 2.35 |
| IFNa2_R149A | | | | |

Affinity for hClec9A

Affinity of the linker-length variants for human Clec9A was determined using the bio-layer interferometry (BLI) technology on an Octet instrument (ForteBio). Recombinant Clec9A was therefore biotinylated and trapped on a Streptavidin sensors (ForteBio). Loaded tips were incubated with a serial dilution Fc-fusions. In this experiment, 3LEC89 and R1CHCL50 hole constructs were combined with 5*GGS or 3*GGS linkers with the 10*GGS knob construct and compared with the original 3LEC89 configuration with two 20*GGS linkers. Shifts in the interference pattern were used to quantify the association and dissociation rates (and thus the affinity). Data, summarized in Table 7, show that shortening of the linkers does not have a negative impact on the affinity for hClec9A.

TABLE 7
Affinity of linker-length variants for hClec9A

| | KD (M) | KD Error | ka (1/Ms) | ka Error | kdis (1/s) | kdis Error |
|---|---|---|---|---|---|---|
| 3LEC89-20*GGS-Fc1 + | 7.50E−09 | 3.72E−11 | 1.07E+05 | 3.26E+02 | 8.00E−04 | 3.15E−06 |
| Fc2-20*GGS- | | | | | | |
| IFNa2_R149A | | | | | | |
| 3LEC89-5*GGS-Fc1 + | 8.42E+09 | 5.76E−11 | 1.23E+05 | 5.82E+02 | 1.00E−03 | 5.10E−06 |
| Fc2-10*GGS- | | | | | | |
| IFNa2_R149A | | | | | | |
| 3LEC89-3*GGS-Fc1 + | 3.12E−09 | 2.20E−11 | 1.67E+05 | 4.86E+02 | 5.00E−04 | 3.35E−06 |
| Fc2-10*GGS- | | | | | | |
| IFNa2_R149A | | | | | | |
| R1CHCL50-5*GGS-Fc1 + | 7.46E−09 | 3.76E−11 | 1.60E+05 | 5.76E+02 | 1.20E−03 | 4.20E−06 |
| Fc2-10*GGS- | | | | | | |
| IFNa2_R149A | | | | | | |
| R1CHCL50-3*GGS-Fc1 + | 1.20E−08 | 9.07E−11 | 1.39E+05 | 8.59E+02 | 1.70E−03 | 7.26E−06 |
| Fc2-10*GGS- | | | | | | |
| IFNa2_R149A | | | | | | |

### Example 6: Linker Lengths Homodimeric Constructs

In the homodimeric Fc AFN configuration of Examples 1 and 2, 20*GGS linkers between VHH and Fc as well as between Fc and IFN were used. The effect on different lengths of these linkers on biological activity (HL116-hClec9A reporter) was evaluated.

### Constructs:

3LEC89_opt4-10*GGS-Fc-10*GGS-IFNa2_R149A(P-1144)
(SEQ ID NO: 1449)
DVQLVESGGGLVQPGGSLRLSCAASGRIFSVNAMGWYRQAPGKQRELVAAITNQGAPTYADSVKGRFTIS
RDNSKNTVYLQMNSLRPEDTAVYYCKAFTRGDDYWGQGTLVTVSSGGSGGSGGSGGSGGSGGSGGSG
GSGGSGGSDKTHTCPPCPAPEAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALGAPIEKTISKAKGQPREPQVYTLPP
SRDELTKNQVSLTCLVKGFYPSDIAVEWESNGQPENNYKTTPPVLDSDGSFFLYSKLTVDKSRWQQGNVF
SCSVMHEALHNHYTQKSLSLSPGKGGSGGSGGSGGSGGSGGSGGSGGSGGSGGSGCDLPQTHSLGSR
RTLMLLAQMRKISLFSCLKDRHDFGFPQEEFGNQFQKAETIPVLHEMIQQIFNLFSTKDSSAAWDETLLDKF
YTELYQQLNDLEACVIQGVGVTETPLMKEDSILAVRKYFQRITLYLKEKKYSPCAWEVVRAEIMASFSLSTNL
QESLRSKE
3LEC89_opt4-5*GGS-Fc-5*GGS-IFNa2_R149A(P-1145)
(SEQ ID NO: 1450)
DVQLVESGGGLVQPGGSLRLSCAASGRIFSVNAMGWYRQAPGKQRELVAAITNQGAPTYADSVKGRFTIS
RDNSKNTVYLQMNSLRPEDTAVYYCKAFTRGDDYWGQGTLVTVSSGGSGGSGGSGGSGGSDKTHTCPP
CPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQ
KSLSLSPGKGGSGGSGGSGGSGGSGCDLPQTHSLGSRRTLMLLAQMRKISLFSCLKDRHDFGFPQEEFG
NQFQKAETIPVLHEMIQQIFNLFSTKDSSAAWDETLLDKFYTELYQQLNDLEACVIQGVGVTETPLMKEDSIL
AVRKYFQRITLYLKEKKYSPCAWEVVRAEIMASFSLSTNLQESLRSKE
3LEC89_opt4-3*GGS-Fc-3*GGS-IFNa2_R149A(P-1146)
(SEQ ID NO: 1451)
DVQLVESGGGLVQPGGSLRLSCAASGRIFSVNAMGWYRQAPGKQRELVAAITNQGAPTYADSVKGRFTIS
RDNSKNTVYLQMNSLRPEDTAVYYCKAFTRGDDYWGQGTLVTVSSGGSGGSGGSDKTHTCPPCPAPEA
AGGPSVFLFPPKPKDTLMISRTPEVTCVVVDVSHEDPEVKFNWYVDGVEVHNAKTKPREEQYNSTYRVVS
VLTVLHQDWLNGKEYKCKVSNKALGAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKSLSLSP
GKGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
3LE089_opt4-AAA-Fc-AAA+G-IFNa2_R149A(P-1147)
(SEQ ID NO: 1452)
DVQLVESGGGLVQPGGSLRLSCAASGRIFSVNAMGWYRQAPGKQRELVAAITNQGAPTYADSVKGRFTIS
RDNSKNTVYLQMNSLRPEDTAVYYCKAFTRGDDYWGQGTLVTVSSAAADKTHTCPPCPAPEAAGGPSVF
LFPPKPKDTLMISRTPEVTCVVVDVSHEDPEVKFNWYVDGVEVHNAKTKPREEQYNSTYRVVSVLTVLHQD
WLNGKEYKCKVSNKALGAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLSPGKAAAGC
DLPQTHSLGSRRTLMLLAQMRKISLFSCLKDRHDFGFPQEEFGNQFQKAETIPVLHEMIQQIFNLFSTKDSS
AAWDETLLDKFYTELYQQLNDLEACVIQGVGVTETPLMKEDSILAVRKYFQRITLYLKEKKYSPCAWEVVRA
EIMASFSLSTNLQESLRSKE

### Production and Purification of Linker-Length Variants

The different constructs were made by GeneArt (Thermo Fisher) similar to the construct P957 of Example 1 as outlined in FIG. 1A, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well and subsequently stimulated with a serial dilution of Fc AFNs for 6 hours. Luciferase activity was measured in cell lysates. Data in Table 8 illustrate that variations in linker-length had no major effects on biological activity in targeted cells (HL116-hClec9A cells).

TABLE 8
Biological activity of linker-length homodimeric variants on HL116 cells

| | EC₅₀ (ng/ml) |
|---|---|
| 3LEC89(opt4)-10*GGS-Fc-10*GGS-IFNa2_R149A | 1.77 |
| 3LEC89(opt4)-5*GGS-Fc-5*GGS-IFNa2_R149A | 2.80 |
| 3LEC89(opt4)-3*GGS-Fc-3*GGS-IFNa2_R149A | 2.33 |
| 3LEC89(0pt4)-AAA-Fc-AAA-IFNa2_R149A | 2.79 |

### Example 7: Alternative Effector Mutations

To diminish the effector functions, i.e. complement dependent cytotoxicity (CDC) or antibody dependent cellular cytotoxicity (ADCC), of the Fc domain, the L234A_L235A mutations which mainly affect Fc-gamma receptor binding were combined with four extra Fc effector-mutations (P329G, K322Q, K322A or P331S) to further reduce complement binding. These mutations were applied to the 3LEC89-based heterodimeric Fc format with 20*GGS linkers. Resulting proteins were tested for biological activity (HL116-hClec9A reporter) and complement binding (BLI; Octet).

### Constructs:

3LEC89-20*GGS-Fc1_P329G(P-958)
(SEQ ID NO: 1453)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCWVDVSHEDPEVKFNVVYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESNGQPENN
YKTTPPVLDSDGSFFLTSKLTVDKSRWQQGNVFSCSVMHEALHNHYTQKSLSLSPGK
Fc2_P329G-20*GGS-IFNa2_R149A(P-959)
(SEQ ID NO: 1454)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
3LEC89-20*GGS-Fc1_K322Q(P-1074)
(SEQ ID NO: 1455)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCWVDVSHEDPEVKFNVVYVDGVEVHNAKTKPREEQYNSTYRVVSVLTVLHQDWLNGKEY
KCQVSNKALPAPIEKTISKAKGQPREPQVYTLPPSRDELTKNQVSLTCLVKGFYPSDIAVEWESNGQPENN
YKTTPPVLDSDGSFFLTSKLTVDKSRWQQGNVFSCSVMHEALHNHYTQKSLSLSPGK
Fc2_K322Q-20*GGS-IFNa2_R149A(P-1077)
(SEQ ID NO: 1456)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYQCKVSNKALPAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
3LEC89-20*GGS-Fc1_K322A(P-1073)
(SEQ ID NO: 1457)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCAVSNKALPAPIEKTISKAKGQPREPQVYTLPPSRDELTKNQVSLTCLVKGFYPSDIAVEWESNGQPENNY
KTTPPVLDSDGSFFLTSKLTVDKSRWQQGNVFSCSVMHEALHNHYTQKSLSLSPGK
Fc2_K322A-20*GGS-IFNa2_R149A(P-1076)
(SEQ ID NO: 1458)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCAVSNKALPAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
3LEC89-20*GGS-Fc1_P331S(P-1075)
(SEQ ID NO: 1459)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCKVSNKALPASIEKTISKAKGQPREPQVYTLPPSRDELTKNQVSLTCLVKGFYPSDIAVEWESNGQPENNY
KTTPPVLDSDGSFFLTSKLTVDKSRWQQGNVFSCSVMHEALHNHYTQKSLSLSPGK
Fc2_P331S-20*GGS-IFNa2_R149A(P-1078)
(SEQ ID NO: 1460)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALPASIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE

### Production and Purification of Effector Mutations Variants

3LEC89-based Fc AFNs with different effector-mutations were produced in ExpiCHO cells as heterodimeric AFNs outlined in FIG. 7B, and as described in previous Examples. Recombinant proteins were purified from the supernatant using the Pierce Protein A spin plates (Thermo Fisher) according to the manufacturer's guidelines. No major difference in production yields (between 50 and 150 μg/ml) and stability were observed among the different mutants.

### Biological Activity on HL116 Reporter Cell Lines

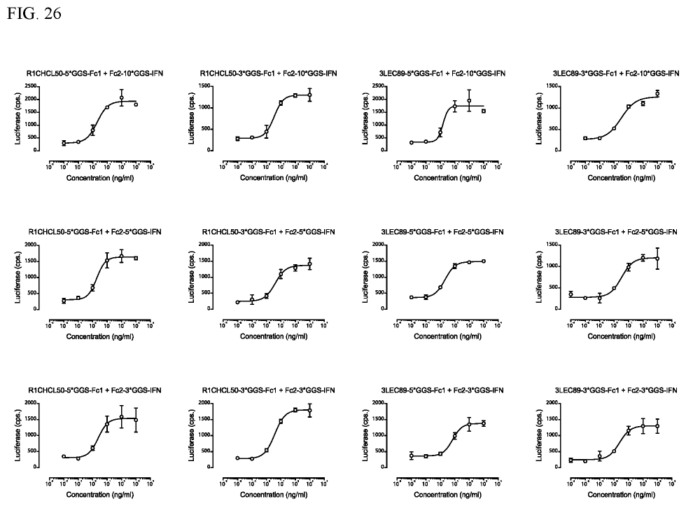

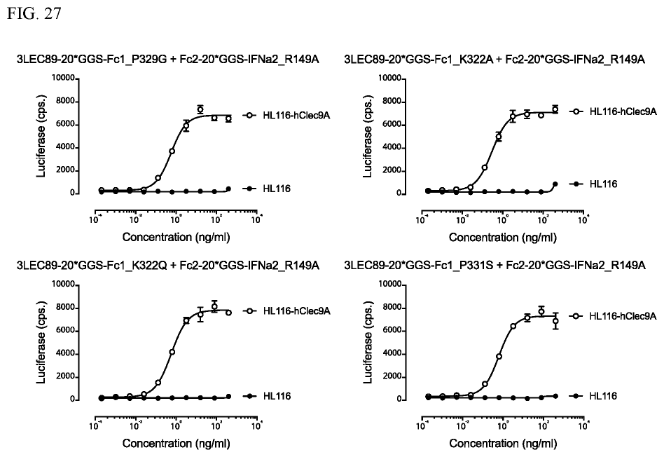

Biological activities of the alternative effector-mutants were measured as described in previous Examples. In brief, parental HL116 and the derived HL116-hClec9A were stimulated for 6 hours with a serial dilution of Fc-AFNs. Luciferase activity was measured and plotted in FIG. 27. Data clearly illustrate that resulting mutants show a very comparable biological activation profile on targeted (HL116-hClec9A) and untargeted (parental HL116) cells with EC₅₀ values varying from 0.3 to 0.65 ng/ml.

### Affinity for Complement (C1q)

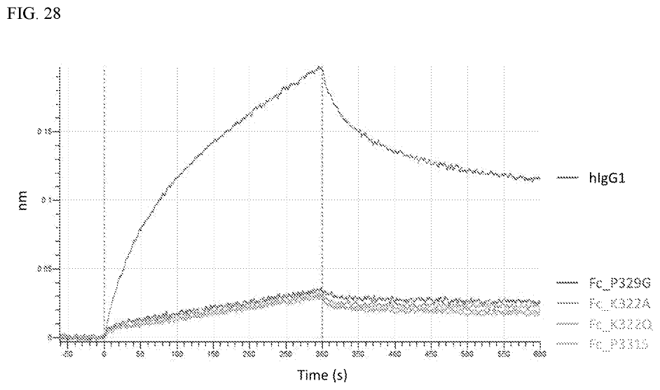

The effect of the different effector-mutations on the affinity for Complement Component C1 q (C1q) was determined using the bio-layer interferometry (BLI) technology on an Octet instrument (ForteBio). Fc AFNs with the different mutations or a hIgG1 as a positive control were loaded onto a Protein A bio-sensor (ForteBio) and subsequently incubated with recombinant C1q (100 nM; ProSpec). Data in FIG. 28 clearly illustrate that all effector-variants greatly lost their ability to bind to C1q, and that no significant differences between mutants could be observed.

### Example 8: IFNα2 Variants

Here, we evaluate the effect of mutation of the hIFNα2 O-glycosylation site on position 106 (T106) and compare biological activity of two natural IFNα2 variants: IFNα2A and IFNα2B.

### Constructs:

R1CHCL50_opt4-5*GGS-Fc1(P-1105)
(SEQ ID NO: 1461)
DVQLVESGGGLVQPGGSLRLSCAASGSFSSINVMGVVYRQAPGKERELVARITNLGLPNYADSVKGRFTIS
RDNSKNTVYLQMNSLRPEDTAVYYCYLVALKAEYWGQGTLVTVSSGGSGGSGGSGGSGGSDKTHTCPP
CPAPEAAGGPSVFLFPPKPKDTLMISRTPEVTCVVVDVSHEDPEVKFNVVYVDGVEVHNAKTKPREEQYNS
TYRWSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
3LEC89_opt4-5*GGS-Fc1(P-1077)
(SEQ ID NO: 1462)
DVQLVESGGGLVQPGGSLRLSCAASGRIFSVNAMGVVYRQAPGKQRELVAAITNQGAPTYADSVKGRFTIS
RDNSKNTVYLQMNSLRPEDTAVYYCKAFTRGDDYWGQGTLVTVSSGGSGGSGGSGGSGGSDKTHTCPP
CPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
Fc2-10*GGS-IFNa2A_R149A(P-1109)
(SEQ ID NO: 1463)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRKISLFSCLKDRHDFGFPQEEFGNQFQKAETIPVLHEMIQQIFNLFSTKDSSAAWDETLLDKFYTELYQQL
NDLEACVIQGVGVTETPLMKEDSILAVRKYFQRITLYLKEKKYSPCAWEVVRAEIMASFSLSTNLQESLRSKE
Fc2-10*GGS-IFNa2A_T106A-R149A(P-1302)
(SEQ ID NO: 1464)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRKISLFSCLKDRHDFGFPQEEFGNQFQKAETIPVLHEMIQQIFNLFSTKDSSAAWDETLLDKFYTELYQQL
NDLEACVIQGVGVAETPLMKEDSILAVRKYFQRITLYLKEKKYSPCAWEVVRAEIMASFSLSTNLQESLRSK
E
Fc2-10*GGS-IFNa2A_T106E-R149A(P-1303)
(SEQ ID NO: 1465)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRKISLFSCLKDRHDFGFPQEEFGNQFQKAETIPVLHEMIQQIFNLFSTKDSSAAWDETLLDKFYTELYQQL
NDLEACVIQGVGVEETPLMKEDSILAVRKYFQRITLYLKEKKYSPCAWEVVRAEIMASFSLSTNLQESLRSK
E
Fc2-10*GGS-IFNa2B_R149A(P-1304)
(SEQ ID NO: 1466)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRRISLFSCLKDRHDFGFPQEEFGNQFQKAETIPVLHEMIQQIFNLFSTKDSSAAWDETLLDKFYTELYQQL
NDLEACVIQGVGVTETPLMKEDSILAVRKYFQRITLYLKEKKYSPCAWEVVRAEIMASFSLSTNLQESLRSKE
Fc2-10*GGS-IFNa2B_T106A-R149A(P-1305)
(SEQ ID NO: 1467)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRRISLFSCLKDRHDFGFPQEEFGNQFQKAETIPVLHEMIQQIFNLFSTKDSSAAWDETLLDKFYTELYQQL
NDLEACVIQGVGVAETPLMKEDSILAVRKYFQRITLYLKEKKYSPCAWEVVRAEIMASFSLSTNLQESLRSK
E
Fc2-10*GGS-IFNa2B_T106E-R149A(P-1306)
(SEQ ID NO: 1468)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRRISLFSCLKDRHDFGFPQEEFGNQFQKAETIPVLHEMIQQIFNLFSTKDSSAAWDETLLDKFYTELYQQL
NDLEACVIQGVGVEETPLMKEDSILAVRKYFQRITLYLKEKKYSPCAWEVVRAEIMASFSLSTNLQESLRSK
E

### Production and Purification of IFNα2 Variants

R1CHCL50-5*GGS-Fc1 or 3LEC89-5*GGS-Fc1 were combined with the R149A mutant of IFNα2A or IFNα2B with or without mutated O-glycosylation site (T106, T106A or T106E) fused to Fc2 via a 10*GGS linker resulting in heterodimeric ActaFerons (AFNs) with a configuration outlined in FIG. 7B. Combinations were transiently expressed in the ExpiCHO expression system (Thermo Fisher) according to the manufacturer's guidelines. Recombinant proteins were purified from the supernatant using the Pierce Protein A spin plates (Thermo Fisher) according to the manufacturer's guidelines. Production-levels of different combinations were comparable and varied between 170 and 420 μg/ml. The loss of O-glycosylation was confirmed using SDS-PAGE.

### Biological Activity on HL116 Reporter Cell-Lines

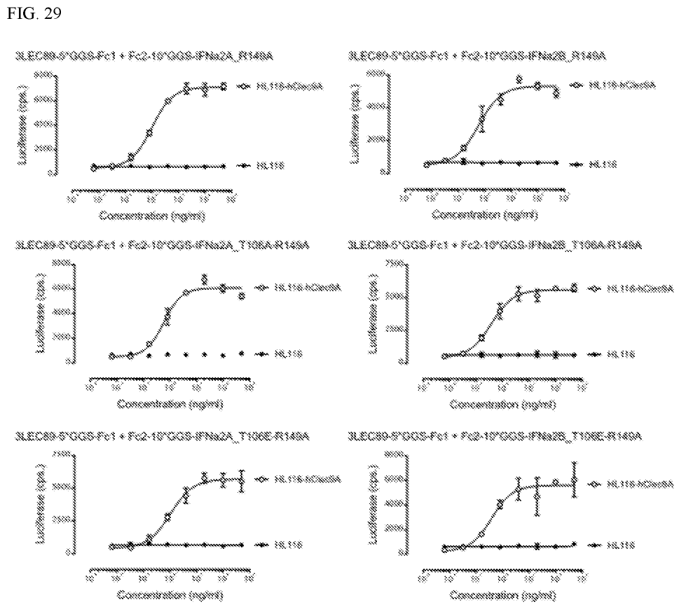

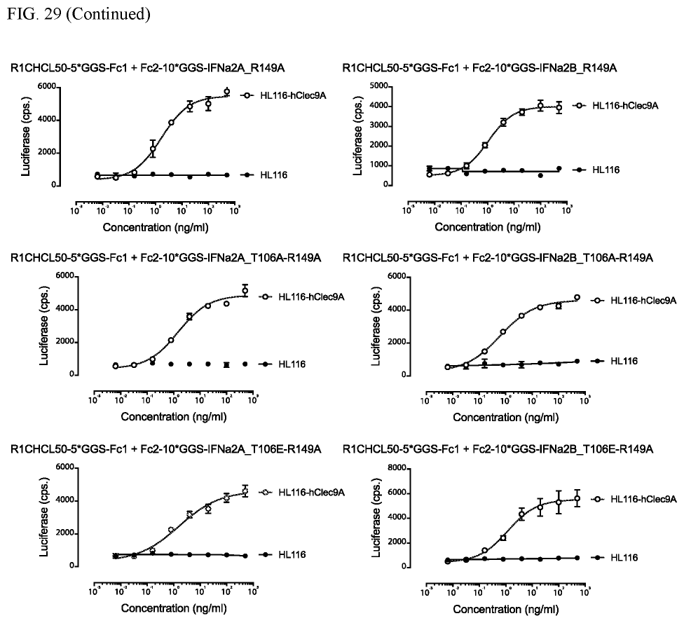

Biological activities of the alternative IFNα2 mutants were measured as described in previous Examples. In brief, parental HL116 and the derived HL116-hClec9A were stimulated for six hours with a serial dilution of Fc-AFNs. Luciferase activity was measured and plotted in FIG. 29 and summarized in Table 9. Data suggest that AFNs based on IFNa2B are only slightly more potent than those with IFNa2A, while mutation of the IFNα2 O-glycosylation site T106 (to A or E) does not affect signaling.

TABLE 9
Effect of IFNa2 mutations on biological
activity. Summary of EC₅₀ (ng/ml)
values of the biological activity
measured in FIG. 29

| | EC₅₀ (ng/ml) | | | |
|---|---|---|---|---|
| | 3LEC89 | | R1CHCL50 | |
| | IFNa2A | IFNa2B | IFNa2A | IFNa2B |
| wild type | 0.98 | 0.58 | 1.68 | 1.07 |
| T106A | 0.58 | 0.37 | 1.49 | 0.61 |
| T106E | 1.04 | 0.38 | 1.58 | 1.18 |

### Affinity for IFNAR2

Here the effect of the IFNa2 mutations on affinity for its receptor IFNAR2 in BLI was evaluated on an Octet instrument. R1CHCL50-based Fc variants with sequence variations in IFNa2 were loaded onto Protein A bio-sensors (Pall) and subsequently incubated with a serial dilution recombinant IFNAR2 (SinoBiological). Shifts in the interference pattern were used to quantify the association and dissociation rates and thus the affinity. These experiments showed that the mutations don't have a major impact on the affinity for IFNAR2 (data not shown).

### Example 9: Additional Fc Formats

In previous Examples, a heteromeric Fc format was used in which the VHH (R1CHCL50 or 3LEC89) is cloned N- and the IFNa2 mutant C-terminally of the Fc portion. In this Example, formats with VHH and IFNa2 mutant on the same side (N- or C-terminally) of the molecule were generated. Resulting AFNs were tested for biological activity (HL116-hClec9A reporter) and hClec9A affinity (bio-layer interferometry (BLI) technology on an Octet instrument).

### Constructs:

3LEC89-20*GGS-Fc1(P-958)
(SEQ ID NO: 1469)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCWVDVSHEDPEVKFNVVYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESNGQPENN
YKTTPPVLDSDGSFFLTSKLTVDKSRWQQGNVFSCSVMHEALHNHYTQKSLSLSPGK
Fc2-20*GGS-IFNa2_R149A(P-959)
(SEQ ID NO: 1470)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
R1CHCL50_opt4-20*GGS-Fc1(P-1213)
(SEQ ID NO: 1471)
DVQLVESGGGLVQPGGSLRLSCAASGSFSSINVMGVVYRQAPGKERELVARITNLGLPNYADSVKGRFTIS
RDNSKNTVYLQMNSLRPEDTAVYYCYLVALKAEYWGQGTLVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESNGQPENN
YKTTPPVLDSDGSFFLTSKLTVDKSRWQQGNVFSCSVMHEALHNHYTQKSLSLSPGK
IFNa2_R149A-20*GGS-Fc2(P-1214)
(SEQ ID NO: 1472)
CDLPQTHSLGSRRTLMLLAQMRKISLFSCLKDRHDFGFPQEEFGNQFQKAETIPVLHEMIQQIFNLFSTKDS
SAAWDETLLDKFYTELYQQLNDLEACVIQGVGVTETPLMKEDSILAVRKYFQRITLYLKEKKYSPCAWEVVR
AEIMASFSLSTNLQESLRSKEGGSGGSGGSGGSGGSGGSGGSGGSGGSGGSGGSGGSGGSGGSGGS
GGSGGSGGSGGSGGSDKTHTCPPCPAPEAAGGPSVFLFPPKPKDTLMISRTPEVTCVVVDVSHEDPEVKF
NVVYVDGVEVHNAKTKPREEQYNSTYRVVSVLTVLHQDWLNGKEYKCKVSNKALGAPIEKTISKAKGQPRE
PQVYTLPPSRDELTKNQVSLYCLVKGFYPSDIAVEWESNGQPENNYKTTPPVLDSDGSFFLYSKLTVDKSR
WQQGNVFSCSVMHEALHNHYTQKSLSLSPGK
Fc1-20*GGS-R1CHCL50_opt4(P-1215)
(SEQ ID NO: 1473)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLTCLVKGFYPSDIAVEWESNGQPENNYKTTPPVLDSDGSFFLTSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSDVQLVESGGGLVQPGGSLRLSCAASGSFSSINVMGWYRQAPGKERELVARITNLGLPN
YADSVKGRFTISRDNSKNTVYLQMNSLRPEDTAVYYCYLVALKAEYWGQGTLVTVSS
Fc1-20*GGS-3LEC89_opt4(P-1216)
(SEQ ID NO: 1474)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLTCLVKGFYPSDIAVEWESNGQPENNYKTTPPVLDSDGSFFLTSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSDVQLVESGGGLVQPGGSLRLSCAASGRIFSVNAMGWYRQAPGKQRELVAAITNQGAP
TYADSVKGRFTISRDNSKNTVYLQMNSLRPEDTAVYYCKAFTRGDDYWGQGTLVTVSS

### Production and Purification of Different Fc Formats

Hole (with the VHH C- or N-terminal) and knob (with the mutant IFNα2 C- or N-terminal) sequence were combined as follows:
3LEC89-20*GGS-Fc1+Fc2-20*GGS-IFNα2_R149A (configuration: FIG. 7B);
R1CHCL50-20*GGS-Fc1+Fc2-20*GGS-IFNα2_R149A (configuration: FIG. 7B);
3LEC89-20*GGS-Fc1+IFNα2_R149A-20*GGS-Fc2 (configuration: FIG. 7C);
R1CHCL50-20*GGS-Fc1+IFNa2_R149A-20*GGS-Fc2 (configuration: FIG. 7C);
Fc1-20*GGS-3LEC89+Fc2-20*GGS-IFNα2_R149A (configuration: FIG. 7D);
Fc1-20*GGS-R1CHCL50+Fc2-20*GGS-IFNα2_R149A (configuration: FIG. 7D);

These sequences were produced in ExpiCHO cells. Recombinant proteins were purified from the supernatant using the Pierce Protein A spin plates (Thermo Fisher) according to the manufacturer's guidelines. Yields of different Fc formats were comparable and ranged from 100 to 250 μg/ml.

### Biological Activity on HL116 Reporter Cell-Lines

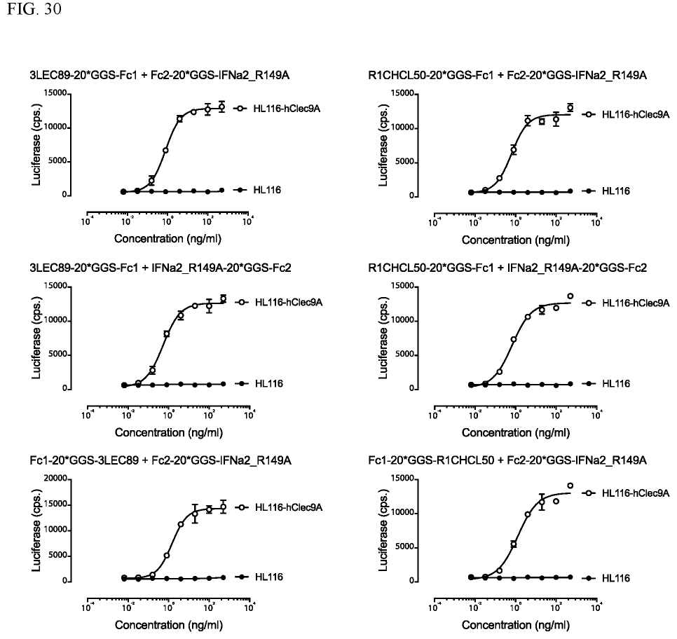

Biological activities of the different Fc-formats were measured as described in previous Examples. In brief, parental HL116 and the derived HL116-hClec9A were stimulated for 6 hours with a serial dilution of Fc-AFNs. Luciferase activity was measured and plotted in FIG. 30 and summarized in Table 10. Data suggest that Fc formats with both VHH and IFNα2_R149A cloned C-terminally are somewhat less active on targeted cells compared to the other two formats. Of note, no signaling was observed in the untargeted cells (parental HL116) for any of the constructs and hence all constructs retain a very high targeting index.

TABLE 10
Biological activity of different
Fc formats. Summary of EC₅₀ (ng/ml)
values of the biological activity
measured in FIG. 30.

| | EC₅₀ (ng/ml) | |
|---|---|---|
| | R1CHCL50 | 3LEC89 |
| VHH-20*GGS-Fc1 + Fc2- | 0.63 | 0.79 |
| 20*GGS-IFNa2_R149A | | |
| VHH-20*GGS-Fc1 + | 0.66 | 0.53 |
| IFNa2_R149A-20*GGS-Fc2 | | |
| Fc1-20*GGS-VHH + Fc2- | 1.28 | 1.47 |
| 20*GGS-IFNa2_R149A | | |

Affinity for hClec9A

Affinity of different Fc formats for human Clec9A was determined as described in Example 1. Data, summarized in Table 11, illustrate that differences in association and dissociation (and thus KD) between the different formats are only marginal.

TABLE 11
Affinity of different Fc formats for Clec9A

| | KD (M) | KD Error | kon (1/Ms) | kon Error | kdis (1/s) | kdis Error |
|---|---|---|---|---|---|---|
| 3LEC89-Fc + | 1.56E−09 | 1.17E−11 | 3.75E+05 | 1.22E+03 | 5.85E−04 | 3.92E−06 |
| Fc-IFN | | | | | | |
| R1CHCL50- | 2.55E−09 | 2.41E−11 | 3.12E+05 | 1.70E+03 | 7.96E−04 | 6.13E−06 |
| Fc + Fc-IFN | | | | | | |
| 3LEC89-Fc + | 1.61E−09 | 1.72E−11 | 2.85E+05 | 1.13E+03 | 4.61E−04 | 4.55E−06 |
| IFN-Fc | | | | | | |
| R1CHCL50- | 2.15E−09 | 2.39E−11 | 2.84E+05 | 1.54E+03 | 6.09E−04 | 5.92E−06 |
| Fc + IFN-Fc | | | | | | |
| Fc-3LEC89 + | 3.23E−09 | 2.77E−11 | 2.85E+05 | 1.55E+03 | 9.19E−04 | 6.13E−06 |
| Fc-IFN | | | | | | |
| Fc-R1CHCL50 + | 1.83E−09 | 1.48E−11 | 2.96E+05 | 1.02E+03 | 5.42E−04 | 3.95E−06 |
| Fc-IFN | | | | | | |

### Example 10: Comparison of Two Different Knob-in-Hole (KiH) Mutations

Here, two variants of the knob-in-hole technology: Ridgway (as used in previous Examples) and Merchant were compared. Sequences are referred to as Fc1 and Fc2 (Ridgway hole and knob, respectively) and Fc3 and Fc4 (Merchant hole and knob, respectively). Fc constructs, with a configuration outlined in FIG. 7B, with both KiHs were made based on VHHs R1CHCL50 and 3LEC89.
Fc1: Ridgway hole: hIgG1 Fc_L234A_L235A_P329G_Y407T
Fc2: Ridgway knob: hIgG1 Fc_L234A_L235A_P329G_T366Y
Fc3: Merchant hole: hIgG1 Fc_L234A_L235A_K322Q_Y349C_T366S_L368A_Y407V
Fc4: Merchant knob: hIgG1 Fc_L234A_L235A_K322Q_S354C_T366W

### Constructs:

3LEC89-20*GGS-Fc1(P-958)
(SEQ ID NO: 1475)
QVQLQESGGGLVQPGGSLRLSCAASGRIFSVNAMGVVYRQAPGKQRELVAAITNQGAPTYADSVKGRFTIS
RDNAGNTVYLQMNSLRPEDTAVYYCKAFTRGDDYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCWVDVSHEDPEVKFNVVYVDGVEVHNAKTKPREEQYNSTYRVVSVLTVLHQDWLNGKEY
KCKVSNKALGAPIEKTISKAKGQPREPQVYTLPPSRDELTKNQVSLTCLVKGFYPSDIAVEWESNGQPENN
YKTTPPVLDSDGSFFLTSKLTVDKSRWQQGNVFSCSVMHEALHNHYTQKSLSLSPGK
Fc2-20*GGS-IFNa2_R149A(P-959)
(SEQ ID NO: 1476)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCKVSNKALGAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGGSGGSGGSGGSGGSGGSG
GSGGSGGSGGSGCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVTETPLMKEDSILAVRKYFQRITLYLK
EKKYSPCAWEVVRAEIMASFSLSTNLQESLRSKE
R1CHCL50_opt4-5*GGS-Fc1(P-1105)
(SEQ ID NO: 1477)
DVQLVESGGGLVQPGGSLRLSCAASGSFSSINVMGVVYRQAPGKERELVARITNLGLPNYADSVKGRFTIS
RDNSKNTVYLQMNSLRPEDTAVYYCYLVALKAEYWGQGTLVTVSSGGSGGSGGSGGSGGSDKTHTCPP
CPAPEAAGGPSVFLFPPKPKDTLMISRTPEVTCVVVDVSHEDPEVKFNVVYVDGVEVHNAKTKPREEQYNS
TYRVVSVLTVLHQDWLNGKEYKCKVSNKALGAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
3LEC89-20*GGS-Fc3(P-1411)
(SEQ ID NO: 1478)
QLQESGGGLVQPGGSLRLSCAASGRIFSVNAMGWYRQAPGKQRELVAAITNQGAPTYADSVKGRFTISRD
NAGNTVYLQMNSLRPEDTAVYYCKAFTRGDDYWGQGTQVTVSSGGSGGSGGSGGSGGSGGSGGSGGS
GGSGGSGGSGGSGGSGGSGGSGGSGGSGGSGGSGGSDKTHTCPPCPAPEAAGGPSVFLFPPKPKDTL
MISRTPEVTCWVDVSHEDPEVKFNVVYVDGVEVHNAKTKPREEQYNSTYRVVSVLTVLHQDWLNGKEYKC
QVSNKALPAPIEKTISKAKGQPREPQVCTLPPSRDELTKNQVSLSCAVKGFYPSDIAVEWESNGQPENNYK
TTPPVLDSDGSFFLVSKLTVDKSRWQQGNVFSCSVMHEALHNHYTQKSLSLSPGK
Fc4-20*GGS-IFNa2_R149A(P-1414)
(SEQ ID NO: 1479)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCQVSNKALPAPIEKTISKAKGQPREPQVYTLPPCRDELTKN
QVSLWCLVKGFYPSDIAVEWESNGQPENNYKTTPPVLDSDGSFFLYSKLTVDKSRWQQGNVFSCSVMHE
ALHNHYTQKSLSLSPGKGGSGGSGGSGGSGGSGGSGGSGGSGGSGGSGGSGGSGGSGGSGGSGGS
GGSGGSGGSGGSCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVEETPLMKEDSILAVRKYFQRITLYLK
EKKYSPCAWEVVRAEIMASFSLSTNLQESLRSKE
R1CHCL50-Fc3-20*GGS-IFNa2_R149A(P-1451)
(SEQ ID NO: 1480)
QVQLVESGGGLVHPGGSLRLSCAASGSFSSINVMGVVYRQAPGKERELVARITNLGLPNYADSVTGRFTIS
RDNAKNTVYLQMNSLKPEDTAVYYCYLVALKAEYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCQVSNKALPAPIEKTISKAKGQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKSLSLSPGK

### Production and Purification of Different Fc Formats

Combinations of knob and hole constructs were produced in ExpiCHO cells as described above. Recombinant proteins were purified from the supernatant using the Pierce Protein A spin plates (Thermo Fisher) according to the manufacturer's guidelines. Use of different knob in holes (KiH's) did not have a major influence on production levels, which varied from 250 to 400 μg/ml.

### Biological Activity on HL116 Reporter Cell-Lines

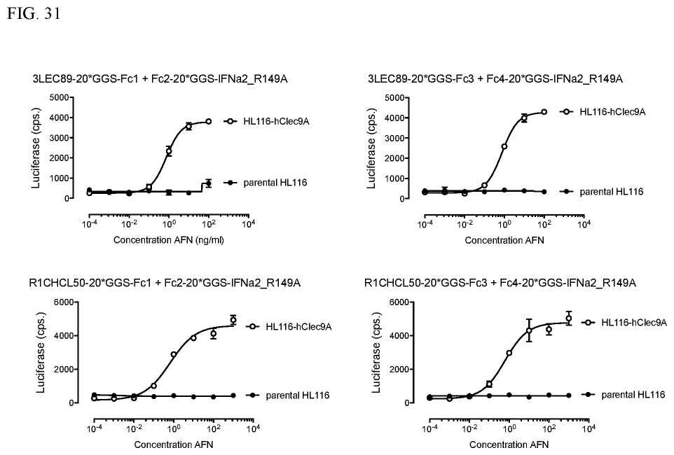

Biological activities of the different KiH Fc AFNs were measured as described in previous Examples. In brief, parental HL116 and the derived HL116-hClec9A were stimulated for six hours with a serial dilution of Fc-AFNs. Luciferase activity was measured and plotted in FIG. 31. Data suggest that the different KiH variations do not affect biological activity in targeted cells (i.e. Clec9A expressing) significantly.

### Example 11: Mono- Vs. Bi-Valent Targeted Fc Formats

In this Example, the biological activity and relative binding of a mono- and bivalent targeted (i.e. molecules with one or two VHHs, respectively) format of R1CHCL50 Fc AFN was compared.

### Constructs:

R1CHCL50-20*GGS-Fc3(P-1451)
(SEQ ID NO: 1481)
QVQLVESGGGLVHPGGSLRLSCAASGSFSSINVMGVVYRQAPGKERELVARITNLGLPNYADSVTGRFTIS
RDNAKNTVYLQMNSLKPEDTAVYYCYLVALKAEYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCQVSNKALPAPIEKTISKAKGQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKSLSLSPGK
Fc4-20*GGS-IFNa2_R149A(P-1414)
(SEQ ID NO: 1482)
DKTHTCPPCPAPEAAGGPSVFLFPPKPKDTLMISRTPEVTCVVVDVSHEDPEVKFNWYVDGVEVHNAKTK
PREEQYNSTYRWSVLTVLHQDWLNGKEYKCQVSNKALPAPIEKTISKAKGQPREPQVYTLPPCRDELTKN
QVSLWCLVKGFYPSDIAVEWESNGQPENNYKTTPPVLDSDGSFFLYSKLTVDKSRWQQGNVFSCSVMHE
ALHNHYTQKSLSLSPGKGGSGGSGGSGGSGGSGGSGGSGGSGGSGGSGGSGGSGGSGGSGGSGGS
GGSGGSGGSGGSCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVEETPLMKEDSILAVRKYFQRITLYLK
EKKYSPCAWEVVRAEIMASFSLSTNLQESLRSKE
R1CHCL50-20*GGS-Fc4-20*GGS-IFNa2_R149A(P-1459)
(SEQ ID NO: 1483)
QVQLVESGGGLVHPGGSLRLSCAASGSFSSINVMGVVYRQAPGKERELVARITNLGLPNYADSVTGRFTIS
RDNAKNTVYLQMNSLKPEDTAVYYCYLVALKAEYWGQGTQVTVSSGGSGGSGGSGGSGGSGGSGGSG
GSGGSGGSGGSGGSGGSGGSGGSGGSGGSGGSGGSGGSDKTHTCPPCPAPEAAGGPSVFLFPPKPKD
TLMISRTPEVTCVVVDVSHEDPEVKFNVVYVDGVEVHNAKTKPREEQYNSTYRVVSVLTVLHQDWLNGKEY
KCQVSNKALPAPIEKTISKAKGQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKSLSLSPGKGGSGGSGGSGGS
GGSGGSGGSGGSGGSGGSGGSGGSGGSGGSGGSGGSGGSGGSGGSGGSCDLPQTHSLGSRRTLMLL
AQMRKISLFSCLKDRHDFGFPQEEFGNQFQKAETIPVLHEMIQQIFNLFSTKDSSAAWDETLLDKFYTELYQ
QLNDLEACVIQGVGVEETPLMKEDSILAVRKYFQRITLYLKEKKYSPCAWEVVRAEIMASFSLSTNLQESLR
SKE

### Production and Purification of Different Fc Formats

A mono- (schematic: FIG. 7B) or bivalent (schematic: FIG. 16A) targeted variants of R1CHCL50 Fc AFN was made by combining the corresponding knob and a hole constructs and express them in ExpiCHO cells as described above. Recombinant proteins were purified from the supernatant using the Pierce Protein A spin plates (Thermo Fisher) according to the manufacturer's guidelines.

### Biological Activity on HL116 Reporter Cell-Lines

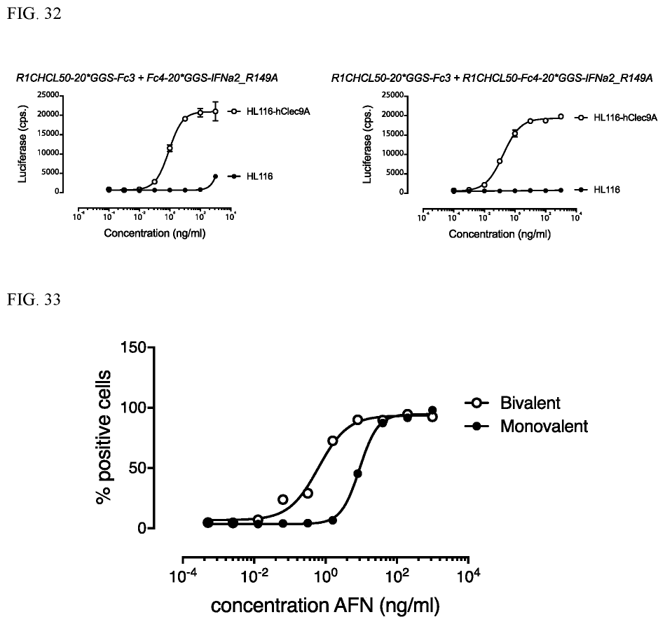

Biological activities of mono- and bivalent targeted Fc AFNs were measured as described in previous Examples. In brief, parental HL116 and the derived HL116-hClec9A were stimulated for six hours with a serial dilution of Fc-AFNs. Luciferase activity was measured and plotted in FIG. 32. Data show that the bivalent targeted AFN is approximately five-fold more active on target cells (HL116-hClec9A). Signaling in non-target cells (parental H L116 cells) was comparable for both variants.

### Relative Affinity in FACS

To measure relative affinities for Clec9A of the mono- and bivalent targeted Fc AFNs, HL116-hClec9A cells were incubated with a serial dilution of AFN. Binding was detected by subsequent incubation with an FITC-coupled anti-human secondary Ab, measured on a MACSQuant X instrument (Miltenyi Biotech) and analysed using the FlowLogic software (Miltenyi Biotech). Data in FIG. 33 illustrates that the bivalent targeted AFN has an about 15-fold lower binding EC50 when compared to the monovalent variant.

### Example 12: Variant Bivalent Targeted Fc Formats

In this Example, additional bivalent but heterodimeric variant Fc formats were generated and the biological activity was tested.

### Constructs:

R1CHCL50-20*GGS-R1CHCL50-20*GGS-Fc3
(SEQ ID NO: 1484)
QVQLVESGGGLVHPGGSLRLSCAASGSFSSINVMGVVYRQAPGKERELVARITNLGLPNYADSVTGRFTIS
RDNAKNTVYLQMNSLKPEDTAVYYCYLVALKAEYWGQGTQVTVSSGGSGGSGGSGGSGGSGGSGGSG
GSGGSGGSGGSGGSGGSGGSGGSGGSGGSGGSGGSGGSQVQLVESGGGLVHPGGSLRLSCAASGSF
SSINVMGVVYRQAPGKERELVARITNLGLPNYADSVTGRFTISRDNAKNTVYLQMNSLKPEDTAVYYCYLVA
LKAEYWGQGTQVTVSSGGSGGSGGSGGSGGSGGSGGSGGSGGSGGSGGSGGSGGSGGSGGSGGSG
GSGGSGGSGGSDKTHTCPPCPAPEAAGGPSVFLFPPKPKDTLMISRTPEVTCVVVDVSHEDPEVKFNVVYV
DGVEVHNAKTKPREEQYNSTYRVVSVLTVLHQDWLNGKEYKCQVSNKALPAPIEKTISKAKGQPREPQVC
TLPPSRDELTKNQVSLSCAVKGFYPSDIAVEWESNGQPENNYKTTPPVLDSDGSFFLVSKLTVDKSRWQQ
GNVFSCSVMHEALHNHYTQKSLSLSPGK
Fc4-20*GGS-IFNa2_R149A(P-1414)
(SEQ ID NO: 1485)
DKTHTCPPCPAPEAAGGPSVFLFPPKPKDTLMISRTPEVTCVWDVSHEDPEVKFNWYVDGVEVHNAKTK
PREEQYNSTYRWSVLTVLHQDWLNGKEYKCQVSNKALPAPIEKTISKAKGQPREPQVYTLPPCRDELTKN
QVSLWCLVKGFYPSDIAVEWESNGQPENNYKTTPPVLDSDGSFFLYSKLTVDKSRWQQGNVFSCSVMHE
ALHNHYTQKSLSLSPGKGGSGGSGGSGGSGGSGGSGGSGGSGGSGGSGGSGGSGGSGGSGGSGGS
GGSGGSGGSGGSCDLPQTHSLGSRRTLMLLAQMRKISLFSCLKDRHDFGFPQEEFGNQFQKAETIPVLHE
MIQQIFNLFSTKDSSAAWDETLLDKFYTELYQQLNDLEACVIQGVGVEETPLMKEDSILAVRKYFQRITLYLK
EKKYSPCAWEVVRAEIMASFSLSTNLQESLRSKE

### Production and Purification of Different Fc Formats

The bivalent (schematic: FIG. 8B) targeted variant of our R1CHCL50 Fc AFN was made by combining the corresponding knob and a hole constructs and expressing them in ExpiCHO cells as described above. Recombinant proteins were purified from the supernatant using the Pierce Protein A spin plates (Thermo Fisher) according to the manufacturer's guidelines.

### Biological Activity on HL116 Reporter Cell-Lines

The biological activity of the bivalent targeted Fc AFNs was measured as described in previous Examples. In brief, parental HL116 and the derived HL116-hClec9A were stimulated for six hours with a serial dilution of Fc-AFNs.

### Example 13: Pharmacokinetic Study in Mouse

To evaluate the pharmacokinetic behavior of the Fc-based AFNs, 4 different molecules were selected all carrying the K322Q mutation:
R1CHCL50(opt)4 combined with IFNα2_R149A using a Ridgway KiH Fc with LALA-K322Q mutation (combining construct 1 and 2)
R1CHCL50(opt)4 combined with IFNα2_R149A-T106E using a Ridgway KiH Fc with LALA-K322Q mutation (combining construct 1 and 3)
R1CHCL50(opt)4 combined with IFNα2_R149A using a Merchant KiH Fc with LALA-K322Q mutation (combining construct 4 and 5)
R1CHCL50(opt)4 combined with IFNα2_R149A-T106E using a Merchant KiH Fc with LALA-K322Q mutation (combining construct 5 and 6)

### Constructs:

1. R1CHCL50(opt4)-5*GGS-Fc1 (P329; K322Q) (P-1477)
(SEQ ID NO: 1486)
DVQLVESGGGLVQPGGSLRLSCAASGSFSSINVMGWYRQAPGKERELVARITNLGLPNYADSVKGRFTIS
RDNSKNTVYLQMNSLRPEDTAVYYCYLVALKAEYWGQGTLVTVSSGGSGGSGGSGGSGGSDKTHTCPP
CPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCQVSNKALPAPIEKTISKAKGQPREPQVYTLPPSRDELTKNQVSLTCLV
KGFYPSDIAVEWESNGQPENNYKTTPPVLDSDGSFFLTSKLTVDKSRWQQGNVFSCSVMHEALHNHYTQ
KSLSLSPGK
2. Fc2 (P329; K322Q)-10*GGS-IFNa2_R149A (P-1481)
(SEQ ID NO: 1487)
DKTHTCPPCPAPEAAGGPSVFLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRRISLFSCLKDRHDFGFPQEEFGNQFQKAETIPVLHEMIQQIFNLFSTKDSSAAWDETLLDKFYTELYQQL
NDLEACVIQGVGVTETPLMKEDSILAVRKYFQRITLYLKEKKYSPCAWEVVRAEIMASFSLSTNLQESLRSKE
3. Fc2 (P329; K322Q)-10*GGS-IFNa2_R149A_T106E (P-1482)
(SEQ ID NO: 1488)
DKTHTCPPCPAPEAAGGPSVFLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAKGQPREPQVYTLPPSRDELTKN
QVSLYCLVKGFYPSDIAVEWESNGQPENNYKTTPPVLDSDGSFFLYSKLTVDKSRWQQGNVFSCSVMHEA
LHNHYTQKSLSLSPGKGGSGGSGGSGGSGGSGGSGGSGGSGGSGGSGCDLPQTHSLGSRRTLMLLAQ
MRRISLFSCLKDRHDFGFPQEEFGNQFQKAETIPVLHEMIQQIFNLFSTKDSSAAWDETLLDKFYTELYQQL
NDLEACVIQGVGVEETPLMKEDSILAVRKYFQRITLYLKEKKYSPCAWEVVRAEIMASFSLSTNLQESLRSK
E
4. RICHCL50(opt4)-5*GGS-Fc3 (P-1479)
(SEQ ID NO: 1489)
DVQLVESGGGLVQPGGSLRLSCAASGSFSSINVMGWYRQAPGKERELVARITNLGLPNYADSVKGRFTIS
RDNSKNTVYLQMNSLRPEDTAVYYCYLVALKAEYWGQGTLVTVSSGGSGGSGGSGGSGGSDKTHTCPP
CPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCQVSNKALPAPIEKTISKAKGQPREPQVCTLPPSRDELTKNQVSLSCAV
KGFYPSDIAVEWESNGQPENNYKTTPPVLDSDGSFFLVSKLTVDKSRWQQGNVFSCSVMHEALHNHYTQ
KSLSLSPGK
5. Fc4-10*GGS-IFNa2_R149A (P-1483)
(SEQ ID NO: 1490)
DKTHTCPPCPAPEAAGGPSVFLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAKGQPREPQVYTLPPCRDELTKN
QVSLWCLVKGFYPSDIAVEWESNGQPENNYKTTPPVLDSDGSFFLYSKLTVDKSRWQQGNVFSCSVMHE
ALHNHYTQKSLSLSPGKGGSGGSGGSGGSGGSGGSGGSGGSGGSGGSGCDLPQTHSLGSRRTLMLLA
QMRRISLFSCLKDRHDFGFPQEEFGNQFQKAETIPVLHEMIQQIFNLFSTKDSSAAWDETLLDKFYTELYQQ
LNDLEACVIQGVGVTETPLMKEDSILAVRKYFQRITLYLKEKKYSPCAWEVVRAEIMASFSLSTNLQESLRSK
E
6. Fc4-10*GGS-IFNa2_R149A_T106E (P-1484)
(SEQ ID NO: 1491)
DKTHTCPPCPAPEAAGGPSVFLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAKGQPREPQVYTLPPCRDELTKN
QVSLWCLVKGFYPSDIAVEWESNGQPENNYKTTPPVLDSDGSFFLYSKLTVDKSRWQQGNVFSCSVMHE
ALHNHYTQKSLSLSPGKGGSGGSGGSGGSGGSGGSGGSGGSGGSGGSGCDLPQTHSLGSRRTLMLLA
QMRRISLFSCLKDRHDFGFPQEEFGNQFQKAETIPVLHEMIQQIFNLFSTKDSSAAWDETLLDKFYTELYQQ
LNDLEACVIQGVGVEETPLMKEDSILAVRKYFQRITLYLKEKKYSPCAWEVVRAEIMASFSLSTNLQESLRSK
E

### Production and Purification of Different Fc Formats

Production was performed in ExpiCHO cells as described above. Recombinant proteins were purified from the supernatant on a HiTrap Protein A HP (GE Healthcare) and eluted proteins were, after neutralization, desalted on a G25 column (GE Healthcare) followed by final and 0.22 μm filtration.

### Biological Activity on HL116 Reporter Cell-Lines

Biological activities of purified Fc-AFNs were measured as described in previous Examples. In brief, parental

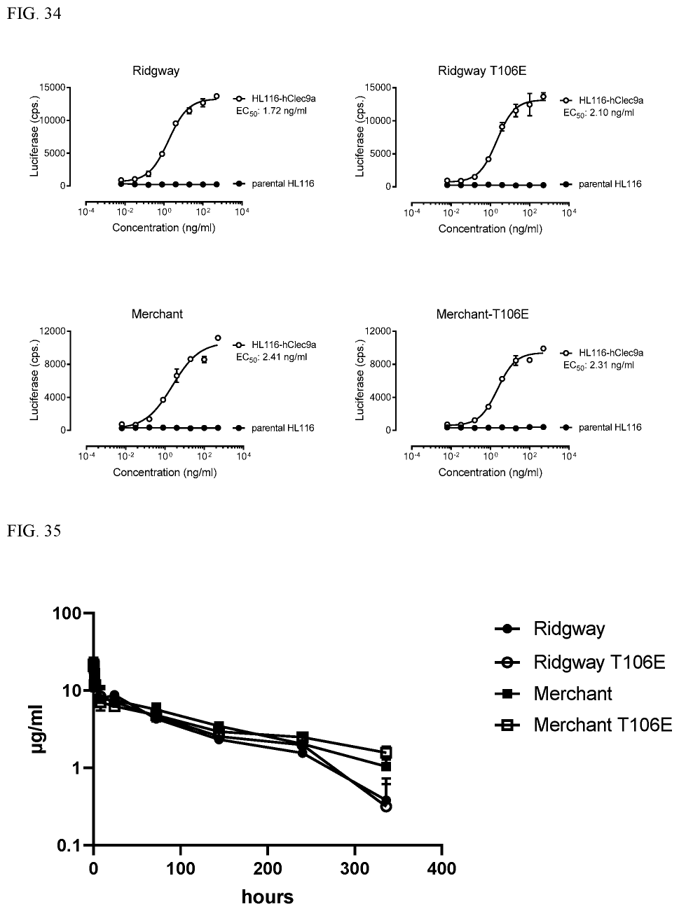

HL116 and the derived HL116-hClec9A were stimulated for six hours with a serial dilution of Fc-AFNs. Luciferase activity was measured and plotted in FIG. 34. Data show that all 4 constructs have a similar potency and specificity for target cells only.

### PK Study in Mouse

In total 9 mice were dosed intravenously at 1 mg/kg with each construct. K-EDTA blood was taken from a first group of 3 mice at 5 minutes, 8 hours and 6 days, from a second group of 3 mice at 15 minutes, 1 day and 10 days and from a third group of 3 mice at 2 hours, 3 days and 14 days. The concentration of intact Fc-AFN was measured by ELISA. In brief the MAXISORP Nunc Immune plates (Thermo Scientific) were coated overnight with anti-human interferon alpha mAb (clone MMHA-13; PBL Assay Science) at 0.5 μg/ml in PBS. After washing the plates four times with PBS+0.05% Tween-20, they were blocked with 0.1% Casein in PBS for at least 1 hour at room temperature. Subsequently, diluted samples and standards were incubated in 0.1% Casein in PBS for 2 hours at room temperature. After another wash cycle a custom made rabbit-anti-VHH (diluted 1/20000 in 0.1% Casein in PBS) was incubated for 2 hours at room temperature followed by an additional wash cycle and incubation with HRP-conjugated goat anti-rabbit (Jackson—111-035-144; 1:5000 in 0.1% Casein) for 1 hour at room temperature. After a final washing cycle, peroxidase activity was measured using KPL substrate (5120-0047; SeraCare) according to the manufacturer's instructions. Concentrations from samples were calculated using GraphPad Prism. Measured concentrations are plotted in FIG. 35 and show that all 4 constructs have a similar PK profile except for a somewhat faster clearance of the Ridgway based Fc-construct at the last sampling time point. Terminal half-life was estimated on average at about 3 days for the Ridgway constructs and 4.5 days for the Merchant constructs.

### Example 14: Efficacy Study in Humanized Mouse

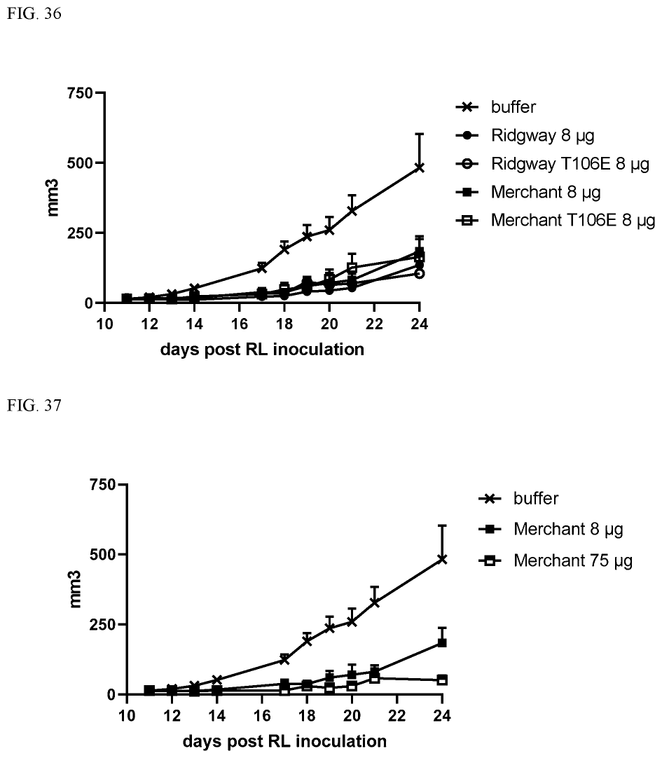

To evaluate the in vivo efficacy of the Fc-based AFNs the same 4 molecules as selected in Example 12 (targeted to human CLEC9A, a highly specific cDC1 marker) were tested in a tumor model in a humanized mouse. In brief, newborn NSG mice (1-2 days of age) were sublethal irradiated with 100 cGy prior to intrahepatic delivery of 1×10⁵ CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated daily intraperitoneally with 30 μg of human Flt3L protein, from day 10 to day 19 after tumor inoculation. Weekly intravenous injection with buffer or Fc-AFN (8 or 75 μg) was initiated at day 11 after tumor inoculation, when a palpable tumor was visible (n=5 mice per group). Tumor size (caliper measurements), body weight and temperature were assessed daily. Data in FIG. 36 and FIG. 37 show the tumor growth until 6 days after the second treatment (mice received weekly injections with either PBS or AFN, the data show tumor growth up to 7 days after the second weekly treatment). FIG. 36 demonstrates that all constructs induced a similar level of tumor growth inhibition at the lower dose of 8 μg. FIG. 37 shows the result of higher doses for a Merchant construct resulting in increasing tumor growth inhibition. Data on body weight and temperature did not show any major difference between buffer treatment and AFN treatment supporting that all AFN treatments were well tolerated.

### Example 15: PD-L1 VHH-Based Fc AFNs

In this Example, PD-L1 (programmed death-ligand 1) targeted Fc AFNs based on a human PD-L1 specific VHH (clone 2LIG99; blocks interaction with PD-1) for targeting to tumour cells or activated immune cells were generated and evaluated.

### Constructs:

2LIG99-_HA-tag_20*GGS-Fc-20*GGS-IFNa2_R149A_H6 (P-991)
(SEQ ID NO: 1492)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSAAAYPYDVPDYGSGGSGG
SGGSGGSGGSGGSGGSGGSGGSGGSGGSGGSGGSGGSGGSGGSGGSGGSGGSGGSDKTHTC
PPCPAPEAAGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALGAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCSV
MHEALHNHYTQKSLSLSPGKGGSGGSGGSGGSGGSGGSGGSGGSGGSGGSGGSGGSGGSGGS
GGSGGSGGSGGSGGSGGSGCDLPQTHSLGSRRTLMLLAQMRKISLFSCLKDRHDFGFPQEEFGNQ
FQKAETIPVLHEMIQQIFNLFSTKDSSAAWDETLLDKFYTELYQQLNDLEACVIQGVGVTETPLMKEDSI
LAVRKYFQRITLYLKEKKYSPCAWEVVRAEIMASFSLSTNLQESLRSKEHHHHHH
2LIG99-20*GGS-Fc1 (P-1040)
(SEQ ID NO: 1493)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSGGSGGSGGSGGSGGSGG
SGGSGGSGGSGGSGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKTKPREEQYNSTYRVVSVL
TVLHQDWLNGKEYKCKVSNKALGAPIEKTISKAKGQPREPQVYTLPPSRDELTKNQVSLTCLVKGFYP
SDIAVEWESNGQPENNYKTTPPVLDSDGSFFLTSKLTVDKSRWQQGNVFSCSVMHEALHNHYTQKS
LSLSPGK
Fc2-20*GGS-IFNa2_R149A (P-959)
(SEQ ID NO: 1494)
DKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALGAPIEKTISKAKGQPREPQVYTLPPS
RDELTKNQVSLYCLVKGFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGGSGGSGGSGGSGGS
GGSGGSGGSGGSGGSGGSGGSGGSGCDLPQTHSLGSRRTLMLLAQMRKISLFSCLKDRHDFGFPQ
EEFGNQFQKAETIPVLHEMIQQIFNLFSTKDSSAAWDETLLDKFYTELYQQLNDLEACVIQGVGVTETP
LMKEDSILAVRKYFQRITLYLKEKKYSPCAWEVVRAEIMASFSLSTNLQESLRSKE
2LIG99-20*GGS-Fc3 (P-1415)
(SEQ ID NO: 1495)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSGGSGGSGGSGGSGGSGG
SGGSGGSGGSGGSGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKTKPREEQYNSTYRVVSVL
TVLHQDWLNGKEYKCQVSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFSCSVMHEALHNHYTQK
SLSLSPGK
2LIG99-20*GGS-Fc4-20*GGS-IFNa2_R149A (P-1412)
(SEQ ID NO: 1496)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSGGSGGSGGSGGSGGSGG
SGGSGGSGGSGGSGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFSCSVMHEALHNHYTQK
SLSLSPGKGGSGGSGGSGGSGGSGGSGGSGGSGGSGGSGGSGGSGGSGGSGGSGGSGGSGGS
GGSGGSCDLPQTHSLGSRRTLMLLAQMRKISLFSCLKDRHDFGFPQEEFGNQFQKAETIPVLHEMIQ
QIFNLFSTKDSSAAWDETLLDKFYTELYQQLNDLEACVIQGVGVEETPLMKEDSILAVRKYFQRITLYLK
EKKYSPCAWEVVRAEIMASFSLSTNLQESLRSKE

### Production and Purification of PD-L1 VHH-Based AFN

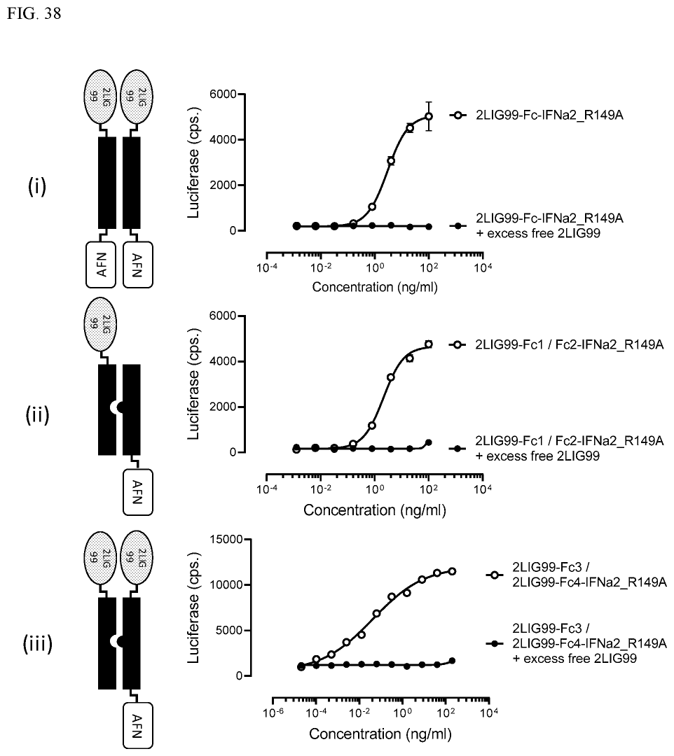

Homodimeric (2LIG99-20*GGS-Fc-20*GGS-IFNα2_R149A; scheme: FIG. 1A), monovalent heterodimeric (2LIG99-20*GGS-Fc1+Fc2-20*GGS-IFNα2_R149A; scheme: FIG. 7B), or the bivalent targeted heterodimeric (2LIG99-20*GGS-Fc3+2LIG99-20*GGS-Fc4-20*GGS-IFNα2_R149A; FIG. 16A) variants of the 2LIG99 Fc AFN (for a schematic representation, see FIG. 38) were produced in ExpiCHO cells by transient transfection of the corresponding constructs according to the manufacturer's guidelines. One week after transfection, supernatant was collected and cells removed by centrifugation. Recombinant proteins were purified from the supernatant using the Pierce Protein A spin plates (Thermo Fisher). The homodimeric 2LIG99-20*GGS-Fc-20*GGS-IFNa2_R149A was considerably less stable after purification with a high tendency to aggregate, while the two other constructs showed good solubility.

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells express PD-L1 endogenously, so targeting was evaluated in the absence or presence of an excess of the corresponding free PD-L1 VHH (2LIG99). Parental HL116 were seeded overnight at 20,000 cells per 96-well, pre-incubated with 2LIG99 (20 μg/ml) and subsequently stimulated with a serial dilution of Fc AFNs for 6 hours. Luciferase activity was measured in cell lysates. Data in the FIG. 38 illustrate that the homodimeric and monovalent heterodimeric variants are comparably active (EC₅₀ values of 2.98 and 2.14 ng/ml respectively), while the bivalent targeted heterodimeric variant is 40-50 times more active. In all three cases, excess of free VHH is sufficient to block signaling thereby illustrating the PD-L1 dependence of the signaling.

### Example 16: Efficacy Study in Humanized Mouse

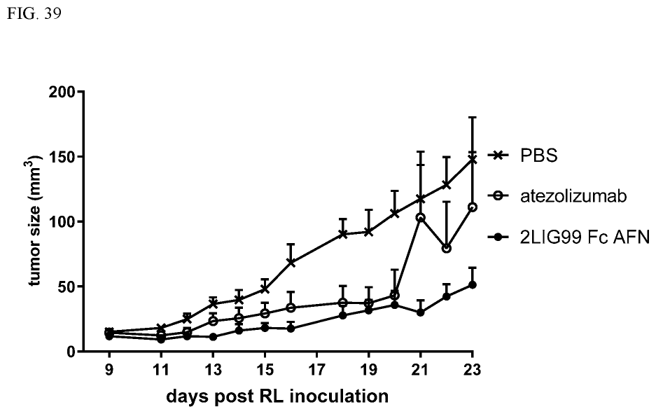

To evaluate the in vivo efficacy of Fc-based PD-L1 targeted AFNs, the homodimer variant (2LIG99-20*GGS-Fc-20*GGS-IFNa2_R149A) as described in Example 15 was tested in a tumor model in a humanized mouse. Protein production was performed in ExpiCHO cells as described above. Recombinant proteins were purified from the supernatant on a HiTrap Protein A HP (GE Healthcare). Low pH-eluted proteins were after neutralization desalted on a G25 column (GE Healthcare) and 0.22 μm filtered. Newborn NSG mice (1-2 days of age) were sublethal irradiated with 100 cGy prior to intrahepatic delivery of 1×10⁵CD34+ human stem cells (from HLA-A2 positive cord bloods). At week 13 after stem cell transfer mice were subcutaneously inoculated with 25×10⁵ human RL follicular lymphoma cells (ATCC CRL-2261; not sensitive to the direct anti-proliferative effect of IFN). Mice were treated daily intraperitoneally with 30 μg of human Flt3L protein, from day 5 to day 18 after tumor inoculation. Weekly perilesional injection with PBS or Fc-AFN (27 μg) or the PD-L1 blocking monoclonal antibody atezolizumab (30 μg; InVivoGen Catalog No.: hpdl1-mab12) was initiated at day 9 after tumor inoculation, when a palpable tumor was present (n=6 mice per group). Tumor size (caliper measurements), body weight and temperature were assessed daily. Data in FIG. 39 show the tumor growth until 7 days after the second treatment (mice received weekly injections with either PBS or AFN, the data show tumor growth up to 7 days after the second weekly treatment), demonstrating the superiority of PD-L1 targeted Fc-AFN versus PBS or even atezolizumab treatment. Data on body weight and temperature did not show any major difference between buffer treatment of AFN treatment supporting that all AFN treatments were well tolerated.

### Example 17: Clec4C VHH-Based Fc AFNs

In this Example, Clec4C targeted Fc AFNs based on a human Clec4C specific VHH (clone 2CL92) for targeting to plasmacytoid dendritic cell were generated and evaluated.

### Constructs:

Clec4C VHH-20*GGS-Fc3 (P-1571)
(SEQ ID NO: 1497)
QVQLQESGGGSVQAGDSLRLSCAASGRTFSGYAMGWFRQAPGKEREFVAT
ISTSGSSTYYADSVKGRFTISRDNAKKSVYLQINSLKTEDAAVYYCAARL
SFDNTAFYTSAIRYSYWGQGTQVTVSSGGSGGSGGSGGSGGSGGSGGSGG
SGGSGGSGGSGGSGGSGGSGGSGGSGGSGGSGGSGGSDKTHTCPPCPAPE
AAGGPSVFLFPPKPKDTLMISRTPEVTCVVVDVSHEDPEVKFNWYVDGVE
VHNAKTKPREEQYNSTYRVVSVLTVLHQDWLNGKEYKCQVSNKALPAPIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVMHEALH
NHYTQKSLSLSPGK
Fc4-20*GGS-IFNa2_R149A (P-1414)
(SEQ ID NO: 1498)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

### Production and Purification of Clec4C VHH-Based AFN

Constructs Clec4C VHH-20*GGS-Fc3 and Fc4-20*GGS-IFNa2_R149A were combined to a heterodimeric AFN (for a schematic representation, see FIG. 7B) and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

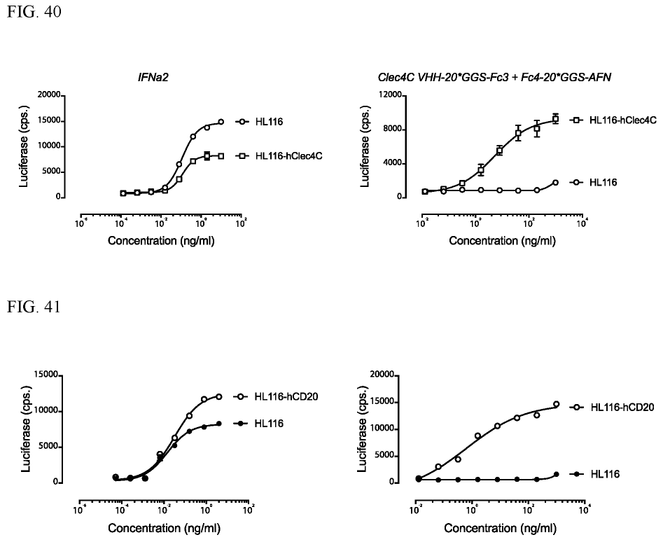

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec4C sequence. Stable transfected clones were selected in G418-containing medium. Parental HL116 and HL116-hClec4C cells were seeded overnight at 20,000 cells per 96-well and subsequently stimulated with a serial dilution of Fc AFNs for 6 hours. Luciferase activity was measured in cell lysates. Data in the FIG. 40 illustrate that both cell-lines are comparably sensitive to wild type IFNα2, while the Clec4C Fc AFN is much more active on targeted cells (HL116-hClec4C) compared to untargeted cells (parental HL116).

### Example 18: CD20 VHH-Based Fc AFNs

In this Example, CD20 targeted Fc AFNs based on a human CD20 specific VHH (clone 2HCD25) for targeting to B-cells and/or B-cell derived tumour cells were generated and evaluated.

### Constructs:

CD20 VHH-20*GGS-Fc3 (P-1570)
(SEQ ID NO: 1499)
QVQLQESGGGLAQAGGSLRLSCAASGRTFSMGWFRQAPGKEREFVAAITY
SGGSPYYASSVRGRFTISRDNAKNTVYLQMNSLKPEDTAVYYCAANPTYG
SDWNAENWGQGTQVTVSSGGSGGSGGSGGSGGSGGSGGSGGSGGSGGSGG
SGGSGGSGGSGGSGGSGGSGGSGGSGGSDKTHTCPPCPAPEAAGGPSVFL
FPPKPKDTLMISRTPEVTCVVVDVSHEDPEVKFNWYVDGVEVHNAKTKPR
EEQYNSTYRVVSVLTVLHQDWLNGKEYKCQVSNKALPAPIEKTISKAKGQ
PREPQVCTLPPSRDELTKNQVSLSCAVKGFYPSDIAVEWESNGQPENNYK
TTPPVLDSDGSFFLVSKLTVDKSRWQQGNVFSCSVMHEALHNHYTQKSLS
LSPGK
Fc4-20*GGS-IFNa2_R149A (P-1414)
(SEQ ID NO: 1500)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

### Production and Purification of CD20 VHH-Based AFN

Constructs CD20 VHH-20*GGS-Fc3 and Fc4-20*GGS-IFNa2_R149A were combined to a heterodimeric AFN (for a schematic representation, see FIG. 7B) and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human CD20 sequence. Stable transfected clones were selected in G418-containing medium. Parental HL116 and HL116-hCD20 cells were seeded overnight at 20,000 cells per 96-well and subsequently stimulated with a serial dilution of Fc AFNs for 6 hours. Luciferase activity was measured in cell lysates. Data in the FIG. 41 illustrate that both cell-lines are comparably sensitive to wild type IFNα2, while the CD20 Fc AFN is much more active on targeted cells (HL116-hCD20) compared to untargeted cells (parental HL116).

### Example 19: CD13 VHH-Based Fc AFNs

In this Example, CD13 targeted Fc AFNs based on a human CD13 specific VHH for targeting to tumour stromal cells were generated and evaluated.

### Constructs:

CD13 VHH-20*GGS-Fc3 (P-1569)
Fc4-20*GGS-IFNa2_R149A (P-1414)
(SEQ ID NO: 1501)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

### Production and Purification of CD13 VHH-Based AFN

Constructs CD13 VHH-20*GGS-Fc3 and Fc4-20*GGS-IFNa2_R149A were combined to a heterodimeric AFN (for a schematic representation, see FIG. 7B) and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

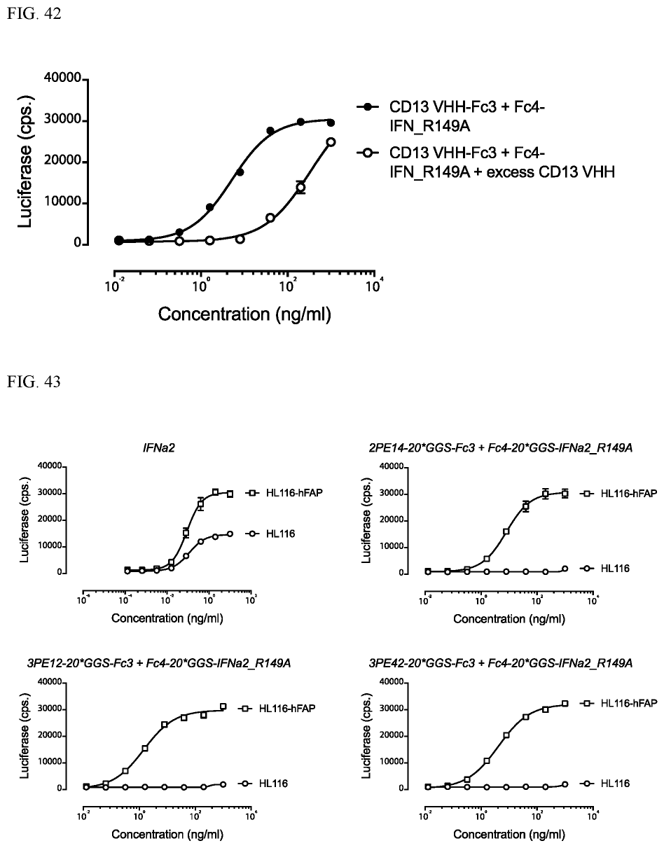

The HL116 clone is derived from the human HT1080 cell line (ATCC CCL-121). It contains the firefly luciferase gene controlled by the IFN-inducible 6-16 promoter. Parental HL116 cells express CD13 endogenously, so targeting will be evaluated in the absence or presence of an excess of the corresponding free CD13 VHH. Parental HL116 were therefor seeded overnight at 20,000 cells per 96-well, pre-incubated with CD13 VHH and subsequently stimulated with a serial dilution of Fc AFNs with excess for 6 hours. Luciferase activity was measured in cell lysates. Data in the FIG. 42 illustrate that stimulation in the presence of an excess CD13 VHH lowers biological activity 60-fold, thereby illustrating that the reconstitution of the IFN signaling is indeed depending on CD13 targeting.

### Example 20: FAP VHH-Based Fc AFNs

In this Example, FAP (fibroblast activation protein) targeted Fc AFNs based on three human FAP specific VHHs (clones 2PE14, 3PE12 and 3PE42) for targeting to tumour stromal cells were generated and evaluated.

### Constructs:

2PE14 VHH-20*GGS-Fc3 (P-1781)
(SEQ ID NO: 1502)
QVQLQESGGGLVQPGGSLRLSCAASGSTFSINAVAWYRQAPGKRRELVAG
ISGGGVTNYPDSVKGRFTISRDNAKNTVYLQMSSLKPEDTAVYYCNLWPP
RASPGGRVYWGQGTQVTVSSGGSGGSGGSGGSGGSGGSGGSGGSGGSGGS
GGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKS
LSLSPGK
3PE12 VHH-20*GGS-Fc3 (P-1782)
(SEQ ID NO: 1503)
QVQLQESGGGLVQPGGSLRLSCAASGSTFSGNAMAWYRQAPGKRRELVAG
ISGGGVTNYPDSVKGRFTISRDNAKNTVYLQMSSLKPEDTAVYYCNLWPP
RVSPGGGVYWGQGTQVTVSSGGSGGSGGSGGSGGSGGSGGSGGSGGSGGS
GGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKS
LSLSPGK
3PE42 VHH-20*GGS-Fc3 (P-1783)
(SEQ ID NO: 1504)
QVQLQESGGGLVQPGESLRLSCAVSGSTSSMNAMAWYRQAPGKRRELVAG
ISGGGATNYPDSVKGRFTISRDNAKNTVYLQMSSLKPEDTAVYYCNLWPP
RASPGGGVYWGQGTQVTVSSGGSGGSGGSGGSGGSGGSGGSGGSGGSGGS
GGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVCTLPPSRDELTKNQVSLSCAVKGFYPSDIAVEWESNGQPENN
YKTTPPVLDSDGSFFLVSKLTVDKSRWQQGNVFSCSVMHEALHNHYTQKS
LSLSPGK
Fc4-20*GGS-IFNa2_R149A (P-1414)
(SEQ ID NO: 1505)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

### Production and Purification of FAP VHH-Based AFN

Constructs 2PE14-20*GGS-Fc3, 3PE12-20*GGS-Fc3, or 3PE42-20*GGS-Fc3 were combined with construct Fc4-20*GGS-IFNa2_R149A to heterodimeric AFNs (for a schematic representation, see FIG. 7B) and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human FAP sequence. Stable transfected clones were selected in G418-containing medium. Parental HL116 and HL116-hFAP cells were seeded overnight at 20,000 cells per 96-well and subsequently stimulated with a serial dilution of Fc AFNs for 6 hours. Luciferase activity was measured in cell lysates. Data in the FIG. 43 illustrates that both cell-lines are comparably sensitive to wild type IFNα2, while the FAP Fc AFNs is much more active on targeted cells (HL116-hFAP) compared to untargeted cells (parental HL116).

### Example 21: CD8 VHH-Based Fc AFNs

In this Example, CD8 targeted Fc AFNs based on a human CD8 specific VHH (clone 1CDA65) for targeting to cytotoxic T-cells were generated and evaluated.

### Constructs:

CD8 VHH-20*GGS-Fc3 (P-1568)
(SEQ ID NO: 1506)
QVQLQESGGGLVHPGGSLRLSCAASGRSFSSYFMGWFRQAPGKEREFVAG
IGWNDGSINYADSVKGRFTISRDNAKNTGYLQMNSLKPEDTAVYYCAASV
SLYGLEKSSAYTSWGQGTQVTVSSGGSGGSGGSGGSGGSGGSGGSGGSGG
SGGSGGSGGSGGSGGSGGSGGSGGSGGSGGSGGSDKTHTCPPCPAPEAAG
GPSVFLFPPKPKDTLMISRTPEVTCVVVDVSHEDPEVKFNWYVDGVEVHN
AKTKPREEQYNSTYRVVSVLTVLHQDWLNGKEYKCQVSNKALPAPIEKTI
SKAKGQPREPQVCTLPPSRDELTKNQVSLSCAVKGFYPSDIAVEWESNGQ
PENNYKTTPPVLDSDGSFFLVSKLTVDKSRWQQGNVFSCSVMHEALHNHY
TQKSLSLSPGK
Fc4-20*GGS-IFNa2_R149A (P-1414)
(SEQ ID NO: 1507)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

### Production and Purification of CD8 VHH-Based AFN

Constructs CD8 VHH-20*GGS-Fc3 and Fc4-20*GGS-IFNα2_R149A were combined to a heterodimeric AFN (for a schematic representation, see FIG. 7B) and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

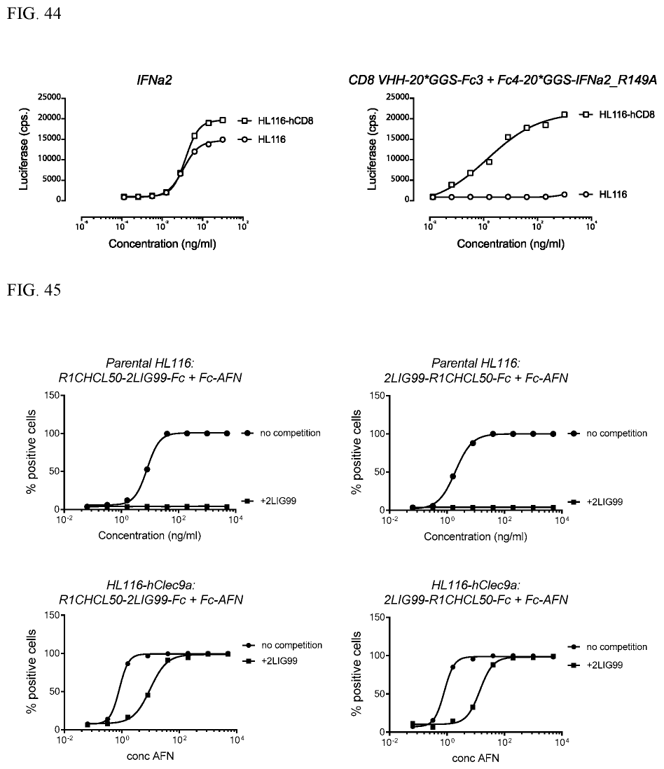

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human CD8 sequence. Stable transfected clones were selected in G418-containing medium. Parental HL116 and HL116-hCD8 cells were seeded overnight at 20,000 cells per 96-well and subsequently stimulated with a serial dilution of Fc AFNs for 6 hours. Luciferase activity was measured in cell lysates. Data in the FIG. 44 illustrate that both cell-lines are comparably sensitive to wild type IFNα2, while the CD8 Fc AFN is much more active on targeted cells (HL116-hCD8) compared to untargeted cells (parental HL116).

### Example 22: Monovalent Clec9A and PD-L1 Targeted Fc AFNs

In this Example, we generated and evaluated Fc AFNs targeting both Clec9A (marker for cDC1 dendritic cells) and PD-L1 (expressed on tumour cells and activated immune cells). For targeting, we used two VHHs: R1CHCL50 (human Clec9A specific) and 2LIG99 (human PD-L1 specific).

### Constructs:

Fc4-20*GGS-IFNa2_R149A (P-1414)
(SEQ ID NO: 1508)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE
R1CHCL50-20*GGS-2LIG99-20*GGS-Fc3 (P-1467)
(SEQ ID NO: 1509)
QVQLVESGGGLVHPGGSLRLSCAASGSFSSINVMGWYRQAPGKERELVAR
ITNLGLPNYADSVTGRFTISRDNAKNTVYLQMNSLKPEDTAVYYCYLVAL
KAEYWGQGTQVTVSSGGSGGSGGSGGSGGSGGSGGSGGSGGSGGSGGSGG
SGGSGGSGGSGGSGGSGGSGGSGGSQVQLQESGGGLVQAGGSLRLSCTAS
GTIFSINRMDWFRQAPGKQRELVALITSDGTPAYADSAKGRFTISRDNTK
KTVSLQMNSLKPEDTAVYYCHVSSGVYNYWGQGTQVTVSSGGSGGSGGSG
GSGGSGGSGGSGGSGGSGGSGGSGGSGGSGGSGGSGGSGGSGGSGGSGGS
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGK
2LIG99-20*GGS-R1CHCL50-20*GGS-Fc3 (P-1469)
(SEQ ID NO: 1510)
QVQLQESGGGLVQAGGSLRLSCTASGTIFSINRMDWFRQAPGKQRELVAL
ITSDGTPAYADSAKGRFTISRDNTKKTVSLQMNSLKPEDTAVYYCHVSSG
VYNYWGQGTQVTVSSGGSGGSGGSGGSGGSGGSGGSGGSGGSGGSGGSGG
SGGSGGSGGSGGSGGSGGSGGSGGSQVQLVESGGGLVHPGGSLRLSCAAS
GSFSSINVMGWYRQAPGKERELVARITNLGLPNYADSVTGRFTISRDNAK
NTVYLQMNSLKPEDTAVYYCYLVALKAEYWGQGTQVTVSSGGSGGSGGSG
GSGGSGGSGGSGGSGGSGGSGGSGGSGGSGGSGGSGGSGGSGGSGGSGGS
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGK

### Production and Purification of Clec9A and PD-L1 VHH-Based AFN

The following combinations were transiently transfected in ExpiCHO cells:
i. R1CHCL50-2LIG99-Fc3+Fc4-IFNα2_R149A (bispecific; monovalent targeting; scheme: FIG. 8B);
ii. 2LIG99-R1CHCL50-Fc3+Fc4-IFNα2_R149A (bispecific; monovalent targeting; scheme: FIG. 8B).

One week after transfection, supernatant was collected and cells removed by centrifugation. Recombinant proteins were purified from the supernatant using the Pierce Protein A spin plates (Thermo Fisher).

### Relative Affinity in FACS

To measure relative affinities for Clec9A and PD-L1 the bispecific targeted Fc AFNs, and HL116-hClec9A cells were incubated with a serial dilution of AFN in the absence or presence of an excess of the competing free VHH 2LIG99. Binding was detected by subsequent incubation with an FITC-coupled anti-human secondary Ab, measured on a MACSQuant X instrument (Miltenyi Biotech) and analysed using the FlowLogic software (Miltenyi Biotech). Data in FIG. 45 illustrates that both bispecific targeted AFNs can bind to both targets simultaneously resulting in increased affinity for cells expressing both targets.

### Example 23: Monovalent and Bivalent Clec9A and PD-L1 Targeted Fc AFNs

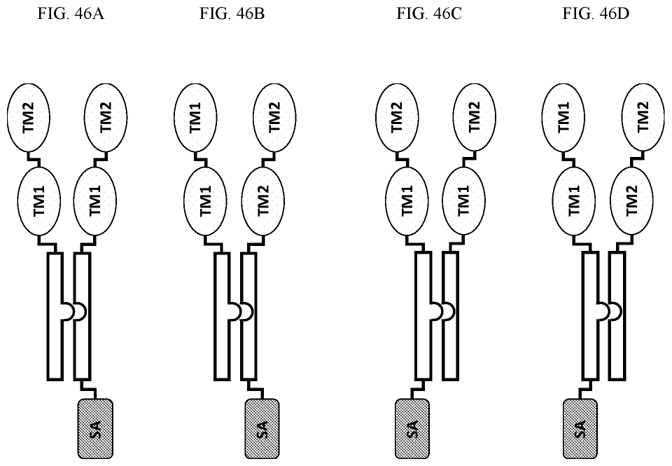

In this Example, we generated and evaluated Fc AFNs targeting both Clec9A (marker for cDC1 dendritic cells) and PD-L1 (expressed on tumour cells and activated immune cells). For targeting, we used two VHHs: R1CHCL50 (human Clec9A specific) and 2LIG99 (human PD-L1 specific). Two bispecific formats (one resulting in monovalent, the other in bivalent targeting) were compared with the monospecific Fc AFNs for biological activity. Bivalent-bispecific targeting is schematically represented in FIGS. 46A-D.

### Constructs:

R1CHCL50-20*GGS-Fc3 (P-1451)
(SEQ ID NO: 1511)
QVQLVESGGGLVHPGGSLRLSCAASGSFSSINVMGWYRQAPGKERELVARITNLGLPNYADSVTGR
FTISRDNAKNTVYLQMNSLKPEDTAVYYCYLVALKAEYWGQGTQVTVSSGGSGGSGGSGGSGGSG
GSGGSGGSGGSGGSGGSGGSGGSGGSGGSGGSGGSGGSGGSGGSDKTHTCPPCPAPEAAGGP
SVFLFPPKPKDTLMISRTPEVTCVVVDVSHEDPEVKFNWYVDGVEVHNAKTKPREEQYNSTYRVVSV
LTVLHQDWLNGKEYKCQVSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFSCSVMHEALHNHYTQK
SLSLSPGK
2LIG99-20*GGS-Fc3 (P-1415)
(SEQ ID NO: 1512)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSGGSGGSGGSGGSGGSGG
SGGSGGSGGSGGSGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKTKPREEQYNSTYRVVSVL
TVLHQDWLNGKEYKCQVSNKALPAPIEKTISKAKGQPREPQVCTLPPSRDELTKNQVSLSCAVKGFY
PSDIAVEWESNGQPENNYKTTPPVLDSDGSFFLVSKLTVDKSRWQQGNVFSCSVMHEALHNHYTQK
SLSLSPGK
Fc4-20*GGS-IFNa2_R149A (P-1414)
(SEQ ID NO: 1513)
DKTHTCPPCPAPEAAGGPSVFLFPPKPKDTLMISRTPEVTCVVVDVSHEDPEVKFNWYVDGVEVHNA
KTKPREEQYNSTYRVVSVLTVLHQDWLNGKEYKCQVSNKALPAPIEKTISKAKGQPREPQVYTLPPC
RDELTKNQVSLWCLVKGFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGGSGGSGGSGGSGGS
GGSGGSGGSGGSGGSGGSGGSGGSCDLPQTHSLGSRRTLMLLAQMRKISLFSCLKDRHDFGFPQE
EFGNQFQKAETIPVLHEMIQQIFNLFSTKDSSAAWDETLLDKFYTELYQQLNDLEACVIQGVGVEETPL
MKEDSILAVRKYFQRITLYLKEKKYSPCAWEVVRAEIMASFSLSTNLQESLRSKE
2LIG99-20*GGS-Fc420*GGS--IFNa2_R149A (P-1412)
(SEQ ID NO: 1514)
QVQLQESGGGLVQAGGSLRLSCTASGTIFSINRMDWFRQAPGKQRELVALITSDGTPAYADSAKGRF
TISRDNTKKTVSLQMNSLKPEDTAVYYCHVSSGVYNYWGQGTQVTVSSGGSGGSGGSGGSGGSGG
SGGSGGSGGSGGSGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKTKPREEQYNSTYRVVSVL
TVLHQDWLNGKEYKCQVSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFSCSVMHEALHNHYTQK
SLSLSPGKGGSGGSGGSGGSGGSGGSGGSGGSGGSGGSGGSGGSGGSGGSGGSGGSGGSGGS
GGSGGSCDLPQTHSLGSRRTLMLLAQMRKISLFSCLKDRHDFGFPQEEFGNQFQKAETIPVLHEMIQ
QIFNLFSTKDSSAAWDETLLDKFYTELYQQLNDLEACVIQGVGVEETPLMKEDSILAVRKYFQRITLYLK
EKKYSPCAWEVVRAEIMASFSLSTNLQESLRSKE
3LEC89-20*GGS-Fc3-20*GGS-2LIG99 (P-1413)
(SEQ ID NO: 1515)
QVQLQESGGGLVQPGGSLRLSCAASGRIFSVNAMGWYRQAPGKQRELVAAITNQGAPTYADSVKG
RFTISRDNAGNTVYLQMNSLRPEDTAVYYCKAFTRGDDYWGQGTQVTVSSGGSGGSGGSGGSGGS
GGSGGSGGSGGSGGSGGSGGSGGSGGSGGSGGSGGSGGSGGSGGSDKTHTCPPCPAPEAAGG
PSVFLFPPKPKDTLMISRTPEVTCVVVDVSHEDPEVKFNWYVDGVEVHNAKTKPREEQYNSTYRVVS
VLTVLHQDWLNGKEYKCQVSNKALPAPIEKTISKAKGQPREPQVCTLPPSRDELTKNQVSLSCAVKGF
YPSDIAVEWESNGQPENNYKTTPPVLDSDGSFFLVSKLTVDKSRWQQGNVFSCSVMHEALHNHYTQ
KSLSLSPGKGGSGGSGGSGGSGGSGGSGGSGGSGGSGGSGGSGGSGGSGGSGGSGGSGGSGG
SGGSGGSQVQLQESGGGLVQAGGSLRLSCTASGTIFSINRMDWFRQAPGKQRELVALITSDGTPAY
ADSAKGRFTISRDNTKKTVSLQMNSLKPEDTAVYYCHVSSGVYNYWGQGTQVTVSS
R1CHCL50-20*GGS-2LIG99-20*GGS-Fc3 (P-1467)
(SEQ ID NO: 1516)
QVQLVESGGGLVHPGGSLRLSCAASGSFSSINVMGWYRQAPGKERELVARITNLGLPNYADSVTGR
FTISRDNAKNTVYLQMNSLKPEDTAVYYCYLVALKAEYWGQGTQVTVSSGGSGGSGGSGGSGGSG
GSGGSGGSGGSGGSGGSGGSGGSGGSGGSGGSGGSGGSGGSGGSQVQLQESGGGLVQAGGSL
RLSCTASGTIFSINRMDWFRQAPGKQRELVALITSDGTPAYADSAKGRFTISRDNTKKTVSLQMNSLK
PEDTAVYYCHVSSGVYNYWGQGTQVTVSSGGSGGSGGSGGSGGSGGSGGSGGSGGSGGSGGS
GGSGGSGGSGGSGGSGGSGGSGGSGGSDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEYKCQV
SNKALPAPIEKTISKAKGQPREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKSLSLSPGK
R1CHCL50-20*GGS-2LIG99-20*GGS-Fc4-20*GGS-IFNa2_R149A (P-1468)
(SEQ ID NO: 1517)
QVQLVESGGGLVHPGGSLRLSCAASGSFSSINVMGWYRQAPGKERELVARITNLGLPNYADSVTGR
FTISRDNAKNTVYLQMNSLKPEDTAVYYCYLVALKAEYWGQGTQVTVSSGGSGGSGGSGGSGGSG
GSGGSGGSGGSGGSGGSGGSGGSGGSGGSGGSGGSGGSGGSGGSQVQLQESGGGLVQAGGSL
RLSCTASGTIFSINRMDWFRQAPGKQRELVALITSDGTPAYADSAKGRFTISRDNTKKTVSLQMNSLK
PEDTAVYYCHVSSGVYNYWGQGTQVTVSSGGSGGSGGSGGSGGSGGSGGSGGSGGSGGSGGS
GGSGGSGGSGGSGGSGGSGGSGGSGGSDKTHTCPPCPAPEAAGGPSVFLFPPKPKDTLMISRTPE
VTCVVVDVSHEDPEVKFNWYVDGVEVHNAKTKPREEQYNSTYRVVSVLTVLHQDWLNGKEYKCQV
SNKALPAPIEKTISKAKGQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKSLSLSPGKGGSGGSGGS
GGSGGSGGSGGSGGSGGSGGSGGSGGSGGSGGSGGSGGSGGSGGSGGSGGSCDLPQTHSLGS
RRTLMLLAQMRKISLFSCLKDRHDFGFPQEEFGNQFQKAETIPVLHEMIQQIFNLFSTKDSSAAWDET
LLDKFYTELYQQLNDLEACVIQGVGVEETPLMKEDSILAVRKYFQRITLYLKEKKYSPCAWEVVRAEIM
ASFSLSTNLQESLRSKE

### Production and Purification of CIec9A and PD-L1 VHH-Based AFN

The following combinations were transiently transfected in ExpiCHO cells:
i. R1CHCL50-Fc3+Fc4-IFNα2_R149A (mono-specific Clec9A; scheme: FIG. 7B);
ii. 2LIG99-Fc3+Fc4-IFNα2_R149A (mono-specific PD-L1; scheme: FIG. 7B);
iii. R1CHCL50-Fc3+2LIG99-Fc4-IFNα2_R149A (bispecific; monovalent targeting; scheme: FIG. 16A);
iv. 3LEC89-Fc3-2LIG99+Fc4-IFNα2_R149A (bispecific; monovalent targeting; scheme: FIG. 8A);
v. R1CHCL50-2LIG99-Fc3+R1CHCL50-2LIG99-Fc4-IFNα2_R149A (bispecific and bi-valent targeting; FIG. 46C).

One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

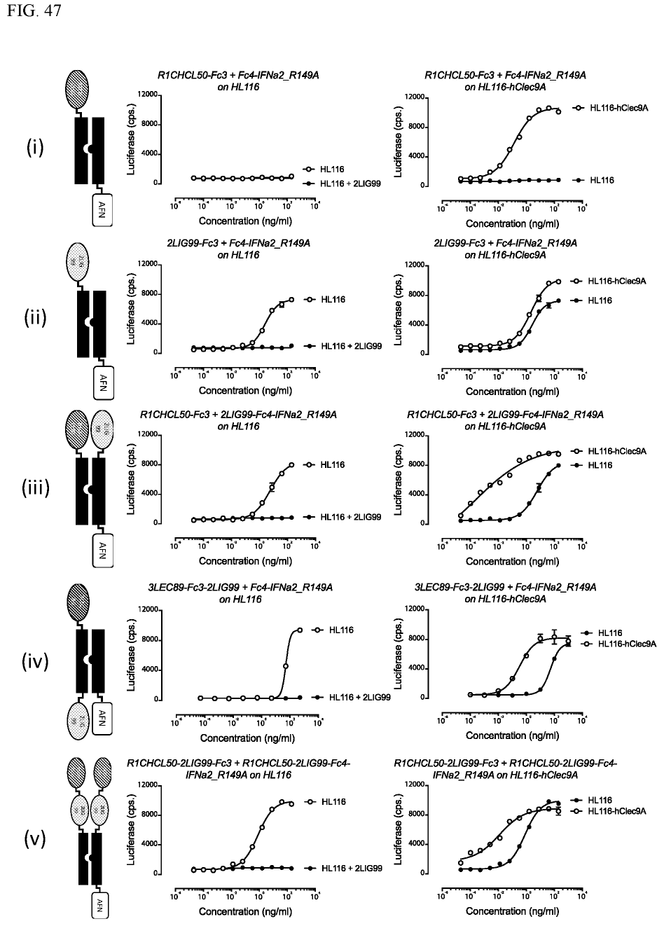

The HL116 clone is derived from the human HT1080 cell line (ATCC CCL-121). It contains the firefly luciferase gene controlled by the IFN-inducible 6-16 promoter. Parental HL116 cells express PD-L1 endogenously and were therefore used to evaluate signaling based on PD-L1 targeting. The ‘untargeted’ situation was mimicked by stimulating cells in the presence of an excess free PD-L1 VHH 2LIG99. Clec9A targeting was evaluated by comparing signaling in parental HL116 vs HL116-hClec9A cells. Parental and derived cells were seeded overnight at 20,000 cells per 96-well, pre-incubated with 2LIG99 (20 μg/ml) (where indicated) and subsequently stimulated with a serial dilution of Fc AFNs for 6 hours. Luciferase activity was measured in cell lysates. Data in FIG. 47 and Table 12 illustrate, inter a/ia, that:
2LIG99 containing Fc AFNs are active on both cell lines as expected, specificity of targeting is demonstrated by the competition with free VHH 2LIG99;
AFNs based on R1CHCL50 only are active only on HL116-hClec9A cells;
Bispecific AFNs are significantly more active on HL116-hClec9A cells (expressing both targets) compared to the monospecific variants or compared to parental HL116 expressing only PD-L1; and
The bispecific and bivalent targeting molecule has the lowest EC50 on the cell line expressing only PD-L1 confirming earlier findings of increased potency by bivalent targeting (Example 15).

TABLE 12
Biological activity of bi-specific
Clec9A-PD-L1 Fc AFN variants as show in FIG. 47.

| | | EC₅₀ (ng/ml) | |
|---|---|---|---|
| | | Parental | HL116- |
| | | HL116 | hClec9A |
| (i) | R1CHCL50-Fc3 + | ND | 0,14 |
| | Fc4-IFNa2_R149A | | |
| (ii) | 2LIG99-Fc3 + | 3,76 | 1,94 |
| | Fc4-IFNa2_R149A | | |
| (iii) | R1CHCL50-Fc3 + | 7,16 | 0,0005 |
| | 2LIG99-Fc4-IFNa2_R149A | | |
| (iv) | 3LEC89-Fc3-2LIG99 +. | 52,12 | 0,33 |
| | Fc4-IFNa2_R149A | | |
| (v) | R1CHCL50-2L1G99-Fc3 + | 0,76 | 0,012 |
| | R1CHCL50-2LIG99-Fc4-IFNa2_R149A | | |

### Example 24: Efficacy Study in Humanized Mouse

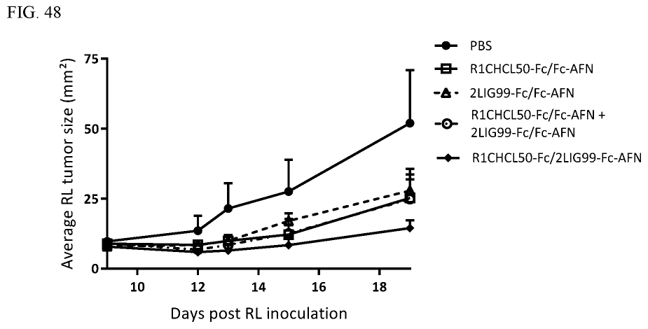

To evaluate the in vivo efficacy of Fc-based PD-L1 and/or CLEC9A targeted AFNs, a molecules (i), (ii), and (iii) as well as the mixture of molecules (i) and (ii) as described in Example 23; Table 12 were tested in a tumor model in a humanized mouse. Protein production was performed in ExpiCHO cells as described above. Recombinant proteins were purified from the supernatant on a HiTrap Protein A HP (GE Healthcare). Bound proteins were eluted from the column at pH 3.5 (25 mM Na-citrate) and neutralized with 1 M Tris pH8.8. Finally, the proteins were desalted on a G25 or desalted and polished on a G200 column (GE Healthcare) to 10 mM NH4-acetate pH5—123.5 mM NaCl—0.02% Tween20 and 0.22 μm filtered. Newborn NSG mice (1-2 days of age) were sublethal irradiated with 100 cGy prior to intrahepatic delivery of 1×10⁵CD34+ human stem cells (from HLA-A2 positive cord bloods). At week 13 after stem cell transfer mice were subcutaneously inoculated with 25×10⁵ human RL follicular lymphoma cells (ATCC CRL-2261). Mice were treated daily intraperitoneally with 30 μg of human Flt3L protein, from day 8 to day 15 after tumor inoculation. Intravenous injection with buffer or Fc-AFN was performed on at days 9 and 15 after tumor inoculation, when a palpable tumor was detected (n=4-5 mice per group). Animals were followed until 4 days after the second treatment. Tumor size (caliper measurements was assessed at regular intervals. FIG. 48 demonstrates the superiority that both the CLEC9A and the PD-L1 targeted AFN reduced tumor growth compared to buffer treatment only. The bispecific AFN targeted to both PD-L1 and CLEC9A appears superior over the single targeted AFNs or the combination of the single-targeted AFNs.

### Example 25: CD8 and Clec4C Targeted Fc AFNs

In this Example, we generated and evaluated Fc AFNs targeting both Clec4C (marker for plasmacytoid dendritic cells) and CD8 (T cell subset). For targeting, we used two VHHs: 2CL92 (human Clec4C specific) and 1CDA65 (human CD8 specific). The bispecific format will be compared with the monospecific Fc AFNs for biological activity on target expressing HL116 cells.

### Constructs:

Clec4C VHH-20*GGS-Fc3 (P-1571)
(SEQ ID NO: 1518)
QVQLQESGGGSVQAGDSLRLSCAASGRTFSGYAMGWFRQAPGKEREFVAT
ISTSGSSTYYADSVKGRFTISRDNAKKSVYLQINSLKTEDAAVYYCAARL
SFDNTAFYTSAIRYSYWGQGTQVTVSSGGSGGSGGSGGSGGSGGSGGSGG
SGGSGGSGGSGGSGGSGGSGGSGGSGGSGGSGGSGGSDKTHTCPPCPAPE
AAGGPSVFLFPPKPKDTLMISRTPEVTCVVVDVSHEDPEVKFNWYVDGVE
VHNAKTKPREEQYNSTYRVVSVLTVLHQDWLNGKEYKCQVSNKALPAPIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVMHEALH
NHYTQKSLSLSPGK
CD8 VHH-20*GGS-Fc3 (P-1568)
(SEQ ID NO: 1519)
QVQLQESGGGLVHPGGSLRLSCAASGRSFSSYFMGWFRQAPGKEREFVAG
IGWNDGSINYADSVKGRFTISRDNAKNTGYLQMNSLKPEDTAVYYCAASV
SLYGLEKSSAYTSWGQGTQVTVSSGGSGGSGGSGGSGGSGGSGGSGGSGG
SGGSGGSGGSGGSGGSGGSGGSGGSGGSGGSGGSDKTHTCPPCPAPEAAG
GPSVFLFPPKPKDTLMISRTPEVTCVVVDVSHEDPEVKFNWYVDGVEVHN
AKTKPREEQYNSTYRVVSVLTVLHQDWLNGKEYKCQVSNKALPAPIEKTI
SKAKGQPREPQVCTLPPSRDELTKNQVSLSCAVKGFYPSDIAVEWESNGQ
PENNYKTTPPVLDSDGSFFLVSKLTVDKSRWQQGNVFSCSVMHEALHNHY
TQKSLSLSPGK
Fc4-20*GGS-IFNa2_R149A (P-1414)
(SEQ ID NO: 1520)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE
CD8 VHH-20*GGS-Fc4-20*GGS-IFNa2_R149A (P-1628)
(SEQ ID NO: 1521)
QVQLQESGGGLVQPGGSLRLSCAASGSIFSINVMGWYRQTPGKERELVAK
ITNFGITSYADSAQGRFTISRGNAKNTVYLQMNSLKPEDTAVYYCNLDTT
GWGPPPYQYWGQGTQVTVSSGGSGGSGGSGGSGGSGGSGGSGGSGGSGGS
GGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKS
LSLSPGKGGSGGSGGSGGSGGSGGSGGSGGSGGSGGSGGSGGSGGSGGSG
GSGGSGGSGGSGGSGGSCDLPQTHSLGSRRTLMLLAQMRKISLFSCLKDR
HDFGFPQEEFGNQFQKAETIPVLHEMIQQIFNLFSTKDSSAAWDETLLDK
FYTELYQQLNDLEACVIQGVGVEETPLMKEDSILAVRKYFQRITLYLKEK
KYSPCAWEVVRAEIMASFSLSTNLQESLRSKE

### Production and Purification of Clec4C and CD8 VHH-Based AFNs

The following combinations were transiently transfected in ExpiCHO cells:
(i) Clec4C VHH-Fc3+Fc4-IFNα2_R149A (Clec4C specific AFN; scheme: FIG. 7B);
(ii) CD8 VHH-Fc3+Fc4-IFNα2_R149A (Clec4C specific AFN; scheme: FIG. 7B);
(iii) Clec4C VHH-Fc3+CD8 VHH-Fc4-IFNα2_R149A (bi-specific AFN; scheme: FIG. 16A).

One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

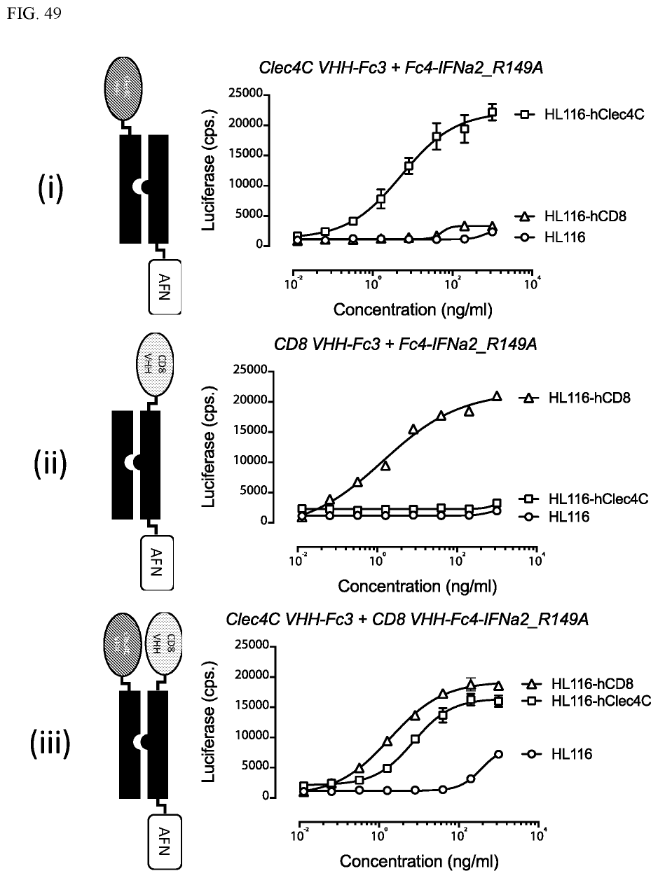

The HL116 clone is derived from the human HT1080 cell line (ATCC CCL-121). It contains the firefly luciferase gene controlled by the IFN-inducible 6-16 promoter. Vectors encoding human Clec4C or human CD8 were stably transfected in these HL116 cells, resulting in the HL116-hClec4C and HL116-hCD8 cell lines. To measure biological activity, parental and derived cells were seeded overnight at 20,000 cells per 96-well and subsequently stimulated with a serial dilution of Fc AFNs for 6 hours. Luciferase activity was measured in cell lysates. Data in FIG. 49 illustrate that mono-specific Fc AFNs are only active on the respective HL116-hClec4C and HL116-hCD8 cell lines, while the bi-specific Fc AFN induces signaling in both cell lines, but not in parental cells.

### Example 26: scFv Xcr1 Ab AFNs

In this Example, we designed and evaluated AFNs based on a scFv variant of the human Xcr1 Ab 5G7 for targeting of conventional dendritic cells type 1 (cDC1) cells.

### Constructs:

scFv Xcr1 Ab-20*GGS-Fc3 (P-1620)
(SEQ ID NO: 1522)
DVVMTQTPLSLPVTLGNQASIFCRSSLGLVHRNGNTYLHWYLQKPGQSPK
LLIYKVSHRFSGVPDRFSGSGSGTDFTLKISRVEAEDLGVYFCSQSTHVP
WTFGGGTKLEIKGGGGSGGGGSGGGGSGGGGSQAYLQQSGAELVRPGASV
KMSCKASGYTFTSHNLHWVKQTPRQGLQWIGAIYPGNGNTAYNQKFKGKA
TLTVDKSSSTAYMQLSSLTSDDSAVYFCARWGSVVGDWYFDVWGTGTTVT
VSSSGGSGGSGGSGGSGGSGGSGGSGGSGGSGGSGGSGGSGGSGGSGGSG
GSGGSGGSGGSGGSDKTHTCPPCPAPEAAGGPSVFLFPPKPKDTLMISRT
PEVTCVVVDVSHEDPEVKFNWYVDGVEVHNAKTKPREEQYNSTYRVVSVL
TVLHQDWLNGKEYKCQVSNKALPAPIEKTISKAKGQPREPQVCTLPPSRD
ELTKNQVSLSCAVKGFYPSDIAVEWESNGQPENNYKTTPPVLDSDGSFFL
VSKLTVDKSRWQQGNVFSCSVMHEALHNHYTQKSLSLSPGK
Fc4-20*GGS-IFNa2_R149A (P-1414)
(SEQ ID NO: 1523)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

Production and Purification of scFv Xcr1 Ab-Based AFN

Constructs scFv Xcr1 Ab-20*GGS-Fc3 and Fc4-20*GGS-IFNa2_R149A were combined to a heterodimeric AFN with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

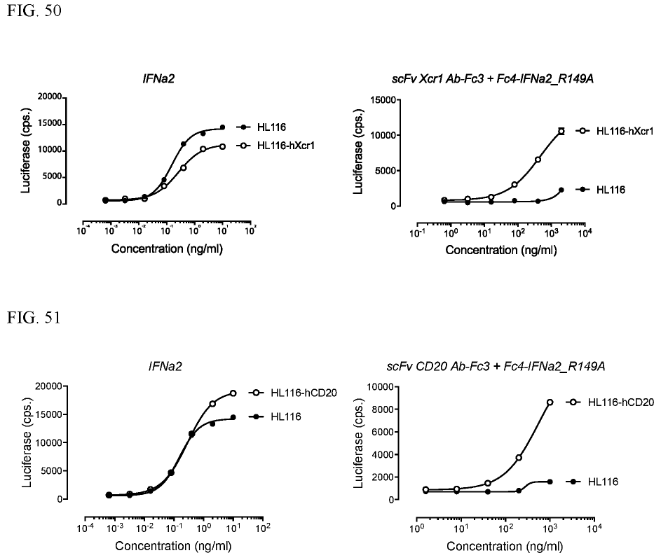

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Xcr1 sequence. Stable transfected clones were selected in G418-containing medium. Parental HL116 and HL116-hXcr1 cells were seeded overnight at 20,000 cells per 96-well and subsequently stimulated with a serial dilution of Fc AFNs for 6 hours. Luciferase activity was measured in cell lysates. Data in the FIG. 50 illustrate that both cell-lines are comparably sensitive to wild type IFNα2, while the hXcr1 Fc AFN is more active on targeted cells (HL116-hXcr1) compared to untargeted cells (parental HL116).

### Example 28: Construction of scFv CD20 Ab AFNs

In this Example, we designed and evaluated AFNs based on a scFv variant of the human CD20 Ab Rituximab for targeting B- and tumor cells.

### Constructs:

scFv CD20 Ab-20*GGS-Fc3 (P-1622)
(SEQ ID NO: 1524)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRGSTGGGGSGGGGSGGGGSQVQLQQPGAELVKPGASVKMSCKAS
GYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSSGGSG
GSGGSGGSGGSGGSGGSGGSGGSGGSGGSGGSGGSGGSGGSGGSGGSGGS
GGSGGSDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWL
NGKEYKCQVSNKALPAPIEKTISKAKGQPREPQVCTLPPSRDELTKNQVS
LSCAVKGFYPSDIAVEWESNGQPENNYKTTPPVLDSDGSFFLVSKLTVDK
SRWQQGNVFSCSVMHEALHNHYTQKSLSLSPGK
Fc4-20*GGS-IFNa2_R149A (P-1414)
(SEQ ID NO: 1525)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE

Production and Purification of scFv CD20 Ab-Based AFN

Constructs scFv CD20 Ab-20*GGS-Fc3 and Fc4-20*GGS-IFNa2_R149A were combined to a heterodimeric AFN with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Example 28: Characterization of scFv CD20 Ab AFNs

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human CD20 sequence. Stable transfected clones were selected in G418-containing medium. Parental HL116 and HL116-hCD20 cells were seeded overnight at 20,000 cells per 96-well and subsequently stimulated with a serial dilution of Fc AFNs for 6 hours. Luciferase activity was measured in cell lysates. Data in the FIG. 51 illustrate that both cell-lines are comparably sensitive to wild type IFNα2, while the hCD20 Fc AFN is more active on targeted cells (HL116-hCD20) compared to untargeted cells (parental HL116).

### Example 29: scFv CD20 Ab AFRs

In this Example, we target AcTafactors (AFRs) to B- or tumor cells using a scFv variant of the human CD20 Ab Rituximab. TNF, bearing the Y87F mutation, will be cloned as a single chain trimer (with GGGGS-linkers) on one Fc arm in the AFR.

### Constructs:

scFv CD20 Ab-20*GGS-Fc3 (P-1622)
(SEQ ID NO: 1526)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRGSTGGGGSGGGGSGGGGSQVQLQQPGAELVKPGASVKMSCKAS
GYTFTSYNMHWVKQTPGRGLEWIGAIYPGNGDTSYNQKFKGKATLTADKS
SSTAYMQLSSLTSEDSAVYYCARSTYYGGDWYFNVWGAGTTVTVSSGGSG
GSGGSGGSGGSGGSGGSGGSGGSGGSGGSGGSGGSGGSGGSGGSGGSGGS
GGSGGSDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWL
NGKEYKCQVSNKALPAPIEKTISKAKGQPREPQVCTLPPSRDELTKNQVS
LSCAVKGFYPSDIAVEWESNGQPENNYKTTPPVLDSDGSFFLVSKLTVDK
SRWQQGNVFSCSVMHEALHNHYTQKSLSLSPGK
Fc4-20*GGS-3*TNF_Y87F (P-1545)
(SEQ ID NO: 1527)
DKTHTCPPCPAPEAAGGPSVFLFPPKPKDTLMISRTPEVTCVVVDVSHED
PEVKFNWYVDGVEVHNAKTKPREEQYNSTYRWSVLTVLHQDWLNGKEYKC
QVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGN
VFSCSVMHEALHNHYTQKSLSLSPGKGGSGGSGGSGGSGGSGGSGGSGGS
GGSGGSGGSGGSGGSGGSGGSGGSGGSGGSGGSGGSSSRTPSDKPVAHVV
ANPQAEGQLQWLNRRANALLANGVELRDNQLVVPSEGLYLIYSQVLFKGQ
GCPSTHVLLTHTISRIAVSFQTKVNLLSAIKSPCQRETPEGAEAKPWYEP
IYLGGVFQLEKGDRLSAEINRPDYLDFAESGQVYFGIIALGGGGSSSRTP
SDKPVAHVVANPQAEGQLQWLNRRANALLANGVELRDNQLVVPSEGLYLI
YSQVLFKGQGCPSTHVLLTHTISRIAVSFQTKVNLLSAIKSPCQRETPEG
AEAKPVVYEPIYLGGVFQLEKGDRLSAEINRPDYLDFAESGQVYFGIIAL
GGGGSSSRTPSDKPVAHVVANPQAEGQLQWLNRRANALLANGVELRDNQL
VVPSEGLYLIYSQVLFKGQGCPSTHVLLTHTISRIAVSFQTKVNLLSAIK
SPCQRETPEGAEAKPWYEPIYLGGVFQLEKGDRLSAEINRPDYLDFAESG
QVYFGIIAL

### Production and Purification of AFRs

Constructs scFv CD20 Ab-20*GGS-Fc3 and Fc4-20*GGS-3*TNF_Y87F were combined to a heterodimeric AFR with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HEK-Dual Reporter Cell-Lines

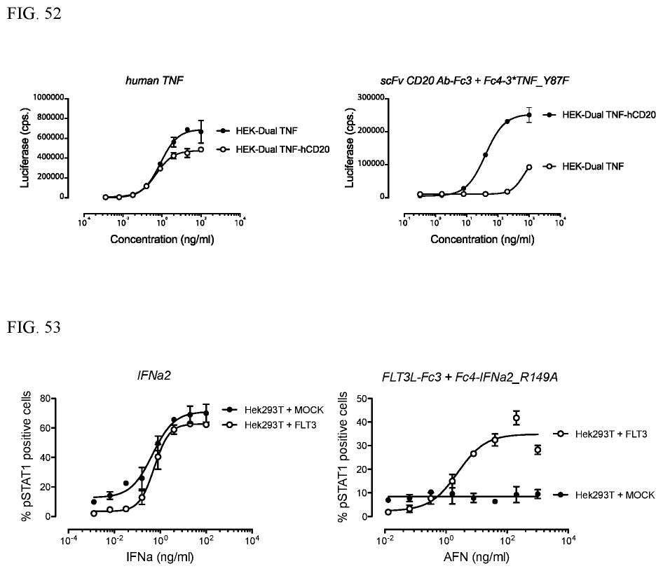

HEK-Dual TNF-α cells (InvivoGen) were derived from the human embryonic kidney 293 (HEK 293) cell line by stable co-transfection of two NF-κB-inducible reporter constructs based on either secreted alkaline phosphatase (SEAP) or a secreted luciferase (Lucia). Parental cells were stably transfected with an expression vector encoding the human CD20 sequence. Stable transfected clones were selected in puromycin-containing medium. Parental HEK-Dual and HEK-Dual-hCD20 cells were seeded at 20,000 cells per 96-well and subsequently stimulated overnight with a serial dilution of Fc AFRs. Secreted Lucia luciferase activity was measured using QUANTI-Luc (InvivoGen). Data in the FIG. 52 illustrate that both cell-lines are comparably sensitive to wild type TNF, while the CD20 Fc AFR is more active on targeted cells (HEK-Dual-hCD20) compared to untargeted cells (HEK-Dual).

### Example 30: FLT3L Fc AFNs

In this Example, we designed and evaluated AFNs based on FMS-like tyrosine kinase 3 ligand (FLT3L) for targeting of hematopoietic (blood) progenitors.

### Constructs

FLT3L-20*GGS-Fc3 (P-1623)
(SEQ ID NO: 1528)
TQDCSFQHSPISSDFAVKIRELSDYLLQDYPVTVASNLQDEELCGGLWRL
VLAQRWMERLKTVAGSKMQGLLERVNTEIHFVTKCAFQPPPSCLRFVQTN
ISRLLQETSEQLVALKPWITRQNFSRCLELQCQPDSSTLPPPWSPRPLEA
TAPTAPQPSGGSGGSGGSGGSGGSGGSGGSGGSGGSGGSGGSGGSGGSGG
SGGSGGSGGSGGSGGSGGSDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCQVSNKALPAPIEKTISKAKGQPREPQVCTL
PPSRDELTKNQVSLSCAVKGFYPSDIAVEWESNGQPENNYKTTPPVLDSD
GSFFLVSKLTVDKSRWQQGNVFSCSVMHEALHNHYTQKSLSLSPGK
Fc4-20*GGS-IFNa2_R149A (P-1414)
(SEQ ID NO: 1529)
DKTHTCPPCPAPEAAGGPSVFLFPPKPKDTLMISRTPEVTCVVVDVSHED
PEVKFNWYVDGVEVHNAKTKPREEQYNSTYRWSVLTVLHQDWLNGKEYKC
QVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGN
VFSCSVMHEALHNHYTQKSLSLSPGKGGSGGSGGSGGSGGSGGSGGSGGS
GGSGGSGGSGGSGGSGGSGGSGGSGGSGGSGGSGGSCDLPQTHSLGSRRT
LMLLAQMRKISLFSCLKDRHDFGFPQEEFGNQFQKAETIPVLHEMIQQIF
NLFSTKDSSAAWDETLLDKFYTELYQQLNDLEACVIQGVGVEETPLMKED
SILAVRKYFQRITLYLKEKKYSPCAWEVVRAEIMASFSLSTNLQESLRSK
E

### Production and Purification of FLT3L-Based AFN

Constructs FLT3L-20*GGS-Fc3 and Fc4-20*GGS-IFNa2_R149A were combined to an AFN variant with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity: STAT1 Phosphorylation in Transient Transfected Hek293T Cells

Hek293T cells were transiently transfected with a FLT3 expression plasmid or an empty vector (MOCK). Two days after transfection, cells were stimulated with a serial dilution (as indicated) wild type IFNa2 or FLT3L-Fc-AFN for 15 minutes at 37° C. After fixation (10 minutes, 37° C., Fix Buffer I; BD Biosciences), permeabilization (30 minutes, on ice, Perm III Buffer I; BD Biosciences) and washing, cells were stained with anti-STAT1 pY701 Ab (BD Biosciences). Samples were acquired with a MACSQuant X instrument (Miltenyi Biotec) and analysed using the FlowLogic software (Miltenyi Biotec). Data in FIG. 53 clearly illustrate that the FLT3L Fc AFN is only capable to induce STAT1 phosphorylation in FLT3 and not MOCK transfected cells, thereby illustrating that targeting using this ligand is possible. Of note, FLT3 or MOCK transfected cells are equally sensitive to wild type IFNa2.

### Example 31: PD-1Ec Fc AFNs

In this Example, we designed and evaluated AFNs based on the extracellular (ec) portion of programmed cell death protein 1 (PD-1) for targeting to PD-L1 expressing T and pro-B cells.

### Constructs:

PD-1ec-20*GGS-Fc3 (P-1829)
(SEQ ID NO: 1530)
PGWFLDSPDRPWNPPTFSPALLWTEGDNATFTCSFSNTSESFVLNWYRMS
PSNQTDKLAAFPEDRSQPGQDCRFRVTQLPNGRDFHMSVVRARRNDSGTY
LCGAISLAPKAQIKESLRAELRVTERRAEVPTAHPSPSPRPAGQFQTLVS
GGSGGSGGSGGSGGSGGSGGSGGSGGSGGSGGSGGSGGSGGSGGSGGSGG
SGGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCQVSNKALPAPIEKTISKAKGQPREPQVCTLPPSRDELTK
NQVSLSCAVKGFYPSDIAVEWESNGQPENNYKTTPPVLDSDGSFFLVSKL
TVDKSRWQQGNVFSCSVMHEALHNHYTQKSLSLSPGK
Fc4-20*GGS-IFNa2_R149A (P-1414)
(SEQ ID NO: 1531)
DKTHTCPPCPAPEAAGGPSVFLFPPKPKDTLMISRTPEVTCVVVDVSHED
PEVKFNWYVDGVEVHNAKTKPREEQYNSTYRWSVLTVLHQDWLNGKEYKC
QVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGN
VFSCSVMHEALHNHYTQKSLSLSPGKGGSGGSGGSGGSGGSGGSGGSGGS
GGSGGSGGSGGSGGSGGSGGSGGSGGSGGSGGSGGSCDLPQTHSLGSRRT
LMLLAQMRKISLFSCLKDRHDFGFPQEEFGNQFQKAETIPVLHEMIQQIF
NLFSTKDSSAAWDETLLDKFYTELYQQLNDLEACVIQGVGVEETPLMKED
SILAVRKYFQRITLYLKEKKYSPCAWEVVRAEIMASFSLSTNLQESLRSK
E

### Production and Purification of PD-1ec AFN

Constructs PD-1ec-20*GGS-Fc3 and Fc4-20*GGS-IFNa2_R149A were combined to a heterodimeric AFN with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells express PD-L1 (the ligand for PD-1) endogenously, so targeting was evaluated in the absence or presence of an excess of the PD-L1 VHH (2LIG99) that interferes with the PD-1/PD-L1 interaction. Cells were seeded overnight at 20,000 cells per 96-well and subsequently stimulated with a serial dilution of PD-1 Fc AFNs for 6 hours in the presence or absence of 2LIG99 VHH (50 μg/ml). Luciferase activity was measured in cell lysates. Data in the FIG. 54 illustrate that the VHH is able to, at least in part, neutralize signaling by the PD-1ec Fc AFN, thereby illustrating that it is possible to target mutated cytokines based on the PD-1/PD-L1 interaction.

### Example 32: PD-L1ec Fc AFNs

In this Example, we designed and evaluated AFNs based on the extracellular portion (ec) of programmed cell death ligand 1 (PD-L1) for targeting of tumor cells or activated immune cells.

### Constructs:

PD-L1ec-20*GGS-Fc3 (P-1830)
(SEQ ID NO: 1532)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNERSGGSGGSGGSGGSGGSGGSGGSGGSGGSGG
SGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKT
KPREEQYNSTYRVVSVLTVLHQDWLNGKEYKCQVSNKALPAPIEKTISKA
KGQPREPQVCTLPPSRDELTKNQVSLSCAVKGFYPSDIAVEWESNGQPEN
NYKTTPPVLDSDGSFFLVSKLTVDKSRWQQGNVFSCSVMHEALHNHYTQK
SLSLSPGK
Fc4-20*GGS-IFNa2_R149A (P-1414)
(SEQ ID NO: 1533)
DKTHTCPPCPAPEAAGGPSVFLFPPKPKDTLMISRTPEVTCVVVDVSHED
PEVKFNWYVDGVEVHNAKTKPREEQYNSTYRWSVLTVLHQDWLNGKEYKC
QVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGN
VFSCSVMHEALHNHYTQKSLSLSPGKGGSGGSGGSGGSGGSGGSGGSGGS
GGSGGSGGSGGSGGSGGSGGSGGSGGSGGSGGSGGSCDLPQTHSLGSRRT
LMLLAQMRKISLFSCLKDRHDFGFPQEEFGNQFQKAETIPVLHEMIQQIF
NLFSTKDSSAAWDETLLDKFYTELYQQLNDLEACVIQGVGVEETPLMKED
SILAVRKYFQRITLYLKEKKYSPCAWEVVRAEIMASFSLSTNLQESLRSK
E

### Production and Purification of PD-L1ec-Based AFN

Constructs PD-L1ec-20*GGS-Fc3 and Fc4-20*GGS-IFNa2_R149A were combined to a heterodimeric AFN with a configuration outlined in FIG. 7B, and transiently expressed in the ExpiCHO expression system (Thermo Fisher) according to the manufacturer's guidelines. One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human PD-1 sequence lacking its cytoplasmic tail. Stable transfected clones were selected in G418-containing medium. Parental HL116 and HL116-hPD-1 cells were seeded overnight at 20,000 cells per 96-well and subsequently stimulated with a serial dilution of Fc AFNs for 6 hours. Luciferase activity was measured in cell lysates. Data in the FIG. 55 illustrate that both cell-lines are comparably sensitive to wild type IFNα2, while the PD-L1ec Fc AFN is more active on targeted cells (HL116-PD-1) compared to untargeted cells (parental HL116).

### Example 33: Bivalent Ligand Targeted AFNs

In this Example, we designed and evaluated AFNs based on the use of bivalent extracellular (ec) portions of programmed cell death protein 1 (PD-1) for targeting to PD-L1 expressing T and pro-B cells or programmed cell death ligand 1 (PD-L1) for targeting of tumor cells or activated immune cells.

### Constructs:

PD-1ec-20*GGS-Fc3 (P-1829)
(SEQ ID NO: 1534)
PGWFLDSPDRPWNPPTFSPALLWTEGDNATFTCSFSNTSESFVLNWYRMS
PSNQTDKLAAFPEDRSQPGQDCRFRVTQLPNGRDFHMSVVRARRNDSGTY
LCGAISLAPKAQIKESLRAELRVTERRAEVPTAHPSPSPRPAGQFQTLVS
GGSGGSGGSGGSGGSGGSGGSGGSGGSGGSGGSGGSGGSGGSGGSGGSGG
SGGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCQVSNKALPAPIEKTISKAKGQPREPQVCTLPPSRDELTK
NQVSLSCAVKGFYPSDIAVEWESNGQPENNYKTTPPVLDSDGSFFLVSKL
TVDKSRWQQGNVFSCSVMHEALHNHYTQKSLSLSPGK
PD-1ec-20*GGS-Fc4-20*GGS-IFNa2_R149A
(SEQ ID NO: 1535)
PGWFLDSPDRPWNPPTFSPALLWTEGDNATFTCSFSNTSESFVLNWYRMS
PSNQTDKLAAFPEDRSQPGQDCRFRVTQLPNGRDFHMSVVRARRNDSGTY
LCGAISLAPKAQIKESLRAELRVTERRAEVPTAHPSPSPRPAGQFQTLVS
GGSGGSGGSGGSGGSGGSGGSGGSGGSGGSGGSGGSGGSGGSGGSGGSGG
SGGSGGSGGSDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCQVSNKALPAPIEKTISKAKGQPREPQVYTLPPCRDELTK
NQVSLWCLVKGFYPSDIAVEWESNGQPENNYKTTPPVLDSDGSFFLYSKL
TVDKSRWQQGNVFSCSVMHEALHNHYTQKSLSLSPGKGGSGGSGGSGGSG
GSGGSGGSGGSGGSGGSGGSGGSGGSGGSGGSGGSGGSGGSGGSGGSCDL
PQTHSLGSRRTLMLLAQMRKISLFSCLKDRHDFGFPQEEFGNQFQKAETI
PVLHEMIQQIFNLFSTKDSSAAWDETLLDKFYTELYQQLNDLEACVIQGV
GVEETPLMKEDSILAVRKYFQRITLYLKEKKYSPCAWEVVRAEIMASFSL
STNLQESLRSKE
PD-L1ec-20*GGS-Fc3 (P-1830)
(SEQ ID NO: 1536)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNERSGGSGGSGGSGGSGGSGGSGGSGGSGGSGG
SGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKT
KPREEQYNSTYRVVSVLTVLHQDWLNGKEYKCQVSNKALPAPIEKTISKA
KGQPREPQVCTLPPSRDELTKNQVSLSCAVKGFYPSDIAVEWESNGQPEN
NYKTTPPVLDSDGSFFLVSKLTVDKSRWQQGNVFSCSVMHEALHNHYTQK
SLSLSPGK
PD-L1ec-20*GGS-Fc4-20*GGS-IFNa2_R149A
(SEQ ID NO: 1537)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNERSGGSGGSGGSGGSGGSGGSGGSGGSGGSGG
SGGSGGSGGSGGSGGSGGSGGSGGSGGSGGSDKTHTCPPCPAPEAAGGPS
VFLFPPKPKDTLMISRTPEVTCVVVDVSHEDPEVKFNWYVDGVEVHNAKT
KPREEQYNSTYRVVSVLTVLHQDWLNGKEYKCQVSNKALPAPIEKTISKA
KGQPREPQVYTLPPCRDELTKNQVSLWCLVKGFYPSDIAVEWESNGQPEN
NYKTTPPVLDSDGSFFLYSKLTVDKSRWQQGNVFSCSVMHEALHNHYTQK
SLSLSPGKGGSGGSGGSGGSGGSGGSGGSGGSGGSGGSGGSGGSGGSGGS
GGSGGSGGSGGSGGSGGSCDLPQTHSLGSRRTLMLLAQMRKISLFSCLKD
RHDFGFPQEEFGNQFQKAETIPVLHEMIQQIFNLFSTKDSSAAWDETLLD
KFYTELYQQLNDLEACVIQGVGVEETPLMKEDSILAVRKYFQRITLYLKE
KKYSPCAWEVVRAEIMASFSLSTNLQESLRSKE

### Production and Purification of Bivalent Ec-AFNs

The following combinations were transiently transfected in ExpiCHO cells:
i. PD-1ec-20*GGS-Fc3 and PD-1ec-20*GGS-Fc4-20*GGS-IFNα2_R149A (bispecific; monovalent targeting; scheme: FIG. 16A);
ii. PD-L1ec-20*GGS-Fc3 and PD-L1ec-20*GGS-Fc4-20*GGS-IFNα2_R149A (bispecific; monovalent targeting; scheme: FIG. 16A).

One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells express PD-L1 (the ligand for PD-1) endogenously, so targeting was evaluated in the absence or presence of an excess of free PD-L1 VHH (2LIG99) that interferes with the PD-1/PD-L1 interaction. In addition, the HL116 cells transfected with the human PD-1 sequence lacking its cytoplasmic tail (Example 32) was used to test the PD-L1 based construct. Cells were seeded overnight at 20,000 cells per 96-well and subsequently stimulated with a serial dilution of PD-1 Fc AFNs for 6 hours in the presence or absence of 2LIG99 VHH (50 μg/ml). Luciferase activity was measured in cell lysates.

### Example 34: NGR Peptide Based Fc-AFNs

In this example, we designed and evaluated AFNs targeted to CD13 in the tumor neovasculature with the NGR-peptide-motif as targeting agent. The sequence was cloned as a cyclic sgcNGRc peptide C-terminal of both Fc arms in the resulting NGR Fc AFN. Biological activity on parental HL116 cells was compared with that of a Clec9A-targeted Fc AFN.

### Constructs:

Fc1-20*GGS-R1CHCL50 (P-1215)
(SEQ ID NO: 1538)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLTCLVK
GFYPSDIAVEWESNGQPENNYKTTPPVLDSDGSFFLTSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSDVQLVESGGGLVQ
PGGSLRLSCAASGSFSSINVMGWYRQAPGKERELVARITNLGLPNYADSV
KGRFTISRDNSKNTVYLQMNSLRPEDTAVYYCYLVALKAEYWGQGTLVTV
SS
IFNa2_R149A-20*GGS-Fc2 (P-1214)
(SEQ ID NO: 1539)
CDLPQTHSLGSRRTLMLLAQMRKISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMAS
FSLSTNLQESLRSKEGGSGGSGGSGGSGGSGGSGGSGGSGGSGGSGGSGG
SGGSGGSGGSGGSGGSGGSGGSGGSDKTHTCPPCPAPEAAGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALGAPIEKTISKAKGQPRE
PQVYTLPPSRDELTKNQVSLYCLVKGFYPSDIAVEWESNGQPENNYKTTP
PVLDSDGSFFLYSKLTVDKSRWQQGNVFSCSVMHEALHNHYTQKSLSLSP
GK
Fc3-spcNGRc (P-1853)
(SEQ ID NO: 1540)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALGAPIEKTISKAKGQPREPQVYTLPPSRDELTKNQVSLTCLVK
GFYPSDIAVEWESNGQPENNYKTTPPVLDSDGSFFLTSKLTVDKSRWQQG
NVFSCSVMHEALHNHYTQKSLSLSPGKSGCNGRC
IFNa2_R149A-20*GGS-Fc4-spcNGRc (P-1854)
(SEQ ID NO: 1541)
CDLPQTHSLGSRRTLMLLAQMRKISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMAS
FSLSTNLQESLRSKEGGSGGSGGSGGSGGSGGSGGSGGSGGSGGSGGSGG
SGGSGGSGGSGGSGGSGGSGGSGGSDKTHTCPPCPAPEAAGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALGAPIEKTISKAKGQPRE
PQVYTLPPSRDELTKNQVSLYCLVKGFYPSDIAVEWESNGQPENNYKTTP
PVLDSDGSFFLYSKLTVDKSRWQQGNVFSCSVMHEALHNHYTQKSLSLSP
GKSGCNGRC

### Production and Purification of AFNs

Constructs were combined as follows and transiently expressed in the ExpiCHO expression system (Thermo Fisher) according to the manufacturer's guidelines:
(i) Clec9A VHH Fc AFN: Fc3-20*GGS-R1CHCL50+IFNα2_R149A-20*GGS-Fc4 (configuration as outlined in FIG. 7A);
(ii) NGR Fc AFN: Fc3-sgNGRc+IFNa2_R149A-20*GGS-Fc4-sgcNGRc (configuration as outlined in FIG. 16B).

One week after transfection, supernatant was collected and cells removed by centrifugation. Recombinant proteins were purified from the supernatant using the Pierce Protein A spin plates (Thermo Fisher).

### Biological Activity on HL116 Reporter Cell-Lines

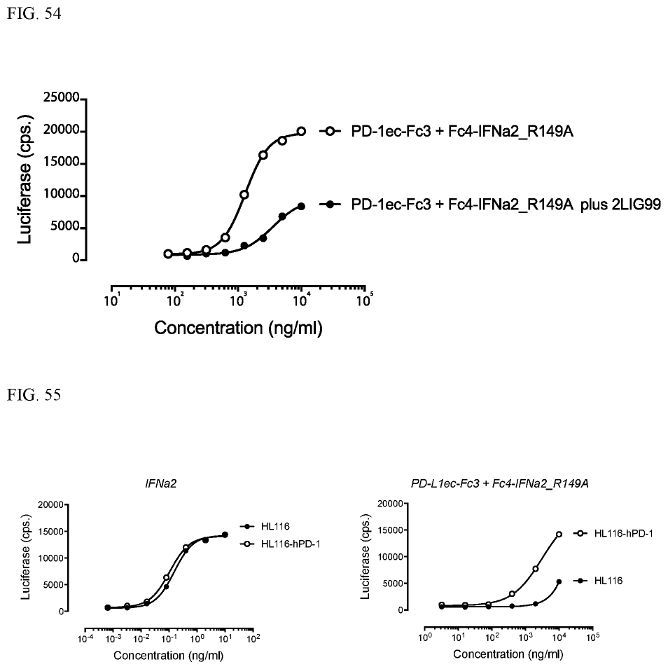

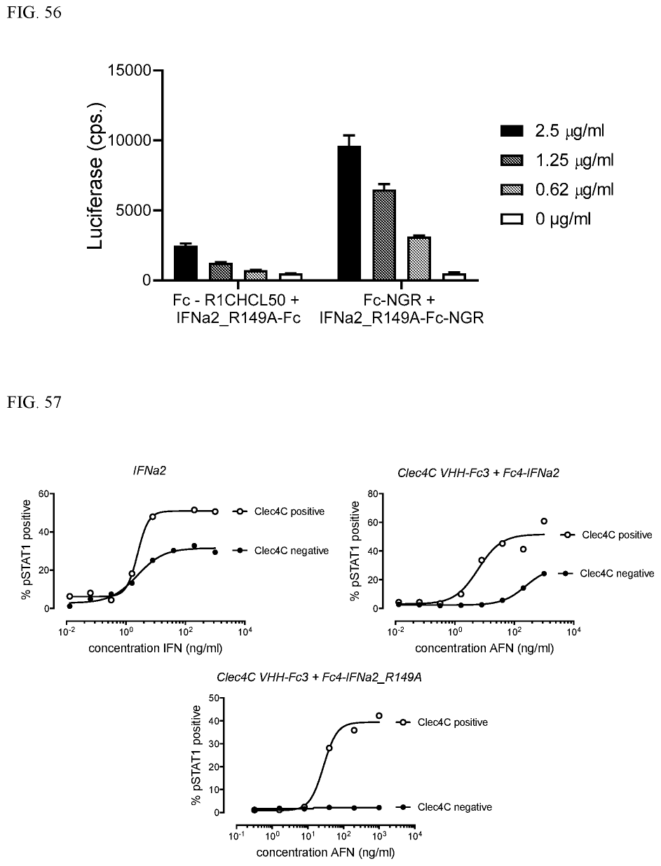

The HL116 clone is derived from the human HT1080 cell line (ATCC CCL-121). It contains the firefly luciferase gene controlled by the IFN-inducible 6-16 promoter. Parental HL116 cells endogenously express the NGR target CD13. Cells were seeded overnight at 20.000 cells per 96-well and subsequently stimulated with a serial dilution of Fc AFNs for 6 hours. Luciferase activity was measured in cell lysates. Data in the FIG. 56 illustrate that the NGR Fc AFN is clearly more potent in inducing IFN-like signaling when compared to a Clec9A targeted Fc AFN showing the specificity of the NGR targeting.

### Example 35: Targeting of Wild Type IFNa2

In this Example, we compared signaling upon targeting of wild type IFNa2 or the AFN mutant IFNa2_R149A to specific populations within peripheral blood mononuclear cells (PBMCs). Cytokine, or mutant thereof, will be targeted using a CD20 VHH (clone 2HCD25), CD8 VHH (clone 1CDA65), or a Clec4C VHH (clone 2CL92) in an Fc context.

### Constructs:

CD20 VHH-20*GGS-Fc3 (P-1570)
(SEQ ID NO: 1542)
QVQLQESGGGLAQAGGSLRLSCAASGRTFSMGWFRQAPGKEREFVAAITY
SGGSPYYASSVRGRFTISRDNAKNTVYLQMNSLKPEDTAVYYCAANPTYG
SDWNAENWGQGTQVTVSSGGSGGSGGSGGSGGSGGSGGSGGSGGSGGSGG
SGGSGGSGGSGGSGGSGGSGGSGGSGGSDKTHTCPPCPAPEAAGGPSVFL
FPPKPKDTLMISRTPEVTCVVVDVSHEDPEVKFNWYVDGVEVHNAKTKPR
EEQYNSTYRVVSVLTVLHQDWLNGKEYKCQVSNKALPAPIEKTISKAKGQ
PREPQVCTLPPSRDELTKNQVSLSCAVKGFYPSDIAVEWESNGQPENNYK
TTPPVLDSDGSFFLVSKLTVDKSRWQQGNVFSCSVMHEALHNHYTQKSLS
LSPGK
CD8 VHH-20*GGS-Fc 3 (P-1568)
(SEQ ID NO: 1543)
QVQLQESGGGLVHPGGSLRLSCAASGRSFSSYFMGWFRQAPGKEREFVAG
IGWNDGSINYADSVKGRFTISRDNAKNTGYLQMNSLKPEDTAVYYCAASV
SLYGLEKSSAYTSWGQGTQVTVSSGGSGGSGGSGGSGGSGGSGGSGGSGG
SGGSGGSGGSGGSGGSGGSGGSGGSGGSGGSGGSDKTHTCPPCPAPEAAG
GPSVFLFPPKPKDTLMISRTPEVTCVVVDVSHEDPEVKFNWYVDGVEVHN
AKTKPREEQYNSTYRVVSVLTVLHQDWLNGKEYKCQVSNKALPAPIEKTI
SKAKGQPREPQVCTLPPSRDELTKNQVSLSCAVKGFYPSDIAVEWESNGQ
PENNYKTTPPVLDSDGSFFLVSKLTVDKSRWQQGNVFSCSVMHEALHNHY
TQKSLSLSPGK
Clec4C VHH-20*GGS-Fc3 (P-1571)
(SEQ ID NO: 1544)
QVQLQESGGGSVQAGDSLRLSCAASGRTFSGYAMGWFRQAPGKEREFVAT
ISTSGSSTYYADSVKGRFTISRDNAKKSVYLQINSLKTEDAAVYYCAARL
SFDNTAFYTSAIRYSYWGQGTQVTVSSGGSGGSGGSGGSGGSGGSGGSGG
SGGSGGSGGSGGSGGSGGSGGSGGSGGSGGSGGSGGSDKTHTCPPCPAPE
AAGGPSVFLFPPKPKDTLMISRTPEVTCVVVDVSHEDPEVKFNWYVDGVE
VHNAKTKPREEQYNSTYRVVSVLTVLHQDWLNGKEYKCQVSNKALPAPIE
KTISKAKGQPREPQVCTLPPSRDELTKNQVSLSCAVKGFYPSDIAVEWES
NGQPENNYKTTPPVLDSDGSFFLVSKLTVDKSRWQQGNVFSCSVMHEALH
NHYTQKSLSLSPGK
Fc4-20*GGS-IFNa2_R149A (P-1414)
(SEQ ID NO: 1545)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE
Fc4-20*GGS-IFNa2 (P-1538)
(SEQ ID NO: 1546)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMRSFSLSTNLQESLRS
KE

### Production and Purification of CD20 and Clec4C VHH-Based AFN

Combinations of these constructs were transiently transfected in ExpiCHO cells and resulted in following AFNs (for a schematic outline, see FIG. 7B):
(i) CD20 VHH-20*GGS-Fc3+Fc4-IFNa2_R149A
(ii) CD20 VHH-20*GGS-Fc3+Fc4-IFNa2
(iii) CD8 VHH-20*GGS-Fc3+Fc4-IFNa2_R149A
(iv) CD8 VHH-20*GGS-Fc3+Fc4-IFNa2
(v) Clec4C VHH-20*GGS-Fc3+Fc4-IFNa2_R149A
(vi) Clec4C VHH-20*GGS-Fc3+Fc4-IFNa2

One week after transfection, supernatant was collected, and cells removed by centrifugation. Recombinant proteins were purified from the supernatant using the Pierce Protein A spin plates (Thermo Fisher).

### Biological Activity Upon Clec4C Targeting: STAT1 Phosphorylation in PBMCs

Biological activity of Clec4C-targeted molecules was tested as follows: PBMCs from buffy coats of healthy donors were isolated using density gradient centrifugation with Ficoll-Paque (GE Healthcare). Dendritic cells were first enriched using the pan-DC enrichment kit (Miltenyi Biotec). Enriched cells were washed twice with FACS buffer (2% FBS, 1 mM EDTA in PBS) and stained with FITC-coupled anti-hClec4C (Miltenyi Biotec) for 20 minutes at 4° C. After two washes, cells were stimulated with a serial dilution of wild type or mutants Fc AFNs for 15 minutes at 37° C. After fixation (10 minutes, 37° C., Fix Buffer I; BD Biosciences) and permeabilization (30 minutes, on ice, Perm III Buffer I; BD Biosciences) and washing, cells were stained with anti-STAT1 pY701 Ab (BD Biosciences). Samples were acquired with a Wacquant X instrument (Miltenyi Biotec) and analysed using the FlowLogic software (Miltenyi Biotec). Data are summarized in FIG. 57. Clec4C positive and negative cells respond with similar potency (ratio of EC50s: ≈1) to wild type IFNa2. On the other hand, wild type or mutant IFNa2 is much more active when targeted with a Clec4C VHH to CLEC4C positive cells. The respective EC50 ratios are about 40 and >40.

### Biological Activity Upon CD8 Targeting: STAT1 Phosphorylation in PBMCs

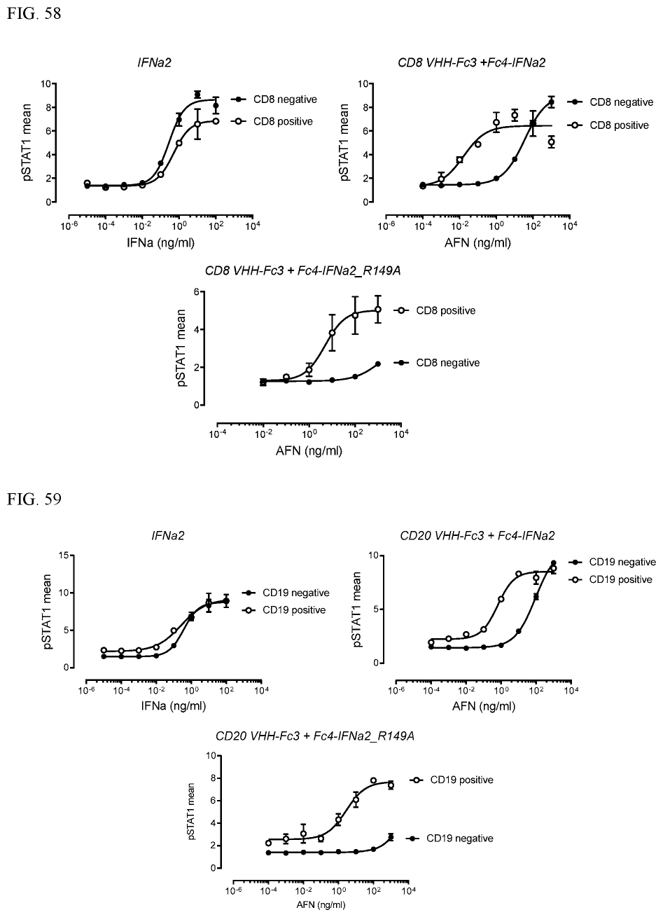

Biological activity of CD8-targeted molecules was tested as follows: PBMCs from buffy coats of healthy donors were isolated using density gradient centrifugation with Ficoll-Paque (GE Healthcare). Cells were washed twice with FACS buffer (2% FBS, 1 mM EDTA in PBS) and stained with FITC-coupled anti-hCD8 (Miltenyi Biotec) for 20 minutes at 4° C. After two washes, cells were stimulated with a serial dilution of wild type or mutants Fc AFNs for 15 minutes at 37° C. After fixation (10 minutes, 37° C., Fix Buffer I; BD Biosciences) and permeabilization (30 minutes, on ice, Perm III Buffer I; BD Biosciences) and washing, cells were stained with anti-STAT1 pY701 Ab (BD Biosciences). Samples were acquired with a MACSQuant X instrument (Miltenyi Biotec) and analysed using the FlowLogic software (Miltenyi Biotec). Data are summarized in FIG. 58. CD8 positive and negative cells respond with similarly potency (ratio of EC50s: ≈1) to wild type IFNa2. On the other hand, wild type or mutant IFNa2 is much more active when targeted with a CD8 VHH to CD8 positive cells. The respective EC50 ratios are about 125 and >125.

### Biological Activity Upon CD20 Targeting: STAT1 Phosphorylation in PBMCs

Biological activity of CD20-targeted molecules was tested as follows: PBMCs from buffy coats of healthy donors were isolated using density gradient centrifugation with Ficoll-Paque (GE Healthcare). Cells were washed twice with FACS buffer (2% FBS, 1 mM EDTA in PBS) and B-cells stained with FITC-coupled anti-hCD19 (SinoBiologics) for 20 minutes at 4° C. After two washes, cells were stimulated with a serial dilution of wild type or mutants Fc AFNs for 15 minutes at 37° C. After fixation (10 minutes, 37° C., Fix Buffer I; BD Biosciences) and permeabilization (30 minutes, on ice, Perm III Buffer I; BD Biosciences) and washing, cells were stained with anti-STAT1 pY701 Ab (BD Biosciences). Samples were acquired with a MACSQuant X instrument (Miltenyi Biotec) and analysed using the FlowLogic software (Miltenyi Biotec). Data are summarized in FIG. 59. CD19 positive (i.e. B-cells) and negative cells respond with similarly potency (ratio of EC50s: ≈1) to wild type IFNa2. On the other hand, wild type or mutant IFNa2 is much more active when targeted with a CD20 VHH to CD19 positive cells. The respective EC50 ratios are about 25 and >500.

### Example 36: Alternative IFNa2 AFN Mutations

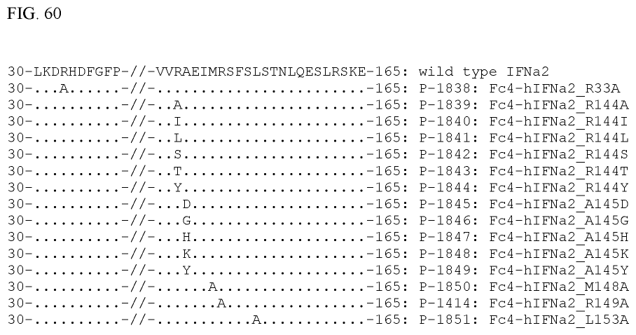

In this Example, several different residues in IFNα2 were mutated in a Clec9A targeted Fc AFN context to evaluate the effect on signaling in targeted and untargeted cells. The positions of the various mutations are shown in FIG. 60 (here, wild type is SEQ ID NO: 2).

### Constructs (FIG. 60)

30-LKDRHDFGFP-//-VVRAEIMRSFSLSTNLQESLRSKE-165: wild type IFNa2
30-...A......-//-........................-165: 0-1838: Fc4-hIFNa2_R33A
30-..........-//-..A.....................-165: 0-1839: Fc4-hIFNa2_R144A
30-..........-//-..I.....................-165: 0-1840: Fc4-hIFNa2_R144I
30-..........-//-..L.....................-165: 0-1841: Fc4-hIFNa2_R144L
30-..........-//-..S.....................-165: 0-1842: Fc4-hIFNa2_R144S
30-..........-//-..T.....................-165: 0-1843: Fc4-hIFNa2_R144T
30-..........-//-..Y.....................-165: 0-1844: Fc4-hIFNa2_R144Y
30-..........-//-...D....................-165: 0-1845: Fc4-hIFNa2_A145D
30-..........-//-...G....................-165: 0-1846: Fc4-hIFNa2_A145G
30-..........-//-...H....................-165: 0-1847: Fc4-hIFNa2_A145H
30-..........-//-...K....................-165: 0-1848: Fc4-hIFNa2_A145K
30-..........-//-...Y....................-165: 0-1849: Fc4-hIFNa2_A145Y
30-..........-//-......A.................-165: 0-1850: Fc4-hIFNa2_M148A
30-..........-//-.......A................-165: 0-1414: Fc4-hIFNa2_R149A
30-..........-//-...........A............-165: 0-1851: Fc4-hIFNa2_L153A

### Production and Purification of AFNs

Different mutation variants of Fc4-20*GGS-IFNa2 were combined with construct R1CHCL50-20*GGS-Fc3, resulting in an AFN with a structure outlined in FIG. 7B. Proteins were transiently expressed in the ExpiCHO expression system (Thermo Fisher) according to the manufacturer's guidelines. One week after transfection, supernatant was collected, and cells removed by centrifugation. Recombinant proteins were purified from the supernatant using the Pierce Protein A spin plates (Thermo Fisher).

### Biological Activity on HL116 Reporter Cell-Lines

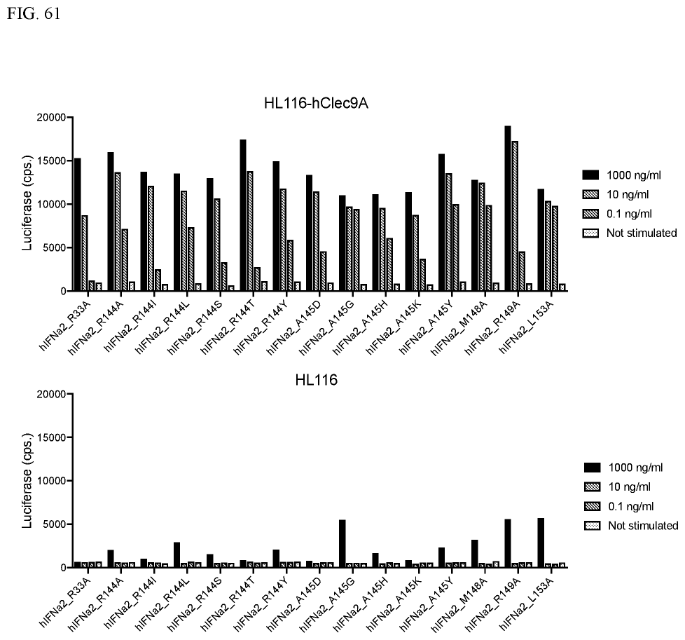

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well and subsequently stimulated with different concentrations of Fc AFNs for 6 hours. Luciferase activity was measured in cell lysates. Data in the FIG. 61 illustrate that all mutations, result in an Fc AFN that preferentially signals in targeted cells (here hClec9A expressing HL116).

### Example 37: Actakines Based on Interferon Alpha 1

This Example evaluates and generate AcTakines based on wild type interferon alpha 1 (IFNα1) and targeted via a human Clec9A specific VHH (clone R1CHCL50).

### Constructs:

R1CHCL50-20*GGS-Fc3 (P-1451)
(SEQ ID NO: 1547)
QVQLVESGGGLVHPGGSLRLSCAASGSFSSINVMGWYRQAPGKERELVAR
ITNLGLPNYADSVTGRFTISRDNAKNTVYLQMNSLKPEDTAVYYCYLVAL
KAEYWGQGTQVTVSSGGSGGSGGSGGSGGSGGSGGSGGSGGSGGSGGSGG
SGGSGGSGGSGGSGGSGGSGGSGGSDKTHTCPPCPAPEAAGGPSVFLFPP
KPKDTLMISRTPEVTCVVVDVSHEDPEVKFNWYVDGVEVHNAKTKPREEQ
YNSTYRVVSVLTVLHQDWLNGKEYKCQVSNKALPAPIEKTISKAKGQPRE
PQVCTLPPSRDELTKNQVSLSCAVKGFYPSDIAVEWESNGQPENNYKTTP
PVLDSDGSFFLVSKLTVDKSRWQQGNVFSCSVMHEALHNHYTQKSLSLSP
GK
Fc4-20*GGS-IFNa1 (P-1852)
(SEQ ID NO: 1548)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPETHSLDNRR
TLMLLAQMSRISPSSCLMDRHDFGFPQEEFDGNQFQKAPAISVLHELIQQ
IFNLFTTKDSSAAWDEDLLDKFCTELYQQLNDLEACVMQEERVGETPLMN
ADSILAVKKYFRRITLYLTEKKYSPCAWEVVRAEIMRSLSLSTNLQERLR
RKE

### Production and Purification of IFNα1 AFN

Constructs R1CHCL50-20*GGS-Fc3 and Fc4-20*GGS-IFNα1 were combined, resulting in an AFN with a structure outlined in FIG. 7B, and transiently expressed in the ExpiCHO expression system (Thermo Fisher) according to the manufacturer's guidelines. One week after transfection, supernatant was collected, and cells removed by centrifugation. Recombinant proteins were purified from the supernatant using the Pierce Protein A spin plates (Thermo Fisher).

### Biological Activity on HL116 Reporter Cell-Lines

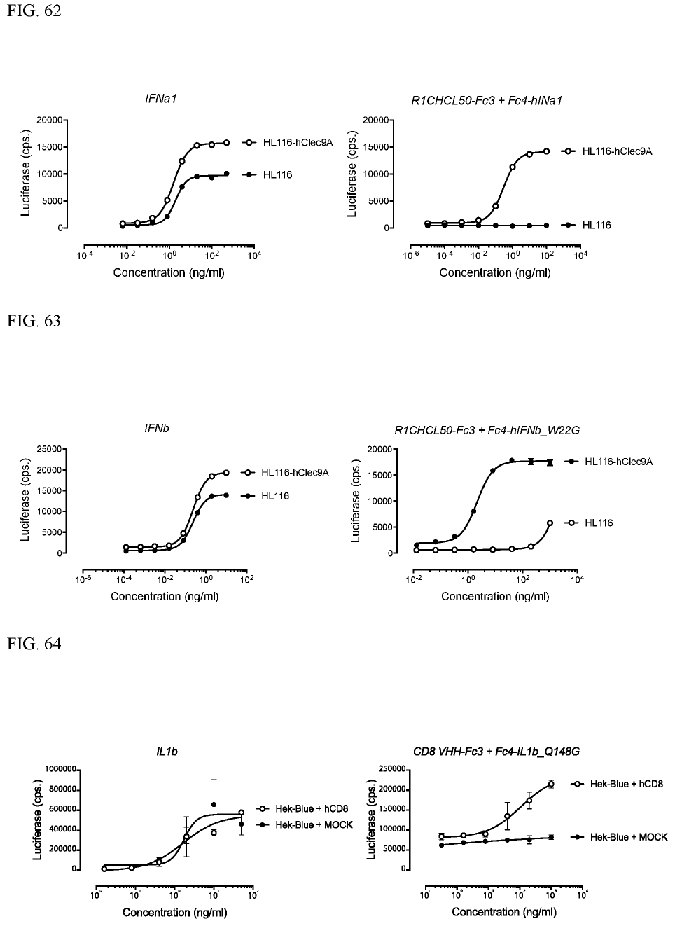

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well and subsequently stimulated with a serial dilution of Fc AFNs for 6 hours. Luciferase activity was measured in cell lysates. Data in the FIG. 62 illustrate that both cell-lines are comparably sensitive to wild type IFNα1. The EC50 of IFNα1-based Fc AFN signaling in targeted cells is 0.3 ng/ml, while no luciferase induction could be measured in parental HL116 cells.

### Example 38: Actakines Based on Interferon ß

In this Example, we generated and evaluated AcTakines based on interferon beta with the W22G mutation (IFNb_W22G) and targeted via a human Clec9A specific VHH (clone R1CHCL50).

### Constructs:

R1CHCL50-20*GGS-Fc3 (P-1451)
(SEQ ID NO: 1549)
QVQLVESGGGLVHPGGSLRLSCAASGSFSSINVMGWYRQAPGKERELVAR
ITNLGLPNYADSVTGRFTISRDNAKNTVYLQMNSLKPEDTAVYYCYLVAL
KAEYWGQGTQVTVSSGGSGGSGGSGGSGGSGGSGGSGGSGGSGGSGGSGG
SGGSGGSGGSGGSGGSGGSGGSGGSDKTHTCPPCPAPEAAGGPSVFLFPP
KPKDTLMISRTPEVTCVVVDVSHEDPEVKFNWYVDGVEVHNAKTKPREEQ
YNSTYRVVSVLTVLHQDWLNGKEYKCQVSNKALPAPIEKTISKAKGQPRE
PQVCTLPPSRDELTKNQVSLSCAVKGFYPSDIAVEWESNGQPENNYKTTP
PVLDSDGSFFLVSKLTVDKSRWQQGNVFSCSVMHEALHNHYTQKSLSLSP
GK
Fc4-20*GGS-IFNb_W22G (P-1855)
(SEQ ID NO: 1550)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSMSYNLLGFLQRSS
NFQCQKLLGQLNGRLEYCLKDRMNFDIPEEIKQLQQFQKEDAALTIYEML
QNIFAIFRQDSSSTGWNETIVENLLANVYHQINHLKTVLEEKLEKEDFTR
GKLMSSLHLKRYYGRILHYLKAKEYSHCAWTIVRVEILRNFYFINRLTGY
LRN

### Production and Purification of IFNb AFN

Constructs R1CHCL50-20*GGS-Fc3 and Fc4-20*GGS-IFNb_W22G were combined, resulting in an AFN with a structure outlined in FIG. 7B, and transiently expressed in the ExpiCHO expression system (Thermo Fisher) according to the manufacturer's guidelines. One week after transfection, supernatant was collected, and cells removed by centrifugation. Recombinant proteins were purified from the supernatant using the Pierce Protein A spin plates (Thermo Fisher).

### Biological Activity on HL116 Reporter Cell-Lines

The HL116 clone is derived from the human HT1080 cell line (ATCC CCL-121). It contains the firefly luciferase gene controlled by the IFN-inducible 6-16 promoter. Parental HL116 cells were transfected with an expression vector encoding the human Clec9A sequence. Stable transfected clones were selected in G418-containing medium. Parental HL116 and HL116-hClec9A cells were seeded overnight at 20,000 cells per 96-well and subsequently stimulated with a serial dilution of Fc AFNs for 6 hours. Luciferase activity was measured in cell lysates. Data in the FIG. 63 illustrate that both cell-lines are comparably sensitive to wild type IFNb, while the Clec9A-targeted Fc AFN is much more active on targeted cells (HL116-hClec9A) compared to untargeted cells (parental HL116).

### Example 39: Actakines Based on Interleukin-1

In this Example, we generated and evaluated AcTakines based on human interleukin-1 with the Q148G (hIL1b_Q148G) mutation and targeted via a human CD8 specific VHH (clone 1CDA65). The resulting AcTakine will be referred to as the AcTaleukin (ALN) hereafter.

### Constructs:

CD8 VHH-20*GGS-Fc3 (P-1568)
(SEQ ID NO: 1551)
QVQLQESGGGLVHPGGSLRLSCAASGRSFSSYFMGWFRQAPGKEREFVAGI
GWNDGSINYADSVKGRFTISRDNAKNTGYLQMNSLKPEDTAVYYCAASVSL
YGLEKSSAYTSWGQGTQVTVSSGGSGGSGGSGGSGGSGGSGGSGGSGGSGG
SGGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTKP
REEQYNSTYRVVSVLTVLHQDWLNGKEYKCQVSNKALPAPIEKTISKAKGQ
PREPQVCTLPPSRDELTKNQVSLSCAVKGFYPSDIAVEWESNGQPENNYKT
TPPVLDSDGSFFLVSKLTVDKSRWQQGNVFSCSVMHEALHNHYTQKSLSLS
PGK
Fc4-20*GGS-IL1b_Q148G (P-1626)
(SEQ ID NO: 1552)
DKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCQ
VSNKALPAPIEKTISKAKGQPREPQVYTLPPCRDELTKNQVSLWCLVKGFY
PSDIAVEWESNGQPENNYKTTPPVLDSDGSFFLYSKLTVDKSRWQQGNVFS
CSVMHEALHNHYTQKSLSLSPGKGGSGGSGGSGGSGGSGGSGGSGGSGGSG
GSGGSGGSGGSGGSGGSGGSGGSGGSGGSGGSAPVRSLNCTLRDSQQKSLV
MSGPYELKALHLGGQDMEQQVVFSMSFVQGEESNDKIPVALGLKEKNLYLS
CVLKDDKPTLQLESVDPKNYPKKKMEKRFVFNKIEINNKLEFESAQFPNWY
ISTSQAENMPVFLGGTKGGQDITDFTMQFVSS

### Production and Purification of ALN

Constructs CD8 VHH-20*GGS-Fc3 and Fc4-20*GGS-IL1b_Q148G were combined and transiently expressed in the ExpiCHO expression system (Thermo Fisher) according to the manufacturer's guidelines. Resulting ALN has a structure as outlined in FIG. 7B. One week after transfection, supernatant was collected and cells removed by centrifugation. Recombinant proteins were purified from the supernatant using the Pierce Protein A spin plates (Thermo Fisher).

### Biological Activity on HEK-Blue IL-1β Cells

Biological activity of the resulting ALN was measured on HEK-Blue™ IL-1β Cells (InvivoGen) with an NF-κB/AP-1-inducible SEAP (secreted alkaline phosphatase) reporter. Therefore, cells were transiently transfected with an empty vector or an expression-plasmid encoding human CD8. 36 hours post transfection, cells were resuspended and stimulated overnight with a serial dilution wild type IL-1β or ALN. SEAP was measured using the Phospha-Light SEAP Reporter Gene Assay System (ThermoFisher) according to the manufacturer's guidelines. Data in FIG. 64 illustrate that MOCK or hCD8 transfected cells are comparably sensitive to wild type IL-1β, while ALN is specifically signals in cells expressing the target (here hCD8).

### Example 40: Actakines Based on Tumor Necrosis Factor

In this Example, we generated and evaluated AcTakines based on tumour necrosis factor with the Y87F mutation (TNF_Y87F) and targeted via a human Clec9A specific VHH (clone R1CHCL50). TNF_Y87F will be cloned as a single chain trimer (with GGGGS-linkers) on one Fc arm, and the resulting AcTakine will be referred to as the AcTafactor (AFR) hereafter.

### Constructs:

CD20 VHH-20*GGS-Fc3 (P-1570)
(SEQ ID NO: 1553)
QVQLQESGGGLAQAGGSLRLSCAASGRTFSMGWFRQAPGKEREFVAAITY
SGGSPYYASSVRGRFTISRDNAKNTVYLQMNSLKPEDTAVYYCAANPTYG
SDWNAENWGQGTQVTVSSGGSGGSGGSGGSGGSGGSGGSGGSGGSGGSGG
SGGSGGSGGSGGSGGSGGSGGSGGSGGSDKTHTCPPCPAPEAAGGPSVFL
FPPKPKDTLMISRTPEVTCVVVDVSHEDPEVKFNWYVDGVEVHNAKTKPR
EEQYNSTYRVVSVLTVLHQDWLNGKEYKCQVSNKALPAPIEKTISKAKGQ
PREPQVCTLPPSRDELTKNQVSLSCAVKGFYPSDIAVEWESNGQPENNYK
TTPPVLDSDGSFFLVSKLTVDKSRWQQGNVFSCSVMHEALHNHYTQKSLS
LSPGK
Fc4-20*GGS-3*TNF_Y87F (P-1545)
(SEQ ID NO: 1554)
DKTHTCPPCPAPEAAGGPSVFLFPPKPKDTLMISRTPEVTCVVVDVSHED
PEVKFNWYVDGVEVHNAKTKPREEQYNSTYRWSVLTVLHQDWLNGKEYKC
QVSNKALPAPIEKTISKAKGQPREPQVYTLPPCRDELTKNQVSLWCLVKG
FYPSDIAVEWESNGQPENNYKTTPPVLDSDGSFFLYSKLTVDKSRWQQGN
VFSCSVMHEALHNHYTQKSLSLSPGKGGSGGSGGSGGSGGSGGSGGSGGS
GGSGGSGGSGGSGGSGGSGGSGGSGGSGGSGGSGGSSSRTPSDKPVAHVV
ANPQAEGQLQWLNRRANALLANGVELRDNQLVVPSEGLYLIYSQVLFKGQ
GCPSTHVLLTHTISRIAVSFQTKVNLLSAIKSPCQRETPEGAEAKPWYEP
IYLGGVFQLEKGDRLSAEINRPDYLDFAESGQVYFGIIALGGGGSSSRTP
SDKPVAHVVANPQAEGQLQWLNRRANALLANGVELRDNQLVVPSEGLYLI
YSQVLFKGQGCPSTHVLLTHTISRIAVSFQTKVNLLSAIKSPCQRETPEG
AEAKPVVYEPIYLGGVFQLEKGDRLSAEINRPDYLDFAESGQVYFGIIAL
GGGGSSSRTPSDKPVAHVVANPQAEGQLQWLNRRANALLANGVELRDNQL
VVPSEGLYLIYSQVLFKGQGCPSTHVLLTHTISRIAVSFQTKVNLLSAIK
SPCQRETPEGAEAKPWYEPIYLGGVFQLEKGDRLSAEINRPDYLDFAESG
QVYFGIIAL

### Production and Purification of Human CD20 VHH-Based AFRs

Constructs were combined transiently expressed in the ExpiCHO expression system as follows:
(i) CD20 VHH-20*GGS-Fc3+Fc4-20*GGS-3*TNF_Y87F (AFR; see scheme 7B)

One week after transfection, supernatant was collected and cells removed by centrifugation. Recombinant proteins were purified from the supernatant using the Pierce Protein A spin plates (Thermo Fisher).

### Biological Activity on HEK-Dual Reporter Cell-Lines

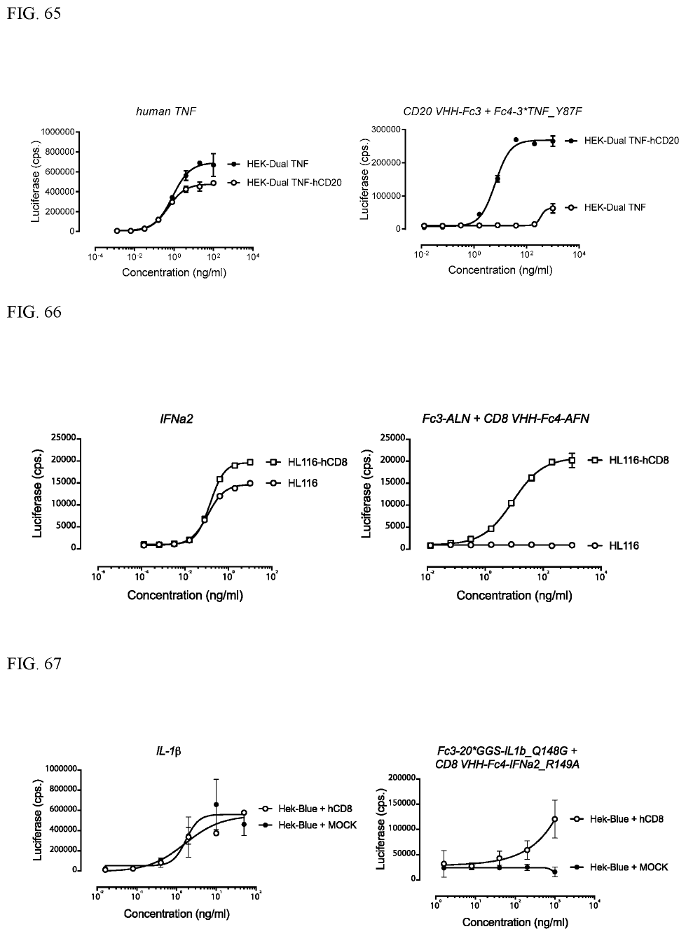

HEK-Dual TNF-α cells (InvivoGen) were derived from the human embryonic kidney 293 cell line by stable co-transfection of two NF-κB-inducible reporter constructs. This allows the TNF-α-induced NF-κB activation by monitoring the activity of either secreted alkaline phosphatase (SEAP) or a secreted luciferase (Lucia). Parental cells were stably transfected with an expression vector encoding the human CD20 sequence. Stable transfected clones were selected in puromycin-containing medium. Parental HEK-Dual and HEK-Dual-hCD20 cells were seeded at 20,000 cells per 96-well and subsequently stimulated overnight with a serial dilution of Fc AFRs. Secreted Lucia luciferase activity was measured using QUANTI-Luc (InvivoGen). Data in the FIG. 65 illustrate that both cell-lines are comparably sensitive to wild type TNF, while the CD20 Fc AFR is more active on targeted cells (HEK-Dual-hCD20) compared to untargeted cells (HEK-Dual).

### Example 41: Bi-AcTakines

In this Example, we generated and evaluated a bi-AcTakine containing both IFNa2_R149A and hIL1b_Q148Q mutants and targeted via a human CD8 specific VHH (clone 1CDA65).

### Constructs:

Fc3-20*GGS-IL1b_Q 148G (P-1627)
(SEQ ID NO: 1555)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSAPVRSLNCTLRDS
QQKSLVMSGPYELKALHLGGQDMEQQVVFSMSFVQGEESNDKIPVALGLK
EKNLYLSCVLKDDKPTLQLESVDPKNYPKKKMEKRFVFNKIEINNKLEFE
SAQFPNWYISTSQAENMPVFLGGTKGGQDITDFTMQFVSS
CD8 VHH-20*GGS-Fc4-20*GGS-IFNa2_R149A (P-1628)
(SEQ ID NO: 1556)
QVQLQESGGGLVQPGGSLRLSCAASGSIFSINVMGWYRQTPGKERELVAK
ITNFGITSYADSAQGRFTISRGNAKNTVYLQMNSLKPEDTAVYYCNLDTT
GWGPPPYQYWGQGTQVTVSSGGSGGSGGSGGSGGSGGSGGSGGSGGSGGS
GGSGGSGGSGGSGGSGGSGGSGGSGGSGGSDKTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCQVSNKALPAPIEKTISKAK
GQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKS
LSLSPGKGGSGGSGGSGGSGGSGGSGGSGGSGGSGGSGGSGGSGGSGGSG
GSGGSGGSGGSGGSGGSCDLPQTHSLGSRRTLMLLAQMRKISLFSCLKDR
HDFGFPQEEFGNQFQKAETIPVLHEMIQQIFNLFSTKDSSAAWDETLLDK
FYTELYQQLNDLEACVIQGVGVEETPLMKEDSILAVRKYFQRITLYLKEK
KYSPCAWEVVRAEIMASFSLSTNLQESLRSKE

### Production and Purification of the Bi-AcTakine

Constructs Fc3-20*GGS-IL1b_Q148G and CD8 VHH-20*GGS-Fc4-20*GGS-IFNa2_R149A were combined and transiently expressed in the ExpiCHO expression system (Thermo Fisher) according to the manufacturer's guidelines. The resulting bi-AcTakine has a structure as outlined in FIG. 6A. One week after transfection, supernatant was collected, and cells removed by centrifugation. Recombinant proteins were purified from the supernatant using the Pierce Protein A spin plates (Thermo Fisher).

### Biological Activity

The HL116 reporter cell-line was used to test the bi-AcTakine for IFN-like signaling. Parental HL116 cells were transfected with an expression vector encoding the human CD8 sequence. Stable transfected clones were selected in G418-containing medium. Parental HL116 and HL116-hCD8 cells were seeded overnight at 20,000 cells per 96-well and subsequently stimulated with a serial dilution of Fc AFNs for 6 hours. Luciferase activity was measured in cell lysates. Data in the FIG. 66 illustrate that both cell-lines are comparably sensitive to wild type IFNα2, while the bi-AcTakine is much more active on targeted cells (HL116-hCD8) compared to untargeted cells (parental H L116).

IL-1β-like signaling of the bi-AcTakine was measured on HEK-BIue™ IL-13 Cells (InvivoGen) with an NF-κB/AP-1-inducible SEAP (secreted alkaline phosphatase) reporter. Therefore, cells were transiently transfected with an empty vector or an expression-plasmid encoding human CD8. 36 hours post transfection, cells were resuspended and stimulated overnight with a serial dilution wild type IL-13 or bi-AcTakine. SEAP was measured using the Phospha-Light SEAP Reporter Gene Assay System (ThermoFisher) according to the manufacturer's guidelines. Data in FIG. 67 illustrate that MOCK or CD8 transfected HEK-Blue cells are comparably sensitive to wild type IL-1β, while the bi-AcTakine is specifically capable to induce signaling in target (here CD8) expressing cells and not in MOCK transfected cells.

### Example 42: Clec9A Fc AFNs Based on Human IgG4

In this Example, we designed and evaluated Clec9A-targeted (via VHH R1CHCL50) AFNs based on human IgG1 or human IgG4. The human IgG4 used has a S228P mutation to avoid in vivo exchange of half-molecules. In addition, either a knob or hole was engineered in the respective Fc-chains.

### Constructs:

R1CHCL50-20*GGS-Fc3 (P-1451)
(SEQ ID NO: 1557)
QVQLVESGGGLVHPGGSLRLSCAASGSFSSINVMGWYRQAPGKERELVAR
ITNLGLPNYADSVTGRFTISRDNAKNTVYLQMNSLKPEDTAVYYCYLVAL
KAEYWGQGTQVTVSSGGSGGSGGSGGSGGSGGSGGSGGSGGSGGSGGSGG
SGGSGGSGGSGGSGGSGGSGGSGGSDKTHTCPPCPAPEAAGGPSVFLFPP
KPKDTLMISRTPEVTCVVVDVSHEDPEVKFNWYVDGVEVHNAKTKPREEQ
YNSTYRVVSVLTVLHQDWLNGKEYKCQVSNKALPAPIEKTISKAKGQPRE
PQVCTLPPSRDELTKNQVSLSCAVKGFYPSDIAVEWESNGQPENNYKTTP
PVLDSDGSFFLVSKLTVDKSRWQQGNVFSCSVMHEALHNHYTQKSLSLSP
GK
Fc4-20*GGS-IFNa2_R149A (P-1414)
(SEQ ID NO: 1558)
DKTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEYK
CQVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNHYTQKSLSLSPGKGGSGGSGGSGGSGGSGGSGGSGG
SGGSGGSGGSGGSGGSGGSGGSGGSGGSGGSGGSGGSCDLPQTHSLGSRR
TLMLLAQMRKISLFSCLKDRHDFGFPQEEFGNQFQKAETIPVLHEMIQQI
FNLFSTKDSSAAWDETLLDKFYTELYQQLNDLEACVIQGVGVEETPLMKE
DSILAVRKYFQRITLYLKEKKYSPCAWEVVRAEIMASFSLSTNLQESLRS
KE
R1CHCL50-20*GGS-hIgG4 Fc (P-1754) (hole)
(SEQ ID NO: 1559)
SQVQLVESGGGLVHPGGSLRLSCAASGSFSSINVMGWYRQAPGKERELVA
RITNLGLPNYADSVTGRFTISRDNAKNTVYLQMNSLKPEDTAVYYCYLVA
LKAEYWGQGTQVTVSSGGSGGSGGSGGSGGSGGSGGSGGSGGSGGSGGSG
GSGGSGGSGGSGGSGGSGGSGGSGGSDKTHTCPPCPAPEFLGGPSVFLFP
PKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPR
EPQVYTLPPSQEEMTKNQVSLSCAVKGFYPSDIAVEWESNGQPENNYKTT
PPVLDSDGSFFLVSRLTVDKSRWQEGNVFSCSVMHEALHNHYTQKSLSLS
LGK
hIgG4 Fc-20*GGS-IFNa2_R149A (P-1755) (knob)
(SEQ ID NO: 1560)
DKTHTCPPCPAPEFLGGPSVFLFPPKPKDTLMISRTPEVTCVVVDVSQED
PEVQFNWYVDGVEVHNAKTKPREEQFNSTYRWSVLTVLHQDWLNGKEYKC
KVSNKGLPSSIEKTISKAKGQPREPQVYTLPPSQEEMTKNQVSLWCLVKG
FYPSDIAVEWESNGQPENNYKTTPPVLDSDGSFFLYSRLTVDKSRWQEGN
VFSCSVMHEALHNHYTQKSLSLSLGKGGSGGSGGSGGSGGSGGSGGSGGS
GGSGGSGGSGGSGGSGGSGGSGGSGGSGGSGGSGGSCDLPQTHSLGSRRT
LMLLAQMRKISLFSCLKDRHDFGFPQEEFGNQFQKAETIPVLHEMIQQIF
NLFSTKDSSAAWDETLLDKFYTELYQQLNDLEACVIQGVGVEETPLMKED
SILAVRKYFQRITLYLKEKKYSPCAWEVVRAEIMASFSLSTNLQESLRSK
E

### Production and Purification of Clec9A and PD-L1 VHH-Based AFN

The following combinations were transiently transfected in ExpiCHO cells:
(i) R1CHCL50-Fc3+Fc4-IFNa2_R149A (IgG1 based AFN)
(ii) R1CHCL50-hIgG4 Fc+hIgG4 Fc-IFNa2_R149A (IgG4 based AFN)

One week after transfection, supernatant was collected and cells removed by centrifugation. The resulting AcTakines have a structure as outlined in FIG. 7B. Recombinant proteins were purified from the supernatant using the Pierce Protein A spin plates (Thermo Fisher).

### Biological Activity on HL116 Reporter Cell-Lines

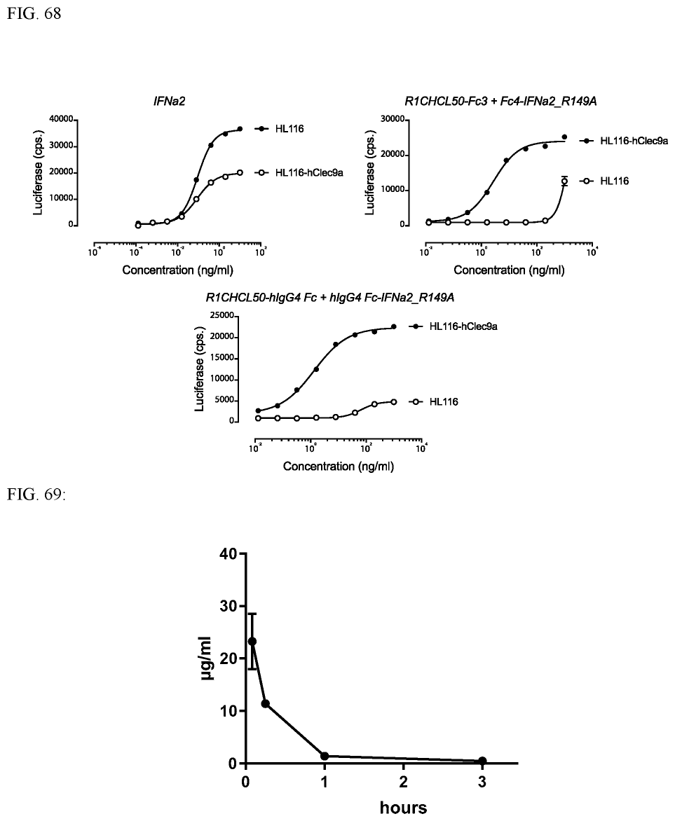

The HL116 clone is derived from the human HT1080 cell line (ATCC CCL-121). It contains the firefly luciferase gene controlled by the IFN-inducible 6-16 promoter. Clec9A targeting was tested using the stable HL116-hClec9A cell-line. Parental and derived cells were seeded overnight at 20,000 cells per 96-well and subsequently stimulated with a serial dilution of Fc AFNs for 6 hours. Luciferase activity was measured in cell lysates. Data in FIG. 68 show that both HL116 and HL116-hClec9A respond similarly to wild type IFNa2. Furthermore, Clec9A targeted Fc AFNs based on human IgG1 or IgG4 have comparable EC50 values on targeted HL116-hClec9A, while signaling on parental cells could only be observed at very high concentrations.

### Example 43: Comparative PK Between Chimera Lacking an Fc and the Present PC-Based Chimeric Protein Complexes

This Example is related to the PK (pharmacokinetics) of an AFN without Fc (3LEC89-20*GGS-huIFNa2_R149A-his6) in mice. This chimera has the sequence of:

P-602 sequence
(SEQ ID NO: 1561)
QVQLQESGGGLVQPGGSLRLSCAASGRIFSVNAMGWYRQAPGKQRELVAA
ITNQGAPTYADSVKGRFTISRDNAGNTVYLQMNSLRPEDTAVYYCKAFTR
GDDYWGQGTQVTVSSVDGGSGGSGGSGGSGGSGGSRSGGSGGSGGSGGSG
GSGGSGGSGGSGGSGGSGGSGGSGGSAAAMCDLPQTHSLGSRRTLMLLAQ
MRRISLFSCLKDRHDFGFPQEEFGNQFQKAETIPVLHEMIQQIFNLFSTK
DSSAAWDETLLDKFYTELYQQLNDLEACVIQGVGVTETPLMKEDSILAVR
KYFQRITLYLKEKKYSPCAWEVVRAEIMASFSLSTNLQESLRSKELEHHH
HHH

Nine animals were dosed intravenously at 3 mg/kg. K-EDTA blood was taken from a first group of 3 mice at 5 minutes and 1 hour, from a second group of 3 mice at 15 minutes and 3 hours and finally from the last group at 8 hours. The concentration in the plasma samples was measured using the same ELISA as described for the Fc-fusion proteins. The measured concentration (FIG. 69) show a fast clearing of this type of molecules resulting in a concentration below detection limit (0.12 μg/ml) at the 8-hour time point. The estimated terminal half-life is in the range of 2 hours.

A comparable study with the same IFN and anti-Clec9A components—but now in the Fc-format (i.e. replacing the GGS linker with the described Fc) was described in Example 3 (See FIG. 24). The half-life was dramatically increased—to multiple days (FIG. 24).

### Example 44: scFv PD-L1-Based Fc AFNs

In this Example, PD-L1 (programmed death-ligand 1) targeted Fc AFNs based on a human PD-L1 specific scFv for targeting to tumour cells or activated immune cells is generated and evaluated.

Several constructs including scFv directed to PD-L1 as the targeting agent and wild type IFNα2 as the signaling agent are constructed. Some illustrative configurations of these PD-L1 targeted constructs are shown, e.g., in FIGS. 4A-D, where the targeting agent is a scFv directed towards PD-L1 and signaling agent is wild type IFNα2.

Production and Purification of PD-L1 scFv-Based AFN

The constructs are produced in ExpiCHO cells by transient transfection of the corresponding constructs according to the manufacturer's guidelines. One week after transfection, supernatant is collected and cells removed by centrifugation. Recombinant proteins are purified from the supernatant using the Pierce Protein A spin plates (Thermo Fisher).

### Biological Activity on HL116 Reporter Cell-Lines

Biological activity of these constructs are studied in HL116 reporter cell-lines. The HL116 clone is derived from the human HT1080 cell line (ATCC CCL-121). It contains the firefly luciferase gene controlled by the IFN-inducible 6-16 promoter. Parental HL116 cells express PD-L1 endogenously, so targeting is evaluated in the absence or presence of an excess of the corresponding free PD-L1 scFv.

Construction and characterization of IFNα-armed anti-PD-L1 is performed. Flow cytometry is used to show binding of indicated protein in IFNAR1−/−A20 cells and PD-L1−/−A20 cells at the concentration of 80 nM.

Numbers indicate the mean fluorescent intensity (MFI). The bioactivity of scFv PD-L1-based Fc AFNs was measured by an anti-viral infection biological assay. L929 cells were cultured with each protein overnight before being infected with VSV-GFP virus. After another 30 h of culture, the percentage of virus-infected cells was determined by flow cytometry.

Balb/c mice (n=5) are inoculated with 3×10⁶ A20 cells. After tumor is established, mice are treated with 20 μg of control, anti-PD-L1, IFNα-Fc, or scFv PD-L1-based Fc AFNs by i.t. or i.v. (day 11 and 15) injection.

Tumor size is measured twice per week. C57BL/6 mice (n=4-8) are inoculated with 5×10⁵ MC38 cells. Mice are treated i.v. with 25 μg control or scFv PD-L1-based Fc AFNs on days 14 and 18. Mice are injected i.v. with 25 μg of indicated protein. Protein concentrations in tumor tissues or serum at different time points is measured by ELISA.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections

What is claimed is:

1. An Fc-based chimeric protein complex comprising:
(a) a targeting moiety comprising a recognition domain that recognizes and/or binds to a target;
(b) a signaling agent, wherein the signaling agent is:
i) a wild type signaling agent; or
ii) a modified signaling agent that has one or more mutations that confer improved safety relative to the wild type signaling agent; and
(c) an Fc domain, the Fc domain comprising an Fc chain and optionally having one or more mutations that reduce or eliminate one or more effector functions of the Fc domain, promotes Fc chain pairing of the Fc domain, and/or stabilizes a hinge region in the Fc domain.

2. The Fc-based chimeric protein complex of claim 1, further comprising one or more linkers.

3. The Fc-based chimeric protein complex of claim 1 or 2, wherein the Fc domain is selected from IgG, IgA, IgD, IgM, or IgE.

4. The Fc-based chimeric protein complex of claim 3, wherein the IgG is selected from IgG1, IgG2, IgG3, or IgG4.

5. The Fc-based chimeric protein complex of claim 3, wherein the Fc domain is selected from human IgG, IgA, IgD, IgM, or IgE.

6. The Fc-based chimeric protein complex of claim 5, wherein the human IgG is selected from human IgG1, IgG2, IgG3, or IgG4.

7. The Fc-based chimeric protein complex of any one of claims 1-6, wherein the signaling agent is a modified signaling agent and has reduced affinity or activity at the signaling agent's receptor relative to a wild type signaling agent.

8. The Fc-based chimeric protein complex of claim 7, wherein the signaling agent is a modified signaling agent and the targeting moiety restores the modified signaling agent's affinity or activity at the signaling agent's receptor.

9. The Fc-based chimeric protein complex of any one of claims 1-8, wherein the Fc chain pairing is promoted by ionic pairing and/or a knob-in-hole pairing.

10. The Fc-based chimeric protein complex of any one of claims 1-9, wherein the one or more mutations to the Fc domain results in an ionic pairing between the Fc chains in the Fc domain.

11. The Fc-based chimeric protein complex of any one of claims 1-10, wherein the one or more mutations to the Fc domain results in a knob-in-hole pairing of the Fc domain.

12. The Fc-based chimeric protein complex of any one of claims 1-11, wherein the one or more mutations to the Fc domain results in the reduction or elimination of the effector function of the Fc domain.

13. The Fc-based chimeric protein complex of any one of claims 1-12, wherein the targeting moiety comprise a recognition domain that recognizes and/or binds an antigen or receptor on a tumor cell, and/or tumor stroma, and/or ECM, and/or immune cell.

14. The Fc-based chimeric protein complex of claim 13, wherein the immune cell is selected from a T cell, a B cell, a dendritic cell, a macrophage, a neutrophil, and a NK cell.

15. The Fc-based chimeric protein complex of any one of the above claims, wherein the targeting moiety comprises a single-domain antibody, a recombinant heavy-chain-only antibody (VHH), a single-chain antibody (scFv), a shark heavy-chain-only antibody (VNAR), a microprotein, a darpin, an anticalin, an adnectin, an aptamer, a Fv, a Fab, a Fab′, a F(ab′)₂, a peptide mimetic molecule, a natural ligand for a receptor, or a synthetic molecule.

16. The Fc-based chimeric protein complex of any one of the above claims, wherein the targeting moiety comprises a VHH.

17. The Fc-based chimeric protein complex of any one of the above claims, wherein the targeting moiety recognizes and/or binds to its target without substantially neutralizing the target's activity or wherein the targeting moiety recognizes and/or binds to its target and substantially neutralizes the target's activity.

18. The Fc-based chimeric protein complex of any one of the above claims, wherein the targeting moiety directly or indirectly recruits immune cells to tumor cells or to the tumor microenvironment.

19. The Fc-based chimeric protein complex of any one of the above claims, wherein the targeting moiety enhances antigen presentation.

20. The Fc-based chimeric protein complex of any one of the above claims, wherein the targeting moiety enhances tumor antigen presentation, optionally by dendritic cells.

21. The Fc-based chimeric protein complex of any one of the above claims, wherein the targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

22. The Fc-based chimeric protein complex of any one of the above claims, wherein the signaling agent is a modified signaling agent and the mutations in the modified signaling agent allow for attenuation of activity.

23. The Fc-based chimeric protein complex of claim 22, wherein agonistic or antagonistic activity is attenuated.

24. The Fc-based chimeric protein complex of any one of the above claims, wherein the signaling agent is a modified signaling agent and the modified signaling agent is selected from an interferon, an interleukin, and a tumor necrosis factor.

25. The Fc-based chimeric protein complex of any one of the above claims, wherein the signaling agent is a modified signaling agent and the modified signaling agent is selected from human: IFNα2, IFNα1, IFNβ, IFNγ, consensus interferon, TNF, TNFR, TGF-α, TGF-β, VEGF, EGF, PDGF, FGF, TRAIL, IL-1β, IL-2, IL-3, IL-4, IL-6, IL-10, IL-12, IL-13, IL-15, IL-18, IL-33, IGF-1, or EPO.

26. The Fc-based chimeric protein complex of claim 25, wherein the human IFNα2 comprises one or more mutations selected from R33A, T106X₃, R120E, R144X₁ A145X₂, M148A, R149A, and L153A and with respect to the amino acid sequence of SEQ ID NO: 1 or 2, wherein X₁ is selected from A, S, T, Y, L, and I, wherein X₂ is selected from G, H, Y, K, and D, and wherein X₃ is selected from A and E.

27. The Fc-based chimeric protein complex of claim 25, wherein the human IFNβ comprises one or more mutations selected from W22G, R27G, L32A, L32G, R35A, R35G, V148G, L151G, R152A, and R152G with respect to the amino acid sequence of SEQ ID NO: 3.

28. The Fc-based chimeric protein complex of claim 25, wherein the human IL-1β comprises one or more mutations selected from A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, I172A, V174A, K208E, K209A, K209D, K209A/K210A, K219S, K219Q, E221S, E221K, E221S/N224A, N224S/K225S, E244K, and N245Q with respect to the amino acid sequence of SEQ ID NO: 17.

29. The Fc-based chimeric protein complex of claim 25, wherein the human IL-2 comprises one or more mutations selected from R38A, F42A, Y45A, E62A, N88R, N88I, N88G, D20H, Q126L, Q126F, D109, and C125 with respect to the amino acid sequence of SEQ ID NO: 18.

30. The Fc-based chimeric protein complex of claim 25, wherein the human TNFα comprises one or more mutations selected from R32G, N34G, Q67G, H73G, L75G, L75A, L75S, T77A, S86G, Y870, Y87L, Y87A, Y87F, V91G, V91A, I97A, I97Q, I97S, T105G, P106G, A109Y, P113G, Y115G, Y115A, E127G, N137G, D143N, A145G, A145T, and Y87Q/I97A with respect to the amino acid sequence of SEQ ID NO: 14.

31. The Fc-based chimeric protein complex of any one of claims 1-30, wherein the Fc domain is homodimeric.

32. The Fc-based chimeric protein complex of any one of claims 1, 2, and 7-30, wherein the Fc domain is heterodimeric.

33. The Fc-based chimeric protein complex of claim 25, wherein the signaling agent is a modified IFNα2, optionally having a R149A mutation with respect to the amino acid sequence of SEQ ID NO: 1 or 2.

34. The Fc-based chimeric protein complex of claim 26 or 33, wherein the targeting moiety binds to Clec9A and the signaling agent is modified IFNα2, optionally having one or more of the following mutations: R33A, R144A, R144I, R144L, R144S, R144T, R144Y, A145D, A145G, A145H, A145K, A145Y, M148A, and L153A.

35. The Fc-based chimeric protein complex of claim 33, wherein the targeting moiety binds to PD-L1 and the signaling agent is modified IFNα2.

36. The Fc-based chimeric protein complex of claim 33, wherein the targeting moiety binds to PD-1 and the signaling agent is modified IFNα2.

37. The Fc-based chimeric protein complex of claim 33, wherein:
(i) the targeting moiety binds to Clec4c and the signaling agent is modified IFNα2, or
(ii) the targeting moiety binds to XCR1 and the signaling agent is modified IFNα2.

38. The Fc-based chimeric protein complex of claim 33, wherein the targeting moiety binds to CD20 and the signaling agent is modified IFNα2.

39. The Fc-based chimeric protein complex of claim 33, wherein the targeting moiety binds to CD13 and the signaling agent is modified IFNα2.

40. The Fc-based chimeric protein complex of claim 33, wherein the targeting moiety binds to FAP and the signaling agent is modified IFNα2.

41. The Fc-based chimeric protein complex of claim 33, wherein the targeting moiety binds to CD8 and the signaling agent is modified IFNα2.

42. The Fc-based chimeric protein complex of claim 33, wherein the targeting moiety binds to Flt3 and optionally comprises the extracellular domain of Flt3L, or a functional portion thereof and the signaling agent is modified IFNα2.

43. The Fc-based chimeric protein complex of claim 21, wherein the targeting moiety is an scFv against PD-L1 and the signaling agent is wild type IFNα2.

44. The Fc-based chimeric protein complex of claim 33, wherein the targeting moiety comprises the extracellular domain of PD-L1, or a functional portion thereof, and the signaling agent is modified IFNα2.

45. The Fc-based chimeric protein complex of claim 33, wherein the targeting moiety comprises the extracellular domain of PD-1, or a functional portion thereof, and the signaling agent is modified IFNα2.

46. The Fc-based chimeric protein complex of claim 33, wherein the targeting moiety comprises the NGR peptide and the signaling agent is modified IFNα2.

47. The Fc-based chimeric protein complex of claim 25, wherein the signaling agent is a wildtype IFNβ or a modified IFNβ.

48. The Fc-based chimeric protein complex of claim 47, wherein the targeting moiety binds to Clec9A and the signaling agent is modified IFNβ.

49. The Fc-based chimeric protein complex of claim 25, wherein the signaling agent is a wildtype IL-1β or a modified IL-1β.

50. The Fc-based chimeric protein complex of claim 49, wherein the targeting moiety binds to CD8 and the signaling agent is modified IL-1β.

51. The Fc-based chimeric protein complex of claim 25, wherein the signaling agent is a wildtype TNF or a modified TNF.

52. The Fc-based chimeric protein complex of claim 51, wherein the targeting moiety binds to CD20 and the signaling agent is modified TNF.

53. The Fc-based chimeric protein complex of any one of claims 1-52, wherein the chimeric protein complex further comprises a second targeting moiety.

54. The Fc-based chimeric protein complex of claim 53, wherein the second targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

55. The Fc-based chimeric protein complex of any one of claims 1-54, wherein the chimeric protein complex further comprises a second signaling agent.

56. The Fc-based chimeric protein complex of claim 55, wherein the second signaling agent is a wild type or modified signaling agent.

57. The Fc-based chimeric protein complex of claim 56, wherein the second signaling agent is selected from human: IFNα2, IFNα1, IFNβ, IFNγ, consensus interferon, TNF, TNFR, TGF-α, TGF-β, VEGF, EGF, PDGF, FGF, TRAIL, IL-1β, IL-2, IL-3, IL-4, IL-6, IL-10, IL-12, IL-13, IL-15, IL-18, IL-33, IGF-1, or EPO.

58. A method for treating or preventing cancer, comprising administering to a patient in need thereof an effective amount of the Fc-based chimeric protein complex of any one of claims 1-57.

59. A use of the Fc-based chimeric protein complex of any one of claims 1-57 for treating or preventing cancer.

60. A use of the Fc-based chimeric protein complex of any one of claims 1-57 for the preparation of a medicament for the treatment of prevention of cancer.

61. The method of claim 58 or the use of claim 59 or claim 60, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

62. A method for treating or preventing an autoimmune disease, neurodegenerative disease, metabolic disease, and/or cardiovascular disease, comprising administering to a patient in need thereof an effective amount of effective amount of the Fc-based chimeric protein complex of any one of claims 1-57.

63. A use of the Fc-based chimeric protein complex of any one of claims 1-57 for treating or preventing an autoimmune disease, neurodegenerative disease, metabolic disease, and/or cardiovascular disease.

64. A use of the Fc-based chimeric protein complex of any one of claims 1-57 for the preparation of a medicament for the treatment of prevention of an autoimmune disease, neurodegenerative disease, metabolic disease, and/or cardiovascular disease.

65. The method of claim 62 or the use of claim 63 or claim 64, wherein the autoimmune disease, neurodegenerative disease, metabolic disease, and/or cardiovascular disease is selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

66. An Fc-based chimeric protein complex where the complex is a homodimer comprising:
(a) a targeting moiety comprising a recognition domain that recognizes and/or binds to a target;
(b) a signaling agent, wherein the signaling agent is:
i) a wild type signaling agent; or
ii) a modified signaling agent that has one or more mutations that confer improved safety relative to the wild type signaling agent; and
(c) an Fc domain, the Fc domain comprising an Fc chain and optionally having one or more mutations that reduce or eliminate one or more effector functions of the Fc domain and/or stabilizes a hinge region in the Fc domain.

67. The Fc-based chimeric protein complex of claim 66, further comprising one or more linkers.

68. The Fc-based chimeric protein complex of any one of claim 66 or 67, wherein the Fc domain is selected from IgG, IgA, IgD, IgM, or IgE.

69. The Fc-based chimeric protein complex of claim 68, wherein the IgG is selected from IgG1, IgG2, IgG3, or IgG4.

70. The Fc-based chimeric protein complex of claim 68, wherein the Fc domain is selected from human IgG, IgA, IgD, IgM, or IgE.

71. The Fc-based chimeric protein complex of claim 70, wherein the human IgG is selected from human IgG1, IgG2, IgG3, or IgG4.

72. The Fc-based chimeric protein complex of any one of claims 66-71, wherein the signaling agent is a modified signaling agent and the modified signaling agent has reduced affinity or activity at the signaling agent's receptor relative to a wild type signaling agent.

73. The Fc-based chimeric protein complex of claim 72, wherein signaling agent is a modified signaling agent and the targeting moiety restores the modified signaling agent's affinity or activity at the signaling agent's receptor.

74. The Fc-based chimeric protein complex of any one of claims 66-73, wherein the one or more mutations to the Fc domain results in the reduction or elimination of the effector function of the Fc domain.

75. The Fc-based chimeric protein complex of any one of claims 66-74, wherein the targeting moiety comprise a recognition domain that recognizes and/or binds an antigen or receptor on a tumor cell, and/or tumor stroma, and/or ECM, and/or immune cell.

76. The Fc-based chimeric protein complex of claim 75, wherein the immune cell is selected from a T cell, a B cell, a dendritic cell, a macrophage, a neutrophil, and a NK cell.

77. The Fc-based chimeric protein complex of any one of claims 66-76, wherein the targeting moiety comprises a single-domain antibody, a recombinant heavy-chain-only antibody (VHH), a single-chain antibody (scFv), a shark heavy-chain-only antibody (VNAR), a microprotein, a darpin, an anticalin, an adnectin, an aptamer, a Fv, a Fab, a Fab′, a F(ab′)₂, a peptide mimetic molecule, a natural ligand for a receptor, or a synthetic molecule.

78. The Fc-based chimeric protein complex of any one of claims 66-77, wherein the targeting moiety comprises a VHH.

79. The Fc-based chimeric protein complex of any one of claims 66-78, wherein the targeting moiety recognizes and/or binds to its target without substantially neutralizing the target's activity or wherein the targeting moiety recognizes and/or binds to its target and substantially neutralizes the target's activity.

80. The Fc-based chimeric protein complex of any one of claims 66-79, wherein the targeting moiety directly or indirectly recruits immune cells to tumor cells or to the tumor microenvironment.

81. The Fc-based chimeric protein complex of any one of claims 66-80, wherein the targeting moiety enhances antigen presentation.

82. The Fc-based chimeric protein complex of any one of claims 66-81, wherein the targeting moiety enhances tumor antigen presentation, optionally by dendritic cells.

83. The Fc-based chimeric protein complex of any one of claims 66-82, wherein the targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

84. The Fc-based chimeric protein complex of any one of claims 66-83, wherein the signaling agent is a modified signaling agent and the mutations in the modified signaling agent allow for attenuation of activity.

85. The Fc-based chimeric protein complex of claim 84, wherein agonistic or antagonistic activity is attenuated.

86. The Fc-based chimeric protein complex of any one of claims 66-85, wherein the modified signaling agent is selected from an interferon, an interleukin, and a tumor necrosis factor.

87. The Fc-based chimeric protein complex of any one of claims 66-86, wherein the modified signaling agent is selected from human: IFNα2, IFNα1, IFNβ, IFNγ, consensus interferon, TNF, TNFR, TGF-α, TGF-β, VEGF, EGF, PDGF, FGF, TRAIL, IL-1β, IL-2, IL-3, IL-4, IL-6, IL-10, IL-12, IL-13, IL-15, IL-18, IL-33, IGF-1, or EPO.

88. The Fc-based chimeric protein complex of claim 87, wherein the human IFNα2 comprises one or more mutations selected from R33A, T106X₃, R120E, R144X₁ A145X₂, M148A, R149A, and L153A and with respect to the amino acid sequence of SEQ ID NO: 1 or 2, wherein X₁ is selected from A, S, T, Y, L, and I, wherein X₂ is selected from G, H, Y, K, and D, and wherein X₃ is selected from A and E.

89. The Fc-based chimeric protein complex of claim 87, wherein the human IFNβ comprises one or more mutations selected from W22G, R27G, L32A, L32G, R35A, R35G, V148G, L151G, R152A, and R152G with respect to the amino acid sequence of SEQ ID NO: 3.

90. The Fc-based chimeric protein complex of claim 87, wherein the human IL-13 comprises one or more mutations selected from A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, I172A, V174A, K208E, K209A, K209D, K209A/K210A, K219S, K219Q, E221S, E221K, E221S/N224A, N224S/K225S, E244K, and N245Q with respect to the amino acid sequence of SEQ ID NO: 17.

91. The Fc-based chimeric protein complex of claim 87, wherein the human IL-2 comprises one or more mutations selected from R38A, F42A, Y45A, E62A, N88R, N88I, N88G, D20H, Q126L, Q126F, D109, and C125 with respect to the amino acid sequence of SEQ ID NO: 18.

92. The Fc-based chimeric protein complex of claim 87, wherein the human TNFα comprises one or more mutations selected from R32G, N34G, Q67G, H73G, L75G, L75A, L75S, T77A, S86G, Y870, Y87L, Y87A, Y87F, V91G, V91A, I97A, I97Q, I97S, T105G, P106G, A109Y, P113G, Y115G, Y115A, E127G, N137G, D143N, A145G, A145T, and Y87Q/I97A with respect to the amino acid sequence of SEQ ID NO: 14.

93. The Fc-based chimeric protein complex of claim 87, wherein the signaling agent is a modified IFNα2, optionally with a R149A mutation with respect to the amino acid sequence of SEQ ID NO: 1 or 2.

94. The Fc-based chimeric protein complex of claim 88 or 93, wherein the targeting moiety binds to Clec9A and the signaling agent is modified IFNα2, optionally having one or more of the following mutations: R33A, R144A, R144I, R144L, R144S, R144T, R144Y, A145D, A145G, A145H, A145K, A145Y, M148A, and L153A.

95. The Fc-based chimeric protein complex of claim 93, wherein the targeting moiety binds to PD-L1 and the signaling agent is modified IFNα2.

96. The Fc-based chimeric protein complex of claim 93, wherein the targeting moiety binds to PD-1 and the signaling agent is modified IFNα2.

97. The Fc-based chimeric protein complex of claim 93, wherein:
(i) the targeting moiety binds to Clec4c and the signaling agent is modified IFNα2, or
(ii) the targeting moiety binds to XCR1 and the signaling agent is modified IFNα2.

98. The Fc-based chimeric protein complex of claim 93, wherein the targeting moiety binds to CD20 and the signaling agent is modified IFNα2.

99. The Fc-based chimeric protein complex of claim 93, wherein the targeting moiety binds to CD13 and the signaling agent is modified IFNα2.

100. The Fc-based chimeric protein complex of claim 93, wherein the targeting moiety binds to FAP and the signaling agent is modified IFNα2.

101. The Fc-based chimeric protein complex of claim 93, wherein the targeting moiety binds to CD8 and the signaling agent is modified IFNα2.

102. The Fc-based chimeric protein complex of claim 93, wherein the targeting moiety binds to Flt3 and optionally comprises the extracellular domain of Flt3L, or a functional portion thereof, and the signaling agent is modified IFNα2.

103. The Fc-based chimeric protein complex of claim 83, wherein the targeting moiety is an scFv against PD-L1 and the signaling agent is wild type IFNα2.

104. The Fc-based chimeric protein complex of claim 93, wherein the targeting moiety comprises the extracellular domain of PD-L1, or a functional portion thereof, and the signaling agent is modified IFNα2.

105. The Fc-based chimeric protein complex of claim 93, wherein the targeting moiety comprises the extracellular domain of PD-1, or a functional portion thereof, and the signaling agent is modified IFNα2.

106. The Fc-based chimeric protein complex of claim 93, wherein the targeting moiety comprises the NGR peptide and the signaling agent is modified IFNα2.

107. The Fc-based chimeric protein complex of claim 87, wherein the signaling agent is a wildtype IFNβ or a modified IFNβ.

108. The Fc-based chimeric protein complex of claim 107, wherein the targeting moiety binds to Clec9A and the signaling agent is modified IFNβ.

109. The Fc-based chimeric protein complex of claim 87, wherein the signaling agent is a wildtype IL-1β or a modified IL-1β.

110. The Fc-based chimeric protein complex of claim 109, wherein the targeting moiety binds to CD8 and the signaling agent is modified IL-1β.

111. The Fc-based chimeric protein complex of claim 87, wherein the signaling agent is a wildtype TNFα or a modified TNFα.

112. The Fc-based chimeric protein complex of claim 111, wherein the targeting moiety binds to CD20 and the signaling agent is modified TNFα.

113. The Fc-based chimeric protein complex of any one of claims 66-112, wherein the chimeric protein complex further comprises a second targeting moiety.

114. The Fc-based chimeric protein complex of claim 113, wherein the second targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

115. The Fc-based chimeric protein complex of any one of claims 66-114, wherein the chimeric protein complex further comprises a second signaling agent.

116. The Fc-based chimeric protein complex of claim 115, wherein the second signaling agent is a wild type or modified signaling agent.

117. The Fc-based chimeric protein complex of claim 116, wherein the second signaling agent is selected from human: IFNα2, IFNα1, IFNβ, IFNγ, consensus interferon, TNFα, TNFR, TGF-α, TGF-β, VEGF, EGF, PDGF, FGF, TRAIL, IL-13, IL-2, IL-3, IL-4, IL-6, IL-10, IL-12, IL-13, IL-15, IL-18, IL-33, IGF-1, or EPO.

118. A method for treating or preventing cancer, comprising administering to a patient in need thereof an effective amount of the Fc-based chimeric protein complex of any one of claims 66-117.

119. A use of the Fc-based chimeric protein complex of any one of claims 66-117 for treating or preventing cancer.

120. A use of the Fc-based chimeric protein complex of any one of claims 66-117 for the preparation of a medicament for the treatment of prevention of cancer.

121. The method of claim 118 or the use of claim 119 or claim 120, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

122. A method for treating or preventing an autoimmune disease, neurodegenerative disease, metabolic disease, and/or cardiovascular disease, comprising administering to a patient in need thereof an effective amount of effective amount of the Fc-based chimeric protein complex of any one of claims 66-117.

123. The use of the Fc-based chimeric protein complex of any one of claims 66-117 for treating or preventing an autoimmune disease, neurodegenerative disease, metabolic disease, and/or cardiovascular disease.

124. The use of the Fc-based chimeric protein complex of any one of claims 66-117 for the preparation of a medicament for the treatment of prevention of an autoimmune disease, neurodegenerative disease, metabolic disease, and/or cardiovascular disease.

125. The method of claim 122 or the use of claim 123 or claim 124, wherein the autoimmune disease, neurodegenerative disease, metabolic disease, and/or cardiovascular disease is selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

126. An Fc-based chimeric protein complex, where the complex is a heterodimer comprising:
(a) a targeting moiety comprising a recognition domain that recognizes and/or binds to a target;
(b) a signaling agent, wherein the signaling agent is:
i) a wild type signaling agent; or
ii) a modified signaling agent that has one or more mutations that confer improved safety relative to the wild type signaling agent; and
(c) an Fc domain, the Fc domain comprising an Fc chain and optionally having one or more mutations promoting Fc chain pairing of the Fc domain and optionally further having one or more mutations that reduce or eliminate one or more effector functions of the Fc domain, and/or stabilizes a hinge region in the Fc domain.

127. The Fc-based chimeric protein complex of claim 126, further comprising one or more linkers.

128. The Fc-based chimeric protein complex of any one of claim 126 or 127, wherein the Fc chains of the Fc domain are selected from IgG, IgA, IgD, IgM, or IgE.

129. The Fc-based chimeric protein complex of claim 128, wherein the IgG is selected from IgG1, IgG2, IgG3, or IgG4.

130. The Fc-based chimeric protein complex of claim 128, wherein the Fc chains of the Fc domain are selected from human IgG, IgA, IgD, IgM, or IgE.

131. The Fc-based chimeric protein complex of claim 130, wherein the human IgG is selected from human IgG1, IgG2, IgG3, or IgG4.

132. The Fc-based chimeric protein complex of any one of claims 126-131, wherein the signaling agent is a modified signaling agent and the modified signaling agent has reduced affinity or activity at the signaling agent's receptor relative to a wild type signaling agent.

133. The Fc-based chimeric protein complex of claim 132, wherein the signaling agent is a modified signaling agent and the targeting moiety restores the modified signaling agent's affinity or activity at the signaling agent's receptor.

134. The Fc-based chimeric protein complex of any one of claims 126-133, wherein the Fc chain pairing is promoted by ionic pairing and/or a knob-in-hole pairing.

135. The Fc-based chimeric protein complex of any one of claims 126-134 wherein the one or more mutations to the Fc domain results in an ionic pairing between the Fc chains in the Fc domain.

136. The Fc-based chimeric protein complex of any one of claims 126-135, wherein the one or more mutations to the Fc domain results in a knob-in-hole pairing of the Fc domain.

137. The Fc-based chimeric protein complex of any one of claims 126-136, wherein the one or more mutations to the Fc domain results in the reduction or elimination of the effector function of the Fc domain.

138. The Fc-based chimeric protein complex of any one of claims 126-137, wherein the targeting moiety comprise a recognition domain that recognizes and/or binds an antigen or receptor on a tumor cell, and/or tumor stroma, and/or ECM, and/or immune cell.

139. The Fc-based chimeric protein complex of claim 138, wherein the immune cell is selected from a T cell, a B cell, a dendritic cell, a macrophage, a neutrophil, and a NK cell.

140. The Fc-based chimeric protein complex of any one of claims 126-139, wherein the targeting moiety comprises a single-domain antibody, a recombinant heavy-chain-only antibody (VHH), a single-chain antibody (scFv), a shark heavy-chain-only antibody (VNAR), a microprotein, a darpin, an anticalin, an adnectin, an aptamer, a Fv, a Fab, a Fab′, a F(ab′)₂, a peptide mimetic molecule, a natural ligand for a receptor, or a synthetic molecule.

141. The Fc-based chimeric protein complex of any one of claims 126-140, wherein the targeting moiety comprises a VHH.

142. The Fc-based chimeric protein complex of any one of claims 126-141, wherein the targeting moiety recognizes and/or binds to its target without substantially neutralizing the target's activity or wherein the targeting moiety recognizes and/or binds to its target and substantially neutralizes the target's activity.

143. The Fc-based chimeric protein complex of any one of claims 126-142, wherein the targeting moiety directly or indirectly recruits immune cells to tumor cells or to the tumor microenvironment.

144. The Fc-based chimeric protein complex of any one of claims 126-143, wherein the targeting moiety enhances antigen presentation.

145. The Fc-based chimeric protein complex of any one of claims 126-144, wherein the targeting moiety enhances tumor antigen presentation, optionally by dendritic cells.

146. The Fc-based chimeric protein complex of any one of claims 126-145, wherein the targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

147. The Fc-based chimeric protein complex of any one of claims 126-146, wherein the signaling agent is a modified signaling agent and the mutations in the modified signaling agent allows for attenuation of activity.

148. The Fc-based chimeric protein complex of claim 147, wherein agonistic or antagonistic activity is attenuated.

149. The Fc-based chimeric protein complex of any one of claims 126-148, wherein the signaling agent is a modified signaling agent and the modified signaling agent is selected from an interferon, an interleukin, and a tumor necrosis factor.

150. The Fc-based chimeric protein complex of any one of claims 126-149, wherein the modified signaling agent is selected from human: IFNα2, IFNα1, IFNβ, IFNγ, consensus interferon, TNFα, TNFR, TGF-α, TGF-β, VEGF, EGF, PDGF, FGF, TRAIL, IL-13, IL-2, IL-3, IL-4, IL-6, IL-10, IL-12, IL-13, IL-15, IL-18, IL-33, IGF-1, or EPO.

151. The Fc-based chimeric protein complex of claim 150, wherein the human IFNα2 comprises one or more mutations selected from R33A, T106X₃, R120E, R144X₁ A145X₂, M148A, R149A, and L153A and with respect to the amino acid sequence of SEQ ID NO: 1 or 2, wherein X₁ is selected from A, S, T, Y, L, and I, wherein X₂ is selected from G, H, Y, K, and D, and wherein X₃ is selected from A and E.

152. The Fc-based chimeric protein complex of claim 150, wherein the human IFNβ comprises one or more mutations selected from W22G, R27G, L32A, L32G, R35A, R35G, V148G, L151G, R152A, and R152G with respect to the amino acid sequence of SEQ ID NO: 3.

153. The Fc-based chimeric protein complex of claim 150, wherein the human IL-1β comprises one or more mutations selected from A117G/P118G, R120G, R120A, L122A, T125G/L126G, R127G, Q130A, Q130W, Q131G, K132A, S137G/Q138Y, L145G, H146A, H146G, H146E, H146N, H146R, L145A/L147A, Q148E, Q148G, Q148L, Q148G/Q150G, Q150G/D151A, M152G, F162A, F162A/Q164E, F166A, Q164E/E167K, N169G/D170G, I172A, V174A, K208E, K209A, K209D, K209A/K210A, K219S, K219Q, E221S, E221K, E221S/N224A, N224S/K225S, E244K, and N245Q with respect to the amino acid sequence of SEQ ID NO: 17.

154. The Fc-based chimeric protein complex of claim 150, wherein the human IL-2 comprises one or more mutations selected from R38A, F42A, Y45A, E62A, N88R, N88I, N88G, D20H, Q126L, Q126F, D109, and C125 with respect to the amino acid sequence of SEQ ID NO: 18.

155. The Fc-based chimeric protein complex of claim 150, wherein the human TNFα comprises one or more mutations selected from R32G, N34G, Q67G, H73G, L75G, L75A, L75S, T77A, S86G, Y870, Y87L, Y87A, Y87F, V91G, V91A, 197A, 197Q, 197S, T105G, P106G, A109Y, P113G, Y115G, Y115A, E127G, N137G, D143N, A145G, A145T, and Y87Q/197A with respect to the amino acid sequence of SEQ ID NO: 14.

156. The Fc-based chimeric protein complex of claim 150, wherein the signaling agent is a modified IFNα2, optionally with a R149A mutation with respect to the amino acid sequence of SEQ ID NO: 1 or 2.

157. The Fc-based chimeric protein complex of claim 151 or 156, wherein the targeting moiety binds to Clec9A and the signaling agent is modified IFNα2, optionally having one or more of the following mutations: R33A, R144A, R144I, R144L, R144S, R144T, R144Y, A145D, A145G, A145H, A145K, A145Y, M148A, and L153A.

158. The Fc-based chimeric protein complex of claim 156, wherein the targeting moiety binds to PD-L1 and the signaling agent is modified IFNα2.

159. The Fc-based chimeric protein complex of claim 156, wherein the targeting moiety binds to PD-1 and the signaling agent is modified IFNα2.

160. The Fc-based chimeric protein complex of claim 156, wherein:
(i) the targeting moiety binds to Clec4c and the signaling agent is modified IFNα2, or
(ii) the targeting moiety binds to XCR1 and the signaling agent is modified IFNα2.

161. The Fc-based chimeric protein complex of claim 156, wherein the targeting moiety binds to CD20 and the signaling agent is modified IFNα2.

162. The Fc-based chimeric protein complex of claim 156, wherein the targeting moiety binds to CD13 and the signaling agent is modified IFNα2.

163. The Fc-based chimeric protein complex of claim 156, wherein the targeting moiety binds to FAP and the signaling agent is modified IFNα2.

164. The Fc-based chimeric protein complex of claim 156, wherein the targeting moiety binds to CD8 and the signaling agent is modified IFNα2.

165. The Fc-based chimeric protein complex of claim 156, wherein the targeting moiety binds to Flt3 and optionally comprises the extracellular domain of Flt3L, or a functional portion thereof, and the signaling agent is modified IFNα2.

166. The Fc-based chimeric protein complex of claim 146, wherein the targeting moiety is an scFv against PD-L1 and the signaling agent is wild type IFNα2.

167. The Fc-based chimeric protein complex of claim 156, wherein the targeting moiety comprises the extracellular domain of PD-L1, or a functional portion thereof, and the signaling agent is modified IFNα2.

168. The Fc-based chimeric protein complex of claim 156, wherein the targeting moiety comprises the extracellular domain of PD-1, or a functional portion thereof, and the signaling agent is modified IFNα2.

169. The Fc-based chimeric protein complex of claim 156, wherein the targeting moiety binds comprises the NGR peptide and the signaling agent is modified IFNα2.

170. The Fc-based chimeric protein complex of claim 150, wherein the signaling agent is a wildtype IFNβ or a modified IFNβ.

171. The Fc-based chimeric protein complex of claim 170, wherein the targeting moiety binds to Clec9A and the signaling agent is modified IFNβ.

172. The Fc-based chimeric protein complex of claim 150, wherein the signaling agent is a wildtype IL-1β or a modified IL-1β.

173. The Fc-based chimeric protein complex of claim 172, wherein the targeting moiety binds to CD8 and the signaling agent is modified IL-1β.

174. The Fc-based chimeric protein complex of claim 150, wherein the signaling agent is a wildtype TNFα or a modified TNFα.

175. The Fc-based chimeric protein complex of claim 174, wherein the targeting moiety binds to CD20 and the signaling agent is modified TNFα.

176. The Fc-based chimeric protein complex of any one of claims 126-175, wherein the chimeric protein complex further comprises a second targeting moiety.

177. The Fc-based chimeric protein complex of claim 176, wherein the second targeting moiety binds to one of the following targets: CD8, CD13, CD20, Clec9A, Clec4c, PD-1, PD-L1, PD-L2, SIRP1α, FAP, XCR1, tenascin CA1, Flt3, or an ECM protein.

178. The Fc-based chimeric protein complex of any one of claims 126-177, wherein the chimeric protein complex further comprises a second signaling agent.

179. The Fc-based chimeric protein complex of claim 178, wherein the second signaling agent is a wild type or modified signaling agent.

180. The Fc-based chimeric protein complex of claim 179, wherein the second signaling agent is selected from human: IFNα2, IFNα1, IFNβ, IFNγ, consensus interferon, TNFα, TNFR, TGF-α, TGF-β, VEGF, EGF, PDGF, FGF, TRAIL, IL-1β, IL-2, IL-3, IL-4, IL-6, IL-10, IL-12, IL-13, IL-15, IL-18, IL-33, IGF-1, or EPO.

181. A method for treating or preventing cancer, comprising administering to a patient in need thereof an effective amount of the Fc-based chimeric protein complex of any one of claims 126-180.

182. A use of the Fc-based chimeric protein complex of any one of claims 126-180 for treating or preventing cancer.

183. A use of the Fc-based chimeric protein complex of any one of claims 126-180 for the preparation of a medicament for the treatment of prevention of cancer.

184. The method of claim 181 or the use of claim 182 or claim 183, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

185. A method for treating or preventing an autoimmune disease, neurodegenerative disease, metabolic disease, and/or cardiovascular disease, comprising administering to a patient in need thereof an effective amount of effective amount of the Fc-based chimeric protein complex of any one of claims 126-180.

186. A use of the Fc-based chimeric protein complex of any one of claims 126-180 for treating or preventing an autoimmune disease, neurodegenerative disease, metabolic disease, and/or cardiovascular disease.

187. A use of the Fc-based chimeric protein complex of any one of claims 126-180 for the preparation of a medicament for the treatment of prevention of an autoimmune disease, neurodegenerative disease, metabolic disease, and/or cardiovascular disease.

188. The method of claim 185 or the use of claim 186 or claim 187, wherein the autoimmune disease, neurodegenerative disease, metabolic disease, and/or cardiovascular disease is selected from multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, myasthenia gravis, Reiter's syndrome, and Grave's disease.

189. A nucleic acid encoding an Fc-based chimeric protein complex of any one of claim 1-57, 66-117, or 126-180, or a constituent thereof.

190. The Fc-based chimeric protein complex of any one claim 1-57, 66-117, or 126-180, wherein the Fc-based chimeric protein complex is a complex of two proteins.

191. The Fc-based chimeric protein complex of claim 190, wherein the complex comprises one or more fusion proteins.

192. The Fc-based chimeric protein complex of any one claim 1-57, 66-117, 126-180, or 190-191, wherein Fc-based chimeric protein complex has a configuration and/or orientation as shown in any one of FIGS. 1A-F, 2A-H, 3A-H, 4A-D, 5A-F, 6A-J, 7A-D, 8A-F, 9A-J, 10A-F, 11A-L, 12A-L, 13A-F, 14A-L, 15A-L, 16A-J, 17A-J, 18A-F, 19A-F, 20A-E, 38, 46A-D, 47, and 49.

193. The Fc-based chimeric protein complex of claim 192, wherein Fc-based chimeric protein complex has a configuration and/or orientation as shown in FIG. 7B.

194. The Fc-based chimeric protein complex of any one claim 1-57 or 126-180, wherein the Fc-based chimeric protein complex has a trans orientation/configuration, as relates to any targeting moiety and signaling agent, relative to each other, and/or any targeting moieties relative to each other, and/or any signaling agents relative to each other.

195. The Fc-based chimeric protein complex of any one claim 1-57 or 126-180, wherein the Fc-based chimeric protein complex has a cis orientation/configuration, as relates to any targeting moiety and signaling agent, relative to each other, and/or any targeting moieties relative to each other, and/or any signaling agents relative to each other.

196. The Fc-based chimeric protein complex of any one claim 1-57, 66-117, 126-180, or 190-195, wherein the Fc comprises L234A, L235A, and one additional mutation selected from K322A, K322Q, D265A, P32G, and P331S substitutions in human IgG1, wherein the numbering is based on the EU convention.

197. The Fc-based chimeric protein complex of any one claim 1-57, 66-117, 126-180, or 190-195, wherein the Fc comprises a S228P substitution in human IgG4, wherein the numbering is based on the EU convention.
